SEQUENCE LISTING (121 sequences)

SEQ ID NO: 1 - DNA, 30 bp, Artificial Sequence
  primer for cloning of his4
gatctcctgatgactgactcactgataata

SEQ ID NO: 2 - DNA, 30 bp, Artificial Sequence
  primer for cloning of his4
taattaaataagtcccagtttctccatacg

SEQ ID NO: 3 - DNA, 25 bp, Artificial Sequence
  primer for cloning of arg4
acgaaaatatggtacctgccctcac

SEQ ID NO: 4 - DNA, 30 bp, Artificial Sequence
  primer for cloning of arg4
gttctatctacccgaggaaaccgatacata

SEQ ID NO: 5 - DNA, 27 bp, Artificial Sequence
  primer for cloning of aox1 promoter
agatctaacatccaaagacgaaaggtt

SEQ ID NO: 6 - DNA, 29 bp, Artificial Sequence
  primer for cloning of aox1 promoter
atccaccacctagaactaggatatcaaac

SEQ ID NO: 7 - DNA, 25 bp, Artificial Sequence
  primer for cloning of per3 promoter
ctaagggtctcactggtgtttcagc

SEQ ID NO: 8 - DNA, 24 bp, Artificial Sequence
  primer for cloning of per3 promoter
agttccttgcaactgtagtggtcg

SEQ ID NO: 9 - DNA, 27 bp, Artificial Sequence
  primer for cloning of per3 promoter with addition of restriction enzyme site
aaccgcggctcgtcactatcgtcgttg

SEQ ID NO: 10 - DNA, 35 bp, Artificial Sequence
  primer for cloning of per3 promoter with addition of restriction enzyme site
cgaacgttacctgaagataggtaaaaaaaaattgc

SEQ ID NO: 11 - DNA, 32 bp, Artificial Sequence
  primer for cloning of aox2 promoter with addition of restriction enzyme site
aaccgcggctagtagaactttgacatctgcta

SEQ ID NO: 12 - DNA, 31 bp, Artificial Sequence
  primer for cloning of aox2 promoter with addition of restriction enzyme site
cgaacgttttgatttgtttgtggggatttag

SEQ ID NO: 13 - DNA, 24 bp, Artificial Sequence
  primer for cloning of fld1 promoter
gcagtgttggctaacgtctattcg

SEQ ID NO: 14 - DNA, 23 bp, Artificial Sequence
  primer for cloning of fld1 promoter
acttcatgggctcttggaggaag

SEQ ID NO: 15 - DNA, 31 bp, Artificial Sequence
  primer for cloning of fld1 promoter with addition of restriction enzyme site
aaccgcggcctgaataccgtaacatagtgac

SEQ ID NO: 16 - DNA, 33 bp, Artificial Sequence
  primer for cloning of fld1 promoter with addition of restriction enzyme site
cgaacgtttcaagaattgtatgaacaagcaaag

SEQ ID NO: 17 - DNA, 26 bp, Artificial Sequence
  primer for cloning of aox1 terminator
ccttagacatgactgttcctcagttc

SEQ ID NO: 18 - DNA, 24 bp, Artificial Sequence
  primer for cloning of aox1 terminator
gcacaaacgaacgtctcacttaat

SEQ ID NO: 19 - DNA, 30 bp, Artificial Sequence
  primer for cloning of aox1 3′ non coding region
tcgagtatctatgattggaagtatgggaat

SEQ ID NO: 20 - DNA, 30 bp, Artificial Sequence
  primer for cloning of aox1 3′ non coding region
gatcttgagataaatttcacgtttaaaatc

SEQ ID NO: 21 - DNA, 32 bp, Artificial Sequence
  primer for cloning of alpha factor
tcaaacaagaagattacaaactatcaatttca

SEQ ID NO: 22 - DNA, 29 bp, Artificial Sequence
  primer for cloning of alpha factor
gtacgagctaaaagtacagtgggaacaaa

SEQ ID NO: 23 - DNA, 27 bp, Artificial Sequence
  primer for cloning of human col1a1
cagccacaaagagtctacatgtctagg

SEQ ID NO: 24 - DNA, 20 bp, Artificial Sequence
  primer for cloning of human col1a1
aggttgggatggagggagtt

SEQ ID NO: 25 - DNA, 37 bp, Artificial Sequence
  primer for cloning of human col1a2
gccaagcttgcatgctcagctttgtggatacgcggac

SEQ ID NO: 26 - DNA, 44 bp, Artificial Sequence
  primer for cloning of human col1a2
cggtacccggggatccttatttgaaacagactgggccaatgtcc

SEQ ID NO: 27 - DNA, 20 bp, Artificial Sequence
  primer for cloning of human col3a1
ggctgagttttatgacgggc

SEQ ID NO: 28 - DNA, 20 bp, Artificial Sequence
  primer for cloning of human col3a1
gacaagattagaacaagagg

SEQ ID NO: 29 - DNA, 35 bp, Artificial Sequence
  primer for cloning of aox1 terminator with addition of restriction enzyme site
tcgactagtttagacatgactgttcctcagttcaa

SEQ ID NO: 30 - DNA, 30 bp, Artificial Sequence
  primer for cloning of aox1 terminator with addition of restriction enzyme site
aactgcaggcacaaacgaacgtctcactta

SEQ ID NO: 31 - DNA, 35 bp, Artificial Sequence
  linker 1 for construction of psn005
tattcgaaacgcatatgtgaccggcagactagtgg

SEQ ID NO: 32 - DNA, 35 bp, Artificial Sequence
  linker 1 for construction of psn005
ccactagtctgccggtcacatatgggtttcgaata

SEQ ID NO: 33 - DNA, 35 bp, Artificial Sequence
  primer for construction of expression vector of alpha pre pro sequence with addition of restriction enzyme site
ggttcgaaacgatgagatttccttcaatttttact

SEQ ID NO: 34 - DNA, 31 bp, Artificial Sequence
  primer for construction of expression vector of alpha pre pro sequence with addition of restriction enzyme site
tcgactagtagcttcagcctctcttttatcc

SEQ ID NO: 35 - DNA, 25 bp, Artificial Sequence
  primer for cloning of p4hb with addition of restriction enzyme site
ttactagtgacgcccccgaggagga

SEQ ID NO: 36 - DNA, 35 bp, Artificial Sequence
  primer for cloning of p4hb with addition of restriction enzyme site
ttactagtttacagttcatctttcacagctttctg

SEQ ID NO: 37 - DNA, 35 bp, Artificial Sequence
  primer for cloning of expression unit with addition of restriction enzyme site
aaccgcggtctaacatccaaagacgaaaggttgaa

SEQ ID NO: 38 - DNA, 35 bp, Artificial Sequence
  primer for cloning of expression unit with addition of restriction enzyme site
aacccggggcacaaacgaacgtctcacttaatctt

SEQ ID NO: 39 - DNA, 35 bp, Artificial Sequence
  linker 3 for construction of psn007
tattcgaaacgacgcgtgtcagctagcactagtgc

SEQ ID NO: 40 - DNA, 35 bp, Artificial Sequence
  linker 3 for construction of psn007
gcactagtgctagctgacacgcgtcgtttcgaata

SEQ ID NO: 41 - DNA, 35 bp, Artificial Sequence
  primer for cloning of p4ha1 with addition of restriction enzyme site
tattcgaaacgatgatctggtatatattaattata

SEQ ID NO: 42 - DNA, 35 bp, Artificial Sequence
  primer for cloning of p4ha1 with addition of restriction enzyme site
ttgctagctcattccaattctgacaacgtacaagg

SEQ ID NO: 43 - DNA, 35 bp, Artificial Sequence
  primer for cloning of expression unit with addition of restriction enzyme site
aacccgggtctaacatccaaagacgaaaggttgaa

SEQ ID NO: 44 - DNA, 30 bp, Artificial Sequence
  primer for cloning of arg4 with addition of restriction enzyme site
aactcgagacgaaaatatggtacctgccct

SEQ ID NO: 45 - DNA, 32 bp, Artificial Sequence
  primer for cloning of arg4 with addition of restriction enzyme site
ccatcgatacagaggtatcatccaatgattcc

SEQ ID NO: 46 - DNA, 32 bp, Artificial Sequence
  primer for cloning of hsp47 gene with addition of restriction enzyme site
tattcgaaacgatgcgctccctcctgcttctc

SEQ ID NO: 47 - DNA, 34 bp, Artificial Sequence
  primer for cloning of hsp47 gene with addition of restriction enzyme site
ttactagttataactcgtctcgcatcttgtcacc

SEQ ID NO: 48 - DNA, 27 bp, Artificial Sequence
  primer for cloning of zeor gene with addition of restriction enzyme site
ttatcgatcccacacaccatagcttca

SEQ ID NO: 49 - DNA, 29 bp, Artificial Sequence
  primer for cloning of zeor gene with addition of restriction enzyme site
tgatcgatagcttgcaaattaaagccttc

SEQ ID NO: 50 - DNA, 32 bp, Artificial Sequence
  primer for cloning of his4 with addition of restriction enzyme site
ggaagcttgatctcctgatgactgactcactg

SEQ ID NO: 51 - DNA, 34 bp, Artificial Sequence
  primer for cloning of his4 with addition of restriction enzyme site
ccctgcagtaattaaataagtcccagtttctcca

SEQ ID NO: 52 - DNA, 34 bp, Artificial Sequence
  primer for cloning of aox1 3′ non coding region with addition of restriction enzyme site
gcatcgattcgagtatctatgattggaagtatgg

SEQ ID NO: 53 - DNA, 35 bp, Artificial Sequence
  primer for cloning of aox1 3′ non coding region with addition of restriction enzyme site
aagggcccgatcttgagataaatttcacgtttaaa

SEQ ID NO: 54 - DNA, 35 bp, Artificial Sequence
  linker 3 for construction of psn006
tattcgaaacgcatatggtaccggcagactagtgg

SEQ ID NO: 55 - DNA, 35 bp, Artificial Sequence
  linker 3 for construction of psn006
ccactagtcgcctaggcgacatatggtttcgaata

SEQ ID NO: 56 - DNA, 35 bp, Artificial Sequence
  primer for cloning of expression unit with addition of restriction enzyme site
aacggccgtctaacatccaaagacgaaaggttgaa

SEQ ID NO: 57 - DNA, 35 bp, Artificial Sequence
  primer for cloning of expression unit with addition of restriction enzyme site
aacggccggcacaaacgaacgtctcacttaatctt

SEQ ID NO: 58 - DNA, 17 bp, Artificial Sequence
  primer for subcloning of human col1a1
cggcttaccagcctcgc

SEQ ID NO: 59 - DNA, 16 bp, Artificial Sequence
  primer for subcloning of human col1a1
ggaccaccagggccgc

SEQ ID NO: 60 - DNA, 19 bp, Artificial Sequence
  primer for subcloning of human col1a2
gggtttacctgggtggccg

SEQ ID NO: 61 - DNA, 17 bp, Artificial Sequence
  primer for subcloning of human col1a2
ggacctcctggcccacc

SEQ ID NO: 62 - DNA, 19 bp, Artificial Sequence
  primer for subcloning of human col1a1
ggtccacagggtccaggag

SEQ ID NO: 63 - DNA, 24 bp, Artificial Sequence
  primer for subcloning of human col1a1
atcagctcctggtgatcccttttc

SEQ ID NO: 64 - DNA, 21 bp, Artificial Sequence
  primer for subcloning of human col1a1
tggtggaccttgaaaaccctg

SEQ ID NO: 65 - DNA, 16 bp, Artificial Sequence
  primer for subcloning of human col1a1
ggaccccagggccccg

SEQ ID NO: 66 - DNA, 22 bp, Artificial Sequence
  primer for subcloning of human col1a1
atcagcaccaggggatcctttc

SEQ ID NO: 67 - DNA, 16 bp, Artificial Sequence
  primer for subcloning of human col1a2
gggccagttggcgcag

SEQ ID NO: 68 - DNA, 24 bp, Artificial Sequence
  primer for subcloning of human col1a2
tgcttctccagatggtcctttctc

SEQ ID NO: 69 - DNA, 17 bp, Artificial Sequence
  primer for subcloning of human col1a2
tgcgggtccttggaatc

SEQ ID NO: 70 - DNA, 17 bp, Artificial Sequence
  primer for subcloning of human col1a2
ggccctgttggtgctgc

SEQ ID NO: 71 - DNA, 21 bp, Artificial Sequence
  primer for subcloning of human col1a2
agcctctccagagggaccctt

SEQ ID NO: 72 - DNA, 35 bp, Artificial Sequence
  primer for cloning of human col3a1 with addition of restriction enzyme site
tattcgaaacgatgatgagctttgtgcaaaagggg

SEQ ID NO: 73 - DNA, 34 bp, Artificial Sequence
  primer for cloning of human col3a1 with addition of restriction enzyme site
ttactagtttataaaaagcaaacagggccaacgt

SEQ ID NO: 74 - protein, 222 aa, Homo sapiens
MPKTMHFLFRFIVFFYLWGLFTAQRQKKEESTEEVKIEVLHRPENCSKTSKKGDLLNAHYDGYLAKDGSKFYCSRTQNEGHPKWFVLGVGQVIKGLDIAMTDMCPGEKRKVVIPPSFAYGKEGYAEGKIPPDATLIFEIELYAVTKGPRSIETFKQIDMDNDRQLSKAEINLYLQREFEKDEKPRDKSYQDAVLEDIFKKNDHDGDGFISPKEYNVYQHDEL

SEQ ID NO: 75 - protein, 201 aa, Homo sapiens
MTLRPSLLPLHLLLLLLLSAAVCRAEAGLETESPVRTLQVETLVEPPEPCAEPAAFGDTLHIHYTGSLVDGRIIDTSLTRDPLVIELGQKQVIPGLEQSLLDMCVGEKRRAIIPSHLAYGKRGFPPSVPADAVVQYDVELIALIRANYWLKLVKGILPLVGMAMVPALLGLIGYHLYRKANRPKVSKKKLKEEKRNKSKKK

SEQ ID NO: 76 - DNA, 669 bp, Artificial Sequence
  synthesized DNA of human FKBP23 ORF having codon optimized for yeast
atgccaaagactatgcactttctatttcgatttatcgtgttcttttatctctggggactattcactgcccagcgtcaaaaaaaggaagagagtaccgaggaagttaagattgaagttctgcacaggcctgaaaattgctccaagacgagcaagaaaggcgatctgcttaatgcccattacgatgggtacttggctaaggatggttcaaaattctactgttctcgtacccaaaacgagggacacccaaagtggtttgtcttgggtgtcggacaggttattaaaggtttggatatcgctatgacagatatgtgtcccggcgaaaagagaaaagttgttataccgccctcttttgcttatggaaaagagggttatgccgaaggtaaaatacctcctgacgctacattgatcttcgagattgaattatatgctgtcactaagggaccaagatcgattgaaactttcaaacaaattgacatggataacgacagacaactttccaaggcagaaattaacctttacctgcaaagagaattcgagaaagacgagaaacctagagacaagtcataccaagacgcagtgttggaagatatctttaagaagaatgaccatgatggtgatggttttatttctccaaaagagtataacgtataccagcatgatgagttataa

SEQ ID NO: 77 - DNA, 669 bp, Homo sapiens
atgccaaaaaccatgcatttcttattcagattcattgttttcttttatctgtggggcctttttactgctcagagacaaaagaaagaggagagcaccgaagaagtgaaaatagaagttttgcatcgtccagaaaactgctctaagacaagcaagaagggagacctactaaatgcccattatgacggctacctggctaaagacggctcgaaattctactgcagccggacacaaaatgaaggccaccccaaatggtttgttcttggtgttgggcaagtcataaaaggcctagacattgctatgacagatatgtgccctggagaaaagcgaaaagtagttatacccccttcatttgcatacggaaaggaaggctatgcagaaggcaagattccaccggatgctacattgatttttgagattgaactttatgctgtgaccaaaggaccacggagcattgagacatttaaacaaatagacatggacaatgacaggcagctctctaaagccgagataaacctctacttgcaaagggaatttgaaaaagatgagaagccacgtgacaagtcatatcaggatgcagttttagaagatatttttaagaagaatgaccatgatggtgatggcttcatttctcccaaggaatacaatgtataccaacacgatgaactatag

SEQ ID NO: 78 - DNA, 606 bp, Artificial Sequence
  synthesized DNA of human FKBP19 ORF having codon optimized for yeast
atgactttgagaccatccttactacctttacatctattgttactcctgctcttgtctgctgctgtgtgccgtgctgaagctgggcttgaaacagagtcaccagtgcgaacccttcaagtcgagaccctggttgaacctccggaaccttgtgccgaaccagccgcttttggtgatacgctccatatccactatactggatctttggtcgatggacgaattatcgacacatcccttactagggaccctttggttattgaattgggtcagaaacaagttattcctggcttggagcaaagtctgttggatatgtgtgtaggtgagaagcgtagagcaataatcccatcacacttggcatacggtaaaagaggtttccccccaagtgttcctgccgacgccgtcgtacagtacgatgtcgagctaattgcattgattagagctaactattggttaaagcttgtgaaaggtattctgccacttgttggcatggctatggttccagccctattaggattgatcggataccatctgtatagaaaagcaaatagacccaaggtttctaagaagaaattgaaagaagagaagaggaacaagagcaagaagaaataa

SEQ ID NO: 79 - DNA, 606 bp, Homo sapiens
atgaccctgcgcccctcactcctcccgctccatctgctgctgctgctgctgctcagtgcggcggtgtgccgggctgaggctgggctcgaaaccgaaagtcccgtccggaccctccaagtggagaccctggtggagcccccagaaccatgtgccgagcccgctgcttttggagacacgcttcacatacactacacgggaagcttggtagatggacgtattattgacacctccctgaccagagaccctctggttatagaacttggccaaaagcaggtgattccaggtctggagcagagtcttctcgacatgtgtgtgggagagaagcgaagggcaatcattccttctcacttggcctatggaaaacggggatttccaccatctgtcccagcggatgcagtggtgcagtatgacgtggagctgattgcactaatccgagccaactactggctaaagctggtgaagggcattttgcctctggtagggatggccatggtgccagccctcctgggcctcattgggtatcacctatacagaaaggccaatagacccaaagtctccaaaaagaagctcaaggaagagaaacgaaacaagagcaaaaagaaataa

SEQ ID NO: 80 - protein, 534 aa, Homo sapiens
MIWYILIIGILLPQSLAHPGFFTSIGQMTDLIHTEKDLVTSLKDYIKAEEDKLEQIKKWAEKLDRLTSTATKDPEGFVGHPVNAFKLMKRLNTEWSELENLVLKDMSDGFISNLTIQRQYFPNDEDQVGAAKALLRLQDTYNLDTDTISKGNLPGVKHKSFLTAEDCFELGKVAYTEADYYHTELWMEQALRQLDEGEISTIDKVSVLDYLSYAVYQQGDLDKALLLTKKLLELDPEHQRANGNLKYFEYIMAKEKDVNKSASDDQSDQKTTPKKKGVAVDYLPERQKYEMLCRGEGIKMTPRRQKKLFCRYHDGNRNPKFILAPAKQEDEWDKPRIIRFHDIISDAEIEIVKDLAKPRLRRATISNPITGDLETVHYRISKSAWLSGYENPVVSRINMRIQDLTGLDVSTAEELQVANYGVGGQYEPHFDFARKDEPDAFKELGTGNRIATWLFYMSDVSAGGATVFPEVGASVWPKKGTAVFWYNLFASGEGDYSTRHAACPVLVGNKWVSNKWLHERGQEFRRPCTLSELE

SEQ ID NO: 81 - protein, 533 aa, Homo sapiens
MKLWVSALLMAWFGVLSCVQAEFFTSIGHMTDLIYAEKELVQSLKEYILVEEAKLSKIKSWANKMEALTSKSAADAEGYLAHPVNAYKLVKRLNTDWPALEDLVLQDSAAGFIANLSVQRQFFPTDEDEIGAAKALMRLQDTYRLDPGTISRGELPGTKYQAMLSVDDCFGMGRSAYNEGDYYHTVLWMEQVLKQLDAGEEATTTKSQVLDYLSYAVFQLGDLHRALELTRRLLSLDPSHERAGGNLRYFEQLLEEEREKTLTNQTEAELATPEGIYERPVDYLPERDVYESLCRGEGVKLTPRRQKRLFCRYHHGNRAPQLLIAPFKEEDEWDSPHIVRYYDVMSDEEIERIKEIAKPKLARATVRDPKTGVLTVASYRVSKSSWLEEDDDPVVARVNRRMQHITGLTVKTAELLQVANYGVGGQYEPHFDFSRRPFDSGLKTEGNRLATFLNYMSDVEAGGATVFPDLGAAIWPKKGTAVFWYNLLRSGEGDYRTRHAACPVLVGCKWVSNKWFHERGQEFLRPCGSTEVD

SEQ ID NO: 82 - protein, 544 aa, Homo sapiens
MGPGARLAALLAVLALGTGDPERAAARGDTFSALTSVARALAPERRLLGLLRRYLRGEEARLRDLTRFYDKVLSLHEDSTTPVANPLLAFTLIKRLQSDWRNVVHSLEASENIRALKDGYEKVEQDLPAFEDLEGAARALMRLQDVYMLNVKGLARGVFQRVTGSAITDLYSPKRLFSLTGDDCFQVGKVAYDMGDYYHAIPWLEEAVSLFRGSYGEWKTEDEASLEDALDHLAFAYFRAGNVSCALSLSREFLLYSPDNKRMARNVLKYERLLAESPNHVVAEAVIQRPNIPHLQTRDTYEGLCQTLGSQPTLYQIPSLYCSYETNSNAYLLLQPIRKEVIHLEPYIALYHDFVSDSEAQKIRELAEPWLQRSVVASGEKQLQVEYRISKSAWLKDTVDPKLVTLNHRIAALTGLDVRPPYAEYLQVVNYGIGGHYEPHFDHATSPSSPLYRMKSGNRVATFMIYLSSVEAGGATAFIYANLSVPVVRNAALFWWNLHRSGEGDSDTLHAGCPVLVGDKWVANKWIHEYGQEFRRPCSSSPED

SEQ ID NO: 83 - protein, 508 aa, Homo sapiens
MLRRALLCLAVAALVRADAPEEEDHVLVLRKSNFAEALAAHKYLLVEFYAPWCGHCKALAPEYAKAAGKLKAEGSEIRLAKVDATEESDLAQQYGVRGYPTIKFFRNGDTASPKEYTAGREADDIVNWLKKRTGPAATTLPDGAAAESLVESSEVAVIGFFKDVESDSAKQFLQAAEAIDDIPFGITSNSDVFSKYQLDKDGVVLFKKFDEGRNNFEGEVTKENLLDFIKHNQLPLVIEFTEQTAPKIFGGEIKTHILLFLPKSVSDYDGKLSNFKTAAESFKGKILFIFIDSDHTDNQRILEFFGLKKEECPAVRLITLEEEMTKYKPESEELTAERITEFCHRFLEGKIKPHLMSQELPEDWDKQPVKVLVGKNFEDVAFDEKKNVFVEFYAPWCGHCKQLAPIWDKLGETYKDHENIVIAKMDSTANEVEAVKVHSFPTLKFFPASADRTVIDYNGERTLDGFKKFLESGGQDGAGDDDDLEDLEEAEEPDMEEDDDQKAVKDEL

SEQ ID NO: 84 - DNA, 1605 bp, Homo sapiens
atgatctggtatatattaattataggaattctgcttccccagtctttggctcatccaggcttttttacttcaattggtcagatgactgatttgatccatactgagaaagatctggtgacttctctgaaagattatattaaggcagaagaggacaagttagaacaaataaaaaaatgggcagagaagttagatcggctaactagtacagcgacaaaagatccagaaggatttgttgggcatccagtaaatgcattcaaattaatgaaacgtctgaatactgagtggagtgagttggagaatctggtccttaaggatatgtcagatggctttatctctaacctaaccattcagagacagtactttcctaatgatgaagatcaggttggggcagccaaagctctgttacgtctccaggatacctacaatttggatacagataccatctcaaagggtaatcttccaggagtgaaacacaaatcttttctaacggctgaggactgctttgagttgggcaaagtggcctatacagaagcagattattaccatacggaactgtggatggaacaagccctaaggcaactggatgaaggcgagatttctaccatagataaagtctctgttctagattatttgagctatgcggtatatcagcagggagacctggataaggcacttttgctcacaaagaagcttcttgaactagatcctgaacatcagagagctaatggtaacttaaaatattttgagtatataatggctaaagaaaaagatgtcaataagtctgcttcagatgaccaatctgatcagaaaactacaccaaagaaaaaaggggttgctgtggattacctgccagagagacagaagtacgaaatgctgtgccgtggggagggtatcaaaatgacccctcggagacagaaaaaactcttttgccgctaccatgatggaaaccgtaatcctaaatttattctggctccagctaaacaggaggatgaatgggacaagcctcgtattattcgcttccatgatattatttctgatgcagaaattgaaatcgtcaaagacctagcaaaaccaaggctgaggcgagccaccatttcaaacccaataacaggagacttggagacggtacattacagaattagcaaaagtgcctggctctctggctatgaaaatcctgtggtgtctcgaattaatatgagaatacaagatctaacaggactagatgtttccacagcagaggaattacaggtagcaaattatggagttggaggacagtatgaaccccattttgactttgcacggaaagatgagccagatgctttcaaagagctggggacaggaaatagaattgctacatggctgttttatatgagtgatgtgtctgcaggaggagccactgtttttcctgaagttggagctagtgtttggcccaaaaaaggaactgctgttttctggtataatctgtttgccagtggagaaggagattatagtacacggcatgcagcctgtccagtgctagttggcaacaaatgggtatccaataaatggctccatgaacgtggacaagaatttcgaagaccttgtacgttgtcagaattggaatga

SEQ ID NO: 85 - DNA, 1602 bp, Homo sapiens
atgaaactctgggtgtctgcattgctgatggcctggtttggtgtcctgagctgtgtgcaggccgaattcttcacctctattgggcacatgactgacctgatttatgcagagaaagagctggtgcagtctctgaaagagtacatccttgtggaggaagccaagctttccaagattaagagctgggccaacaaaatggaagccttgactagcaagtcagctgctgatgctgagggctacctggctcaccctgtgaatgcctacaaactggtgaagcggctaaacacagactggcctgcgctggaggaccttgtcctgcaggactcagctgcaggttttatcgccaacctctctgtgcagcggcagttcttccccactgatgaggacgagataggagctgccaaagccctgatgagacttcaggacacatacaggctggacccaggcacaatttccagaggggaacttccaggaaccaagtaccaggcaatgctgagtgtggatgactgctttgggatgggccgctcggcctacaatgaaggggactattatcatacggtgttgtggatggagcaggtgctaaagcagcttgatgccggggaggaggccaccacaaccaagtcacaggtgctggactacctcagctatgctgtcttccagttgggtgatctgcaccgtgccctggagctcacccgccgcctgctctcccttgacccaagccacgaacgagctggagggaatctgcggtactttgagcagttattggaggaagagagagaaaaaacgttaacaaatcagacagaagctgagctagcaaccccagaaggcatctatgagaggcctgtggactacctgcctgagagggatgtttacgagagcctctgtcgtggggagggtgtcaaactgacaccccgtagacagaagaggcttttctgtaggtaccaccatggcaacagggccccacagctgctcattgcccccttcaaagaggaggacgagtgggacagcccgcacatcgtcaggtactacgatgtcatgtctgatgaggaaatcgagaggatcaaggagatcgcaaaacctaaacttgcacgagccaccgttcgtgatcccaagacaggagtcctcactgtcgccagctaccgggtttccaaaagctcctggctagaggaagatgatgaccctgttgtggcccgagtaaatcgtcggatgcagcatatcacagggttaacagtaaagactgcagaattgttacaggttgcaaattatggagtgggaggacagtatgaaccgcacttcgacttctctaggcgaccttttgacagcggcctcaaaacagaggggaataggttagcgacgtttcttaactacatgagtgatgtagaagctggtggtgccaccgtcttccctgatctgggggctgcaatttggcctaagaagggtacagctgtgttctggtacaacctcttgcggagcggggaaggtgactaccgaacaagacatgctgcctgccctgtgcttgtgggctgcaagtgggtctccaataagtggttccatgaacgaggacaggagttcttgagaccttgtggatcaacagaagttgactga

SEQ ID NO: 86 - DNA, 1635 bp, Homo sapiens
atgggtcctggggcgcggctggcggcgctgctggcggtgctggcgctcgggacaggagacccagaaagggctgcggctcggggcgacacgttctcggcgctgaccagcgtggcgcgcgccctggcgcccgagcgccggctgctggggctgctgaggcggtacctgcgcggggaggaggcgcggctgcgggacctgactagattctacgacaaggtactttctttgcatgaggattcaacaacccctgtggctaaccctctgcttgcatttactctcatcaaacgcctgcagtctgactggaggaatgtggtacatagtctggaggccagtgagaacatccgagctctgaaggatggctatgagaaggtggagcaagaccttccagcctttgaggaccttgagggagcagcaagggccctgatgcggctgcaggacgtgtacatgctcaatgtgaaaggcctggcccgaggtgtctttcagagagtcactggctctgccatcactgacctgtacagccccaaacggctcttttctctcacaggggatgactgcttccaagttggcaaggtggcctatgacatgggggattattaccatgccattccatggctggaggaggctgtcagtctcttccgaggatcttacggagagtggaagacagaggatgaggcaagtctagaagatgccttggatcacttggcctttgcttatttccgggcaggaaatgtttcgtgtgccctcagcctctctcgggagtttcttctctacagcccagataataagaggatggccaggaatgtcttgaaatatgaaaggctcttggcagagagccccaaccacgtggtagctgaggctgtcatccagaggcccaatataccccacctgcagaccagagacacctacgaggggctatgtcagaccctgggttcccagcccactctctaccagatccctagcctctactgttcctatgagaccaattccaacgcctacctgctgctccagcccatccggaaggaggtcatccacctggagccctacattgctctctaccatgacttcgtcagtgactcagaggctcagaaaattagagaacttgcagaaccatggctacagaggtcagtggtggcatcaggggagaagcagttacaagtggagtaccgcatcagcaaaagtgcctggctgaaggacactgttgacccaaaactggtgaccctcaaccaccgcattgctgccctcacaggccttgatgtccggcctccctatgcagagtatctgcaggtggtgaactatggcatcggaggacactatgagcctcactttgaccatgctacgtcaccaagcagccccctctacagaatgaagtcaggaaaccgagttgcaacatttatgatctatctgagctcggtggaagctggaggagccacagccttcatctatgccaacctcagcgtgcctgtggttaggaatgcagcactgttttggtggaacctgcacaggagtggtgaaggggacagtgacacacttcatgctggctgtcctgtcctggtgggagataagtgggtggccaacaagtggatacatgagtatggacaggaattccgcagaccctgcagctccagccctgaagactga

SEQ ID NO: 87 - DNA, 1527 bp, Homo sapiens
atgctgcgccgcgctctgctgtgcctggccgtggccgccctggtgcgcgccgacgcccccgaggaggaggaccacgtcctggtgctgcggaaaagcaacttcgcggaggcgctggcggcccacaagtacctgctggtggagttctatgccccttggtgtggccactgcaaggctctggcccctgagtatgccaaagccgctgggaagctgaaggcagaaggttccgagatcaggttggccaaggtggacgccacggaggagtctgacctggcccagcagtacggcgtgcgcggctatcccaccatcaagttcttcaggaatggagacacggcttcccccaaggaatatacagctggcagagaggctgatgacatcgtgaactggctgaagaagcgcacgggcccggctgccaccaccctgcctgacggcgcagctgcagagtccttggtggagtccagcgaggtggctgtcatcggcttcttcaaggacgtggagtcggactctgccaagcagtttttgcaggcagcagaggccatcgatgacataccatttgggatcacttccaacagtgacgtgttctccaaataccagctcgacaaagatggggttgtcctctttaagaagtttgatgaaggccggaacaactttgaaggggaggtcaccaaggagaacctgctggactttatcaaacacaaccagctgccccttgtcatcgagttcaccgagcagacagccccgaagatttttggaggtgaaatcaagactcacatcctgctgttcttgcccaagagtgtgtctgactatgacggcaaactgagcaacttcaaaacagcagccgagagcttcaagggcaagatcctgttcatcttcatcgacagcgaccacaccgacaaccagcgcatcctcgagttctttggcctgaagaaggaagagtgcccggccgtgcgcctcatcaccctggaggaggagatgaccaagtacaagcccgaatcggaggagctgacggcagagaggatcacagagttctgccaccgcttcctggagggcaaaatcaagccccacctgatgagccaggagctgccggaggactgggacaagcagcctgtcaaggtgcttgttgggaagaactttgaagacgtggcttttgatgagaaaaaaaacgtctttgtggagttctatgccccatggtgtggtcactgcaaacagttggctcccatttgggataaactgggagagacgtacaaggaccatgagaacatcgtcatcgccaagatggactcgactgccaacgaggtggaggccgtcaaagtgcacagcttccccacactcaagttctttcctgccagtgccgacaggacggtcattgattacaacggggaacgcacgctggatggttttaagaaattcctggagagcggtggccaggatggggcaggggatgatgacgatctcgaggacctggaagaagcagaggagccagacatggaggaagacgatgatcagaaagctgtgaaagatgaactgtaa

SEQ ID NO: 88 - protein, 1464 aa, Homo sapiens
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 89 - protein, 1366 aa, Homo sapiens [sequence omitted]

SEQ ID NO: 90 - protein, 1487 aa, Homo sapiens
MIRLGAPQTLVLLTLLVAAVLRCQGQDVQEAGSCVQDGQRYNDKDVWKPEPCRICVCDTGTVLCDDIICEDVKDCLSPEIPFGECCPICPTDLATASGQPGPKGQKGEPGDIKDIVGPKGPPGPQGPAGEQGPRGDRGDKGEKGAPGPRGRDGEPGTPGNPGPPGPPGPPGPPGLGGNFAAQMAGGFDEKAGGAQLGVMQGPMGPMGPRGPPGPAGAPGPQGFQGNPGEPGEPGVSGPMGPRGPPGPPGKPGDDGEAGKPGKAGERGPPGPQGARGFPGTPGLPGVKGHRGYPGLDGAKGEAGAPGVKGESGSPGENGSPGPMGPRGLPGERGRTGPAGAAGARGNDGQPGPAGPPGPVGPAGGPGFPGAPGAKGEAGPTGARGPEGAQGPRGEPGTPGSPGPAGASGNPGTDGIPGAKGSAGAPGIAGAPGFPGPRGPPGPQGATGPLGPKGQTGEPGIAGFKGEQGPKGEPGPAGPQGAPGPAGEEGKRGARGEPGGVGPIGPPGERGAPGNRGFPGQDGLAGPKGAPGERGPSGLAGPKGANGDPGRPGEPGLPGARGLTGRPGDAGPQGKVGPSGAPGEDGRPGPPGPQGARGQPGVMGFPGPKGANGEPGKAGEKGLPGAPGLRGLPGKDGETGAAGPPGPAGPAGERGEQGAPGPSGFQGLPGPPGPPGEGGKPGDQGVPGEAGAPGLVGPRGERGFPGERGSPGAQGLQGPRGLPGTPGTDGPKGASGPAGPPGAQGPPGLQGMPGERGAAGIAGPKGDRGDVGEKGPEGAPGKDGGRGLTGPIGPPGPAGANGEKGEVGPPGPAGSAGARGAPGERGETGPPGPAGFAGPPGADGQPGAKGEQGEAGQKGDAGAPGPQGPSGAPGPQGPTGVTGPKGARGAQGPPGATGFPGAAGRVGPPGSNGNPGPPGPPGPSGKDGPKGARGDSGPPGRAGEPGLQGPAGPPGEKGEPGDDGPSGAEGPPGPQGLAGQRGIVGLPGQRGERGFPGLPGPSGEPGKQGAPGASGDRGPPGPVGPPGLTGPAGEPGREGSPGADGPPGRDGAAGVKGDRGETGAVGAPGAPGPPGSPGPAGPTGKQGDRGEAGAQGPMGPSGPAGARGIQGPQGPRGDKGEAGEPGERGLKGHRGFTGLQGLPGPPGPSGDQGASGPAGPSGPRGPPGPVGPSGKDGANGIPGPIGPPGPRGRSGETGPAGPPGNPGPPGPPGPPGPGIDMSAFAGLGPREKGPDPLQYMRADQAAGGLRQHDAEVDATLKSLNNQIESIRSPEGSRKNPARTCRDLKLCHPEWKSGDYWIDPNQGCTLDAMKVFCNMETGETCVYPNPANVPKKNWWSSKSKEKKHIWFGETINGGFHFSYGDDNLAPNTANVQMTFLRLLSTEGSQNITYHCKNSIAYLDEAAGNLKKALLIQGSNDVEIRAEGNSRFTYTALKDGCTKHTGKWGKTVIEYRSQKTSRLPIIDIAPMDIGGPEQEFGVDIGPVCFL

SEQ ID NO: 91 - protein, 1466 aa, Homo sapiens
MMSFVQKGSWLLLALLHPTIILAQQEAVEGGCSHLGQSYADRDVWKPEPCQICVCDSGSVLCDDIICDDQELDCPNPEIPFGECCAVCPQPPTAPTRPPNGQGPQGPKGDPGPPGIPGRNGDPGIPGQPGSPGSPGPPGICESCPTGPQNYSPQYDSYDVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPGLAGAPGLRGGAGPPGPEGGKGAAGPPGPPGAAGTPGLQGMPGERGGLGSPGPKGDKGEPGGPGADGVPGKDGPRGPTGPIGPPGPAGQPGDKGEGGAPGLPGIAGPRGSPGERGETGPPGPAGFPGAPGQNGEPGGKGERGAPGEKGEGGPPGVAGPPGGSGPAGPPGPQGVKGERGSPGGPGAAGFPGARGLPGPPGSNGNPGPPGPSGSPGKDGPPGPAGNTGAPGSPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGPAGKSGDRGESGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRGERGSEGSPGHPGQPGPPGPPGAPGPCCGGVGAAAIAGIGGEKAGGFAPYYGDEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVQLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

SEQ ID NO: 92 - protein, 525 aa, Homo sapiens
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 93 - protein, 427 aa, Homo sapiens
MLSFVDTRTLLLLAVTLCLATCQSLQEETVRKGPAGDRGPRGERGPPGPPGRDGEDGPTGPPGPPGPPGPPGLGGNFAAQYDGKGVGLGPGPMGLMGPRGPPGAAGAPGPQGFQGPAGEPGEPGQTGPAGARGPAGPPGKAGEDGHPGKPGPPGPPGPPGPPGVSGGGYDFGYDGDFYRADQPRSAPSLRPKDYEVDATLKSLNNQIETLLTPEGSRKNPARTCRDLRLSHPEWSSGYYWIDPNQGCTMDAIKVYCDFSTGETCIRAQPENIPAKNWYRSSKDKKHVWLGETINAGSQFEYNVEGVTSKEMATQLAFMRLLANYASQNITYHCKNSIAYMDEETGNLKKAVILQGSNDVELVAEGNSRFTYTVLVDGCSKKTNEWGKTIIEYKTNKPSRLPFLDIAPLDIGGADQEFFVDIGPVCFK

SEQ ID NO: 94 - protein, 1014 aa, Bos taurus
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 95 - protein, 916 aa, Homo sapiens
MLSFVDTRTLLLLAVTLCLATCQSLQEETVRKGPAGDRGPRGERGPPGPPGRDGEDGPTGPPGPPGPPGPPGLGGNFAAQYDGKGVGLGPGPMGLMGPRGPPGAAGAPGPQGFQGPAGPVGAAGATGARGLVGEPGPAGSKGESGNKGEPGSAGPQGPPGPSGEEGKRGPNGEAGSAGPPGPPGLRGSPGSRGLPGADGRAGVMGPPGSRGASGPAGVRGPNGDAGRPGEPGLMGPRGLPGSPGNIGPAGKEGPVGLPGIDGRPGPIGPAGARGEPGNIGFPGPKGPTGDPGKNGDKGHAGLAGARGAPGPDGNNGAQGPPGPQGVQGGKGEQGPAGPPGFQGLPGPSGPAGEVGKPGERGLHGEFGLPGPAGPRGERGPPGESGAAGPTGPIGSRGPSGPPGPDGNKGEPGVVGAVGTAGPSGPSGLPGERGAAGIPGGKGEKGEPGLRGEIGNPGRDGARGAPGAVGAPGPAGATGDRGEAGAAGPAGPAGPRGSPGERGEVGPAGPNGFAGPAGAAGQPGAKGERGAKGPKGENGVVGPTGPVGAAGPAGPNGPPGPAGSRGDGGPPGMTGFPGAAGRTGPPGPSGISGPPGPPGPAGKEGLRGPRGDQGPVGRTGEVGAVGPPGFAGEKGPSGEAGPPGPPGPPGPPGVSGGGYDFGYDGDFYRADQPRSAPSLRPKDYEVDATLKSLNNQIETLLTPEGSRKNPARTCRDLRLSHPEWSSGYYWIDPNQGCTMDAIKVYCDFSTGETCIRAQPENIPAKNWYRSSKDKKHVWLGETINAGSQFEYNVEGVTSKEMATQLAFMRLLANYASQNITYHCKNSIAYMDEETGNLKKAVILQGSNDVELVAEGNSRFTYTVLVDGCSKKTNEWGKTIIEYKTNKPSRLPFLDIAPLDIGGADQEFFVDIGPVCFK

SEQ ID NO: 96 - protein, 1536 aa, Homo sapiens
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 97 - protein, 1438 aa, Homo sapiens
MLSFVDTRTLLLLAVTLCLATCQSLQEETVRKGPAGDRGPRGERGPPGPPGRDGEDGPTGPPGPPGPPGPPGLGGNFAAQYDGKGVGLGPGPMGLMGPRGPPGAAGAPGPQGFQGPAGPVGAAGATGARGLVGEPGPAGSKGESGNKGEPGSAGPQGPPGPSGEEGKRGPNGEAGSAGPPGPPGLRGSPGSRGLPGADGRAGVMGPPGSRGASGPAGVRGPNGDAGRPGEPGLMGPRGLPGSPGNIGPAGKEGPVGLPGIDGRPGPIGPAGARGEPGNIGFPGPKGPTGDPGKNGDKGHAGLAGARGAPGPDGNNGAQGPPGPQGVQGGKGEQGPAGPPGFQGLPGPSGPAGEVGKPGERGLHGEFGLPGPAGPRGERGPPGESGAAGPTGPIGSRGPSGPPGPDGNKGEPGVVGAVGTAGPSGPSGLPGERGAAGIPGGKGEKGEPGLRGEIGNPGRDGARGAPGAVGAPGPAGATGDRGEAGAAGPAGPAGPRGSPGERGEVGPAGPNGFAGPAGAAGQPGAKGERGAKGPKGENGVVGPTGPVGAAGPAGPNGPPGPAGSRGDGGPPGMTGFPGAAGRTGPPGPSGISGPPGPPGPAGKEGLRGPRGDQGPVGRTGEVGAVGPPGFAGEKGPSGEAGPVGAAGATGARGLVGEPGPAGSKGESGNKGEPGSAGPQGPPGPSGEEGKRGPNGEAGSAGPPGPPGLRGSPGSRGLPGADGRAGVMGPPGSRGASGPAGVRGPNGDAGRPGEPGLMGPRGLPGSPGNIGPAGKEGPVGLPGIDGRPGPIGPAGARGEPGNIGFPGPKGPTGDPGKNGDKGHAGLAGARGAPGPDGNNGAQGPPGPQGVQGGKGEQGPAGPPGFQGLPGPSGPAGEVGKPGERGLHGEFGLPGPAGPRGERGPPGESGAAGPTGPIGSRGPSGPPGPDGNKGEPGVVGAVGTAGPSGPSGLPGERGAAGIPGGKGEKGEPGLRGEIGNPGRDGARGAPGAVGAPGPAGATGDRGEAGAAGPAGPAGPRGSPGERGEVGPAGPNGFAGPAGAAGQPGAKGERGAKGPKGENGVVGPTGPVGAAGPAGPNGPPGPAGSRGDGGPPGMTGFPGAAGRTGPPGPSGISGPPGPPGPAGKEGLRGPRGDQGPVGRTGEVGAVGPPGFAGEKGPSGEAGPPGPPGPPGPPGVSGGGYDFGYDGDFYRADQPRSAPSLRPKDYEVDATLKSLNNQIETLLTPEGSRKNPARTCRDLRLSHPEWSSGYYWIDPNQGCTMDAIKVYCDFSTGETCIRAQPENIPAKNWYRSSKDKKHVWLGETINAGSQFEYNVEGVTSKEMATQLAFMRLLANYASQNITYHCKNSIAYMDEETGNLKKAVILQGSNDVELVAEGNSRFTYTVLVDGCSKKTNEWGKTIIEYKTNKPSRLPFLDIAPLDIGGADQEFFVDIGPVCFK

SEQ ID NO: 98 - DNA, 4395 bp, Artificial Sequence
  synthesized DNA of human collagen ORF having codon optimized for yeast
atgttttctttcgtagatttgcgactcctcttgttgcttgctgctaccgctctccttactcatggacaggaagaaggccaggtggaaggacaggacgaagacattcctcctataacttgtgtgcagaacggactgaggtatcatgacagagatgtgtggaagccagagccttgtcgaatatgcgtttgtgacaacggaaaggtgctatgtgatgatgttatctgtgatgagacaaagaactgtcctggcgctgaagttccagagggagaatgttgtccagtttgtcctgatggatcggagtcacccactgatcaagagactacgggagtagaaggtccaaaaggtgacactggtcccagaggcccaagaggacctgccggcccccctggtagggatggcattcctggtcaacccggcctaccaggccccccaggtccacctggccccccaggaccccctggtttaggaggtaacttcgctcctcaactgtcttacggttacgatgaaaagtctactggtggaatctctgttcctggaccaatgggtccgtctggtccaaggggtctacctggtcctcccggtgcacctggtccgcagggttttcaaggtccaccaggtgaaccgggagaaccgggagcttcaggtccaatgggtcccaggggtcctcctggccctcctggaaagaacggcgatgatggcgaggctggtaagccgggaagacctggtgaaagaggcccacctggaccacagggtgctagaggtcttccaggaaccgcaggattgcctggtatgaagggacaccgtggcttcagtggtttggacggagctaaaggagatgcaggtccggctggtcctaaaggtgagcccggttccccaggtgaaaacggtgctcccggccaaatgggaccacgaggactgccaggcgagagaggtagaccaggcgctccaggtccagctggagctagaggaaatgatggtgccactggtgcagcaggacctcccggaccaactgggcccgctggacctcctggttttccaggagcagttggagcaaagggagaggctggtcctcaagggcctagaggaagcgaaggacctcaaggggttaggggtgaaccaggtcctccaggaccggctggcgctgcaggcccagctggaaatcccggagctgacggtcaaccaggtgctaaaggagctaacggtgctccagggatcgcaggagctcccggttttcctggtgctaggggacccagtggtccacagggtccaggaggtcctcctggacctaaaggaaatagtggagaaccaggagctccagggagtaaaggagacactggtgctaagggagagccaggaccagtgggggttcaaggtcctcccggacccgctggagaagagggaaaaagaggtgcacgtggtgagcctggtcctactggtctgccaggtccacctggggaaaggggtggtcctgggtcaagaggcttccccggtgctgacggagttgctggtccgaaaggaccagccggagagcgaggatccccaggtcctgcaggccctaaggggtcccctggtgaggcaggaagaccgggtgaggctggtttgcccggtgccaaaggtctcacgggctccccaggctctcctggcccagacggcaaaactggcccacctggccctgccggacaagacggaagacctggacctccaggaccacctggtgccagaggtcaggccggtgtgatggggtttccaggtcccaagggtgctgcaggtgaaccagggaaagccggtgaacgaggtgtccccggtcctccaggtgctgttgggccggctggaaaggatggtgaggccggtgcacaagggcctccaggccctgctggtcctgccggtgaaagaggagaacagggaccagccggttctcctggctttcaaggtctgcccggacctgccgggcctccaggagaagctggtaaaccaggagaacaaggtgtcccaggagatttgggagcccctgggccctcaggcgctagaggtgagaggggatttcctggagaacgtggtgtacaaggtcccccgggtccagcaggtccgaggggagctaatggagctccaggaaacgacggtgctaaaggcgacgcaggtgctccaggagcaccaggatcacagggtgccccaggtttgcaaggcatgccaggggaaagaggagcagcaggacttccaggccccaaaggcgacaggggtgacgccggtccaaaaggggccgatggttctcctggcaaagacggcgttcgaggcttgacaggtccgatcggtccacctggacccgccggtgcaccaggtgataagggagaatctggtccttcaggccctgccggtccaaccggggctagaggcgctcctggggatagaggtgaacccggtccaccagggccagccggcttcgctggaccaccaggtgctgatggacagcccggagctaagggtgagccaggtgatgctggtgctaaaggtgacgctggacctccgggtcccgcaggacctgctggtccccctgggccaattggtaatgttggtgccccaggagcaaaaggagccagaggttctgcaggcccaccaggagccacaggattccccggtgctgctggaagagtcggaccaccgggtccatccgggaacgccggacctccaggtcctccgggtcctgctggtaaagaaggaggtaagggtccaagaggtgaaacagggcctgctggccgtcctggtgaagtcggacctcctggcccacccggtccagctggggaaaagggatcaccaggagctgatggtcctgcaggtgctcctggaacccctggtcctcaaggcatcgctggtcaaagaggagtcgtggggttgccagggcaacgaggtgagcgaggatttccaggattaccaggtccatctggagagcccggtaagcagggtccctcgggagccagtggtgagcgtggtccacccggacctatggggcctcctgggctagctgggccaccaggtgagtctggtcgtgagggagcacctggagccgaaggatcaccagggagggatggctcacctggagccaagggtgatagaggcgaaaccggacccgcaggccctcctggtgcacctggagcaccaggagcccccggtccggtaggtccagcaggcaagtcgggtgacagaggagaaactggtccagctggtccagccggcccagttggacctgtgggtgcccgtggtccagcaggaccacaaggtccaagaggggataagggtgaaactggtgagcagggagacagggggattaagggacatagaggcttttcaggtttacaaggtccgcctgggcctccaggcagcccaggggagcaggggccgagtggtgcatccggtccagccggaccacgtgggccacctggttctgctggtgctcctggtaaagacggactaaacggtttgccgggaccaataggcccacccgggccaagagggcgtacaggtgacgccggacctgtcggaccaccagggccgcctggtcctcctggtccacctggtccgcccagtgcaggattcgacttttccttcttaccccaaccgccacaggaaaaggcacacgatggtggtagatattaccgtgctgatgatgccaatgtcgtgagagatagagacttagaagttgatacgacattaaaatcactttctcaacaaattgagaacattagatctccggagggtagcagaaagaacccggccaggacttgccgtgatcttaagatgtgccactccgattggaagtcgggagagtactggatcgatcctaatcaaggttgtaacttggatgcaataaaagtcttctgtaacatggagacaggagaaacttgcgtttacccaactcaaccaagtgttgcacagaaaaattggtatatttccaagaatccaaaggataagcgtcatgtttggtttggagaatctatgaccgatggctttcaattcgagtatggaggtcagggtagcgacccagctgatgtcgctattcaattgacctttttgagacttatgtccacagaagcttcgcaaaacattacatatcactgtaagaatagcgtagcctacatggaccaacagactggtaatctgaaaaaggccctgttgttgcaaggcagtaatgaaatcgaaattcgtgccgaaggaaattccagattcacatactctgttaccgttgacggctgcacctctcataccggtgcctggggtaaaactgtaattgagtataaaacgacaaagacctcccgtcttcctatcattgatgttgctccattagatgtcggtgcccctgaccaggagtttggttttgacgtaggccccgtctgcttcctgtaa

SEQ ID NO: 99 - DNA, 4395 bp, Homo sapiens
atgttcagctttgtggacctccggctcctgctcctcttagcggccaccgccctcctgacgcacggccaagaggaaggccaagtcgagggccaagacgaagacatcccaccaatcacctgcgtacagaacggcctcaggtaccatgaccgagacgtgtggaaacccgagccctgccggatctgcgtctgcgacaacggcaaggtgttgtgcgatgacgtgatctgtgacgagaccaagaactgccccggcgccgaagtccccgagggcgagtgctgtcccgtctgccccgacggctcagagtcacccaccgaccaagaaaccaccggcgtcgagggacccaagggagacactggcccccgaggcccaaggggacccgcaggcccccctggccgagatggcatccctggacagcctggacttcccggaccccccggaccccccggacctcccggaccccctggcctcggaggaaactttgctccccagctgtcttatggctatgatgagaaatcaaccggaggaatttccgtgcctggccccatgggtccctctggtcctcgtggtctccctggcccccctggtgcacctggtccccaaggcttccaaggtccccctggtgagcctggcgagcctggagcttcaggtcccatgggtccccgaggtcccccaggtccccctggaaagaatggagatgatggggaagctggaaaacctggtcgtcctggtgagcgtgggcctcctgggcctcagggtgctcgaggattgcccggaacagctggcctccctggaatgaagggacacagaggtttcagtggtttggatggtgccaagggagatgctggtcctgctggtcctaagggtgagcctggcagccctggtgaaaatggagctcctggtcagatgggcccccgtggcctgcctggtgagagaggtcgccctggagcccctggccctgctggtgctcgtggaaatgatggtgctactggtgctgccgggccccctggtcccaccggccccgctggtcctcctggcttccctggtgctgttggtgctaagggtgaagctggtccccaagggccccgaggctctgaaggtccccagggtgtgcgtggtgagcctggcccccctggccctgctggtgctgctggccctgctggaaaccctggtgctgatggacagcctggtgctaaaggtgccaatggtgctcctggtattgctggtgctcctggcttccctggtgcccgaggcccctctggaccccagggccccggcggccctcctggtcccaagggtaacagcggtgaacctggtgctcctggcagcaaaggagacactggtgctaagggagagcctggccctgttggtgttcaaggaccccctggccctgctggagaggaaggaaagcgaggagctcgaggtgaacccggacccactggcctgcccggaccccctggcgagcgtggtggacctggtagccgtggtttccctggcgcagatggtgttgctggtcccaagggtcccgctggtgaacgtggttctcctggccctgctggccccaaaggatctcctggtgaagctggtcgtcccggtgaagctggtctgcctggtgccaagggtctgactggaagccctggcagccctggtcctgatggcaaaactggcccccctggtcccgccggtcaagatggtcgccccggacccccaggcccacctggtgcccgtggtcaggctggtgtgatgggattccctggacctaaaggtgctgctggagagcccggcaaggctggagagcgaggtgttcccggaccccctggcgctgtcggtcctgctggcaaagatggagaggctggagctcagggaccccctggccctgctggtcccgctggcgagagaggtgaacaaggccctgctggctcccccggattccagggtctccctggtcctgctggtcctccaggtgaagcaggcaaacctggtgaacagggtgttcctggagaccttggcgcccctggcccctctggagcaagaggcgagagaggtttccctggcgagcgtggtgtgcaaggtccccctggtcctgctggtccccgaggggccaacggtgctcccggcaacgatggtgctaagggtgatgctggtgcccctggagctcccggtagccagggcgcccctggccttcagggaatgcctggtgaacgtggtgcagctggtcttccagggcctaagggtgacagaggtgatgctggtcccaaaggtgctgatggctctcctggcaaagatggcgtccgtggtctgaccggccccattggtcctcctggccctgctggtgcccctggtgacaagggtgaaagtggtcccagcggccctgctggtcccactggagctcgtggtgcccccggagaccgtggtgagcctggtccccccggccctgctggctttgctggcccccctggtgctgacggccaacctggtgctaaaggcgaacctggtgatgctggtgctaaaggcgatgctggtccccctggccctgccggacccgctggaccccctggccccattggtaatgttggtgctcctggagccaaaggtgctcgcggcagcgctggtccccctggtgctactggtttccctggtgctgctggccgagtcggtcctcctggcccctctggaaatgctggaccccctggccctcctggtcctgctggcaaagaaggcggcaaaggtccccgtggtgagactggccctgctggacgtcctggtgaagttggtccccctggtccccctggccctgctggcgagaaaggatcccctggtgctgatggtcctgctggtgctcctggtactcccgggcctcaaggtattgctggacagcgtggtgtggtcggcctgcctggtcagagaggagagagaggcttccctggtcttcctggcccctctggtgaacctggcaaacaaggtccctctggagcaagtggtgaacgtggtccccctggtcccatgggcccccctggattggctggaccccctggtgaatctggacgtgagggggctcctggtgccgaaggttcccctggacgagacggttctcctggcgccaagggtgaccgtggtgagaccggccccgctggaccccctggtgctcctggtgctcctggtgcccctggccccgttggccctgctggcaagagtggtgatcgtggtgagactggtcctgctggtcccgccggtcctgtcggccctgttggcgcccgtggccccgccggaccccaaggcccccgtggtgacaagggtgagacaggcgaacagggcgacagaggcataaagggtcaccgtggcttctctggcctccagggtccccctggccctcctggctctcctggtgaacaaggtccctctggagcctctggtcctgctggtccccgaggtccccctggctctgctggtgctcctggcaaagatggactcaacggtctccctggccccattgggccccctggtcctcgcggtcgcactggtgatgctggtcctgttggtccccccggccctcctggacctcctggtccccctggtcctcccagcgctggtttcgacttcagcttcctgccccagccacctcaagagaaggctcacgatggtggccgctactaccgggctgatgatgccaatgtggttcgtgaccgtgacctcgaggtggacaccaccctcaagagcctgagccagcagatcgagaacatccggagcccagagggcagccgcaagaaccccgcccgcacctgccgtgacctcaagatgtgccactctgactggaagagtggagagtactggattgaccccaaccaaggctgcaacctggatgccatcaaagtcttctgcaacatggagactggtgagacctgcgtgtaccccactcagcccagtgtggcccagaagaactggtacatcagcaagaaccccaaggacaagaggcatgtctggttcggcgagagcatgaccgatggattccagttcgagtatggcggccagggctccgaccctgccgatgtggccatccagctgaccttcctgcgcctgatgtccaccgaggcctcccagaacatcacctaccactgcaagaacagcgtggcctacatggaccagcagactggcaacctcaagaaggccctgctcctccagggctccaacgagatcgagatccgcgccgagggcaacagccgcttcacctacagcgtcactgtcgatggctgcacgagtcacaccggagcctggggcaagacagtgattgaatacaaaaccaccaagacctcccgcctgcccatcatcgatgtggcccccttggacgttggtgccccagaccaggaattcggcttcgacgttggccctgtctgcttcctgtaa

SEQ ID NO: 100 - DNA, 4101 bp, Artificial Sequence
  synthesized DNA of human collagen type I alpha 2 precursor ORF having codon optimized for yeast
atgctctctttcgttgatactagaactctattgctgttagcagttactttgtgtttagcaacctgtcaatccttgcaagaagaaaccgttagaaaaggtcctgcaggagatagaggtccaagaggcgagagagggcccccgggacccccagggcgagatggtgaggatggtccgacaggtccaccaggacctcctggacctcccggacctccgggactcggtggtaacttcgctgctcaatatgatggtaaaggcgttggtcttggaccgggaccaatgggtctgatgggtcctagaggaccaccaggggcagcaggggcacctggtccccaaggattccaaggacccgcaggggaacccggggagccaggacagacaggacctgcaggggctagaggacccgccggtccaccaggtaaggctggtgaggacggccacccaggtaaacccggtcgtcccggtgaaaggggagtagttggtccacagggtgcaagaggttttccaggaaccccaggattgcctggatttaagggaatacgtggtcataacggtcttgatggccttaaaggtcaacctggagccccaggtgtaaaaggagaacccggcgctcccggggaaaacggaacaccaggtcagaccggagcccgaggtttgccaggagaacgaggacgagttggtgctcccggtcctgcaggtgctagaggctccgatggatcggttggtccagtgggaccagctggcccaataggtagtgcaggtcctccagggttccctggagctcctggacctaagggagaaataggggctgtgggcaatgcaggacccgctgggcctgctggaccccgtggcgaagttggtttacccggattgagtggtcctgttggtcctccaggtaaccctggcgctaatggtctaactggtgccaagggtgctgcaggacttccaggtgtagccggagctcctgggttgccaggtcctcgtggtatcccagggccagttggcgcagctggtgctacgggagctaggggtctagtgggcgaaccaggtccagcagggtctaagggtgaatccggtaacaagggtgaacctggaagtgccggtcctcagggacctcctggtccatcaggggaggaaggaaagagaggccctaatggcgaggctggtagtgcaggaccaccgggtcctcctggattgagaggttcccccgggtctcgtggactacctggtgccgacggtagagctggcgttatgggtcctcccggaagcagaggagcttctggacccgcaggtgtcaggggtccaaacggtgatgctggaaggccaggtgaaccaggattgatgggtccacgtggattgcccggctctcctggaaacattggacctgccggtaaagaaggccctgtgggtttgccaggtattgatggcagaccaggtccaatcggcccagcaggcgctagaggagagcctggtaacataggattccctggtcctaagggaccaactggtgatccaggcaagaacggtgataagggtcatgccgggctagctggggctagaggtgcacctggcccagatgggaacaacggagcccaagggccaccgggtccacagggggtacaaggtggcaagggcgaacagggtccagctggcccgcctggttttcagggacttccgggtcctagcggtcccgctggagaagttggaaagcctggtgaacgtggactacatggagaattcggactgcctggtcctgctggtccacgtggagaaagaggtcctcccggggagagtggagctgctggtcctaccggaccaattgggtcacgtggaccctccggtcctccaggaccagatgggaataagggtgaaccaggagtcgtaggggcagttggtacagctggcccttccggtccttcgggccttccaggagaaagaggagccgccggaatccctggtggtaaaggcgaaaaaggagaacctggtttaaggggtgagattggaaatccaggtagagatggtgcccgaggagccccaggtgccgtcggtgctcccgggccagccggagccactggcgataggggagaagccggagctgctggaccagccggtccagctggaccaagaggatccccgggtgagagaggagaagttgggccagccggtccgaacggtttcgccggaccagccggtgcagccggtcaaccaggtgctaagggcgagagaggtgctaaaggccctaaaggtgagaatggtgtcgttggccccactgggcctgttggagcagctggacccgctggtccaaacggaccacccggtcctgctggatcgaggggtgacggtggtccaccaggtatgacgggatttccaggtgcagctggtagaactggtccccctggtccgtccgggatttcaggtccacctgggcctccaggacctgctggaaaagagggcttgaggggtccgagaggagatcaaggccctgtaggaagaaccggggaagttggtgccgttggacccccgggttttgctggggagaaaggaccatctggagaagcaggtacagcaggaccacctggtactccaggaccgcagggtctcttaggtgcacccggtatcttgggcctgcccggatctcgtggcgagcgaggattacctggtgtggcaggtgctgtgggtgagcccggaccactgggtatcgctggccctccaggtgcaagaggaccccctggtgccgtcggatctccaggtgtaaatggagctcccggagaggccggtagggacggtaacccggggaacgatggccctcctggaagagatggtcaaccggggcataagggagagagaggctaccctggtaatatcggtcctgtcggtgctgctggtgccccaggaccacatggtccggttggacctgctggtaagcacggtaatcgaggagagactggtccgtcaggtcctgttgggccagcaggtgccgtgggacctaggggtccatcggggccacaaggaatccgaggtgataaaggtgagcctggcgaaaaaggcccgagaggattgccaggactcaagggtcacaacggtttgcagggtttgccaggcattgccggtcatcacggggaccaaggtgctcctggatctgtcggcccagctggtcctcgtggaccagctggaccatctggcccagcaggtaaggatggtagaacaggccacccaggcactgttggacctgcaggtattagaggcccacagggacaccaaggtccagccggacctcctggcccaccaggtcctccgggtccacctggagtttcaggcggaggttacgactttggatatgacggagatttttatagagctgatcaacctcgttcagccccctctttgagaccaaaagactatgaagtggacgctacattgaagagtctcaacaatcagatcgaaaccttgcttactccggaaggttcaagaaaaaatcctgctaggacctgcagagacctgagactttctcatccagagtggtcttccggctattattggattgatcctaaccaaggctgtacaatggatgctattaaggtctactgcgacttttccaccggtgagacatgtataagagctcaacctgaaaacattcctgctaaaaattggtacagatcaagtaaagacaaaaagcatgtgtggttgggtgaaactattaatgccggctctcagtttgaatacaatgtcgagggagtcacttctaaagagatggctacgcaacttgctttcatgcgtttattagccaattacgccagtcaaaatatcacgtaccactgcaagaatagcattgcttacatggacgaggagactggtaaccttaaaaaggccgttattctgcaaggtagcaatgacgtagagctggtcgctgaaggtaactcacgattcacttatactgtcctggttgacggttgttcaaaaaagaccaacgaatggggaaaaacgattatcgagtacaagacaaataagccaagcaggctgccttttttagacattgcaccactagacataggcggggctgatcaggaatttttcgtggacattggtcccgtctgttttaagtaa

SEQ ID NO: 101 - DNA, 4101 bp, Homo sapiens
atgctcagctttgtggatacgcggactttgttgctgcttgcagtaaccttatgcctagcaacatgccaatctttacaagaggaaaccgtaagaaagggcccagccggagatagaggaccacgtggagaaaggggtccaccaggccccccaggcagagatggtgaagatggtcccacaggccctcctggtccacctggtcctcctggcccccctggtctcggtgggaactttgctgctcagtatgacggaaaaggagttggacttggccctggaccaatgggcttaatgggacctagaggcccacctggtgcagctggagccccaggccctcaaggtttccaaggacctgctggtgagcctggtgaacctggtcaaactggtcctgcaggtgctcgtggtccagctggccctcctggcaaggctggtgaagatggtcaccctggaaaacccggacgacctggtgagagaggagttgttggaccacagggtgctcgtggtttccctggaactcctggacttcctggcttcaaaggcattaggggacacaatggtctggatggattgaagggacagcccggtgctcctggtgtgaagggtgaacctggtgcccctggtgaaaatggaactccaggtcaaacaggagcccgtgggcttcctggtgagagaggacgtgttggtgcccctggcccagctggtgcccgtggcagtgatggaagtgtgggtcccgtgggtcctgctggtcccattgggtctgctggccctccaggcttcccaggtgcccctggccccaagggtgaaattggagctgttggtaacgctggtcctgctggtcccgccggtccccgtggtgaagtgggtcttccaggcctctccggccccgttggacctcctggtaatcctggagcaaacggccttactggtgccaagggtgctgctggccttcccggcgttgctggggctcccggcctccctggaccccgcggtattcctggccctgttggtgctgccggtgctactggtgccagaggacttgttggtgagcctggtccagctggctccaaaggagagagcggtaacaagggtgagcccggctctgctgggccccaaggtcctcctggtcccagtggtgaagaaggaaagagaggccctaatggggaagctggatctgccggccctccaggacctcctgggctgagaggtagtcctggttctcgtggtcttcctggagctgatggcagagctggcgtcatgggccctcctggtagtcgtggtgcaagtggccctgctggagtccgaggacctaatggagatgctggtcgccctggggagcctggtctcatgggacccagaggtcttcctggttcccctggaaatatcggccccgctggaaaagaaggtcctgtcggcctccctggcatcgacggcaggcctggcccaattggcccagctggagcaagaggagagcctggcaacattggattccctggacccaaaggccccactggtgatcctggcaaaaacggtgataaaggtcatgctggtcttgctggtgctcggggtgctccaggtcctgatggaaacaatggtgctcagggacctcctggaccacagggtgttcaaggtggaaaaggtgaacagggtcccgctggtcctccaggcttccagggtctgcctggcccctcaggtcccgctggtgaagttggcaaaccaggagaaaggggtctccatggtgagtttggtctccctggtcctgctggtccaagaggggaacgcggtcccccaggtgagagtggtgctgccggtcctactggtcctattggaagccgaggtccttctggacccccagggcctgatggaaacaagggtgaacctggtgttgttggtgctgtgggcactgctggtccatctggtcctagtggactcccaggagagaggggtgctgctggcatacctggaggcaagggagaaaagggtgaacctggtctcagaggtgaaattggtaaccctggcagagatggtgctcgtggtgctcctggtgctgtaggtgcccctggtcctgctggagccacaggtgaccggggcgaagctggggctgctggtcctgctggtcctgctggtcctcggggaagccctggtgaacgtggtgaggtcggtcctgctggccccaatggatttgctggtcctgctggtgctgctggtcaacctggtgctaaaggagaaagaggagccaaagggcctaagggtgaaaacggtgttgttggtcccacaggccccgttggagctgctggcccagctggtccaaatggtccccccggtcctgctggaagtcgtggtgatggaggcccccctggtatgactggtttccctggtgctgctggacggactggtcccccaggaccctctggtatttctggccctcctggtccccctggtcctgctgggaaagaagggcttcgtggtcctcgtggtgaccaaggtccagttggccgaactggagaagtaggtgcagttggtccccctggcttcgctggtgagaagggtccctctggagaggctggtactgctggacctcctggcactccaggtcctcagggtcttcttggtgctcctggtattctgggtctccctggctcgagaggtgaacgtggtctaccaggtgttgctggtgctgtgggtgaacctggtcctcttggcattgccggccctcctggggcccgtggtcctcctggtgctgtgggtagtcctggagtcaacggtgctcctggtgaagctggtcgtgatggcaaccctgggaacgatggtcccccaggtcgcgatggtcaacccggacacaagggagagcgcggttaccctggcaatattggtcccgttggtgctgcaggtgcacctggtcctcatggccccgtgggtcctgctggcaaacatggaaaccgtggtgaaactggtccttctggtcctgttggtcctgctggtgctgttggcccaagaggtcctagtggcccacaaggcattcgtggcgataagggagagcccggtgaaaaggggcccagaggtcttcctggcttaaagggacacaatggattgcaaggtctgcctggtatcgctggtcaccatggtgatcaaggtgctcctggctccgtgggtcctgctggtcctaggggccctgctggtccttctggccctgctggaaaagatggtcgcactggacatcctggtacagttggacctgctggcattcgaggccctcagggtcaccaaggccctgctggcccccctggtccccctggccctcctggacctccaggtgtaagcggtggtggttatgactttggttacgatggagacttctacagggctgaccagcctcgctcagcaccttctctcagacccaaggactatgaagttgatgctactctgaagtctctcaacaaccagattgagacccttcttactcctgaaggctctagaaagaacccagctcgcacatgccgtgacttgagactcagccacccagagtggagcagtggttactactggattgaccctaaccaaggatgcactatggatgctatcaaagtatactgtgatttctctactggcgaaacctgtatccgggcccaacctgaaaacatcccagccaagaactggtataggagctccaaggacaagaaacacgtctggctaggagaaactatcaatgctggcagccagtttgaatataatgtagaaggagtgacttccaaggaaatggctacccaacttgccttcatgcgcctgctggccaactatgcctctcagaacatcacctaccactgcaagaacagcattgcatacatggatgaggagactggcaacctgaaaaaggctgtcattctacagggctctaatgatgttgaacttgttgctgagggcaacagcaggttcacttacactgttcttgtagatggctgctctaaaaagacaaatgaatggggaaagacaatcattgaatacaaaacaaataagccatcacgcctgcccttccttgatattgcacctttggacatcggtggtgctgaccaggaattctttgtggacattggcccagtctgtttcaaataa

SEQ ID NO: 102 - DNA, 4464 bp, Artificial Sequence
  synthesized DNA of human collagen type II alpha 1 precursor ORF having codon optimized for yeast
atgatacgattaggcgccccacagactttggttttacttacattgcttgtggctgccgtactaaggtgtcaaggtcaagatgttcaagaagctgggtcctgtgtgcaagatggtcaaagatacaatgacaaggatgtctggaagcccgagccgtgtaggatctgcgtttgtgataccggcactgtgctgtgtgatgatattatctgcgaagatgtcaaagattgcttgtccccggagatcccttttggtgagtgttgtccgatctgtcctacggatttggcaactgcttcgggacaaccggggcctaaaggacaaaaaggagaaccgggtgatattaaggacatcgtcggtccgaaaggtccccccggtccgcagggtcccgctggtgagcaaggaccgagaggtgacaggggagataagggggaaaagggagctccggggcctagaggacgtgacggcgagccaggtacccctggtaatccgggacctccaggaccgcccggacctccgggtcccccaggtctaggaggcaacttcgctgctcaaatggctggtgggttcgacgagaaagctggaggtgcccagttgggcgttatgcaagggccgatgggtccgatggggcctcgtggacctccagggccggctggcgcacccggacctcagggttttcagggaaatccgggtgaaccaggtgagcccggtgttagcggtccgatgggtccacgtggccctcctggtccccctggtaaaccaggagacgatggagaggcaggaaagccgggaaaggctggtgaacgaggtccaccgggcccacaaggtgcaagaggttttcccggtactccaggtttgccgggagttaagggacaccgaggatacccaggattggacggagcaaagggagaggctggggctcccggtgttaaaggagagtctggttctcctggtgaaaatggttcgccgggcccaatgggaccccgaggattgccgggcgagcgtggacgtactggaccagccggagctgcaggagccagaggaaacgatggtcagcctggaccggctggtcctcccggtcctgttggcccagccggcggtccaggcttcccaggcgctccaggggccaaaggcgaagctggcccaaccggcgcacgtggacctgagggcgctcagggtcccagaggggagcctggtaccccaggtagtcccggaccagctggtgcttcaggtaatcctggtacagatggtattccaggtgccaagggttcagccggagctcctggtatagccggtgcccctggatttccaggtccaaggggtccaccaggaccccaaggggcaacaggacctttgggtccaaagggacaaactggagaacccggtattgctggttttaaaggtgagcaagggcctaagggcgaaccaggaccagctgggccacagggtgctcccggacctgcaggtgaggaaggtaaacgtggagctaggggtgagccaggtggcgtgggaccaattggaccacccggtgagaggggagcaccaggtaacagaggtttcccagggcaggatggactggctggtcccaaaggagcaccaggagaaagaggaccatcaggtcttgccggtcctaagggagctaacggagatcccggtagaccaggtgaaccaggcttgcctggtgccagaggacttactggtagacccggtgatgccggtcctcaaggtaaggtgggcccctctggtgcacccggagaagatggcagaccaggaccaccaggacctcagggtgcaagagggcagccaggagttatgggctttccagggcccaaaggtgctaacggagaacctggtaaagccggtgaaaagggtctgcctggcgcacctggccttcgtgggctacccggcaaggacggagaaacaggtgccgctggacctcccggccctgccggtcccgccggtgagcgtggtgaacaaggagccccaggaccatctgggtttcaaggtctgcctgggcctccaggtccaccaggtgagggtggaaagccaggagatcagggagttccaggtgaggctggagcccctggtttggttgggccaaggggagaacgtggtttccctggcgaaaggggtagtccaggtgcccaaggcttacagggtcctagaggacttccaggtactcctggaactgacggacctaaaggagccagtggtcctgccggacctcctggcgctcaagggccaccagggctccaaggaatgcctggagaaagaggagcagcaggaatcgctggacctaagggtgatagaggtgacgtaggagaaaagggtccagaaggtgcccctggaaaggatggtggtagaggcttaactggtcctattggaccacctgggccagcaggcgcaaacggagagaaaggtgaggttggtcctccaggacccgctggttccgctggggcacgaggtgctccaggggaaagaggtgaaacaggaccccctggtcctgctggctttgcagggccacctggtgctgacggacaacctggagctaaaggcgagcaaggtgaagctggtcaaaagggagatgccggcgctcctggaccacaaggtccatctggtgccccaggtccacagggtccaaccggtgttactggtccaaaaggagctagaggtgcacagggaccacctggtgcaacggggttcccaggtgcagccggtagagttggtccacccggctctaatggtaacccaggccctcctggtcctcctggaccttccggtaaagatggtcccaagggagcccgtggtgattccggtccccctggaagggctggtgagccaggattgcaaggacctgcagggcctcccggagaaaagggtgaacctggtgatgacggaccatctggagctgagggtccacctgggcctcagggattagcaggccaaagaggtatcgttggacttcctggacagagaggcgagaggggtttccctggactcccaggtccatccggggaacctggtaagcagggcgcacctggtgctagcggggacagaggtcctccaggcccagtcggaccacctggactcaccggacctgccggagaaccaggaagagaaggatccccaggtgcagacggacctccaggtcgagacggcgccgccggtgtcaaaggggatcgaggagaaactggagccgtcggagctccaggagcccctggacctcctggatctcctggtccagcaggccctaccggaaagcaaggcgacagaggagaagccggtgctcagggtcctatgggcccttcaggtcctgctggtgctagaggtatccagggtcctcaaggaccaagaggtgataaaggggaagctggggaaccaggtgagagaggcctgaaagggcatagaggatttactggtctgcaaggattgccaggacctcctggcccttcgggggatcagggtgccagcgggccagcaggtccaagcggtccccgaggacccccaggtcctgtcggtccatccggcaaggatggagctaatggaattcccggtcccattgggccccctggcccaagaggcagatctggtgagacaggtcctgcaggtccacccggtaacccaggaccacccggtccacctggtccaccaggcccaggcattgacatgtcagctttcgctggtttgggtccacgagaaaagggacccgacccattgcaatacatgcgtgcagatcaagccgcaggagggctcagacagcacgatgcagaggttgacgcaacactaaaatctctgaacaaccaaatagaatcgattaggtcacctgagggatccagaaagaatccagctagaacttgtcgagacttgaaattatgtcacccagagtggaaatctggagattattggatagatccaaaccaaggctgcaccttggatgctatgaaggtattttgtaatatggagacaggcgaaacttgtgtatacccaaacccagctaatgtgcctaagaaaaattggtggtcatctaagagtaaagaaaagaaacatatctggtttggtgagactattaacggtggcttccatttcagttacggcgacgacaatctagcacccaacactgctaacgtgcaaatgacgtttttacgactgttgagtacagaaggttcccagaatattacctatcactgtaaaaattctatagcctatttagacgaagctgcaggcaatctgaagaaagctttgcttattcaaggttcaaacgatgtagaaattagggcagaaggtaactcaagatttacatacacagccttgaaagacgggtgcactaagcatacgggtaaatggggtaagactgtcatcgagtatagaagtcagaagacctctcgtcttcctattatcgacattgctccaatggatataggcggaccagagcaagaattcggagtagacattggaccagtctgctttctataa

SEQ ID NO: 103 - DNA, 4464 bp, Homo sapiens
atgattcgcctcggggctccccagacgctggtgctgctgacgctgctcgtcgccgctgtccttcggtgtcagggccaggatgtccaggaggctggcagctgtgtgcaggatgggcagaggtataatgataaggatgtgtggaagccggagccctgccggatctgtgtctgtgacactgggactgtcctctgcgacgacataatctgtgaagacgtgaaagactgcctcagccctgagatccccttcggagagtgctgccccatctgcccaactgacctcgccactgccagtgggcaaccaggaccaaagggacagaaaggagaacctggagacatcaaggatattgtaggacccaaaggacctcctgggcctcagggacctgcaggggaacaaggacccagaggggatcgtggtgacaaaggtgaaaaaggtgcccctggacctcgtggcagagatggagaacctgggacccctggaaatcctggcccccctggtcctcccggcccccctggtccccctggtcttggtggaaactttgctgcccagatggctggaggatttgatgaaaaggctggtggcgcccagttgggagtaatgcaaggaccaatgggccccatgggacctcgaggacctccaggccctgcaggtgctcctgggcctcaaggatttcaaggcaatcctggtgaacctggtgaacctggtgtctctggtcccatgggtccccgtggtcctcctggtccccctggaaagcctggtgatgatggtgaagctggaaaacctggaaaagctggtgaaaggggtccgcctggtcctcagggtgctcgtggtttcccaggaaccccaggccttcctggtgtcaaaggtcacagaggttatccaggcctggacggtgctaagggagaggcgggtgctcctggtgtgaagggtgagagtggttccccgggtgagaacggatctccgggcccaatgggtcctcgtggcctgcctggtgaaagaggacggactggccctgctggcgctgcgggtgcccgaggcaacgatggtcagccaggccccgcagggcctccgggtcctgtcggtcctgctggtggtcctggcttccctggtgctcctggagccaagggtgaagccggccccactggtgcccgtggtcctgaaggtgctcaaggtcctcgcggtgaacctggtactcctgggtcccctgggcctgctggtgcctccggtaaccctggaacagatggaattcctggagccaaaggatctgctggtgctcctggcattgctggtgctcctggcttccctgggccacggggccctcctggccctcaaggtgcaactggtcctctgggcccgaaaggtcagacgggtgaacctggtattgctggcttcaaaggtgaacaaggccccaagggagaacctggccctgctggcccccagggagcccctggacccgctggtgaagaaggcaagagaggtgcccgtggagagcctggtggcgttgggcccatcggtccccctggagaaagaggtgctcccggcaaccgcggtttcccaggtcaagatggtctggcaggtcccaagggagcccctggagagcgagggcccagtggtcttgctggccccaagggagccaacggtgaccctggccgtcctggagaacctggccttcctggagcccggggtctcactggccgccctggtgatgctggtcctcaaggcaaagttggcccttctggagcccctggtgaagatggtcgtcctggacctccaggtcctcagggggctcgtgggcagcctggtgtcatgggtttccctggccccaaaggtgccaacggtgagcctggcaaagctggtgagaagggactgcctggtgctcctggtctgaggggtcttcctggcaaagatggtgagacaggtgctgcaggaccccctggccctgctggacctgctggtgaacgaggcgagcagggtgctcctgggccatctgggttccagggacttcctggccctcctggtcccccaggtgaaggtggaaaaccaggtgaccagggtgttcccggtgaagctggagcccctggcctcgtgggtcccaggggtgaacgaggtttcccaggtgaacgtggctctcccggtgcccagggcctccagggtccccgtggcctccccggcactcctggcactgatggtcccaaaggtgcatctggcccagcaggcccccctggggctcagggccctccaggtcttcagggaatgcctggcgagaggggagcagctggtatcgctgggcccaaaggcgacaggggtgacgttggtgagaaaggccctgagggagcccctggaaaggatggtggacgaggcctgacaggtcccattggcccccctggcccagctggtgctaatggcgagaagggagaagttggacctcctggtcctgcaggaagtgctggtgctcgtggcgctccgggtgaacgtggagagactgggccccccggaccagcgggatttgctgggcctcctggtgctgatggccagcctggggccaagggtgagcaaggagaggccggccagaaaggcgatgctggtgcccctggtcctcagggcccctctggagcacctgggcctcagggtcctactggagtgactggtcctaaaggagcccgaggtgcccaaggccccccgggagccactggattccctggagctgctggccgcgttggacccccaggctccaatggcaaccctggaccccctggtccccctggtccttctggaaaagatggtcccaaaggtgctcgaggagacagcggcccccctggccgagctggtgaacccggcctccaaggtcctgctggaccccctggcgagaagggagagcctggagatgacggtccctctggtgccgaaggtccaccaggtccccagggtctggctggtcagagaggcatcgtcggtctgcctgggcaacgtggtgagagaggattccctggcttgcctggcccgtcgggtgagcccggcaagcagggtgctcctggagcatctggagacagaggtcctcctggccccgtgggtcctcctggcctgacgggtcctgcaggtgaacctggacgagagggaagccccggtgctgatggcccccctggcagagatggcgctgctggagtcaagggtgatcgtggtgagactggtgctgtgggagctcctggagcccctgggccccctggctcccctggccccgctggtccaactggcaagcaaggagacagaggagaagctggtgcacaaggccccatgggaccctcaggaccagctggagcccggggaatccagggtcctcaaggccccagaggtgacaaaggagaggctggagagcctggcgagagaggcctgaagggacaccgtggcttcactggtctgcagggtctgcccggccctcctggtccttctggagaccaaggtgcttctggtcctgctggtccttctggccctagaggtcctcctggccccgtcggtccctctggcaaagatggtgctaatggaatccctggccccattgggcctcctggtccccgtggacgatcaggcgaaaccggccctgctggtcctcctggaaatcctggaccccctggtcctccaggtccccctggccctggcatcgacatgtccgcctttgctggcttaggcccgagagagaagggccccgaccccctgcagtacatgcgggccgaccaggcagccggtggcctgagacagcatgacgccgaggtggatgccacactcaagtccctcaacaaccagattgagagcatccgcagccccgagggctcccgcaagaaccctgctcgcacctgcagagacctgaaactctgccaccctgagtggaagagtggagactactggattgaccccaaccaaggctgcaccttggacgccatgaaggttttctgcaacatggagactggcgagacttgcgtctaccccaatccagcaaacgttcccaagaagaactggtggagcagcaagagcaaggagaagaaacacatctggtttggagaaaccatcaatggtggcttccatttcagctatggagatgacaatctggctcccaacactgccaacgtccagatgaccttcctacgcctgctgtccacggaaggctcccagaacatcacctaccactgcaagaacagcattgcctatctggacgaagcagctggcaacctcaagaaggccctgctcatccagggctccaatgacgtggagatccgggcagagggcaatagcaggttcacgtacactgccctgaaggatggctgcacgaaacataccggtaagtggggcaagactgttatcgagtaccggtcacagaagacctcacgcctccccatcattgacattgcacccatggacataggagggcccgagcaggaattcggtgtggacatagggccggtctgcttcttgtaa

SEQ ID NO: 104 - DNA, 4401 bp, Artificial Sequence
  synthesized DNA of human collagen type III alpha 1 precursor ORF having codon optimized for yeast
atgatgagcttcgttcaaaaaggatcgtggctcctcctggctctcttgcacccgaccattattttggctcaacaagaggccgtggagggggggtgtagccatctcggacaaagctacgcagatcgagatgtttggaagcccgagccctgtcaaatatgcgtttgtgatagtggtagcgttctatgcgacgacatcatctgcgatgaccaagagcttgattgtccgaatccagaaattccttttggagaatgttgtgctgtgtgcccgcagccacctaccgcacctaccaggccgcctaatggtcaaggcccacagggtccaaaaggtgaccctggtccgcctggaatccctggacgtaatggagatccgggaattccaggtcaacccggatcaccgggaagcccaggccctccaggaatctgcgagtcttgtccaaccggaccgcaaaattacagtccgcaatacgacagctatgatgtcaagtcaggggttgccgtggggggattggcagggtacccaggtcctgcaggaccgcctggtccgccaggtcccccaggaacatccggccacccaggaagcccagggtcgccgggttatcagggaccaccgggtgagccaggccaggcaggtccgtctggtcctccgggtccgcccggtgctatcggaccatctggaccggctggtaaagatggggaatccggtcgtccgggtcgtccgggtgagcgtggacttcctggtccaccgggtattaagggtccggctggaatacctggtttccctggcatgaaaggccatagaggtttcgacggtaggaacggtgagaagggagagactggtgctcctggtcttaagggtgaaaatggattgccaggagaaaatggtgcaccgggacctatgggaccgagaggcgcaccgggagagagaggaagaccaggtttacccggagcagccggtgcacgtggtaacgacggagcacgtggctctgatggtcagcctggtcctcctggacctcccggtaccgccgggttccctggttctccgggtgccaaaggagaggtcggtcctgctggatctcccggttctaatggcgccccaggtcagagaggagaacctggtccccaaggtcacgctggcgctcaaggccctcctggaccaccaggtatcaacggttctcctggaggcaagggtgagatgggccctgctggtataccaggggccccaggacttatgggagctagaggacccccaggtcctgctggtgctaacggtgccccaggattgcgtggtggagctggtgaaccagggaaaaatggagctaaaggagaacctgggcctagaggtgaaagaggtgaagcaggtatccctggcgttcctggtgctaagggagaagatggaaaagatgggtcgccaggggagccaggcgcaaacggtttacccggtgctgctggagagagaggagctccaggctttaggggtccagctggtccaaacggtattcccggtgaaaaaggtccagccggtgaacgaggagcaccaggtccagccggtccaagaggtgcagcaggtgaaccaggtcgagacggagtcccaggtggtcccggaatgaggggaatgccaggttcgccaggtggacccggctcagacggaaagccaggtccaccaggtagtcaaggagagtcagggcgaccaggaccaccaggaccatccggtcctcgtgggcagcccggtgttatgggtttccctggtcccaagggcaatgatggcgctcctggtaagaatggtgagagaggagggcctggcggtcccggtccccagggccctccaggcaagaacggggaaactggtccacagggacctccaggaccaaccggtccaggaggagacaaaggagatacaggaccaccaggtccacaaggtttgcaaggattaccaggaacgggtgggccaccaggggagaacggcaagcctggagaaccaggtcctaagggtgatgctggtgcacctggtgctcctggcggaaaaggtgatgctggagctcctggggaaagaggacctcctggattggctggggcaccaggactgagaggtggtgctggccctccagggcctgaaggaggaaaaggtgccgctggaccacccggtcctccaggcgcagccggaactcctggtctacaggggatgcctggtgaaagaggtggtcttggtagtcctgggcctaaaggcgacaaaggtgaaccaggaggtcctggtgctgatggtgttccaggtaaggacgggccacgaggacctacgggtccaatcggtcctccaggacctgctggtcagccaggtgataaaggggaaggaggtgcacccggtttacctggtattgccggacctcgaggtagtcctggtgagaggggagaaactggcccacctgggcctgctggttttccaggtgcccctggtcagaacggagaacctggtggtaagggggagagaggtgcaccaggagaaaagggtgaaggaggcccacccggagttgcaggacctccaggaggatctgggccagctggtccaccaggacctcaaggtgtaaaaggtgagcgaggatcgccaggtggtccaggtgccgcaggctttccaggggcaagggggttacccggaccccctggttccaatggcaacccaggacctcccggtccatccggatcccctgggaaggatggtccccctggtcccgctggtaacacaggtgctcctggttctcccggcgtctcaggacccaaaggggatgctggtcaaccaggtgaaaagggatccccaggtgctcaaggtcccccaggtgcaccagggcccttgggtattgctggaattacaggtgctagaggtttggctggtccacccggcatgccaggtcctaggggatctcctggaccacagggagttaagggcgagtccggaaaaccaggtgccaacggactttcaggtgaaaggggtcccccaggccctcaaggcctgccaggattagccggaactgctggagaacccggacgtgatggaaacccaggttctgacggtttgcccggtagggatggaagtccaggaggcaagggcgacagaggagagaatggatcaccaggagcccctggagcaccaggacatccaggaccacctggccctgtaggtccagccggtaaatcaggagacagaggagaatcaggtcccgccggtccagctggcgcccctggccccgctggatcaagaggtgccccaggtcctcaaggaccaagaggcgataaaggagaaaccggagaaagaggtgctgctggaattaagggccacagaggtttccctgggaaccctggagccccagggagtccaggtcctgcaggtcagcaaggggccattggtagtccaggaccagccggtcccagaggtcctgtgggaccatctggtcctccagggaaggatggcacttctggtcaccccggccctatcggtccccctggaccaagaggcaacagaggcgagaggggctcagagggatcccctggtcatcctggacaacctggccctccagggcctcccggcgctccaggtccatgctgtggaggtgtcggtgctgctgctattgctggcatcggaggtgaaaaagccggtggatttgccccttattatggcgacgaaccaatggatttcaagattaacactgacgaaattatgacatctttgaagtccgtaaacggtcaaatagagtccttgatttcacctgatggttctcgtaagaacccagcccgaaactgtcgtgatcttaaattttgccatcctgaattgaagagtggtgagtactgggtggaccctaatcaaggttgtaagctagacgccataaaagtgttctgtaatatggaaactggagagacttgtatttccgccaaccccttgaatgtgcctaggaaacattggtggaccgattcctcagctgagaagaagcatgtttggtttggtgaatctatggacgggggctttcagttttcctacggcaatcctgaactaccagaggatgtcctggacgttcaactggccttcctgagattattgtccagtagagcctctcaaaacatcacataccactgtaagaattcaattgcctatatggaccaggcctctgggaatgtcaaaaaggctctgaagctgatgggcagtaatgaaggcgaatttaaagcagagggcaactctaaatttacttacactgttcttgaagatggctgtacaaaacatactggtgaatggtctaagacggtattcgagtatagaactagaaaagcagttagattgcctatagttgatatcgccccttatgatattggaggtcccgaccaagagtttggtgtcgatgtaggaccagtttgtttcctataa

SEQ ID NO: 105 - DNA, 4401 bp, Homo sapiens
atgatgagctttgtgcaaaaggggagctggctacttctcgctctgcttcatcccactattattttggcacaacaggaagctgttgaaggaggatgttcccatcttggtcagtcctatgcggatagagatgtctggaagccagaaccatgccaaatatgtgtctgtgactcaggatccgttctctgcgatgacataatatgtgacgatcaagaattagactgccccaacccagaaattccatttggagaatgttgtgcagtttgcccacagcctccaactgctcctactcgccctcctaatggtcaaggacctcaaggccccaagggagatccaggccctcctggtattcctgggagaaatggtgaccctggtattccaggacaaccagggtcccctggttctcctggcccccctggaatctgtgaatcatgccctactggtcctcagaactattctccccagtatgattcatatgatgtcaagtctggagtagcagtaggaggactcgcaggctatcctggaccagctggccccccaggccctcccggtccccctggtacatctggtcatcctggttcccctggatctccaggataccaaggaccccctggtgaacctgggcaagctggtccttcaggccctccaggacctcctggtgctataggtccatctggtcctgctggaaaagatggagaatcaggtagacccggacgacctggagagcgaggattgcctggacctccaggtatcaaaggtccagctgggatacctggattccctggtatgaaaggacacagaggcttcgatggacgaaatggagaaaagggtgaaacaggtgctcctggattaaagggtgaaaatggtcttccaggcgaaaatggagctcctggacccatgggtccaagaggggctcctggtgagcgaggacggccaggacttcctggggctgcaggtgctcggggtaatgacggtgctcgaggcagtgatggtcaaccaggccctcctggtcctcctggaactgccggattccctggatcccctggtgctaagggtgaagttggacctgcagggtctcctggttcaaatggtgcccctggacaaagaggagaacctggacctcagggacacgctggtgctcaaggtcctcctggccctcctgggattaatggtagtcctggtggtaaaggcgaaatgggtcccgctggcattcctggagctcctggactgatgggagcccggggtcctccaggaccagccggtgctaatggtgctcctggactgcgaggtggtgcaggtgagcctggtaagaatggtgccaaaggagagcccggaccacgtggtgaacgcggtgaggctggtattccaggtgttccaggagctaaaggcgaagatggcaaggatggatcacctggagaacctggtgcaaatgggcttccaggagctgcaggagaaaggggtgcccctgggttccgaggacctgctggaccaaatggcatcccaggagaaaagggtcctgctggagagcgtggtgctccaggccctgcagggcccagaggagctgctggagaacctggcagagatggcgtccctggaggtccaggaatgaggggcatgcccggaagtccaggaggaccaggaagtgatgggaaaccagggcctcccggaagtcaaggagaaagtggtcgaccaggtcctcctgggccatctggtccccgaggtcagcctggtgtcatgggcttccccggtcctaaaggaaatgatggtgctcctggtaagaatggagaacgaggtggccctggaggacctggccctcagggtcctcctggaaagaatggtgaaactggacctcaaggacccccagggcctactgggcctggtggtgacaaaggagacacaggaccccctggtccacaaggattacaaggcttgcctggtacaggtggtcctccaggagaaaatggaaaacctggggaaccaggtccaaagggtgatgccggtgcacctggagctccaggaggcaagggtgatgctggtgcccctggtgaacgtggacctcctggattggcaggggccccaggacttagaggtggagctggtccccctggtcccgaaggaggaaagggtgctgctggtcctcctgggccacctggtgctgctggtactcctggtctgcaaggaatgcctggagaaagaggaggtcttggaagtcctggtccaaagggtgacaagggtgaaccaggcggcccaggtgctgatggtgtcccagggaaagatggcccaaggggtcctactggtcctattggtcctcctggcccagctggccagcctggagataagggtgaaggtggtgcccccggacttccaggtatagctggacctcgtggtagccctggtgagagaggtgaaactggccctccaggacctgctggtttccctggtgctcctggacagaatggtgaacctggtggtaaaggagaaagaggggctccgggtgagaaaggtgaaggaggccctcctggagttgcaggaccccctggaggttctggacctgctggtcctcctggtccccaaggtgtcaaaggtgaacgtggcagtcctggtggacctggtgctgctggcttccctggtgctcgtggtcttcctggtcctcctggtagtaatggtaacccaggacccccaggtcccagcggttctccaggcaaggatgggcccccaggtcctgcgggtaacactggtgctcctggcagccctggagtgtctggaccaaaaggtgatgctggccaaccaggagagaagggatcgcctggtgcccagggcccaccaggagctccaggcccacttgggattgctgggatcactggagcacggggtcttgcaggaccaccaggcatgccaggtcctaggggaagccctggccctcagggtgtcaagggtgaaagtgggaaaccaggagctaacggtctcagtggagaacgtggtccccctggaccccagggtcttcctggtctggctggtacagctggtgaacctggaagagatggaaaccctggatcagatggtcttccaggccgagatggatctcctggtggcaagggtgatcgtggtgaaaatggctctcctggtgcccctggcgctcctggtcatccaggcccacctggtcctgtcggtccagctggaaagagtggtgacagaggagaaagtggccctgctggccctgctggtgctcccggtcctgctggttcccgaggtgctcctggtcctcaaggcccacgtggtgacaaaggtgaaacaggtgaacgtggagctgctggcatcaaaggacatcgaggattccctggtaatccaggtgccccaggttctccaggccctgctggtcagcagggtgcaatcggcagtccaggacctgcaggccccagaggacctgttggacccagtggacctcctggcaaagatggaaccagtggacatccaggtcccattggaccaccagggcctcgaggtaacagaggtgaaagaggatctgagggctccccaggccacccagggcaaccaggccctcctggacctcctggtgcccctggtccttgctgtggtggtgttggagccgctgccattgctgggattggaggtgaaaaagctggcggttttgccccgtattatggagatgaaccaatggatttcaaaatcaacaccgatgagattatgacttcactcaagtctgttaatggacaaatagaaagcctcattagtcctgatggttctcgtaaaaaccccgctagaaactgcagagacctgaaattctgccatcctgaactcaagagtggagaatactgggttgaccctaaccaaggatgcaaattggatgctatcaaggtattctgtaatatggaaactggggaaacatgcataagtgccaatcctttgaatgttccacggaaacactggtggacagattctagtgctgagaagaaacacgtttggtttggagagtccatggatggtggttttcagtttagctacggcaatcctgaacttcctgaagatgtccttgatgtgcagctggcattccttcgacttctctccagccgagcttcccagaacatcacatatcactgcaaaaatagcattgcatacatggatcaggccagtggaaatgtaaagaaggccctgaagctgatggggtcaaatgaaggtgaattcaaggctgaaggaaatagcaaattcacctacacagttctggaggatggttgcacgaaacacactggggaatggagcaaaacagtctttgaatatcgaacacgcaaggctgtgagactacctattgtagatattgcaccctatgacattggtggtcctgatcaagaatttggtgtggacgttggccctgtttgctttttataa

SEQ ID NO: 106 - DNA, 1578 bp, Homo sapiens
atgttttctttcgtagatttgcgactcctcttgttgcttgctgctaccgctctccttactcatggacaggaagaaggccaggtggaaggacaggacgaagacattcctcctataacttgtgtgcagaacggactgaggtatcatgacagagatgtgtggaagccagagccttgtcgaatatgcgtttgtgacaacggaaaggtgctatgtgatgatgttatctgtgatgagacaaagaactgtcctggcgctgaagttccagagggagaatgttgtccagtttgtcctgatggatcggagtcacccactgatcaagagactacgggagtagaaggtccaaaaggtgacactggtcccagaggcccaagaggacctgccggcccccctggtagggatggcattcctggtcaacccggcctaccaggccccccaggtccacctggccccccaggaccccctggtttaggaggtaacttcgctcctcaactgtcttacggttacgatgaaaagtctactggtggaatctctgttcctggaccaatgggtccgtctggtccaaggggtctacctggtcctcccggtgcacctggtccgcagggttttcaaggtccaccaggtgaaccgggagaaccgggagcttcaggtccaatgggtcccaggggtcctcctggccctcctggaaagaacggcgatgatggcgaggctggtaagccgggaccaccagggccgcctggtcctcctggtccacctggtccgcccagtgcaggattcgacttttccttcttaccccaaccgccacaggaaaaggcacacgatggtggtagatattaccgtgctgatgatgccaatgtcgtgagagatagagacttagaagttgatacgacattaaaatcactttctcaacaaattgagaacattagatctccggagggtagcagaaagaacccggccaggacttgccgtgatcttaagatgtgccactccgattggaagtcgggagagtactggatcgatcctaatcaaggttgtaacttggatgcaataaaagtcttctgtaacatggagacaggagaaacttgcgtttacccaactcaaccaagtgttgcacagaaaaattggtatatttccaagaatccaaaggataagcgtcatgtttggtttggagaatctatgaccgatggctttcaattcgagtatggaggtcagggtagcgacccagctgatgtcgctattcaattgacctttttgagacttatgtccacagaagcttcgcaaaacattacatatcactgtaagaatagcgtagcctacatggaccaacagactggtaatctgaaaaaggccctgttgttgcaaggcagtaatgaaatcgaaattcgtgccgaaggaaattccagattcacatactctgttaccgttgacggctgcacctctcataccggtgcctggggtaaaactgtaattgagtataaaacgacaaagacctcccgtcttcctatcattgatgttgctccattagatgtcggtgcccctgaccaggagtttggttttgacgtaggccccgtctgcttcctgtaa

SEQ ID NO: 107 - DNA, 1284 bp, Homo sapiens
atgctctctttcgttgatactagaactctattgctgttagcagttactttgtgtttagcaacctgtcaatccttgcaagaagaaaccgttagaaaaggtcctgcaggagatagaggtccaagaggcgagagagggcccccgggacccccagggcgagatggtgaggatggtccgacaggtccaccaggacctcctggacctcccggacctccgggactcggtggtaacttcgctgctcaatatgatggtaaaggcgttggtcttggaccgggaccaatgggtctgatgggtcctagaggaccaccaggggcagcaggggcacctggtccccaaggattccaaggacccgcaggggaacccggggagccaggacagacaggacctgcaggggctagaggacccgccggtccaccaggtaaggctggtgaggacggccacccaggtaaacccggacctcctggcccaccaggtcctccgggtccacctggagtttcaggcggaggttacgactttggatatgacggagatttttatagagctgatcaacctcgttcagccccctctttgagaccaaaagactatgaagtggacgctacattgaagagtctcaacaatcagatcgaaaccttgcttactccggaaggttcaagaaaaaatcctgctaggacctgcagagacctgagactttctcatccagagtggtcttccggctattattggattgatcctaaccaaggctgtacaatggatgctattaaggtctactgcgacttttccaccggtgagacatgtataagagctcaacctgaaaacattcctgctaaaaattggtacagatcaagtaaagacaaaaagcatgtgtggttgggtgaaactattaatgccggctctcagtttgaatacaatgtcgagggagtcacttctaaagagatggctacgcaacttgctttcatgcgtttattagccaattacgccagtcaaaatatcacgtaccactgcaagaatagcattgcttacatggacgaggagactggtaaccttaaaaaggccgttattctgcaaggtagcaatgacgtagagctggtcgctgaaggtaactcacgattcacttatactgtcctggttgacggttgttcaaaaaagaccaacgaatggggaaaaacgattatcgagtacaagacaaataagccaagcaggctgccttttttagacattgcaccactagacataggcggggctgatcaggaatttttcgtggacattggtcccgtctgttttaagtaa

SEQ ID NO: 108 - DNA, 3045 bp, Homo sapiens
atgttttctttcgtagatttgcgactcctcttgttgcttgctgctaccgctctccttactcatggacaggaagaaggccaggtggaaggacaggacgaagacattcctcctataacttgtgtgcagaacggactgaggtatcatgacagagatgtgtggaagccagagccttgtcgaatatgcgtttgtgacaacggaaaggtgctatgtgatgatgttatctgtgatgagacaaagaactgtcctggcgctgaagttccagagggagaatgttgtccagtttgtcctgatggatcggagtcacccactgatcaagagactacgggagtagaaggtccaaaaggtgacactggtcccagaggcccaagaggacctgccggcccccctggtagggatggcattcctggtcaacccggcctaccaggccccccaggtccacctggccccccaggaccccctggtttaggaggtaacttcgctcctcaactgtcttacggttacgatgaaaagtctactggtggaatctctgttcctggaccaatgggtccgtctggtccaaggggtctacctggtcctcccggtgcacctggtccgcagggttttcaaggtccaccaggtccacagggtccaggaggtcctcctggacctaaaggaaatagtggagaaccaggagctccagggagtaaaggagacactggtgctaagggagagccaggaccagtgggggttcaaggtcctcccggacccgctggagaagagggaaaaagaggtgcacgtggtgagcctggtcctactggtctgccaggtccacctggggaaaggggtggtcctgggtcaagaggcttccccggtgctgacggagttgctggtccgaaaggaccagccggagagcgaggatccccaggtcctgcaggccctaaggggtcccctggtgaggcaggaagaccgggtgaggctggtttgcccggtgccaaaggtctcacgggctccccaggctctcctggcccagacggcaaaactggcccacctggccctgccggacaagacggaagacctggacctccaggaccacctggtgccagaggtcaggccggtgtgatggggtttccaggtcccaagggtgctgcaggtgaaccagggaaagccggtgaacgaggtgtccccggtcctccaggtgctgttgggccggctggaaaggatggtgaggccggtgcacaagggcctccaggccctgctggtcctgccggtgaaagaggagaacagggaccagccggttctcctggctttcaaggtctgcccggacctgccgggcctccaggagaagctggtaaaccaggagaacaaggtgtcccaggagatttgggagcccctgggccctcaggcgctagaggtgagaggggatttcctggagaacgtggtgtacaaggtcccccgggtccagcaggtccgaggggagctaatggagctccaggaaacgacggtgctaaaggcgacgcaggtgctccaggagcaccaggatcacagggtgccccaggtttgcaaggcatgccaggggaaagaggagcagcaggacttccaggccccaaaggcgacaggggtgacgccggtccaaaaggggccgatggttctcctggcaaagacggcgttcgaggcttgacaggtccgatcggtccacctggacccgccggtgcaccaggtgataagggagaatctggtccttcaggccctgccggtccaaccggggctagaggcgctcctggggatagaggtgaacccggtccaccagggccagccggcttcgctggaccaccaggtgctgatggacagcccggagctaagggtgagccaggtgatgctggtgctaaaggtgacgctggacctccgggtcccgcaggacctgctggtccccctgggccaattggtaatgttggtgccccaggagcaaaaggagccagaggttctgcaggcccaccaggagccacaggattccccggtgctgctggaagagtcggaccaccgggtccatccgggaacgccggacctccaggtcctccgggtcctgctggtaaagaaggaggtaagggtccaagaggtgaaacagggcctgctggccgtcctggtgaagtcggacctcctggcccacccggtccagctggggaaaagggatcaccaggagctgatggaccaccagggccgcctggtcctcctggtccacctggtccgcccagtgcaggattcgacttttccttcttaccccaaccgccacaggaaaaggcacacgatggtggtagatattaccgtgctgatgatgccaatgtcgtgagagatagagacttagaagttgatacgacattaaaatcactttctcaacaaattgagaacattagatctccggagggtagcagaaagaacccggccaggacttgccgtgatcttaagatgtgccactccgattggaagtcgggagagtactggatcgatcctaatcaaggttgtaacttggatgcaataaaagtcttctgtaacatggagacaggagaaacttgcgtttacccaactcaaccaagtgttgcacagaaaaattggtatatttccaagaatccaaaggataagcgtcatgtttggtttggagaatctatgaccgatggctttcaattcgagtatggaggtcagggtagcgacccagctgatgtcgctattcaattgacctttttgagacttatgtccacagaagcttcgcaaaacattacatatcactgtaagaatagcgtagcctacatggaccaacagactggtaatctgaaaaaggccctgttgttgcaaggcagtaatgaaatcgaaattcgtgccgaaggaaattccagattcacatactctgttaccgttgacggctgcacctctcataccggtgcctggggtaaaactgtaattgagtataaaacgacaaagacctcccgtcttcctatcattgatgttgctccattagatgtcggtgcccctgaccaggagtttggttttgacgtaggccccgtctgcttcctgtaa

SEQ ID NO: 109 - DNA, 2751 bp, Homo sapiens
atgctctctttcgttgatactagaactctattgctgttagcagttactttgtgtttagcaacctgtcaatccttgcaagaagaaaccgttagaaaaggtcctgcaggagatagaggtccaagaggcgagagagggcccccgggacccccagggcgagatggtgaggatggtccgacaggtccaccaggacctcctggacctcccggacctccgggactcggtggtaacttcgctgctcaatatgatggtaaaggcgttggtcttggaccgggaccaatgggtctgatgggtcctagaggaccaccaggggcagcaggggcacctggtccccaaggattccaaggacccgcagggccagttggcgcagctggtgctacgggagctaggggtctagtgggcgaaccaggtccagcagggtctaagggtgaatccggtaacaagggtgaacctggaagtgccggtcctcagggacctcctggtccatcaggggaggaaggaaagagaggccctaatggcgaggctggtagtgcaggaccaccgggtcctcctggattgagaggttcccccgggtctcgtggactacctggtgccgacggtagagctggcgttatgggtcctcccggaagcagaggagcttctggacccgcaggtgtcaggggtccaaacggtgatgctggaaggccaggtgaaccaggattgatgggtccacgtggattgcccggctctcctggaaacattggacctgccggtaaagaaggccctgtgggtttgccaggtattgatggcagaccaggtccaatcggcccagcaggcgctagaggagagcctggtaacataggattccctggtcctaagggaccaactggtgatccaggcaagaacggtgataagggtcatgccgggctagctggggctagaggtgcacctggcccagatgggaacaacggagcccaagggccaccgggtccacagggggtacaaggtggcaagggcgaacagggtccagctggcccgcctggttttcagggacttccgggtcctagcggtcccgctggagaagttggaaagcctggtgaacgtggactacatggagaattcggactgcctggtcctgctggtccacgtggagaaagaggtcctcccggggagagtggagctgctggtcctaccggaccaattgggtcacgtggaccctccggtcctccaggaccagatgggaataagggtgaaccaggagtcgtaggggcagttggtacagctggcccttccggtccttcgggccttccaggagaaagaggagccgccggaatccctggtggtaaaggcgaaaaaggagaacctggtttaaggggtgagattggaaatccaggtagagatggtgcccgaggagccccaggtgccgtcggtgctcccgggccagccggagccactggcgataggggagaagccggagctgctggaccagccggtccagctggaccaagaggatccccgggtgagagaggagaagttgggccagccggtccgaacggtttcgccggaccagccggtgcagccggtcaaccaggtgctaagggcgagagaggtgctaaaggccctaaaggtgagaatggtgtcgttggccccactgggcctgttggagcagctggacccgctggtccaaacggaccacccggtcctgctggatcgaggggtgacggtggtccaccaggtatgacgggatttccaggtgcagctggtagaactggtccccctggtccgtccgggatttcaggtccacctgggcctccaggacctgctggaaaagagggcttgaggggtccgagaggagatcaaggccctgtaggaagaaccggggaagttggtgccgttggacccccgggttttgctggggagaaaggaccatctggagaagcaggacctcctggcccaccaggtcctccgggtccacctggagtttcaggcggaggttacgactttggatatgacggagatttttatagagctgatcaacctcgttcagccccctctttgagaccaaaagactatgaagtggacgctacattgaagagtctcaacaatcagatcgaaaccttgcttactccggaaggttcaagaaaaaatcctgctaggacctgcagagacctgagactttctcatccagagtggtcttccggctattattggattgatcctaaccaaggctgtacaatggatgctattaaggtctactgcgacttttccaccggtgagacatgtataagagctcaacctgaaaacattcctgctaaaaattggtacagatcaagtaaagacaaaaagcatgtgtggttgggtgaaactattaatgccggctctcagtttgaatacaatgtcgagggagtcacttctaaagagatggctacgcaacttgctttcatgcgtttattagccaattacgccagtcaaaatatcacgtaccactgcaagaatagcattgcttacatggacgaggagactggtaaccttaaaaaggccgttattctgcaaggtagcaatgacgtagagctggtcgctgaaggtaactcacgattcacttatactgtcctggttgacggttgttcaaaaaagaccaacgaatggggaaaaacgattatcgagtacaagacaaataagccaagcaggctgccttttttagacattgcaccactagacataggcggggctgatcaggaatttttcgtggacattggtcccgtctgttttaagtaa

SEQ ID NO: 110 - DNA, 4611 bp, Homo sapiens
atgttttctttcgtagatttgcgactcctcttgttgcttgctgctaccgctctccttactcatggacaggaagaaggccaggtggaaggacaggacgaagacattcctcctataacttgtgtgcagaacggactgaggtatcatgacagagatgtgtggaagccagagccttgtcgaatatgcgtttgtgacaacggaaaggtgctatgtgatgatgttatctgtgatgagacaaagaactgtcctggcgctgaagttccagagggagaatgttgtccagtttgtcctgatggatcggagtcacccactgatcaagagactacgggagtagaaggtccaaaaggtgacactggtcccagaggcccaagaggacctgccggcccccctggtagggatggcattcctggtcaacccggcctaccaggccccccaggtccacctggccccccaggaccccctggtttaggaggtaacttcgctcctcaactgtcttacggttacgatgaaaagtctactggtggaatctctgttcctggaccaatgggtccgtctggtccaaggggtctacctggtcctcccggtgcacctggtccgcagggttttcaaggtccaccaggtccacagggtccaggaggtcctcctggacctaaaggaaatagtggagaaccaggagctccagggagtaaaggagacactggtgctaagggagagccaggaccagtgggggttcaaggtcctcccggacccgctggagaagagggaaaaagaggtgcacgtggtgagcctggtcctactggtctgccaggtccacctggggaaaggggtggtcctgggtcaagaggcttccccggtgctgacggagttgctggtccgaaaggaccagccggagagcgaggatccccaggtcctgcaggccctaaggggtcccctggtgaggcaggaagaccgggtgaggctggtttgcccggtgccaaaggtctcacgggctccccaggctctcctggcccagacggcaaaactggcccacctggccctgccggacaagacggaagacctggacctccaggaccacctggtgccagaggtcaggccggtgtgatggggtttccaggtcccaagggtgctgcaggtgaaccagggaaagccggtgaacgaggtgtccccggtcctccaggtgctgttgggccggctggaaaggatggtgaggccggtgcacaagggcctccaggccctgctggtcctgccggtgaaagaggagaacagggaccagccggttctcctggctttcaaggtctgcccggacctgccgggcctccaggagaagctggtaaaccaggagaacaaggtgtcccaggagatttgggagcccctgggccctcaggcgctagaggtgagaggggatttcctggagaacgtggtgtacaaggtcccccgggtccagcaggtccgaggggagctaatggagctccaggaaacgacggtgctaaaggcgacgcaggtgctccaggagcaccaggatcacagggtgccccaggtttgcaaggcatgccaggggaaagaggagcagcaggacttccaggccccaaaggcgacaggggtgacgccggtccaaaaggggccgatggttctcctggcaaagacggcgttcgaggcttgacaggtccgatcggtccacctggacccgccggtgcaccaggtgataagggagaatctggtccttcaggccctgccggtccaaccggggctagaggcgctcctggggatagaggtgaacccggtccaccagggccagccggcttcgctggaccaccaggtgctgatggacagcccggagctaagggtgagccaggtgatgctggtgctaaaggtgacgctggacctccgggtcccgcaggacctgctggtccccctgggccaattggtaatgttggtgccccaggagcaaaaggagccagaggttctgcaggcccaccaggagccacaggattccccggtgctgctggaagagtcggaccaccgggtccatccgggaacgccggacctccaggtcctccgggtcctgctggtaaagaaggaggtaagggtccaagaggtgaaacagggcctgctggccgtcctggtgaagtcggacctcctggcccacccggtccagctggggaaaagggatcaccaggagctgatggaccccagggccccggcggccctcctggtcccaagggtaacagcggtgaacctggtgctcctggcagcaaaggagacactggtgctaagggagagcctggccctgttggtgttcaaggaccccctggccctgctggagaggaaggaaagcgaggagctcgaggtgaacccggacccactggcctgcccggaccccctggcgagcgtggtggacctggtagccgtggtttccctggcgcagatggtgttgctggtcccaagggtcccgctggtgaacgtggttctcctggccctgctggccccaaaggatctcctggtgaagctggtcgtcccggtgaagctggtctgcctggtgccaagggtctgactggaagccctggcagccctggtcctgatggcaaaactggcccccctggtcccgccggtcaagatggtcgccccggacccccaggcccacctggtgcccgtggtcaggctggtgtgatgggattccctggacctaaaggtgctgctggagagcccggcaaggctggagagcgaggtgttcccggaccccctggcgctgtcggtcctgctggcaaagatggagaggctggagctcagggaccccctggccctgctggtcccgctggcgagagaggtgaacaaggccctgctggctcccccggattccagggtctccctggtcctgctggtcctccaggtgaagcaggcaaacctggtgaacagggtgttcctggagaccttggcgcccctggcccctctggagcaagaggcgagagaggtttccctggcgagcgtggtgtgcaaggtccccctggtcctgctggtccccgaggggccaacggtgctcccggcaacgatggtgctaagggtgatgctggtgcccctggagctcccggtagccagggcgcccctggccttcagggaatgcctggtgaacgtggtgcagctggtcttccagggcctaagggtgacagaggtgatgctggtcccaaaggtgctgatggctctcctggcaaagatggcgtccgtggtctgaccggccccattggtcctcctggccctgctggtgcccctggtgacaagggtgaaagtggtcccagcggccctgctggtcccactggagctcgtggtgcccccggagaccgtggtgagcctggtccccccggccctgctggctttgctggcccccctggtgctgacggccaacctggtgctaaaggcgaacctggtgatgctggtgctaaaggcgatgctggtccccctggccctgccggacccgctggaccccctggccccattggtaatgttggtgctcctggagccaaaggtgctcgcggcagcgctggtccccctggtgctactggtttccctggtgctgctggccgagtcggtcctcctggcccctctggaaatgctggaccccctggccctcctggtcctgctggcaaagaaggcggcaaaggtccccgtggtgagactggccctgctggacgtcctggtgaagttggtccccctggtccccctggccctgctggcgagaaaggatcccctggtgctgatggaccaccagggccgcctggtcctcctggtccacctggtccgcccagtgcaggattcgacttttccttcttaccccaaccgccacaggaaaaggcacacgatggtggtagatattaccgtgctgatgatgccaatgtcgtgagagatagagacttagaagttgatacgacattaaaatcactttctcaacaaattgagaacattagatctccggagggtagcagaaagaacccggccaggacttgccgtgatcttaagatgtgccactccgattggaagtcgggagagtactggatcgatcctaatcaaggttgtaacttggatgcaataaaagtcttctgtaacatggagacaggagaaacttgcgtttacccaactcaaccaagtgttgcacagaaaaattggtatatttccaagaatccaaaggataagcgtcatgtttggtttggagaatctatgaccgatggctttcaattcgagtatggaggtcagggtagcgacccagctgatgtcgctattcaattgacctttttgagacttatgtccacagaagcttcgcaaaacattacatatcactgtaagaatagcgtagcctacatggaccaacagactggtaatctgaaaaaggccctgttgttgcaaggcagtaatgaaatcgaaattcgtgccgaaggaaattccagattcacatactctgttaccgttgacggctgcacctctcataccggtgcctggggtaaaactgtaattgagtataaaacgacaaagacctcccgtcttcctatcattgatgttgctccattagatgtcggtgcccctgaccaggagtttggttttgacgtaggccccgtctgcttcctgtaa

SEQ ID NO: 111 - DNA, 4317 bp, Homo sapiens
atgctctctttcgttgatactagaactctattgctgttagcagttactttgtgtttagcaacctgtcaatccttgcaagaagaaaccgttagaaaaggtcctgcaggagatagaggtccaagaggcgagagagggcccccgggacccccagggcgagatggtgaggatggtccgacaggtccaccaggacctcctggacctcccggacctccgggactcggtggtaacttcgctgctcaatatgatggtaaaggcgttggtcttggaccgggaccaatgggtctgatgggtcctagaggaccaccaggggcagcaggggcacctggtccccaaggattccaaggacccgcagggccagttggcgcagctggtgctacgggagctaggggtctagtgggcgaaccaggtccagcagggtctaagggtgaatccggtaacaagggtgaacctggaagtgccggtcctcagggacctcctggtccatcaggggaggaaggaaagagaggccctaatggcgaggctggtagtgcaggaccaccgggtcctcctggattgagaggttcccccgggtctcgtggactacctggtgccgacggtagagctggcgttatgggtcctcccggaagcagaggagcttctggacccgcaggtgtcaggggtccaaacggtgatgctggaaggccaggtgaaccaggattgatgggtccacgtggattgcccggctctcctggaaacattggacctgccggtaaagaaggccctgtgggtttgccaggtattgatggcagaccaggtccaatcggcccagcaggcgctagaggagagcctggtaacataggattccctggtcctaagggaccaactggtgatccaggcaagaacggtgataagggtcatgccgggctagctggggctagaggtgcacctggcccagatgggaacaacggagcccaagggccaccgggtccacagggggtacaaggtggcaagggcgaacagggtccagctggcccgcctggttttcagggacttccgggtcctagcggtcccgctggagaagttggaaagcctggtgaacgtggactacatggagaattcggactgcctggtcctgctggtccacgtggagaaagaggtcctcccggggagagtggagctgctggtcctaccggaccaattgggtcacgtggaccctccggtcctccaggaccagatgggaataagggtgaaccaggagtcgtaggggcagttggtacagctggcccttccggtccttcgggccttccaggagaaagaggagccgccggaatccctggtggtaaaggcgaaaaaggagaacctggtttaaggggtgagattggaaatccaggtagagatggtgcccgaggagccccaggtgccgtcggtgctcccgggccagccggagccactggcgataggggagaagccggagctgctggaccagccggtccagctggaccaagaggatccccgggtgagagaggagaagttgggccagccggtccgaacggtttcgccggaccagccggtgcagccggtcaaccaggtgctaagggcgagagaggtgctaaaggccctaaaggtgagaatggtgtcgttggccccactgggcctgttggagcagctggacccgctggtccaaacggaccacccggtcctgctggatcgaggggtgacggtggtccaccaggtatgacgggatttccaggtgcagctggtagaactggtccccctggtccgtccgggatttcaggtccacctgggcctccaggacctgctggaaaagagggcttgaggggtccgagaggagatcaaggccctgtaggaagaaccggggaagttggtgccgttggacccccgggttttgctggggagaaaggaccatctggagaagcaggccctgttggtgctgccggtgctactggtgccagaggacttgttggtgagcctggtccagctggctccaaaggagagagcggtaacaagggtgagcccggctctgctgggccccaaggtcctcctggtcccagtggtgaagaaggaaagagaggccctaatggggaagctggatctgccggccctccaggacctcctgggctgagaggtagtcctggttctcgtggtcttcctggagctgatggcagagctggcgtcatgggccctcctggtagtcgtggtgcaagtggccctgctggagtccgaggacctaatggagatgctggtcgccctggggagcctggtctcatgggacccagaggtcttcctggttcccctggaaatatcggccccgctggaaaagaaggtcctgtcggcctccctggcatcgacggcaggcctggcccaattggcccagctggagcaagaggagagcctggcaacattggattccctggacccaaaggccccactggtgatcctggcaaaaacggtgataaaggtcatgctggtcttgctggtgctcggggtgctccaggtcctgatggaaacaatggtgctcagggacctcctggaccacagggtgttcaaggtggaaaaggtgaacagggtcccgctggtcctccaggcttccagggtctgcctggcccctcaggtcccgctggtgaagttggcaaaccaggagaaaggggtctccatggtgagtttggtctccctggtcctgctggtccaagaggggaacgcggtcccccaggtgagagtggtgctgccggtcctactggtcctattggaagccgaggtccttctggacccccagggcctgatggaaacaagggtgaacctggtgttgttggtgctgtgggcactgctggtccatctggtcctagtggactcccaggagagaggggtgctgctggcatacctggaggcaagggagaaaagggtgaacctggtctcagaggtgaaattggtaaccctggcagagatggtgctcgtggtgctcctggtgctgtaggtgcccctggtcctgctggagccacaggtgaccggggcgaagctggggctgctggtcctgctggtcctgctggtcctcggggaagccctggtgaacgtggtgaggtcggtcctgctggccccaatggatttgctggtcctgctggtgctgctggtcaacctggtgctaaaggagaaagaggagccaaagggcctaagggtgaaaacggtgttgttggtcccacaggccccgttggagctgctggcccagctggtccaaatggtccccccggtcctgctggaagtcgtggtgatggaggcccccctggtatgactggtttccctggtgctgctggacggactggtcccccaggaccctctggtatttctggccctcctggtccccctggtcctgctgggaaagaagggcttcgtggtcctcgtggtgaccaaggtccagttggccgaactggagaagtaggtgcagttggtccccctggcttcgctggtgagaagggtccctctggagaggctggacctcctggcccaccaggtcctccgggtccacctggagtttcaggcggaggttacgactttggatatgacggagatttttatagagctgatcaacctcgttcagccccctctttgagaccaaaagactatgaagtggacgctacattgaagagtctcaacaatcagatcgaaaccttgcttactccggaaggttcaagaaaaaatcctgctaggacctgcagagacctgagactttctcatccagagtggtcttccggctattattggattgatcctaaccaaggctgtacaatggatgctattaaggtctactgcgacttttccaccggtgagacatgtataagagctcaacctgaaaacattcctgctaaaaattggtacagatcaagtaaagacaaaaagcatgtgtggttgggtgaaactattaatgccggctctcagtttgaatacaatgtcgagggagtcacttctaaagagatggctacgcaacttgctttcatgcgtttattagccaattacgccagtcaaaatatcacgtaccactgcaagaatagcattgcttacatggacgaggagactggtaaccttaaaaaggccgttattctgcaaggtagcaatgacgtagagctggtcgctgaaggtaactcacgattcacttatactgtcctggttgacggttgttcaaaaaagaccaacgaatggggaaaaacgattatcgagtacaagacaaataagccaagcaggctgccttttttagacattgcaccactagacataggcggggctgatcaggaatttttcgtggacattggtcccgtctgttttaagtaa

SEQ ID NO: 112 - DNA, 429 bp, Artificial Sequence
  synthesized DNA of human FKBP13A ORF having codon optimized for yeast
atgcgtctttcctggtttcgtgttcttaccgttttatctatttgcttgtctgcagtagccactgctactggagctgaaggtaaacgaaaactgcaaattggtgttaagaagagagtcgatcattgtcctattaaatccagaaagggtgatgttcttcatatgcactacacgggaaaattggaagatggtacagaatttgacagctcattgccccagaaccaaccatttgttttctcattgggaaccggtcaggtgatcaaagggtgggaccaaggcttgttaggaatgtgtgaaggtgaaaagagaaagttagtgattccaagtgaactaggttatggagagaggggcgccccaccgaaaatccctggtggggctacattggtcttcgaggtcgagctgctcaagatagagagaagaactgagctgtaa

SEQ ID NO: 113 - DNA, 1713 bp, Artificial Sequence
  synthesized DNA of human FKBP63 ORF having codon optimized for yeast
atggcttttagaggatggagacctccacctcctcctttacttttgttgttgctttgggtaaccggacaggccgcacccgtggctggtttgggctctgacgccgagcttcaaattgaaagaaggtttgtgcccgacgagtgcccaagaaccgtaagaagtggagatttcgtccgttaccattacgtcggtacttttccagacggacaaaagttcgattcctcctatgatcgagattctacctttaatgttttcgttggaaaggggcagttaattacaggaatggatcaggccttagtcggtatgtgtgttaatgaacgaagatttgtcaaaattccaccgaagcttgcttatggtaacgaaggtgtttctggtgtcataccacctaatagcgtgttgcactttgatgtcctcttgatggatatttggaatagcgaagatcaggtgcaaatccatacttatttcaagcctcccagttgtccacgtacaattcaagtgtccgactttgtccgttaccactacaatggtacttttttggatggtactctgttcgacagttcgcacaatcgtatgaaaacttatgatacttatgtcggtattggttggttgatcccaggcatggataagggattactaggaatgtgtgtcggtgaaaagagaattatcactattcccccattcctggcctacggtgaagatggtgatggaaaggatataccaggtcaggcttcactcgttttcgatgttgctttgttggatcttcacaacccaaaagattccattagtatcgagaataaggttgtaccagagaattgtgagaggatatctcaatccggagacttcttaagataccactacaacgggactttgttagatgggaccctgttcgactcgtcgtattcaagaaacagaacattcgacacatatatcggccaaggatacgttattccgggaatggacgaaggattattgggcgtatgtataggcgagaagcgaaggatcgtagttcctccacatttgggttatggtgaggaagggcgtggtaatattccagggtcagctgttttggtttttgacattcatgttattgactttcataacccatcagactccatttctatcacatcacactataagccccctgactgctctgtgctttctaaaaagggtgactatctaaagtaccattacaacgcatctcttttggatggtacgttgctagactctacgtggaacttgggtaaaacatacaacatcgttcttggtagcggtcaggtcgttctgggtatggacatgggactacgagagatgtgcgtgggtgagaagaggaccgttataatccctccacatctgggatacggagaagcaggcgtggatggggaagttcctggttcagctgttcttgtgtttgatatagaactgctggagttggtagctggcctgcctgagggatatatgtttatttggaacggcgaagtatccccgaatctattcgaggaaatcgataaagacggtaacggagaagttctcttagaggaattctctgaatacatccatgcacaagttgccagtggtaaaggcaaactagctcctggatttgatgctgaactcattgttaagaatatgtttaccaaccaagatagaaacggtgatggaaaagtcactgccgaagagtttaaactgaaagatcaagaagcaaaacacgatgagttgtaa

SEQ ID NO: 114 - DNA, 1749 bp, Artificial Sequence
  synthesized DNA of human FKBP65 ORF having codon optimized for yeast
atgttccctgctggtcctccttctcattcgttacttcgtctgccgttacttcagttactgcttctggttgtccaggccgtcggtagaggtctaggtagagcctccccagcaggtggacctcttgaggatgtcgttattgagagataccatataccacgtgcttgtccgagagaagtgcaaatgggcgacttcgtccgatatcattacaacggcacttttgaggacggaaagaaattcgatagtagctacgatagaaatacactggttgctatcgttgtaggggttggacgactcatcaccggaatggataggggtttgatgggaatgtgtgtgaacgagaggcgtcgtttaatagtccctccccacctggggtatggatctatcggtttggctggattgattccacctgatgccactctctacttcgatgtcgttttactggatgtatggaacaaggaggatactgttcaagtttccacgttgttgaggccaccgcactgccctaggatggttcaggatggtgatttcgttcgttaccactataatggtactctcttagacggaacctttttcgatacctcttacagcaaaggcggaacttacgatacatatgtgggttctggttggttaatcaagggaatggatcaggggttgttgggtatgtgtccaggcgagagaaggaaaattattattcctccatttcttgcatacggtgaaaagggatacggtactgttattcctccccaagcatcactggtgttccacgttctcctgattgatgtccataacccaaaggatgccgtacaacttgagaccctagagttgcctccaggttgcgttagaagagccggtgcaggtgattttatgagataccattataacggctctctcatggacggtactttgttcgactcttcatattcaagaaaccacacttataatacatacatcggccagggctatatcattccagggatggatcaaggactacagggtgcatgtatgggtgaacgaagaagaattactataccgccacatttggcttatggtgagaatggtacgggtgacaagattcctggatcggctgtgctgatttttaacgtgcatgtgatcgacttccacaatccagcagatgttgtagaaattagaacattgagtagaccctcagaaacatgcaatgaaaccactaaattgggtgactttgtcagataccattacaattgtagtttattggacgggactcaattgttcacgtcccacgactatggtgctccacaagaggctacattaggggctaacaaagttattgaaggtcttgacactggattgcagggaatgtgtgtaggtgagagaagacaattgatcgtcccccctcatttggcccacggcgaaagtggagctcgtggtgttcccggatctgctgtattgctatttgaagtcgaacttgtttccagagaagacggattgccaaccggatatctgtttgtttggcataaggatccacctgctaacttgtttgaagatatggacctaaacaaggatggcgaggtgccacctgaagaattttccacctttattaaggctcaagtgtcagagggaaaaggaaggcttatgcccggtcaagatccagaaaagaccatcggggatatgtttcaaaatcaggaccgaaatcaagatggtaaaataacagttgacgaacttaaactaaaatctgacgaggatgaagaaagagtacacgaagagctataa

SEQ ID NO: 115 - DNA, 1185 bp, Artificial Sequence
  synthesized DNA of Zeocin(TM)-resistant cassette
cggccgcccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggcgggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctcggccg

SEQ ID NO: 116 - protein, 727 aa, Homo sapiens
MRPLLLLALLGWLLLAEAKGDAKPEDNLLVLTVATKETEGFRRFKRSAQFFNYKIQALGLGEDWNVEKGTSAGGGQKVRLLKKALEKHADKEDLVILFTDSYDVLFASGPRELLKKFRQSRSQVVFSAEELIYPDRRLETKYPVVSDGKRFLGSGGFIGYAPNLSKLVAEWEGQDSDSDQLFYTKIFLDPEKREQINITLDHRCRIFQNLDGALDEVVLKFEMGHVRARNLAYDTLPVLIHGNGPTKLQLNYLGNYIPRFWTFETGCTVCDEGLRSLKGIGDEALPTVLVGVFIEQPTPFVSLFFQRLLRLHYPQKHMRLFIHNHEQHHKAQVEEFLAQHGSEYQSVKLVGPEVRMANADARNMGADLCRQDRSCTYYFSVDADVALTEPNSLRLLIQQNKNVIAPLMTRHGRLWSNFWGALSADGYYARSEDYVDIVQGRRVGVWNVPYISNIYLIKGSALRGELQSSDLFHHSKLDPDMAFCANIRQQDVFMFLTNRHTLGHLLSLDSYRTTHLHNDLWEVFSNPEDWKEKYIHQNYTKALAGKLVETPCPDVYWFPIFTEVACDELVEEMEHFGQWSLGNNKDNRIQGGYENVPTIDIHMNQIGFEREWHKFLLEYIAPMTEKLYPGYYTRAQFDLAFVVRYKPDEQPSLMPHHDASTFTINIALNRVGVDYEGGGCRFLRYNCSIRAPRKGWTLMHPGRLTHYHEGLPTTRGTRYIAVSFVDP

SEQ ID NO: 117 - DNA, 2184 bp, Homo sapiens
atgcggcccctgctgctactggccctgctgggctggctgctgctggccgaagcgaagggcgacgccaagccggaggacaaccttttagtcctcacggtggccactaaggagaccgagggattccgtcgcttcaagcgctcagctcagttcttcaactacaagatccaggcgcttggcctaggggaggactggaatgtggagaaggggacgtcggcaggtggagggcagaaggtccggctgctgaagaaagctctggagaagcacgcagacaaggaggatctggtcattctcttcacagacagctatgacgtgctgtttgcatcggggccccgggagctcctgaagaagttccggcagtccaggagccaggtggtcttctctgctgaggagctcatctacccagaccgcaggctggagaccaagtatccggtggtgtccgatggcaagaggttcctgggctctggaggcttcatcggttatgcccccaacctcagcaaactggtggccgagtgggagggccaggacagcgacagcgatcagctgttttacaccaagatcttcttggacccggagaagagggagcagatcaatatcaccctggaccaccgctgccgtatcttccagaacctggatggagccttggatgaggtcgtgctcaagtttgaaatgggccatgtgagagcgaggaacctggcctatgacaccctcccggtcctgatccatggcaacgggccaaccaagctgcagttgaactacctgggcaactacatcccgcgcttctggaccttcgaaacaggctgcaccgtgtgtgacgaaggcttgcgcagcctcaagggcattggggatgaagctctgcccacggtcctggtcggcgtgttcatcgaacagcccacgccgtttgtgtccctgttcttccagcggctcctgcggctccactacccccagaaacacatgcgacttttcatccacaaccacgagcagcaccacaaggctcaggtggaagagttcctggcacagcatggcagcgagtaccagtctgtgaagctggtgggccctgaggtgcggatggcgaatgcagatgccaggaacatgggcgcagacctgtgccggcaggaccgcagctgcacctactacttcagcgtggatgctgacgtggccctgaccgagcccaacagcctgcggctgctgatccaacagaacaagaacgtcattgccccgctgatgacccggcatgggaggctgtggtcgaacttctggggggctctcagtgcagatggctactatgcccgttccgaggactacgtggacattgtgcaggggcggcgtgttggtgtctggaatgtgccctatatttcaaacatctacttgatcaagggcagtgccctgcggggtgagctgcagtcctcagatctcttccaccacagcaagctggaccccgacatggccttctgtgccaacatccggcagcaggatgtgttcatgttcctgaccaaccggcacacccttggccatctgctctccctagacagctaccgcaccacccacctgcacaacgacctctgggaggtgttcagcaaccccgaggactggaaggagaagtacatccaccagaactacaccaaagccctggccgggaagctggtggagacgccctgcccggatgtctattggttccccatcttcacggaggtggcctgtgatgagctggtggaggagatggagcactttggccagtggtctctgggcaacaacaaggacaaccgcatccagggtggctacgagaacgtgccgactattgacatccacatgaaccagatcggctttgagcgggagtggcacaaattcctgctggagtacattgcgcccatgacggagaagctctaccccggctactacaccagggcccagtttgacctggcctttgtcgtccgctacaagcctgatgagcagccctcactgatgccacaccatgatgcctccaccttcaccatcaacatcgccctgaaccgagtcggggtggattacgagggcgggggctgtcggttcctgcgctacaactgttccatccgagccccaaggaagggctggaccctcatgcaccctggacgactcacgcattaccatgaggggctccccaccaccaggggcacccgctacatcgcagtctccttcgtcgatccctaa

SEQ ID NO: 118 - protein, 738 aa, Homo sapiens
MTSSGPGPRFLLLLPLLLPPAASASDRPRGRDPVNPEKLLVITVATAETEGYLRFLRSAEFFNYTVRTLGLGEEWRGGDVARTVGGGQKVRWLKKEMEKYADREDMIIMFVDSYDVILAGSPTELLKKFVQSGSRLLFSAESFCWPEWGLAEQYPEVGTGKRFLNSGGFIGFATTIHQIVRQWKYKDDDDDQLFYTRLYLDPGLREKLSLNLDHKSRIFQNLNGALDEVVLKFDRNRVRIRNVAYDTLPIVVHGNGPTKLQLNYLGNYVPNGWTPEGGCGFCNQDRRTLPGGQPPPRVFLAVFVEQPTPFLPRFLQRLLLLDYPPDRVTLFLHNNEVFHEPHIADSWPQLQDHFSAVKLVGPEEALSPGEARDMAMDLCRQDPECEFYFSLDADAVLTNLQTLRILIEENRKVIAPMLSRHGKLWSNFWGALSPDEYYARSEDYVELVQRKRVGVWNVPYISQAYVIRGDTLRMELPQRDVFSGSDTDPDMAFCKSFRDKGIFLHLSNQHEFGRLLATSRYDTEHLHPDLWQIFDNPVDWKEQYIHENYSRALEGEGIVEQPCPDVYWFPLLSEQMCDELVAEMEHYGQWSGGRHEDSRLAGGYENVPTVDIHMKQVGYEDQWLQLLRTYVGPMTESLFPGYHTKARAVMNFVVRYRPDEQPSLRPHHDSSTFTLNVALNHKGLDYEGGGCRFLRYDCVISSPRKGWALLHPGRLTHYHEGLPTTWGTRYIMVSFVDP

SEQ ID NO: 119 - DNA, 2217 bp, Homo sapiens
atgacctcctcggggcctggaccccggttcctgctgctgctgccgctgctgctgccccctgcggcctcagcctccgaccggccccggggccgagacccggtcaacccagagaagctgctggtgatcactgtggccacagctgaaaccgaggggtacctgcgtttcctgcgctctgcggagttcttcaactacactgtgcggaccctgggcctgggagaggagtggcgagggggtgatgtggctcgaacagttggtggaggacagaaggtccggtggttaaagaaggaaatggagaaatacgctgaccgggaggatatgatcatcatgtttgtggatagctacgacgtgattctggccggcagccccacagagctgctgaagaagttcgtccagagtggcagccgcctgctcttctctgcagagagcttctgctggcccgagtgggggctggcggagcagtaccctgaggtgggcacggggaagcgcttcctcaattctggtggattcatcggttttgccaccaccatccaccaaatcgtgcgccagtggaagtacaaggatgatgacgacgaccagctgttctacacacggctctacctggacccaggactgagggagaaactcagccttaatctggatcataagtctcggatctttcagaacctcaacggggctttagatgaagtggttttaaagtttgatcggaaccgtgtgcgtatccggaacgtggcctacgacacgctccccattgtggtccatggaaacggtcccactaagctgcagctcaactacctgggaaactacgtccccaatggctggactcctgagggaggctgtggcttctgcaaccaggaccggaggacactcccgggggggcagcctcccccccgggtgtttctggccgtgtttgtggaacagcctactccgtttctgccccgcttcctgcagcggctgctactcctggactatccccccgacagggtcacccttttcctgcacaacaacgaggtcttccatgaaccccacatcgctgactcctggccgcagctccaggaccacttctcagctgtgaagctcgtggggccggaggaggctctgagcccaggcgaggccagggacatggccatggacctgtgtcggcaggaccccgagtgtgagttctacttcagcctggacgccgacgctgtcctcaccaacctgcagaccctgcgtatcctcattgaggagaacaggaaggtgatcgcccccatgctgtcccgccacggcaagctgtggtccaacttctggggcgccctgagccccgatgagtactacgcccgctccgaggactacgtggagctggtgcagcggaagcgagtgggtgtgtggaatgtaccatacatctcccaggcctatgtgatccggggtgataccctgcggatggagctgccccagagggatgtgttctcgggcagtgacacagacccggacatggccttctgtaagagctttcgagacaagggcatcttcctccatctgagcaatcagcatgaatttggccggctcctggccacttccagatacgacacggagcacctgcaccccgacctctggcagatcttcgacaaccccgtcgactggaaggagcagtacatccacgagaactacagccgggccctggaaggggaaggaatcgtggagcagccatgcccggacgtgtactggttcccactgctgtcagaacaaatgtgtgatgagctggtggcagagatggagcactacggccagtggtcaggcggccggcatgaggattcaaggctggctggaggctacgagaatgtgcccaccgtggacatccacatgaagcaggtggggtacgaggaccagtggctgcagctgctgcggacgtatgtgggccccatgaccgagagcctgtttcccggttaccacaccaaggcgcgggcggtgatgaactttgtggttcgctaccggccagacgagcagccgtctctgcggccacaccacgactcatccaccttcaccctcaacgttgccctcaaccacaagggcctggactatgagggaggtggctgccgcttcctgcgctacgactgtgtgatctcctccccgaggaagggctgggcactcctgcaccccggccgcctcacccactaccacgaggggctgccaacgacctggggcacacgctacatcatggtgtcctttgtcgacccctga

SEQ ID NO: 120 - protein, 418 aa, Homo sapiens
MRSLLLLSAFCLLEAALAAEVKKPAAAAAPGTAEKLSPKAATLAERSAGLAFSLYQAMAKDQAVENILVSPVVVASSLGLVSLGGKATTASQAKAVLSAEQLRDEEVHAGLGELLRSLSNSTARNVTWKLGSRLYGPSSVSFADDFVRSSKQHYNCEHSKINFRDKRSALQSINEWAAQTTDGKLPEVTKDVERTDGALLVNAMFFKPHWDEKFHHKMVDNRGFMVTRSYTVGVMMMHRTGLYNYYDDEKEKLQIVEMPLAHKLSSLIILMPHHVEPLERLEKLLTKEQLKIWMGKMQKKAVAISLPKGVVEVTHDLQKHLAGLGLTEAIDKNKADLSRMSGKKDLYLASVFHATAFELDTDGNPFDQDIYGREELRSPKLFYADHPFIFLVRDTQSGSLLFIGRLVRPKGDKMRDEL

SEQ ID NO: 121 - DNA, 1257 bp, Homo sapiens
atgcgctccctcctgcttctcagcgccttctgcctcctggaggcggccctggccgccgaggtgaagaaacctgcagccgcagcagctcctggcactgcggagaagttgagccccaaggcggccacgcttgccgagcgcagcgccggcctggccttcagcttgtaccaggccatggccaaggaccaggcagtggagaacatcctggtgtcacccgtggtggtggcctcgtcgctggggctcgtgtcgctgggcggcaaggcgaccacggcgtcgcaggccaaggcagtgctgagcgccgagcagctgcgcgacgaggaggtgcacgccggcctgggcgagctgctgcgctcactcagcaactccacggcgcgcaacgtgacctggaagctgggcagccgactgtacggacccagctcagtgagcttcgctgatgacttcgtgcgcagcagcaagcagcactacaactgcgagcactccaagatcaacttccgcgacaagcgcagcgcgctgcagtccatcaacgagtgggccgcgcagaccaccgacggcaagctgcccgaggtcaccaaggacgtggagcgcacggacggcgccctgttagtcaacgccatgttcttcaagccacactgggatgagaaattccaccacaagatggtggacaaccgtggcttcatggtgactcggtcctataccgtgggtgtcatgatgatgcaccggacaggcctctacaactactacgacgacgagaaggaaaagctgcaaatcgtggagatgcccctggcccacaagctctccagcctcatcatcctcatgccccatcacgtggagcctctcgagcgccttgaaaagctgctaaccaaagagcagctgaagatctggatggggaagatgcagaagaaggctgttgccatctccttgcccaagggtgtggtggaggtgacccatgacctgcagaaacacctggctgggctgggcctgactgaggccattgacaagaacaaggccgacttgtcacgcatgtcaggcaagaaggacctgtacctggccagcgtgttccacgccaccgcctttgagttggacacagatggcaacccctttgaccaggacatctacgggcgcgaggagctgcgcagccccaagctgttctacgccgaccaccccttcatcttcctagtgcgggacacccaaagcggctccctgctattcattgggcgcctggtccggcctaagggtgacaagatgcgagacgagttatag

(19) United States
(12) Patent Application Publication
(10) Pub. No.: US 2013/0090455 A1
(43) Pub. Date: Apr. 11, 2013

(54) TRANSFORMANT WHICH PRODUCES GLYCINE REPEAT SEQUENCE PROTEIN

(71) Applicant: Shoichi Nishio, Ikeda-shi (JP)

(72) Inventor: Shoichi Nishio, Ikeda-shi (JP)

(21) Appl. No.: 13/634,901

(22) PCT Filed: Mar. 14, 2011
(86) PCT No.: PCT/JP2011/055960

§ 371 Date: Sep. 14, 2012

(30) Foreign Application Priority Data
Mar. 15, 2010 (JP) 2010-057484

(51) Int. Cl. C07K 14/78

(52) U.S. Cl. CPC C07K 14/78
USPC 530/356; 435/254.2; 435/69.1

(57) ABSTRACT

Disclosed is a transformant in which polynucleotides comprising a nucleotide sequence encoding the amino acid sequence of all of the following proteins (1) to (3) are transfected into a microbial cell:
(1) a protein that is capable of binding to FK506 and has a molecular weight of 15,000 or more and 60,000 or less;
(2) a prolyl hydroxylase; and
(3) a glycine repeat sequence protein having the following characteristics (A) and (B):
<characteristic (A)> the polypeptide chain of the glycine repeat sequence protein having a continuous, Gly-Xaa-Yaa repeating sequence, and
<characteristic (B)> an imino acid being contained in the continuous, Gly-Xaa-Yaa repeating sequence of the polypeptide chain of the glycine repeat sequence protein.

### TECHNICAL FIELD

The present invention relates to a transformant that produces a glycine repeat sequence protein and to a process for obtaining a glycine repeat sequence protein.

### BACKGROUND ART

A group of proteins having a glycine repeating sequence are present within a living body and are known to play important roles in maintaining the structures of tissues, intercellular adhesion, wound healing etc., in a living organism. The group of these proteins is characterized in that each protein has a high content of imino acid (e.g. proline or hydroxyproline) and possesses a unique triple-helical structure, and is collectively referred to as collagen.

Collagens are contained in various types of body tissues, typically including skin, tendon, cartilage, ligament, blood vessel and bone. Due to high tensile strength of collagens, body tissues containing collagens have a high tolerance for mechanical stress.

Natural or artificial glycine repeat sequence proteins as represented by collagens, which have excellent physical properties as well as a variety of bioactivities, are available as pharmaceuticals, industrial products, cosmetics, or foods, and so, it can be regarded as a commercially valuable versatile material.

Glycine repeat sequence proteins which are commercially produced are mainly natural form collagen, which are produced by purification from animal skins, tendons, bones and others.

Collagens are found in substantially all of the multicellular animals and occupy an approximately 30% of the protein mass present in the body. It is known that higher animals may have 20 or more different types of collagen. Collagens which are present in high amounts in a living organism include Type I collagen, Type II collagen and Type III collagen. Type I collagen is a major fibrillar collagen present in bone, tendon and skin, which is a heterotrimeric molecule comprising two α1(I) chains and one α2(I) chain. Type II collagen is a collagen present in cartilage and vitreous body, which is a homotrimeric molecule comprising three identical α1(II) chains. Type III collagen is a collagen present in skin and blood vessel, which is a homotrimeric molecule comprising three identical α1(III) chains. Type I, Type II and Type III collagens can be purified from body tissues, for example, by the procedures described in Non-Patent Documents 1, 4 and 5.

As techniques for producing glycine repeat sequence proteins with improved physical properties and bioactivities, there have been developed techniques for producing proteins which have a non-naturally occurring, artificial glycine-repeating sequence, as described below. Non-Patent Document 1 discloses a process for producing a glycine repeat sequence protein, in which chemically synthesized peptides are subjected to formation of a triple-helical structure, followed by chemical polymerization. Patent Document 2 discloses a technique for producing a polymer which forms a triple helix by employing self-assembling of chemically synthesized peptides. However, these methods in which chemical synthesis is employed use expensive raw materials and require multistep procedures, and thus there is a need for a simple production process using inexpensive raw materials.

As techniques for producing glycine repeat sequence proteins using inexpensive raw materials, there have been developed production processes employing recombinant cells. Non-Patent Document 6 discloses Type I collagen with hydroxylated proline residues, produced by coexpressing a precursor of Type I collagen and a prolyl hydroxylase in a yeast cell. On the other hand, Non-Patent Document 1 discloses Type I or Type III collagen with hydroxylated proline residues is prepared by coexpressing a precursor of a Type I or Type III collagen and a prolyl hydroxylase in a yeast cell. Patent Document 3 discloses a process for producing a non-naturally occurring, artificial glycine repeat sequence protein using a gene recombinant cell. However, there is a need for a more efficient process for producing a highly functional, glycine repeat sequence protein.

### PRIOR ART DOCUMENTS

### Patent Documents

Patent Document 1: WO 97/38710 A
Patent Document 2: JP 2005-263784 A
Patent Document 3: U.S. Pat. No. 5,710,252

### Non-Patent Documents

Non-Patent Document 1: Masao Tanihara supervised, “Production and Development of Applications of Collagen,” ISBN: 978-4-7813-0071-3, CMC Publishing Co., Ltd.;
Non-Patent Document 2: Volume Editors: Brinckmann, J., Notbohm, H., Muller, P. K., Collagen primer in Structure, Processing and Assembly, Topics in Current Chemistry Vol. 247, 2005;
Non-Patent Document 3: Gelse, K., Poschl, E., Aigner, T., Collagens—structure, function and biosynthesis, Advanced Drug Delivery Reviews, 55:1531-1546 (2003);
Non-Patent Document 4: Miller et al., Methods In Enzymology, 82: 33-64 (1982), Academic Press;
Non-Patent Document 5: Byers et al., Biochemistry, 13:5243-5248 (1974);
Non-Patent Document 6: P. David Toman et al., J. Biol. Chem., Vol. 275, No. 30, July 28, p 23303-23309 (2000).

### DISCLOSURE OF THE INVENTION

### Problems to be Solved by the Invention

There has been a need to find a transformant producing a glycine repeat sequence protein which is usable as a high performance versatile material that is more commercially valuable for pharmaceuticals, industrial products, cosmetics, foods etc., in a state where its inherent physical properties are not impaired (e.g. in a state where changes in the physical properties related to the hydrophilicity of glycine repeat sequence proteins are reduced so as to exhibit more lipophilic properties that are similar to those inherently possessed by glycine repeat sequence proteins), and there has been a need to develop a process for producing a glycine repeat sequence protein using the obtained new transformant.

### Means for Solving the Problems

The present invention provides:

1. A transformant in which all of the following polynucleotides (1), (2) and (3) are transfected into a microbial cell:
(1) a polynucleotide comprising a nucleotide sequence encoding the amino acid sequence of an FK506 binding protein that is capable of binding to FK506 and has a molecular weight of 15,000 or more and 60,000 or less;
(2) a polynucleotide comprising a nucleotide sequence encoding the amino acid sequence of a prolyl hydroxylase; and
(3) a polynucleotide comprising a nucleotide sequence encoding the amino acid sequence of a glycine repeat sequence protein having the following characteristics (A) and (B):
<characteristic (A)> the polypeptide chain of the glycine repeat sequence protein having a continuous, Gly-Xaa-Yaa repeating sequence, and
<characteristic (B)> an imino acid (e.g. proline or hydroxyproline) being contained in the continuous, Gly-Xaa-Yaa repeating sequence of the polypeptide chain of the glycine repeat sequence protein,
wherein Xaa and Yaa each represent any amino acid;
2. The transformant according to the above 1, which produces all of the proteins encoded by the polynucleotides (1), (2) and (3) within the cell;
3. The transformant according to the above 1 or 2, wherein the FK506 binding protein is FKBP23 or FKBP19;
4. The transformant according to the above 1 or 2, wherein the FK506 binding protein is FKBP23;
5. The transformant according to any one of the above 1 to 4, wherein the polynucleotide comprising a nucleotide sequence encoding the amino acid sequence of the FK506 binding protein is linked to downstream of a yeast derived promoter;
6. The transformant according to any one of the above 1 to 5, which further produces a lysyl hydroxylase within the cell;
7. The transformant according to the above 6, wherein the lysyl hydroxylase is at least one lysyl hydroxylase selected from lysyl hydroxylase 1 and lysyl hydroxylase 3;
8. The transformant according to any one of the above 1 to 5, which further produces Hsp47 within the cell;
9. The transformant according to any one of the above 1 to 8, wherein the prolyl hydroxylase is prolyl 4-hydroxylase;
10. The transformant according to any one of the above 1 to 8, wherein the prolyl hydroxylase is an enzyme comprising a prolyl 4-hydroxylase α subunit and a prolyl 4-hydroxylase β subunit;
11. The transformant according to the above 10, wherein a yeast α-factor prepro sequence is fused at the amino terminus of the prolyl 4-hydroxylase β subunit;
12. The transformant according to the above 10 or 11, wherein the prolyl 4-hydroxylase α subunit is an α1 subunit, an α2 subunit, or an α3 subunit;
13. The transformant according to any one of the above 1 to 12, wherein at least one polynucleotide comprising a nucleotide sequence encoding the amino acid sequence of the prolyl hydroxylase protein is linked to downstream of a yeast derived promoter;
14. The transformant according to any one of the above 1 to 13, wherein the glycine repeat sequence protein having the characteristics (A) and (B) is at least one collagen selected from among collagens of Type I to Type XXIX;
15. The transformant according to any one of the above 1 to 13, wherein the glycine repeat sequence protein having the characteristics (A) and (B) is at least one collagen selected from among collagen Type I, collagen Type II and collagen Type III;
16. The transformant according to any one of the above 1 to 15, wherein the microbial cell is an eukaryote cell;
17. The transformant according to the above 16, wherein the eukaryote cell is a yeast cell;
18. The transformant according to the above 17, wherein the yeast cell is a methanol-utilizing yeast cell;
19. The transformant according to the above 18, wherein the methanol-utilizing yeast cell is Komagataella pastoris;
20. A glycine repeat sequence protein having the characteristics (A) and (B) which is obtainable by being produced by the transformant according to any one of the above 1 to 19;
21. A process for producing a glycine repeat sequence protein, comprising:

a first step of transfecting all of the following polynucleotides (1), (2) and (3) into a microbial cell:

(1) a polynucleotide comprising a nucleotide sequence encoding the amino acid sequence of an FK506 binding protein that is capable of binding to FK506 and has a molecular weight of 15,000 or more and 60,000 or less,
(2) a polynucleotide comprising a nucleotide sequence encoding the amino acid sequence of a prolyl hydroxylase, and
(3) a polynucleotide comprising a nucleotide sequence encoding the amino acid sequence of a glycine repeat sequence protein having the following characteristics (A) and (B):
<characteristic (A)> the polypeptide chain of the glycine repeat sequence protein having a continuous, Gly-Xaa-Yaa repeating sequence, and
<characteristic (B)> an imino acid (e.g. proline or hydroxyproline) being contained in the continuous, Gly-Xaa-Yaa repeating sequence of the polypeptide chain of the glycine repeat sequence protein,
wherein Xaa and Yaa each represent any amino acid;

a second step of culturing the transformant resulting from the first step, thereby producing the glycine repeat sequence protein; and

a third step of collecting the glycine repeat sequence protein produced in the second step; and

22. A glycine repeat sequence protein produced by the process according to the above 21.

### Effects of the Invention

According to the present invention, there can be provided a transformant producing a glycine repeat sequence protein which is usable as a high performance versatile material that is more commercially valuable for pharmaceuticals, industrial products, cosmetics, foods etc., in a state where its inherent physical properties are not impaired (e.g. in a state where changes in the physical properties related to the hydrophilicity of glycine repeat sequence proteins are reduced so as to exhibit more lipophilic properties that are similar to those inherently possessed by glycine repeat sequence proteins), and a process for producing a glycine repeat sequence protein using the transformant.

### BRIEF DESCRIPTION OF THE DRAWINGS

FIG. 1 is a flow chart illustrating a process of constructing a plasmid for transfection of an expression cassette for a prolyl 4-hydroxylase α1 subunit and an expression cassette for a prolyl 4-hydroxylase β subunit.

FIG. 2 is a flow chart illustrating a process of constructing a plasmid for transfection of an expression cassette for an FK506 binding protein (FKBP).

FIG. 3 is a flow chart illustrating a process of constructing a plasmid for transfection of an expression cassette for an FK506 binding protein (FKBP).

FIG. 4 is a flow chart illustrating a process of constructing a plasmid for transfection of an expression cassette for an FK506 binding protein (FKBP).

FIG. 5 is a flow chart illustrating a process of constructing a plasmid for transfection of an expression cassette for human collagen Type I α1 and an expression cassette for human collagen Type I α2.

FIG. 6 is a flow chart illustrating a process of constructing a plasmid for transfection of an expression cassette for a fusion polypeptide consisting of the N-terminal non-helix region, 60 N-terminal residues of the helix region, 15 C-terminal residues of the helix region and the C-terminal non-helix region of human collagen Type I α1 and an expression cassette for a fusion polypeptide consisting of the N-terminal non-helix region, 60 N-terminal residues of the helix region, 15 C-terminal residues of the helix region and the C-terminal non-helix region of human collagen Type I α2.

FIG. 7 is a flow chart illustrating a process of constructing a plasmid for transfection of an expression cassette for a fusion polypeptide consisting of the N-terminal non-helix region, 27 N-terminal residues of the helix region, a dimer made of 522 central residues of the helix region, 15 C-terminal residues of the helix region and the C-terminal non-helix region of human collagen Type I α1 and an expression cassette for a fusion polypeptide consisting of the N-terminal non-helix region, 27 N-terminal residues of the helix region, a dimer made of 522 central residues of the helix region, 15 C-terminal residues of the helix region and the C-terminal non-helix region of human collagen Type I α2.

FIG. 8 is a flow chart illustrating a process of constructing a plasmid for introducing an expression cassette for human collagen Type III.

FIG. 9 is a flow chart illustrating a process of constructing a plasmid for transfection of an expression cassette for human collagen Type II.

FIG. 10 is a schematic diagram showing a plasmid for transfection of an expression cassette for a prolyl 4-hydroxylase α1 subunit and an expression cassette for a prolyl 4-hydroxylase β subunit.

FIG. 11 is a schematic diagram showing a plasmid for transfection of an expression cassette for an FKBP.

FIG. 12 is a schematic diagram showing a plasmid for transfection of an expression cassette for an FKBP.

FIG. 13 is a schematic diagram showing a plasmid for transfection of an expression cassette for an FKBP.

FIG. 14 is a schematic diagram showing a plasmid for transfection of an expression cassette for an FKBP.

FIG. 15 is a schematic diagram showing a plasmid for transfection of an expression cassette for human collagen Type I.

FIG. 16 is a schematic diagram showing a plasmid for transfection of an expression cassette for a fusion polypeptide consisting of the N-terminal non-helix region, 60 N-terminal residues of the helix region, 15 C-terminal residues of the helix region and the C-terminal non-helix region of human collagen Type I α1 and an expression cassette for a fusion polypeptide consisting of the N-terminal non-helix region, 60 N-terminal residues of the helix region, 15 C-terminal residues of the helix region and the C-terminal non-helix region of human collagen Type I α2.

FIG. 17 is a schematic diagram showing a plasmid for transfection of an expression cassette for a fusion polypeptide consisting of the N-terminal non-helix region, 27 N-terminal residues of the helix region, a dimer made of 522 central residues of the helix region, 15 C-terminal residues of the helix region and the C-terminal non-helix region of human collagen Type I α1 and an expression cassette for a fusion polypeptide consisting of the N-terminal non-helix region, 27 N-terminal residues of the helix region, a dimer made of 522 central residues of the helix region, 15 C-terminal residues of the helix region and the C-terminal non-helix region of human collagen Type I α2.

FIG. 18 is a schematic diagram showing a plasmid for transfection of an expression cassette for human collagen Type III.

FIG. 19 is a schematic diagram showing a plasmid for transfection of an expression cassette for human collagen Type II.

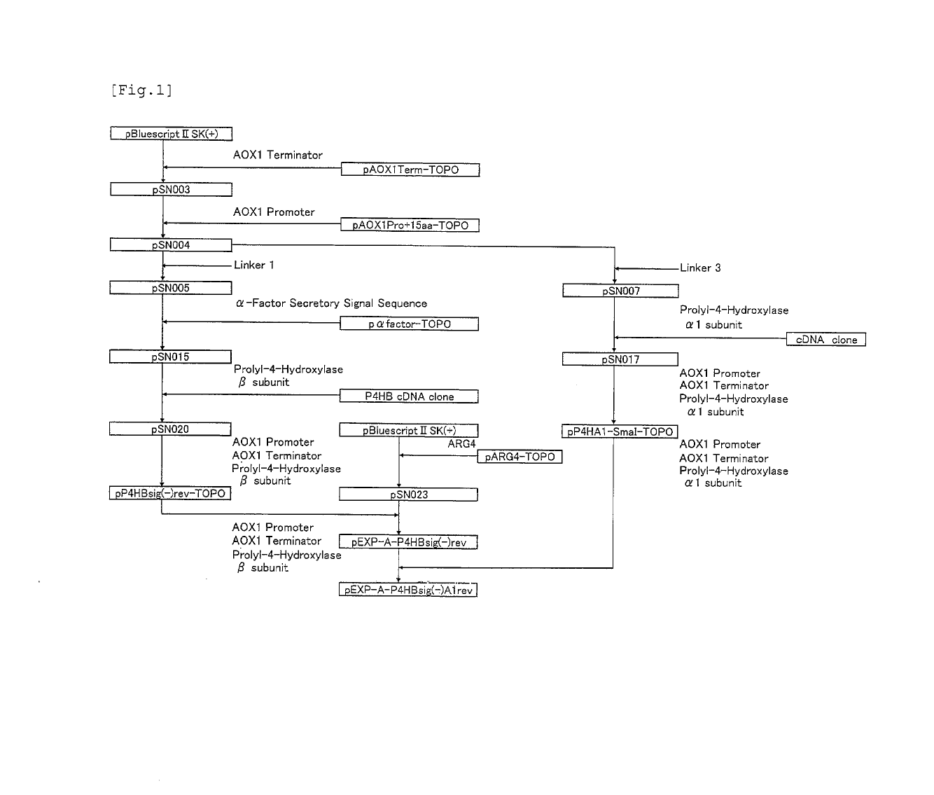

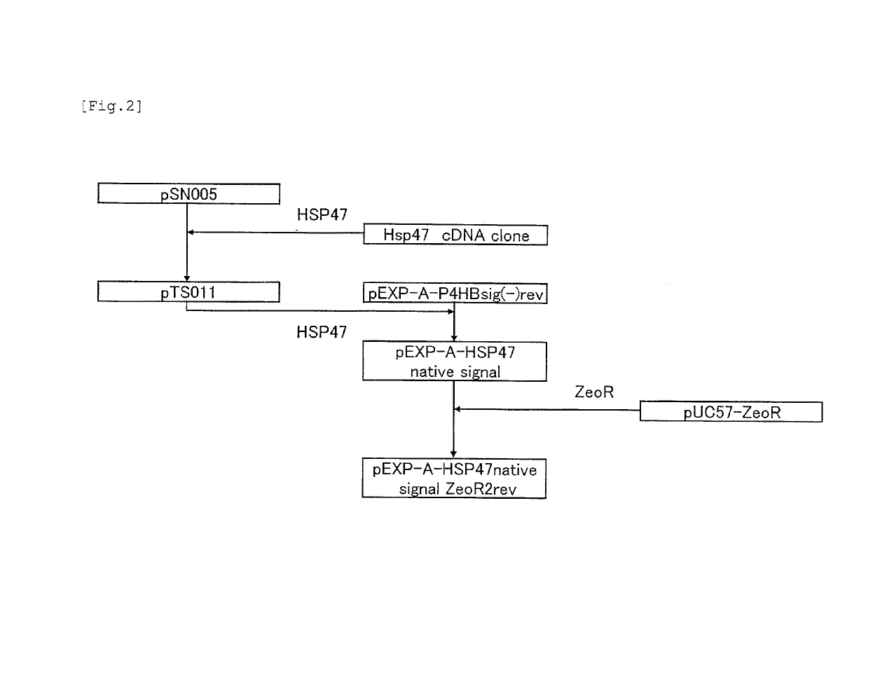

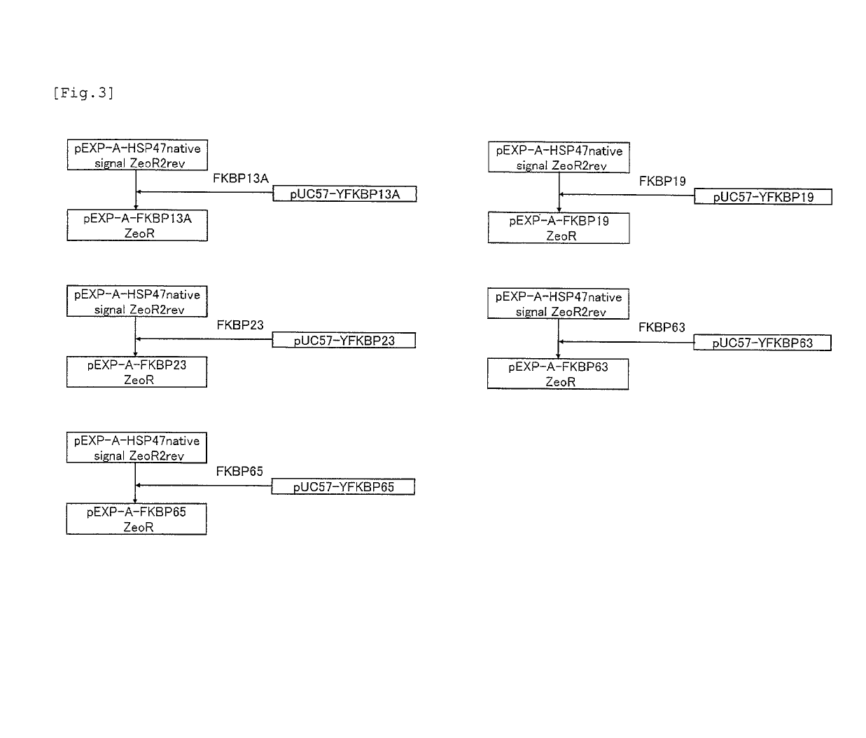

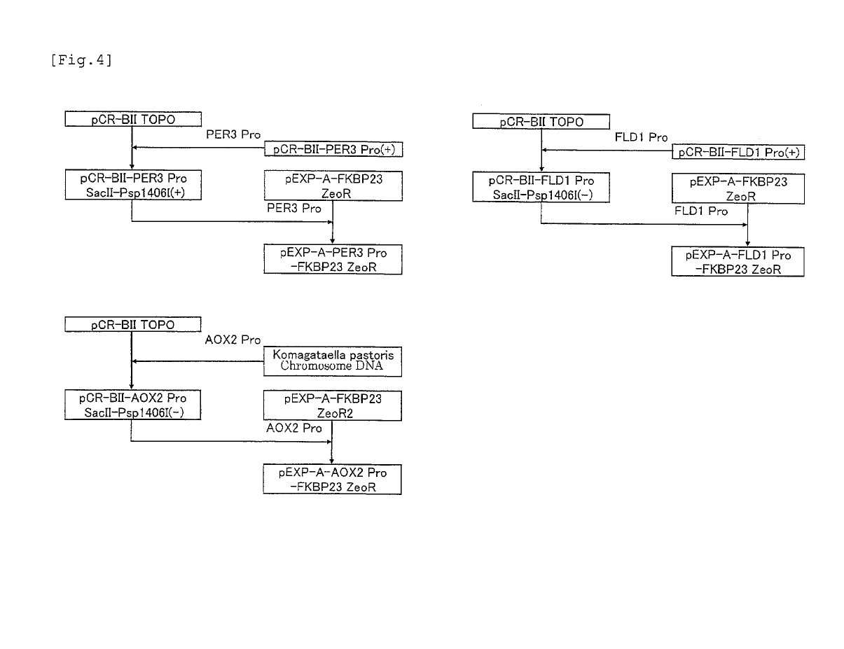

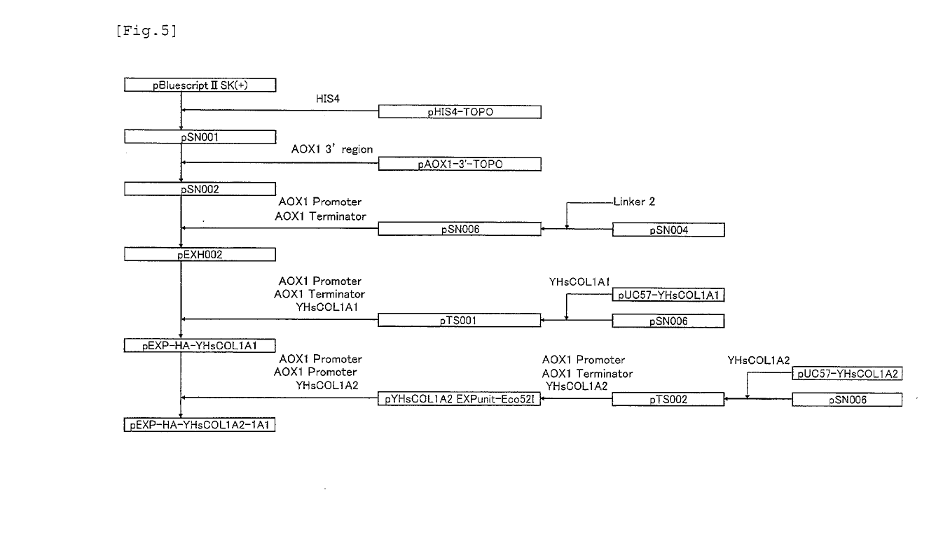

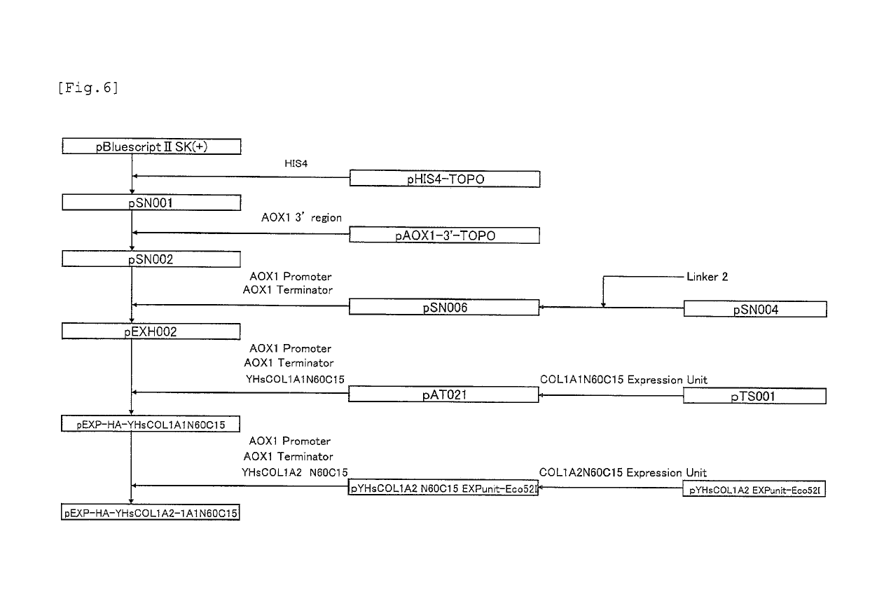

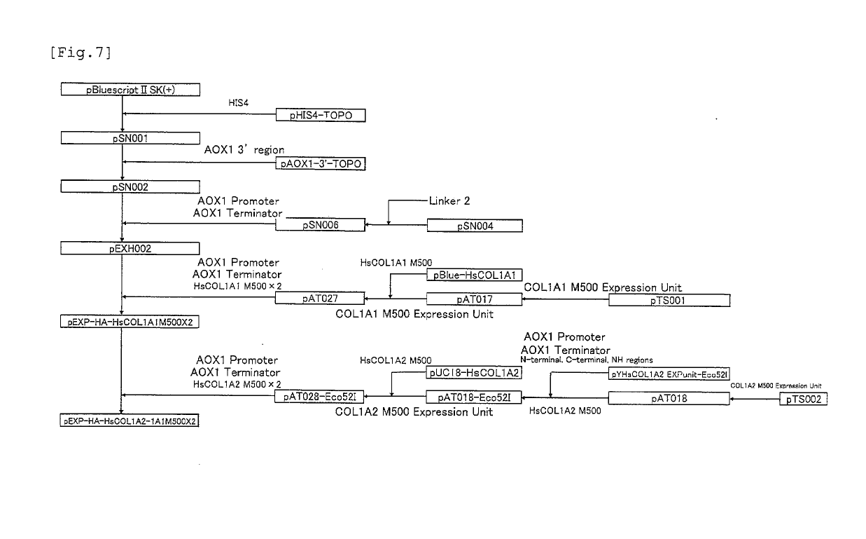

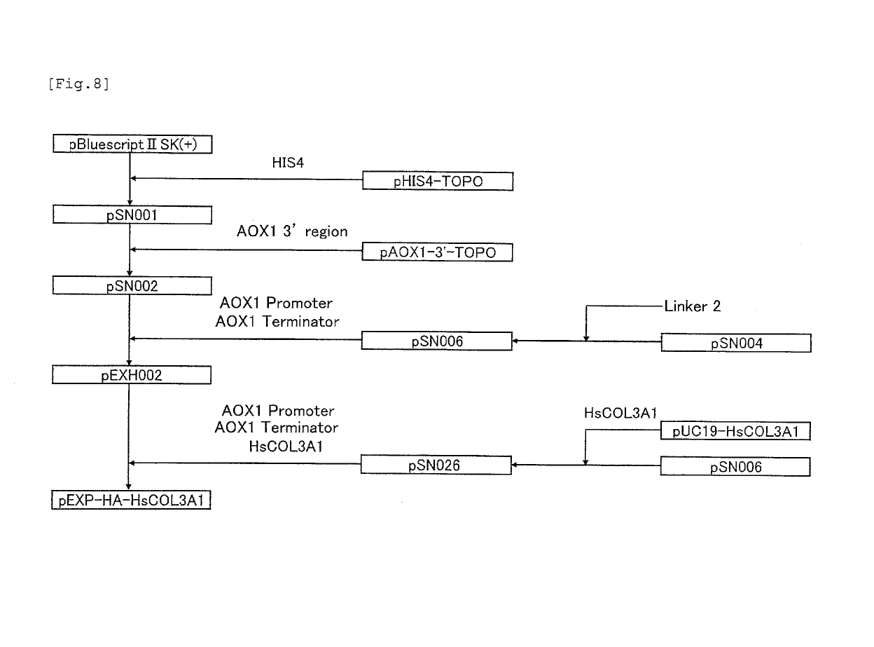

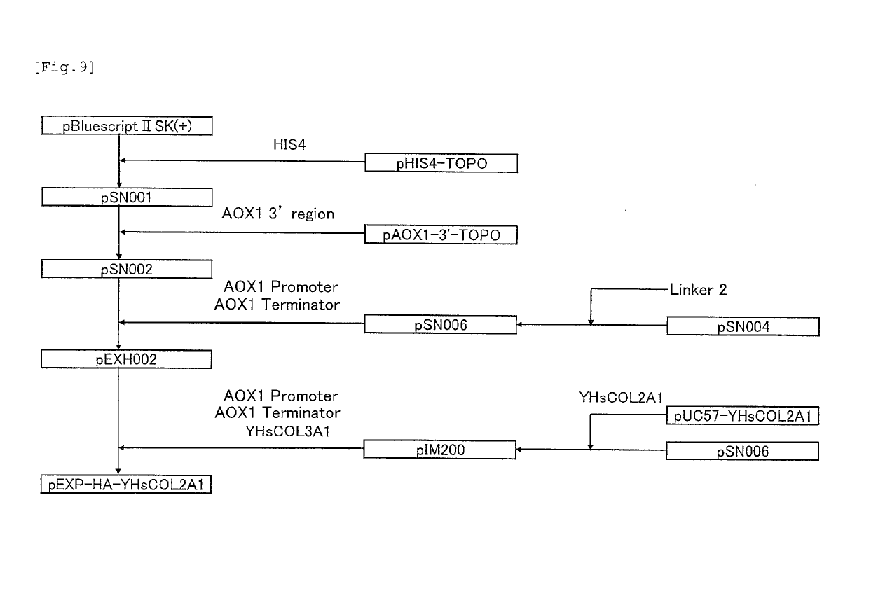

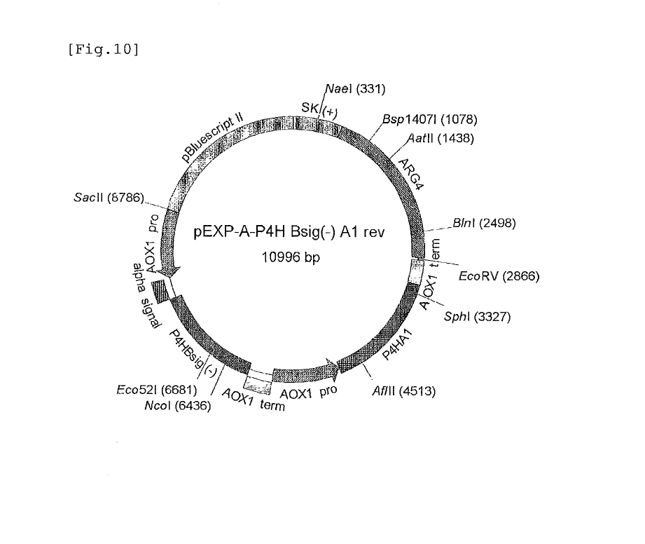

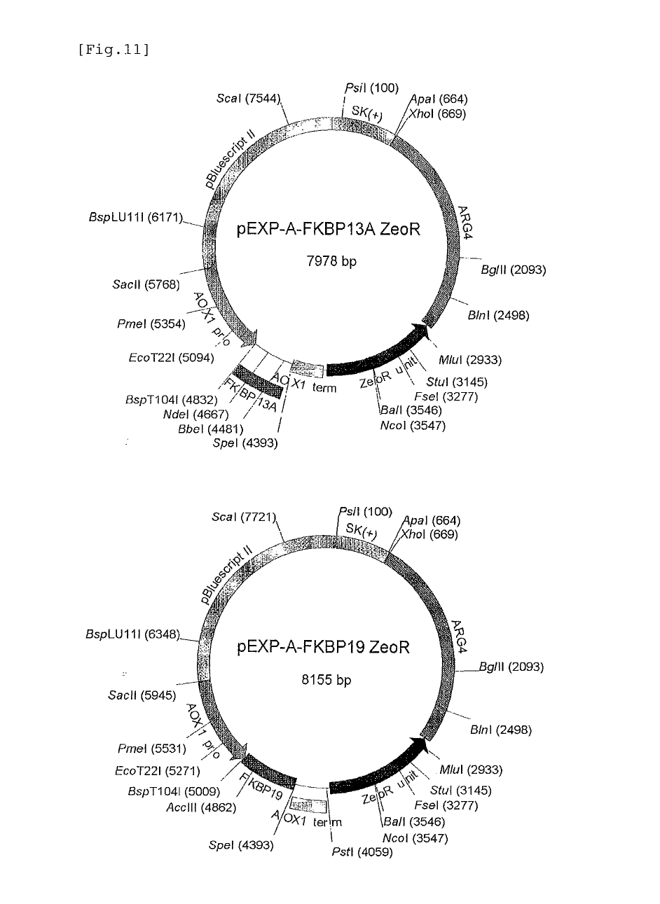

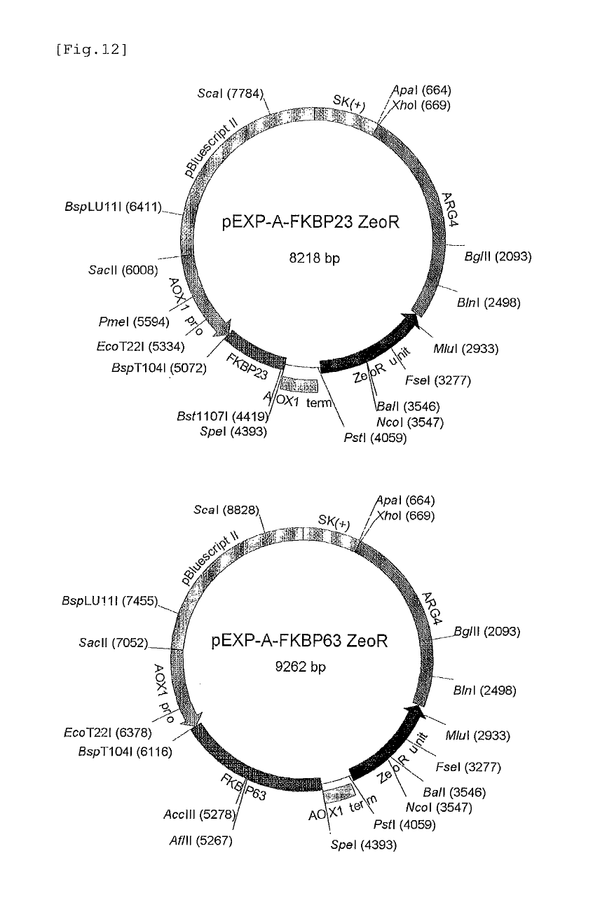

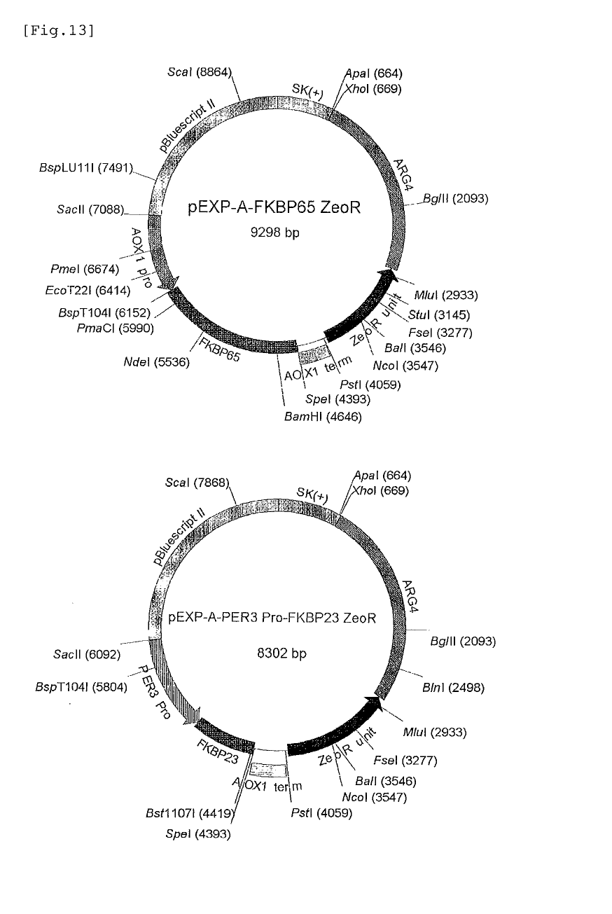

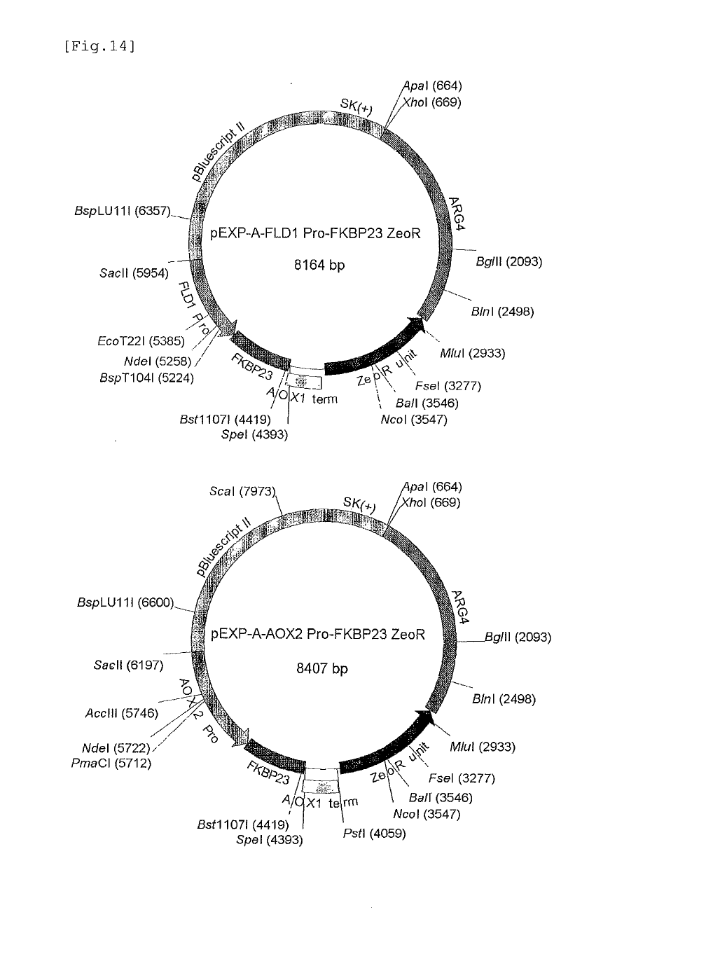

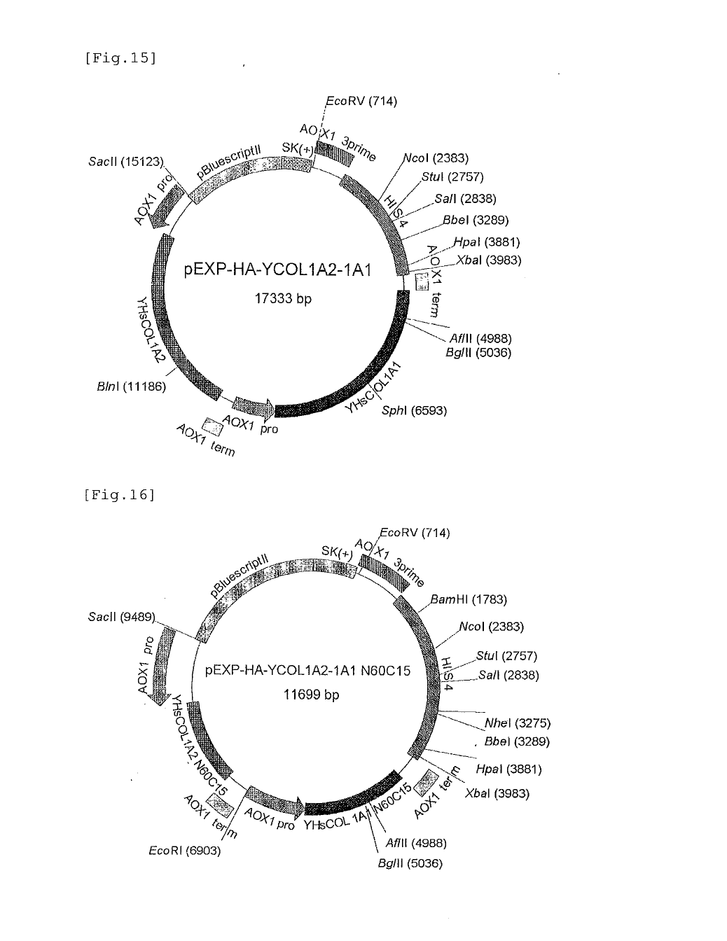

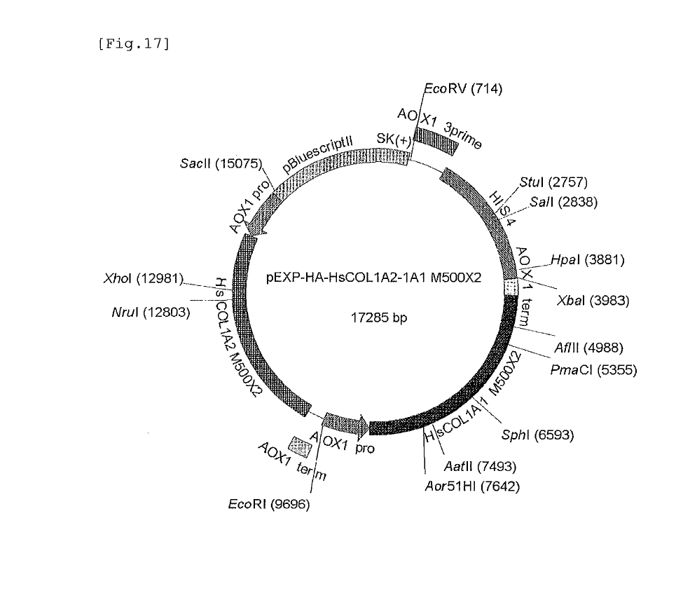

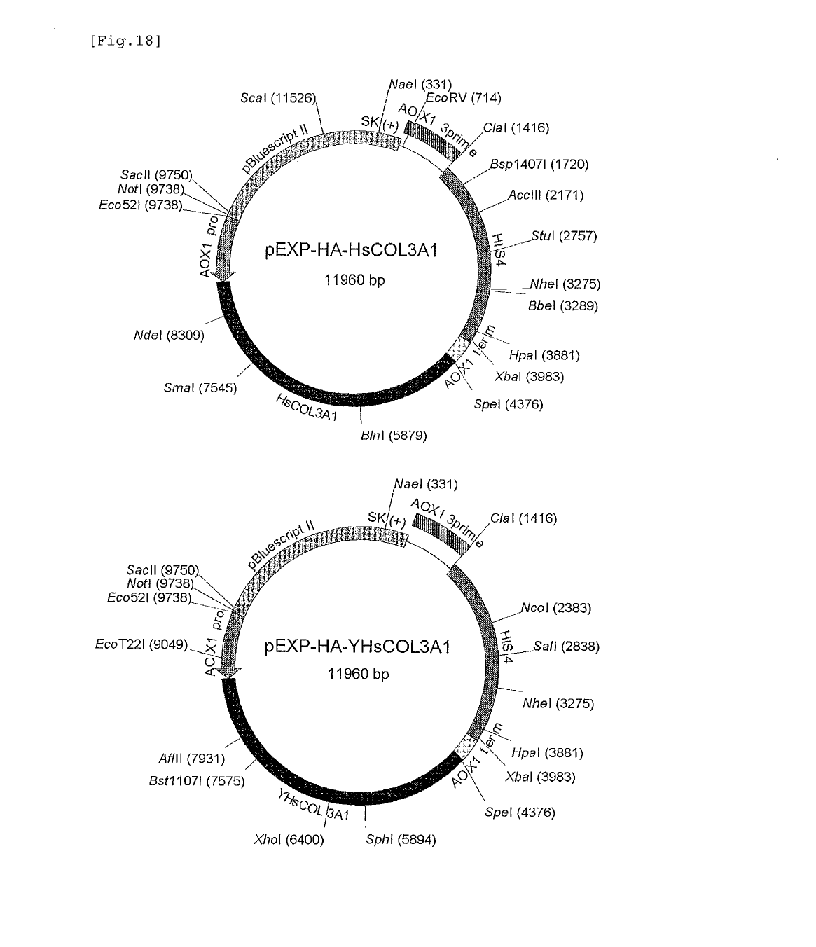

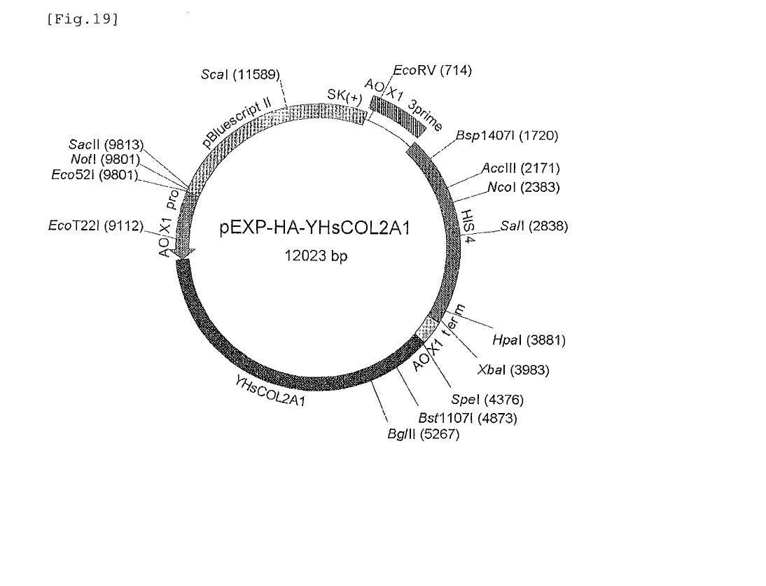

### MODE FOR CARRYING OUT THE INVENTION

It is considered that the invention described herein is not limited to specific methodologies, protocols, and reagents described, but may be modified. It is also considered that the terms used herein are used merely for description of specific embodiments, without intention to limit the scope of the present invention.

Unless otherwise specified, all technical and chemical terms used herein have the same meanings as commonly understood by those with an ordinary skill in the art to which the present invention belongs. Although any methods and materials similar or equivalent to those described herein may be used in the exploitation or verification of the invention, preferred methods, apparatuses and materials are described below.

A transformant of the present invention is characterized in that the transformant obtained by transfecting all of the following polynucleotides (1), (2) and (3) into a microbial cell:

(1) a polynucleotide comprising a nucleotide sequence encoding the amino acid sequence of an FK506 binding protein that is capable of binding to FK506 and has a molecular weight of 15,000 or more and 60,000 or less;
(2) a polynucleotide comprising a nucleotide sequence encoding the amino acid sequence of a prolyl hydroxylase; and
(3) a polynucleotide comprising a nucleotide sequence encoding the amino acid sequence of a glycine repeat sequence protein having the following characteristics (A) and (B):
<characteristic (A)> the polypeptide chain of the glycine repeat sequence protein having a continuous, Gly-Xaa-Yaa repeating sequence, and
<characteristic (B)> an imino acid being contained in the continuous, Gly-Xaa-Yaa repeating sequence of the polypeptide chain of the glycine repeat sequence protein,
wherein Xaa and Yaa each represent any amino acid.

Accordingly, the transformant of the present invention is a microbial cell which has been transformed with the above-mentioned polynucleotides (1), (2) and (3).

An FK506 binding protein (FKBP) refers collectively to proteins comprising the amino acid sequence of an FKBP domain which is found in proteins forming a complex with an immunosuppressant FK506. A polynucleotide comprising a nucleotide sequence encoding the amino acid sequence of an FK506 binding protein which is used for the production of the transformant of the present invention can be a polynucleotide comprising a nucleotide sequence encoding the amino acid sequence of an FK506 binding protein which has a molecular weight of 15,000 or more and 60,000 or less. Further, the FK506 binding protein can be preferably FKBP23 or FKBP19, and more preferably FKBP23. Species from which the FK506 binding protein is derived are not limited in particular, and the FK506 binding protein can be an FK506 binding protein derived from an animal, preferably a homeotherm, more preferably a mammal, further preferably a human.

Examples of the FK506 binding protein can include human FKBP23 comprising the amino acid sequence indicated by SEQ ID NO:74, human FKBP19 comprising the amino acid sequence indicated by SEQ ID NO:75, and an FK506 binding protein having an amino acid deletion, substitution or addition in these amino acid sequences. In addition, the FK506 binding protein may be an FK506 binding protein having an amino acid sequence which has homology with these amino acid sequences, for example, a protein comprising an amino acid sequence derived from an orthologue gene from a different species. Examples of an amino acid sequence derived from an orthologue gene of human FKBP23 can include the amino acid sequence of dog FKBP23 (NCBI accession: XP_{—}545546), chimpanzee FKBP23 (NCBI accession: XP_{—}515942), bovine FKBP23 (NCBI accession: XP_{—}871858), rat FKBP23 (NCBI accession: XP_{—}215758), mouse FKBP23 (NCBI accession: NP_{—}034352), chicken FKBP23 (NCBI accession: XP_{—}421981) and the like. Examples of an amino acid sequence derived from an orthologue gene of human FKBP19 can include the amino acid sequence of dog FKBP19 (NCBI accession: XP_{—}851205), chimpanzee FKBP19 (NCBI accession: XP_{—}001159891), bovine FKBP19 (NCBI accession: NP_{—}001039397), rat FKBP19 (NCBI accession: NP_{—}001013123), mouse FKBP19 (NCBI accession: NP_{—}077131) and the like. Further, the FK506 binding protein may be an FK506 binding protein which has an amino acid deletion, substitution or addition in an amino acid sequence derived from these orthologue genes.

In addition, examples of a polynucleotide comprising a nucleotide sequence encoding the amino acid sequence of the FK506 binding protein can include a polynucleotide comprising the nucleotide sequence indicated by SEQ ID NO:76, a polynucleotide comprising the nucleotide sequence indicated by SEQ ID NO:77, a polynucleotide comprising the nucleotide sequence indicated by SEQ ID NO:78 and a polynucleotide comprising the nucleotide sequence indicated by SEQ ID NO:79, and more preferably, a polynucleotide comprising a nucleotide sequence encoding the amino acid sequence of the FK506 binding protein can be a polynucleotide comprising the nucleotide sequence indicated by SEQ ID NO:76 or a polynucleotide comprising the nucleotide sequence indicated by SEQ ID NO:78. In addition, included can be a polynucleotide having a deletion, substitution, or addition of one or more bases in the nucleotide sequences of these polynucleotides; and a polynucleotide comprising a partial nucleotide sequence of these polynucleotides. Further, included may be a polynucleotide comprising a nucleotide sequence having homology with the nucleotide sequences of these polynucleotides, for example, a polynucleotide comprising a nucleotide sequence derived from the coding sequence (CDS) of an orthologue gene from a different species. Examples of the CDS of an orthologue gene of human FKBP23 can include dog FKBP23 CDS (NCBI accession: XM_{—}545546), chimpanzee FKBP23 CDS (NCBI accession: XM_{—}515942), bovine FKBP23 CDS (NCBI accession: XM_{—}866765), rat FKBP23 CDS (NCBI accession: XM_{—}215758), mouse FKBP23 CDS (NCBI accession: NM_{—}010222), chicken FKBP23 CDS (NCBI accession: XM_{—}421981) and the like. Examples of the CDS of an orthologue gene of human FKBP19 can include dog FKBP19 CDS (NCBI accession: XM_{—}846112), chimpanzee FKBP19 CDS (NCBI accession: XM_{—}001159891), bovine FKBP19 CDS (NCBI accession: NM_{—}001045932), rat FKBP19 CDS (NCBI accession: NM_{—}001013105), mouse FKBP19 CDS (NCBI accession: NM_{—}024169) and the like. Further, included may be a polynucleotide having a deletion, substitution or addition of one or more bases in the nucleotide sequence of a polynucleotide comprising a nucleotide sequence derived from these CDSs; and a polynucleotide comprising a partial nucleotide sequence of a nucleotide sequence derived from these CDSs.

Examples of a polynucleotide comprising a nucleotide sequence encoding a prolyl hydroxylase which is used for the production of the transformant of the present inventions can include a polynucleotide comprising a nucleotide sequence encoding the amino acid sequence of prolyl 4-hydroxylase. Prolyl 4-hydroxylase is an enzyme which catalyzes the reaction of hydroxylation of the 4-position of the proline residues located at Yaa sites in the Gly-Xaa-Yaa repeating sequence, converting the proline to 4-hydroxyproline. A glycine repeat sequence protein forms a unique triple-helical structure. The hydroxylation of the proline residues located at Yaa sites results in an improved stability of the triple-helical structure of the glycine repeat sequence protein. The origin of the prolyl 4-hydroxylase is not limited in particular, and the prolyl 4-hydroxylase can be a prolyl 4-hydroxylase derived from an animal, preferably a higher animal, more preferably a human.

The prolyl 4-hydroxylase can be a prolyl hydroxylase comprising an α subunit and a β subunit, wherein the α subunit can be, for example, an α1 subunit, an α2 subunit, or an α3 subunit, preferably an α1 subunit.

Examples of the prolyl 4-hydroxylase α subunit can include a human prolyl 4-hydroxylase α1 subunit (P4Hα1) comprising the amino acid sequence indicated by SEQ ID NO:80, a human prolyl 4-hydroxylase α2 subunit (P4Hα2) comprising the amino acid sequence indicated by SEQ ID NO:81 and a human prolyl 4-hydroxylase α3 subunit (P4Hα3) comprising the amino acid sequence indicated by SEQ ID NO:82. Examples of the prolyl 4-hydroxylase β subunit (P4Hβ) can include human PH4β comprising the amino acid sequence indicated by SEQ ID NO:83. Further, included may be a protein having an amino acid deletion, substitution or addition in these amino acid sequences and a protein having an amino acid sequence which has homology with these amino acid sequences, for example, a protein comprising an amino acid sequence derived from an orthologue gene from a different species. Examples of an amino acid sequence derived from an orthologue gene of human P4Hα1 can include the amino acid sequence of dog P4Hα1 (NCBI accession: XP_{—}862257), chimpanzee P4Hα1 (NCBI accession: XP_{—}001141043), bovine P4Hα1 (NCBI accession: NP_{—}001069238), rat P4Hα1 (NCBI accession: NP_{—}742059), mouse P4Hα1 (NCBI accession: NP_{—}035160) and chicken P4Hα1 (NCBI accession: XP_{—}421583). Examples of an amino acid sequence derived from an orthologue gene of human P4Hα2 can include the amino acid sequence of dog P4Hα2 (NCBI accession: XP_{—}860637), chimpanzee P4Hα2 (NCBI accession: XP_{—}001162222), bovine P4Hα2 (NCBI accession: NP_{—}001029465), rat P4Hα2 (NCBI accession: XP_{—}340799), mouse P4Hα2 (NCBI accession: NP_{—}035161) and chicken P4Hα2 (NCBI accession: NP_{—}001006155). Examples of an amino acid sequence derived from an orthologue gene of human P4Hα3 can include the amino acid sequence of dog P4Hα3 (NCBI accession: XP_{—}851718), chimpanzee P4Hα3 (NCBI accession: XP_{—}001174896), bovine P4Hα3 (NCBI accession: NP_{—}001001598), rat P4Hα3 (NCBI accession: NP_{—}942070), mouse P4Hα3 (NCBI accession: NP_{—}796135) and chicken P4Hα3 (NCBI accession: XP_{—}417248). Examples of an amino acid sequence derived from an orthologue gene of human P4Hβ can include the amino acid sequence of dog P4Hβ (NCBI accession: XP_{—}540488), chimpanzee P4Hβ (NCBI accession: XP_{—}001164396), bovine P4Hβ (NCBI accession: NP_{—}776560), rat P4Hβ (NCBI accession: NP_{—}037130), mouse P4Hβ (NCBI accession: NP_{—}035162) and chicken P4Hβ (NCBI accession: XP_{—}420095). Further, included may be a prolyl 4-hydroxylase which has an amino acid deletion, substitution or addition in an amino acid sequence derived from these orthologue genes.

In addition, examples of a polynucleotide comprising a nucleotide sequence encoding the amino acid sequence of the prolyl 4-hydroxylase as described above can include the polynucleotide indicated by SEQ ID NO:84, which comprises a nucleotide sequence encoding the amino acid sequence of human P4Hα1; the polynucleotide indicated by SEQ ID NO:85, which comprises a nucleotide sequence encoding the amino acid sequence of human P4Hα2; the polynucleotide indicated by SEQ ID NO:86, which comprises a nucleotide sequence encoding the amino acid sequence of human P4Hα3; and the polynucleotide indicated by SEQ ID NO:87, which comprises a nucleotide sequence encoding the amino acid sequence of human P4Hβ. More preferably, a polynucleotide comprising a nucleotide sequence encoding the amino acid sequence of the prolyl 4-hydroxylase as described above can be the polynucleotide indicated by SEQ ID NO:84 or the polynucleotide indicated by SEQ ID NO:87. In addition, included can be a polynucleotide having a deletion, substitution or addition of one or more bases in the nucleotide sequences of these polynucleotides; and a polynucleotide comprising a partial nucleotide sequence of these polynucleotides. Further, included may be a polynucleotide comprising a nucleotide sequence having homology with the nucleotide sequences of these polynucleotides, for example, a polynucleotide comprising a nucleotide sequence derived from the coding sequence (CDS) of an orthologue gene from a different species. Examples of the CDS of an orthologue gene of human P4Hα1 can include dog P4HA1 CDS (NCBI accession: XM_{—}857164), chimpanzee P4HA1 CDS (NCBI accession: XM_{—}001141043), bovine P4HA1 CDS (NCBI accession: NM_{—}001075770), rat P4HA1 CDS (NCBI accession: NM_{—}172062), mouse P4HA1 CDS (NCBI accession: NM_{—}011030), chicken P4HA1 CDS (NCBI accession: XM_{—}421583) and the like. Examples of the CDS of an orthologue gene of human P4Hα2 can include dog P4HA2 CDS (NCBI accession: XM_{—}855544), chimpanzee P4HA2 CDS (NCBI accession: XM_{—}001162222), bovine P4HA2 CDS (NCBI accession: NM_{—}001034293), rat P4HA2 CDS (NCBI accession: XM_{—}340798), mouse P4HA2 CDS (NCBI accession: NM_{—}011031), chicken P4HA2 CDS (NCBI accession: NM_{—}001006155) and the like. Examples of the CDS of an orthologue gene of human P4Hα3 can include dog P4HA3 CDS (NCBI accession: XM_{—}846625), chimpanzee P4HA3 CDS (NCBI accession: XM_{—}001174896), bovine P4HA3 CDS (NCBI accession: NM_{—}001001598), rat P4HA3 CDS (NCBI accession: NM_{—}198775), mouse P4HA3 CDS (NCBI accession: NM_{—}177161), chicken P4HA3 CDS (NCBI accession: XM_{—}417248) and the like. Examples of the CDS of an orthologue gene of human P4Hβ can include dog P4HB CDS (NCBI accession: XM_{—}540488), chimpanzee P4HB CDS (NCBI accession: XM_{—}001164396), bovine P4HB CDS (NCBI accession: NM_{—}174135), rat P4HB CDS (NCBI accession: NM_{—}012998), mouse P4HB CDS (NCBI accession: NM_{—}011032), chicken P4HB CDS (NCBI accession: XM_{—}420095) and the like. Further, included may be a polynucleotide having a deletion, substitution or addition of one or more bases in the nucleotide sequence of a polynucleotide comprising a nucleotide sequence derived from these CDSs; and a polynucleotide comprising a partial nucleotide sequence of a nucleotide sequence derived from these CDSs.

A protein comprising a glycine-repeating sequence according to the present invention is not limited in particular, as long as the protein is a glycine repeat sequence protein having the characteristics (A) and (B) described below. Such protein according to the present invention can be, for example, at least one collagen selected from among collagens of Type I to Type XXIX, preferably a Fibril-forming collagen (Type I collagen, Type II collagen, Type III collagen, Type V collagen, Type XI collagen, or Type XXIV collagen, Type XXVII collagen), more preferably Type I collagen, Type II collagen, or Type III collagen. Also, included can be a glycine repeat sequence protein which consists of a partial region of these collagens. The origin of the collagens is not limited in particular, and the collagens can be a collagen derived from an animal, preferably a higher animal, further preferably a human.

### <Characteristic (A)>

The polypeptide chain of the glycine repeat sequence protein having a continuous, Gly-Xaa-Yaa repeating sequence.

### <Characteristic (B)>

An imino acid (for example, proline or hydroxyproline) being contained in the continuous, Gly-Xaa-Yaa repeating sequence of the polypeptide chain of the glycine repeat sequence protein,

wherein Xaa and Yaa each represent any amino acid.

In characteristic (A), the number of repeats of the repeating sequence Gly-Xaa-Yaa can be 25 or more and 362 or less, for example. In characteristic (B), the content of the imino acid which is contained in the Gly-Xaa-Yaa repeating sequence can be 19.8% or more and 38.7% or less, for example.

Examples of the Type I collagen can include a human Type I α1 collagen precursor (Hs Type I α1) comprising the amino acid sequence indicated by SEQ ID NO:88 and a human Type I α2 collagen precursor (Hs Type I α2) comprising the amino acid sequence indicated by SEQ ID NO:89. Examples of the Type II collagen can include a human Type II α1 collagen precursor (Hs Type II α1) comprising the amino acid sequence indicated by SEQ ID NO:90. Examples of the Type III collagen can include a human Type III α1 collagen precursor (Hs Type III α1) comprising the amino acid sequence indicated by SEQ ID NO:91. Further, included can be a protein having an amino acid deletion, substitution or addition in these amino acid sequences. In addition, included may be a protein having an amino acid sequence which has homology with these amino acid sequences, for example, a protein comprising an amino acid sequence derived from an orthologue from a different species. Examples of an amino acid sequence derived from an orthologue gene of human Type I α1 can include the amino acid sequence of dog Type I α1 (NCBI accession: NP_{—}001003090), chimpanzee Type I α1 (NCBI accession: XP_{—}001169320), bovine Type I α1 (NCBI accession: NP_{—}001029211), rat Type I α1 (NCBI accession: XP_{—}213440) and mouse Type I α1 (NCBI accession: NP_{—}031768). Examples of an amino acid sequence derived from an orthologue gene of human Type I α2 can include the amino acid sequence of dog Type I α2 (NCBI accession: NP_{—}001003187), chimpanzee Type I α2 (NCBI accession: XP_{—}519207), bovine Type I α2 (NCBI accession: NP_{—}776945), rat Type I α2 (NCBI accession: NP_{—}445808) and mouse Type I α2 (NCBI accession: NP_{—}031769). Examples of an amino acid sequence derived from an orthologue gene of human Type II α1 can include the amino acid sequence of dog Type II α1 (NCBI accession: NP_{—}001006952), chimpanzee Type II α1 (NCBI accession: XP_{—}509026), rat Type II α1 (NCBI accession: NP_{—}037061), mouse Type II α1 (NCBI accession: NP_{—}112440) and chicken Type II α1 (NCBI accession: NP_{—}989757). Examples of an amino acid sequence derived from an orthologue gene of human Type III α1 can include the amino acid sequence of dog Type III α1 (NCBI accession: XP_{—}851009), chimpanzee Type III α1 (NCBI accession: XP_{—}001163665), bovine Type III α1 (NCBI accession: NP_{—}001070299), rat Type III α1 (NCBI accession: NP_{—}114474), mouse Type III α1 (NCBI accession: NP_{—}034060) and chicken Type III α1 (NCBI accession: XP_{—}421847). Further, included may be a protein which has an amino acid deletion, substitution or addition in an amino acid sequence derived from these orthologue genes.

In the case where the glycine repeat sequence protein has been expressed within a yeast and the produced prolyl hydroxylase is present within the yeast, a glycine repeat sequence protein may be produced in which proline residues of the glycine repeat sequence protein are hydroxylated by the prolyl hydroxylase. The degree of hydroxylation of the proline residues of a glycine repeat sequence protein may be determined in known methods for amino acid composition analysis. In addition, the ability for fibril formation of a glycine repeat sequence protein may be determined, for example, by the following procedures. In particular, for example, a solution of a purified glycine repeat sequence protein is readjusted to a salt concentration of 1×D-PBS(−) and a pH of from 7.3 to 7.4 and then kept at a temperature of 37° C., thereby leading to reorientation of the glycine repeat sequence protein molecule and then clouding of the solution. Such clouding may be regarded as an indication of the ability for fibril formation of the glycine repeat sequence protein. Accordingly, by means of this property, the ability for fibril formation of a glycine repeat sequence protein may be determined by keeping a solution of a glycine repeat sequence protein, whose concentration is 0.05%, at a temperature of 37° C., at a salt concentration of 1×D-PBS(−), and at a pH of from 7.3 to 7.4, and measuring the absorbance of the solution over time during the incubation period.

Examples of a glycine repeat sequence protein consisting of a partial region of the above-mentioned collagens can include a precursor of a fusion polypeptide (Hs Type I α1 N60C15) comprising the amino acid sequence indicated by SEQ ID NO:92, which consists of the N-terminal non-helix region, 60 N-terminal residues of the helix region, 15 C-terminal residues of the helix region and the C-terminal non-helix region of human collagen Type I α1; a precursor of a fusion polypeptide (Hs Type I α2 N60-C15) comprising the amino acid sequence indicated by SEQ ID NO:93, which consists of the N-terminal non-helix region, 60 N-terminal residues of the helix region, 15 C-terminal residues of the helix region and the C-terminal non-helix region of human collagen Type I α2; a precursor of a fusion polypeptide (Hs Type I α1 N27-M522-C15) comprising the amino acid sequence indicated by SEQ ID NO:94, which consists of the N-terminal non-helix region, 27 N-terminal residues of the helix region, 522 central residues of the helix region, 15 C-terminal residues of the helix region and the C-terminal non-helix region of human collagen Type I α1; a precursor of a fusion polypeptide (Hs Type I α2 N27-M522-C15) comprising the amino acid sequence indicated by SEQ ID NO:95, which consists of the N-terminal non-helix region, 27 N-terminal residues of the helix region, 522 central residues of the helix region, 15 C-terminal residues of the helix region and the C-terminal non-helix region of human collagen Type I α2; a precursor of a fusion polypeptide (Hs Type I α1 N27-M522X2-C15) comprising the amino acid sequence indicated by SEQ ID NO:96, which consists of the N-terminal non-helix region, 27 N-terminal residues of the helix region, a dimer made of 522 central residues of the helix region, 15 C-terminal residues of the helix region and the C-terminal non-helix region of human collagen Type I α1; and a precursor of a fusion polypeptide (Hs Type I α2 N27-M522x2-C15) comprising the amino acid sequence indicated by SEQ ID NO:97, which consists of the N-terminal non-helix region, 27 N-terminal residues of the helix region, a dimer made of 522 central residues of the helix region, 15 C-terminal residues of the helix region and the C-terminal non-helix region of human collagen Type I α2.

Examples of a polynucleotide comprising a nucleotide sequence encoding a glycine repeat sequence protein which is used for the production of the transformant of the present invention can include a polynucleotide comprising a nucleotide sequence encoding the amino acid sequence of the above-mentioned human Type I α1 collagen precursor, wherein the nucleotide sequence is indicated by SEQ ID NO:98; a polynucleotide comprising a nucleotide sequence encoding the amino acid sequence of the above-mentioned human Type I α1 collagen precursor, wherein the nucleotide sequence is indicated by SEQ ID NO:99; a polynucleotide comprising a nucleotide sequence encoding the amino acid sequence of the above-mentioned human Type I α2 collagen precursor, wherein the nucleotide sequence is indicated by SEQ ID NO:100; a polynucleotide comprising a nucleotide sequence encoding the amino acid sequence of the above-mentioned human Type I α2 collagen precursor, wherein the nucleotide sequence is indicated by SEQ ID NO:101; a polynucleotide comprising a nucleotide sequence encoding the amino acid sequence of the above-mentioned human Type II α1 collagen precursor, wherein the nucleotide sequence is indicated by SEQ ID NO:102; a polynucleotide comprising a nucleotide sequence encoding the amino acid sequence of the above-mentioned human Type II α1 collagen precursor, wherein the nucleotide sequence is indicated by SEQ ID NO:103; a polynucleotide comprising a nucleotide sequence encoding the amino acid sequence of the above-mentioned human Type III α1 collagen precursor, wherein the nucleotide sequence is indicated by SEQ ID NO:104; and a polynucleotide comprising a nucleotide sequence encoding the amino acid sequence of the above-mentioned human Type III α1 collagen precursor, wherein the nucleotide sequence is indicated by SEQ ID NO:105; and more preferably, a polynucleotide comprising a nucleotide sequence encoding a glycine repeat sequence protein which is used for the production of the transformant of the present invention can be a polynucleotide comprising a nucleotide sequence encoding the amino acid sequence of the above-mentioned human Type I α1 collagen precursor, wherein the nucleotide sequence is indicated by SEQ ID NO:98; a polynucleotide comprising a nucleotide sequence encoding the amino acid sequence of the above-mentioned human Type I α2 collagen precursor, wherein the nucleotide sequence is indicated by SEQ ID NO:100; a polynucleotide comprising a nucleotide sequence encoding the amino acid sequence of the above-mentioned human Type II α1 collagen precursor, wherein the nucleotide sequence is indicated by SEQ ID NO:102; or a polynucleotide comprising a nucleotide sequence encoding the amino acid sequence of the above-mentioned human Type III α1 collagen precursor, wherein the nucleotide sequence is indicated by SEQ ID NO:104. In addition, included can be a polynucleotide having a deletion, substitution or addition of one or more bases in the nucleotide sequences of these polynucleotides; and a polynucleotide comprising a partial nucleotide sequence of these polynucleotides. Further, included may be a polynucleotide comprising a nucleotide sequence having homology with the nucleotide sequences of these polynucleotides, for example, a polynucleotide comprising a nucleotide sequence derived from the coding sequence (CDS) of an orthologue gene from a different species.

Examples of the CDS of an orthologue gene of human Type I α1 can include dog Type I α1 CDS (NCBI accession: NM_{—}001003090), chimpanzee Type I α1 CDS (NCBI accession: XM_{—}001169320), bovine Type I α1 CDS (NCBI accession: NM_{—}001034039), rat Type I α1 CDS (NCBI accession: XM_{—}213440), mouse Type I α1 CDS (NCBI accession: NM_{—}007742) and the like. Examples of the CDS of an orthologue gene of human Type I α2 can include dog Type I α2 CDS (NCBI accession: NM_{—}001003187), chimpanzee Type I α2 CDS (NCBI accession: XM_{—}519207), bovine Type I α2 CDS (NCBI accession: NM_{—}174520), rat Type I α2 CDS (NCBI accession: NM_{—}053356), mouse Type I α2 CDS (NCBI accession: NM_{—}007743) and the like. Examples of the CDS of an orthologue gene of human Type II α1 can include dog Type II α1 CDS (NCBI accession: NM_{—}001006951), chimpanzee Type II α1 CDS (NCBI accession: XM_{—}509026), rat Type II α1 CDS (NCBI accession: NM_{—}012929), mouse Type II α1 CDS (NCBI accession: NM_{—}031163), chicken Type II α1 CDS (NCBI accession: NM_{—}204426) and the like. Examples of the CDS of an orthologue gene of human Type III α1 can include dog Type III α1 CDS (NCBI accession: XM_{—}845916), chimpanzee Type III α1 CDS (NCBI accession: XM_{—}001163665), bovine Type III α1 CDS (NCBI accession: NM_{—}001076831), rat Type III α1 CDS (NCBI accession: NM_{—}032085), mouse Type III α1 CDS (NCBI accession: NP_{—}009930), chicken Type III α1 CDS (NCBI accession: XM_{—}421847) and the like. Further, included may be a polynucleotide having a deletion, substitution or addition of one or more bases in the nucleotide sequence of a polynucleotide comprising a nucleotide sequence derived from these CDSs; and a polynucleotide comprising a partial nucleotide sequence of a nucleotide sequence derived from these CDSs.

Examples of a polynucleotide comprising a nucleotide sequence encoding a glycine repeat sequence protein consisting of a partial region of the above-mentioned collagens can include a polynucleotide comprising a nucleotide sequence encoding the amino acid sequence of the Hs Type I α1 N60C15 precursor which is indicated by SEQ ID NO:106, a polynucleotide comprising a nucleotide sequence encoding the amino acid sequence of the Hs Type I α2 N60-C15 precursor which is indicated by SEQ ID NO:107, a polynucleotide comprising a nucleotide sequence encoding the amino acid sequence of the Hs Type I α1 N27-M522-C15 precursor which is indicated by SEQ ID NO:108, a polynucleotide comprising a nucleotide sequence encoding the amino acid sequence of the Hs Type I α2 N27-M522-C15 precursor which is indicated by SEQ ID NO:109, a polynucleotide comprising a nucleotide sequence encoding the amino acid sequence of the Hs Type I α1 N27-M522X2-C15 precursor which is indicated by SEQ ID NO:110, and a polynucleotide comprising a nucleotide sequence encoding the amino acid sequence of the Hs Type I α2 N27-M522x2-C15 precursor which is indicated by SEQ ID NO:111.

The “transformant of the present invention” as described above may be further a transformant which is obtained by transfecting an additional polynucleotide comprising a nucleotide sequence encoding the amino acid sequence of a “lysyl hydroxylase,” that is, a transformant which produces all of the following proteins (1) to (4):

(1) an FK506 binding protein that is capable of binding to FK506 and has a molecular weight of 15,000 or more and 60,000 or less;
(2) a prolyl hydroxylase;
(3) a glycine repeat sequence protein having the following characteristics (A) and (B):
<characteristic (A)> the polypeptide chain of the glycine repeat sequence protein having a continuous, Gly-Xaa-Yaa repeating sequence, and
<characteristic (B)> an imino acid (e.g. proline or hydroxyproline) being contained in the continuous, Gly-Xaa-Yaa repeating sequence in the polypeptide chain of the glycine repeat sequence protein
wherein Xaa and Yaa each represent any amino acid; and
(4) a lysyl hydroxylase.

Such transformant is capable of efficiently producing a glycine repeat sequence protein with hydroxylated lysine residues in the amino acid sequence. Such transformant can produce a glycine repeat sequence protein with an enhanced ability for fibril formation and an increased stability. For example, a transformant which produces all of the above-mentioned proteins (1) to (4) allows the production of a glycine repeat sequence protein in which 20% or more, preferably 40% or more, of the total lysine residues located at Yaa's in the Gly-Xaa-Yaa repeating sequence have been hydroxylated.

A glycine repeat sequence protein with hydroxylated lysine residues is one that exhibits an enhanced ability for fibril formation and has a more stable triple-helical structure, and thus can be one that is usable as a high performance versatile material which is more commercially valuable for pharmaceuticals, industrial products, cosmetics, foods etc.

In the present invention, a “lysyl hydroxylase” can include lysyl hydroxylase 1, lysyl hydroxylase 3 and the like.

The origin of a “lysyl hydroxylase” is not limited in particular, and a “lysyl hydroxylase” is preferably derived, for example, from a higher animal, further preferably from a human.

Lysyl hydroxylase 1 and lysyl hydroxylase 3 are homodimer-forming enzymes and hydroxylate the δ position of the lysines located at Yaa's in the Gly-Xaa-Yaa repeating sequence found in the helix structure of a glycine repeat sequence protein.

Examples of the lysyl hydroxylase can include human lysyl hydroxylase (LH1) comprising the amino acid sequence indicated by SEQ ID NO:116 and human lysyl hydroxylase 3 (LH3) comprising the amino acid sequence indicated by SEQ ID NO:118. Further, included can be a protein having an amino acid deletion, substitution or addition in these amino acid sequences. In addition, included may be a protein homologous to these amino acid sequences, for example, a protein comprising an amino acid sequence derived from an orthologue gene from a different species. Examples of an amino acid sequence derived from an orthologue gene of human LH1 can include the amino acid sequence of dog LH1 (NCBI accession: XP_{—}865470), chimpanzee LH1 (NCBI accession: XP_{—}001142937), bovine LH1 (NCBI accession: NP_{—}776573), rat LH1 (NCBI accession: NP_{—}446279), mouse LH1 (NCBI accession: NP_{—}035252) and chicken LH1 (NCBI accession: NP_{—}001005618). Examples of an amino acid sequence derived from an orthologue gene of human LH3 can include the amino acid sequence of dog LH3 (NCBI accession: XP_{—}858413), chimpanzee LH3 (NCBI accession: XP_{—}001153979), bovine LH3 (NCBI accession: XP_{—}887254), rat LH3 (NCBI accession: NP_{—}835202), mouse LH3 (NCBI accession: NP_{—}036092) and the like. Further, included may be a lysyl hydroxylase having an amino acid deletion, substitution or addition in an amino acid sequence derived from these orthologue genes.

Examples of a polynucleotide comprising a nucleotide sequence encoding the amino acid sequence of a “lysyl hydroxylase” which can be used for the production of the transformant of the present invention can include a polynucleotide comprising a nucleotide sequence encoding lysyl hydroxylase 1 (LH1) and a polynucleotide comprising a nucleotide sequence encoding lysyl hydroxylase 3 (LH3). Examples of a polynucleotide comprising a nucleotide sequence encoding the above-mentioned lysyl hydroxylase 1 can include a polynucleotide comprising a nucleotide sequence encoding the amino acid sequence of human LH1 which is indicated by SEQ ID NO:117 and a polynucleotide comprising a nucleotide sequence encoding the amino acid sequence of human LH3 which is indicated by SEQ ID NO:119. In addition, included can be a polynucleotide having a deletion, substitution or addition of one or more bases in the nucleotide sequences of these polynucleotides; and a polynucleotide comprising a partial nucleotide sequence of these polynucleotides. Further, included may be a polynucleotide comprising a nucleotide sequence homologous to the nucleotide sequences of these polynucleotides, for example, a polynucleotide comprising a nucleotide sequence derived from the coding sequence (CDS) of an orthologue gene from a different species. Examples of the CDS of an orthologue gene of human LH1 can include dog PLOD1 CDS (NCBI accession: XM_{—}860377), chimpanzee PLOD1 CDS (NCBI accession: XM_{—}001142937), bovine PLOD1 CDS (NCBI accession: NM_{—}174148), rat PLOD1 CDS (NCBI accession: NM_{—}053827), mouse PLOD1 CDS (NCBI accession: NM_{—}011122), chicken PLOD1 CDS (NCBI accession: NM_{—}001005618) and the like. Examples of the CDS of an orthologue gene of human LH3 can include dog PLOD3 CDS (NCBI accession: XM_{—}853320), chimpanzee PLOD3 CDS (NCBI accession: XM_{—}001153979), bovine PLOD3 CDS (NCBI accession: XM_{—}882161), rat PLOD3 CDS (NCBI accession: NM_{—}178101), mouse PLOD3 CDS (NCBI accession: NM_{—}011962) and the like. Further, included may be a polynucleotide having a deletion, substitution or addition of one or more bases in the nucleotide sequence of a polynucleotide comprising a nucleotide sequence derived from the CDSs of these orthologue genes; and a polynucleotide comprising a partial nucleotide sequence of a nucleotide sequence derived from the CDSs of these orthologue genes.

Here, the above-mentioned polynucleotide comprising a nucleotide sequence encoding the amino acid sequence of a lysyl hydroxylase is preferably linked, for example, to downstream of a promoter derived from a yeast, for example, the promoter of the alcohol oxidase 1 gene or the like.

The “transformant of the present invention” as described above may be further a transformant which is obtained by additionally transfecting a polynucleotide comprising a nucleotide sequence encoding the amino acid sequence of “Hsp47”, that is, a transformant which produces all of the following proteins (1) to (3) and (5):

(1) an FK506 binding protein that is capable of binding to FK506 and has a molecular weight of 15,000 or more and 60,000 or less:
(2) a prolyl hydroxylase;
(3) a glycine repeat sequence protein having the following characteristics (A) and (B):
<characteristic (A)> the polypeptide chain of the glycine repeat sequence protein having a continuous, Gly-Xaa-Yaa repeating sequence, and
<characteristic (B)> an imino acid (e.g. proline or hydroxyproline) being contained in the continuous, Gly-Xaa-Yaa repeating sequence of the polypeptide chain of the glycine repeat sequence protein
wherein Xaa and Yaa each represent any amino acid; and

### (5) Hsp47.

Such transformant is capable of efficiently producing a protein which has been modified with neutral sugars and has a smaller content of the neutral sugars. Such yeast reduces a change of the physical properties relating to the hydrophilicity of glycine repeat sequence proteins which result from neutral sugars, thereby making it possible to produce a glycine repeat sequence protein having more lipophilic properties that are similar to those inherently possessed by glycine repeat sequence proteins. Such glycine repeat sequence protein is a protein having a more stable triple-helical structure, and thus can be a glycine repeat sequence protein which is usable as a high performance versatile material that is more commercially valuable for pharmaceuticals, industrial products, cosmetics, foods etc.

“Hsp47” is a stress response protein localized in the endoplasmic reticulum lumen, which is generally referred to as Heat shock protein 47.

In the present invention, “Hsp47” is not particularly limited by the origin, and for example, is preferably derived from a higher animal, further preferably from a human. Examples can include human Hsp47, which has the amino acid sequence indicated by SEQ ID NO:120. In addition, included can be a protein having a deletion, substitution or addition of an amino acid sequence in the amino acid sequences of these Hsp47s. Also, included may be a protein having homology with these amino acid sequences, for example, a protein comprising an amino acid sequence derived from an orthologue from a different species. Examples of an amino acid sequence derived from an orthologue gene of human Hsp47 can include the amino acid sequence of dog Hsp47 (NCBI accession: XP_{—}542305), chimpanzee Hsp47 (NCBI accession: XP_{—}001174979), bovine Hsp47 (NCBI accession: NP_{—}001039528), rat Hsp47 (NCBI accession: NP_{—}058869), mouse Hsp47 (NCBI accession: NP_{—}033955), chicken Hsp47 (NCBI accession: NP_{—}990622) and the like. Further, included may be a protein having a deletion, substitution or addition of an amino acid sequence in an amino acid sequence derived from these orthologue genes.

Examples of a polynucleotide comprising a nucleotide sequence encoding the amino acid sequence of “Hsp47” which can be used for the production of the transformant of the present invention can include a polynucleotide comprising the nucleotide sequence indicated by SEQ ID NO:121 and others. In addition, included may be a polynucleotide having an artificial deletion, substitution or addition of one or more bases in the nucleotide sequence of this polynucleotide; and also a polynucleotide comprising a nucleotide sequence encoding an amino acid sequence which has a deletion, substitution or addition in the amino acid sequence of Hsp47. In addition, included may be a polynucleotide comprising a nucleotide sequence having homology with these polynucleotides, for example, a polynucleotide comprising a nucleotide sequence derived from the coding sequence (CDS) of an orthologue gene from a different species. Examples of the CDS of an orthologue gene of human Hsp47 can include dog Hsp47 CDS (NCBI accession: XM_{—}542305), chimpanzee Hsp47 CDS (NCBI accession: XM_{—}001174979), bovine Hsp47 CDS (NCBI accession: NM_{—}001046063), rat Hsp47 CDS (NCBI accession: NM_{—}017173), mouse Hsp47 CDS (NCBI accession: NM_{—}009825), chicken Hsp47 CDS (NCBI accession: NM_{—}205291) and the like. Further, included may be a polynucleotide having a deletion, substitution or addition of one or more bases in the nucleotide sequence of a polynucleotide comprising a nucleotide sequence derived from the CDSs of these orthologue genes; and a polynucleotide comprising a partial nucleotide sequence of a nucleotide sequence derived from the CDSs of these orthologue genes.

Here, the above-mentioned polynucleotide comprising a nucleotide sequence encoding the amino acid sequence of Hsp47 is preferably linked, for example, to downstream of a promoter derived from an yeast, for example, the promoter of the alcohol oxidase 1 gene or the like.

The transformant of the present invention may be produced by transfecting all of the below-mentioned polynucleotides (1), (2) and (3) into a host cell. These polynucleotides may be produced by cDNA cloning, assembly PCR using chemically synthesized oligonucleotides, or genetic engineering procedures. Clones, such as commercially available cDNAs, may be also obtained and used.

(1) a polynucleotide comprising a nucleotide sequence encoding the amino acid sequence of an FK506 binding protein that is capable of binding to FK506 and has a molecular weight of 15,000 or more and 60,000 or less;
(2) a polynucleotide comprising a nucleotide sequence encoding a prolyl hydroxylase; and
(3) a polynucleotide comprising a nucleotide sequence encoding the amino acid sequence of a glycine repeat sequence protein having the following characteristics (A) and (B):
<characteristic (A)> the polypeptide chain of the glycine repeat sequence protein having a continuous, Gly-Xaa repeating sequence; and
<characteristic (B)> an imino acid (e.g. proline or hydroxyproline) being contained in the continuous, Gly-Xaa-Yaa repeating sequence of the polypeptide chain of the glycine repeat sequence protein,
wherein Xaa and Yaa each represent any amino acid.

A transformant in which the above-mentioned polynucleotides are efficiently expressed can be produced by linking each of the polynucleotides, for example, to a promoter, a terminator, a polynucleotide encoding a signal sequence etc. which allows efficient expression of the polynucleotide within a cell, thereby constructing an expression cassette; and transfecting each of the constructed expression cassettes for the polynucleotides into a host cell.

These expression cassettes can be constructed by linking the polynucleotide to downstream of a promoter and to upstream of a terminator and optionally replacing the polynucleotide encoding signal sequence at the amino-terminal, by means of standard genetic engineering procedures.

Host-vector systems which are used for constructing the expression cassettes can include, for example, but not especially limited to, the host-vector system using bacteria such as Escherichia, Bacillus or Pseudomonas as the host, and a bacteriophage, a plasmid or a cosmid as the vector; the host-vector system using the yeast such as Komagataella, Saccharomyces, Hansenula, Candida or Ogataea as the host, and an episomal plasmid, a ARS-CEN plasmid or a plasmid integrated into the chromosome as the vector; or the like. Preferably, Escherichia can be used as a host and plasmid can be used as a vector.

The promoter is not limited in particular, as long as it functions within a host cell which is used for producing the transformant of the present invention. Inducible promoters, of which the transcriptional activity may be induced by specific nutrients or substrates, may be preferably used.

Examples of the promoter can include the promoters of the following genes: the galactose metabolizing enzyme genes (GAL1, GAL10), the inhibitory acid phosphatase gene (PHO5), the glyceraldehyde-3-phosphate dehydrogenase gene (TD), the phosphoglycerate kinase gene (PGK), the alcohol oxidase genes (ADH1, AOX1, AOX2, MOX, AOD1), the formate dehydrogenase genes (FDH1, FMD1), the dihydroxyacetone synthase gene (DAS), the peroxisome membrane protein biogenesis gene (PER3), and the formaldehyde dehydrogenase gene (FLD1). As the terminator, any terminator may be effectively used as long as it functions within a host cell which is used for producing the transformant of the present invention, and examples of the terminator can include the terminators of the alcohol oxidase gene (AOX1) and formaldehyde dehydrogenase (FLD1). As the signal sequence, examples can include the prepro sequence of yeast α-factor, the signal sequence of yeast invertase, the signal sequences of yeast acid phosphatase, and others.

The transformant of the present invention can be produced by transforming a host cell using a plasmid comprising the above-mentioned polynucleotides. The use of a plasmid comprising the above-mentioned polynucleotides which have been formed as their respective expression cassettes will allow one to produce a transformant which efficiently expresses the polynucleotides.

Examples of host-vector systems which are used for producing the transformant of the present invention can include a host-vector system where bacteria (e.g. Escherichia, Bacillus, Pseudomonas) is used as a host and a bacteriophage, a plasmid or a cosmid is used as a vector; a host-vector system where a yeast (e.g. Saccharomyces) is used as a host and an episomal plasmid or an ARS-CEN plasmid is used as a vector; a host-vector system where a yeast (e.g. Komagataella, Saccharomyces, Hansenula, Pichia, Candida, Ogataea) is used as a host and a chromosome integration plasmid is used as a vector; a host-vector system where filamentous fungi (e.g. Aspergillus, Trichoderma) is used as a host and a chromosome integration plasmid is used as a vector. Preferably, the host can be a eukaryotic microorganism (yeast, filamentous fungus).

As a more preferable host which is used for producing the transformant of the present invention, a yeast can be employed, for example, Saccharomyces cerevisiae, Komagataella pastoris, Hansenula polymorpha, Pichia methanolica, Candida Boidini, Ogataea minuta. In cases where Saccharomyces cerevisiae is used as a host, the vector can be a chromosome integration plasmid (YIp type), a plasmid comprising the replication initiation region of the 2-μm DNA yeast endogenous plasmid (YEp type) and a plasmid comprising a self-replication region derived from the chromosome (YCp type). In these cases, a plasmid serving as a shuttle vector, where the replication origin which is replicable in Escherichia coli is inserted into the plasmid, can be used to easily construct the respective plasmid into which the above-mentioned polynucleotides have been introduced. In cases where Komagataella pastoris, Hansenula polymorpha, Pichia methanolica, Candida Boidini or Ogataea minuta are used as a host, the vector can be a chromosome integration plasmid. In these cases, a plasmid serving as a shuttle vector, where the replication origin which is replicable in Escherichia coli cells is inserted into the plasmid can be used to easily construct the respective plasmids into which the above-mentioned polynucleotides have been introduced. Further preferably, Komagataella pastoris, Hansenula polymorpha, Pichia methanolica, Candida Boidini or Ogataea minuta, which are methanol-utilizing yeasts, can be used as a host, and a chromosome integration plasmid can be used as a vector. Particularly, Komagataella pastoris may be suitably used as a host, and plasmids which are commercially available, such as pAO815 and pPIC9K, as well as plasmids produced by incorporating vector elements: a homologous recombination region, a maker, a promoter, a terminator etc. may be suitably employed as a vector.

A chromosome integration plasmid has, on the plasmid, a homologous recombination region which comprises a nucleotide sequence homologous to a nucleotide sequence of the host chromosome, whereby the plasmid is integrated in this region into the host chromosome and thus stably retained in the host cell. Examples of a homologous recombination region include, but not limited to, amino acid synthetic pathway gene, nucleic acid synthetic pathway gene, ribosomal DNAs, Ty (Transposon of Yeast) elements and the like. Markers can include genes inducing transformation by introducing them into a host cell, for example, amino acid synthetic pathway gene, nucleic acid synthetic pathway gene, antibiotic-resistance genes and the like. The amino acid or the nucleic acid synthetic pathway gene can include, but not limited to, for example, LEU2, HIS4, ARG4, TRP1, URA3, ADE2 and the like. Further, the antibiotic-resistant gene can include, but not limited to, for example, genes which induce tolerance to antibiotics such as Zeocyn, Blasticidin S, Geneticin, G418, Chloramphenicol and bleomycin.

The amino acid or the nucleic acid synthetic pathway gene can complement a host deficient in a gene for amino acid synthesis, nucleic acid synthesis or the like so that the gene may be also used as a maker for selecting transformants. In addition, markers may be used to serve as a guide for selecting a transformant in which the chromosome integration plasmid has been incorporated into the host chromosome.

Method for introducing into a host cell a plasmid into which the above-mentioned polynucleotide has been incorporated are not limited in particular, and can include, for example, procedures using calcium chloride, spheroplasts, protoplasts, electroporation, and others, when bacteria (e.g. Escherichia, Bacillus, Pseudomonas) are used as a host. In cases where yeasts (e.g. Komagataella, Saccharomyces, Hansenula, Candida, Ogataea) are used as a host, lithium processes, spheroplast procedures, electroporation techniques, and others can be employed. In cases where filamentous fungi (e.g. Aspergillus, Trichoderma) are used as a host, protoplast-PEG procedures, protoplast-electroporation techniques, and others can be employed.

The transformant of the present invention is characterized in that all of the following proteins (1), (2) and (3) are produced within the cell:

(1) an FK506 binding protein that is capable of binding to FK506 and has a molecular weight of 15,000 or more and 60,000 or less;
(2) a prolyl hydroxylase; and
(3) a glycine repeat sequence protein having the following characteristics (A) and (B):
<characteristic (A)> the polypeptide chain of the glycine repeat sequence protein having a continuous, Gly-Xaa-Yaa repeating sequence, and
<characteristic (B)> an imino acid (e.g. proline or hydroxyproline) being contained in the continuous, Gly-Xaa-Yaa repeating sequence of the polypeptide chain of the glycine repeat sequence protein,
wherein Xaa and Yaa each represent any amino acid.

All of the above-mentioned proteins (1) to (3) can be produced by culturing the transformant of the present invention in accordance with standard microbiological techniques. As a medium used for culturing the transformant of the present invention, ones known in the art can be used, and the culturing can be carried out by methods in accordance with standard microbiological techniques.

The medium is not limited in particular, and either of a synthetic medium or a natural medium can be used. For example, the synthetic medium can include mediums containing not only a carbon source (such as a variety of sugars, glycerol, methanol) and a nitrogen source (such as urea, aqueous ammonia, ammonium salts, or nitrate salts), but also mineral salts (such as mineral salts of Mg, Ca, Fe, Na, K, Mn, Co, Mo, B, Zn, I, Cu, or the like), micronutrients (such as a range of vitamins, a range of amino acids or nucleotides), and others. The natural medium can include mediums which contain as the medium components, for example, yeast extract, peptone, casein, and the like, in addition to the components of the synthetic mediums. Further, in cases where an inducible promoter is employed in an above-mentioned expression cassette used for producing the transformant of the present invention, a nutrient, substrate or the like which activates the inducible promoter, for example, galactose, lactose, sucrose, methanol, IPTG and the like may be added to the medium to activate the promoter.

When the transformant of the present invention is a methanol-utilizing yeast, all of the above-mentioned proteins (1), (2) and (3) can be efficiently expressed by using a methanol containing medium. Examples of the medium can include MM medium (1.34% Yeast Nitrogen Base, 4×10⁻⁵% biotin, 0.5% methanol), BMM medium (100 mM potassium phosphate buffer, pH 6.0, 1.34% Yeast Nitrogen Base, 4×10⁻⁵% biotin, 0.5% methanol), Basal Salt medium (Pichia Protocol, ISBN:0-89603-421-6, Humana Press), FM22 medium (Pichia Protocol, ISBN:0-89603-421-6, Humana Press) and others. The pH of a medium is neutral or weak basic, or weak acidic. Particularly, a medium adjusted to a pH of from 3 to 8, more preferably a pH of from 4 to 7, may be used.

The culturing is preferably carried out in the form of liquid culture and the culturing temperature is preferably between 15° C. and 40° C. The culturing period is preferably between 1 and 1,000 hours. The culturing is preferably carried out under conditions with shaking or stirring and can be performed under aeration with air, oxygen. In addition, the culturing is preferably carried out in batch culture, semibatch culture or continuous culture. During the culturing period, feeding of a concentrated medium component or components can be carried out, if needed.

Further, pre-culture can be performed prior to the above-mentioned culture (main culture), if needed. Examples of mediums for pre-culture can include, but not limited to, YNB medium (0.67% Yeast Nitrogen Base, 4×10⁻⁵% biotin, 2% glucose), YPD medium (1% yeast extract, 2% peptone, 2% glucose), BMGY medium (1% yeast extract, 2% peptone, 100 mM potassium phosphate buffer, pH 6.0, 1.34% Yeast Nitrogen Base, 4×10⁻⁵% biotin, 1% methanol) and others. Culturing conditions for pre-culture are not limited in particular. The culturing period is preferably between 10 and 100 hours, and the culturing temperature is preferably between 15° C. and 40° C. Pre-culture is preferably carried out under conditions with shaking or stirring and can be performed under aeration with air, oxygen. Culturing for pre-culture is preferably carried out in batch culture.

The expression of all of the above-mentioned proteins (1), (2) and (3) can be detected by standard biochemical and protein-engineering procedures, including, for example, methods using immunoassays. Specifically, an enzyme-linked immunosorbent assay (ELISA), a radioimmunoassay (RIA), and an immunobead-trapping assay, a western blot analysis etc. may be used. Preferably, a western blot analysis may be used.

The present invention comprises a glycine repeat sequence protein (referred to as a protein of the present invention) which is characterized in that the protein is obtainable by being produced by the transformant of the present invention. Using the transformant of the present invention allows one to obtain a glycine repeat sequence protein with a small content of neutral sugars. In addition, the protein of the present invention is a high performance versatile material (for example, in which changes in the physical properties related to the hydrophilicity of glycine repeat sequence proteins which result from neutral sugars are reduced so as to exhibit more lipophilic properties that are similar to those inherently possessed by glycine repeat sequence proteins) that is more commercially valuable for pharmaceuticals, industrial products, cosmetics and foods etc.

Quantitative determination of neutral sugars contained in a glycine repeat sequence protein may be carried out by standard biochemical procedures. In particular, neutral sugars may be quantitatively determined by phenol-sulfuric acid method, monosaccharide composition analysis using liquid chromatography or the like.

In addition, the ability for fibril formation of a glycine repeat sequence protein may be determined, for example, by the following procedures. For example, a solution of a purified glycine repeat sequence protein is readjusted to a salt concentration of 1×D-PBS(−) and a pH of from 7.3 to 7.4 and then kept at temperature of 37° C., thereby leading to reorientation of the glycine repeat sequence protein molecule and then clouding of the solution. Such clouding may be regarded as an indication of the ability for fibril formation of the glycine repeat sequence t protein. Accordingly, by means of this property, the ability for fibril formation of a glycine repeat sequence protein may be determined by keeping a solution containing 0.05% of glycine repeat sequence protein at a temperature of 37° C., at a salt concentration of 1×D-PBS(−) and at a pH of from 7.3 to 7.4; and measuring the absorbance of the solution over time during the incubation period.

The present invention comprises a process for obtaining a glycine repeat sequence protein (referred to as an process obtained by the present invention), characterized by comprising the following steps:

a first step of transfecting all of the following polynucleotides (1), (2) and (3) into a microbial cell:

(1) a polynucleotide comprising a nucleotide sequence encoding the amino acid sequence of an FK506 binding protein that is capable of binding to FK506 and has a molecular weight of 15,000 or more and 60,000 or less,
(2) a polynucleotide comprising a nucleotide sequence encoding the amino acid sequence of a prolyl hydroxylase, and
(3) a polynucleotide comprising a nucleotide sequence encoding the amino acid sequence of a glycine repeat sequence protein having the following characteristics (A) and (B):
<characteristic (A)> the polypeptide chain of the glycine repeat sequence protein having a continuous, Gly-Xaa-Yaa repeating sequence, and
<characteristic (B)> an imino acid (e.g. proline or hydroxyproline) being contained in the continuous, Gly-Xaa-Yaa repeating sequence of the polypeptide chain of the glycine repeat sequence protein,
wherein Xaa and Yaa each represent any amino acid;

a second step of culturing the transformant resulting from the first step, thereby producing the glycine repeat sequence protein; and

a third step of collecting the glycine repeat sequence protein produced in the second step.

The first step can be performed by the procedures described for the transformant of the present invention as described above. The second step can be performed in similar ways to those in which the transformant of the present invention is used to produce the proteins encoded by the above-mentioned polynucleotides (1), (2) and (3).

The third step can be performed in accordance with standard biochemical procedures. In particular, the third step can be performed by purifying the glycine repeat sequence protein produced in the second step from the microbial cells and the culture supernatant.

Methods for carrying out the above-mentioned purification are not limited in particular. Purification can be effected, for example, by combining a disrupting and solubilizing step and a fractionating and refining step. Specifically, the disrupting and solubilizing step can include methods by which disruption is physically effected with ultrasound, glass beads or others; methods by which solubilization is effected using enzymes, acids, alkalis, surfactants or others; solvent extraction methods using organic solvents, buffers or others. The fractionating and refining step can include precipitation methods using salting out, solvent precipitation, isoelectric precipitation or others; column chromatographies such as ion-exchange chromatography, gel filtration chromatography, hydrophobic interaction chromatography, affinity chromatography and others, ultrafiltration, lyophilization, crystallization, dialysis and other methods. More specifically, the purification may be effected, for example, using procedures comprising the following steps (a) to (e) of:

(a) disrupting the microbial cells with glass beads;
(b) adding a protease or proteases to the resulting disrupted solution and degrading contaminated proteins;
(c) collecting the supernatant by centrifuging the disrupted solution after the degradation;
(d) salting-out and refining the protein from the collected supernatant under various pH conditions; and
(e) dissolving the precipitate collected by the salting-out, followed by desalting and removing particles.

The glycine repeat sequence protein obtained by the process obtained by the present invention is a versatile material (for example, in which changes in the physical properties related to the hydrophilicity of glycine repeat sequence proteins which result from neutral sugars are reduced so as to exhibit more lipophilic properties that are similar to those inherently possessed by glycine repeat sequence proteins) that is more commercially valuable for pharmaceuticals, industrial products, cosmetics and foods etc. This glycine repeat sequence protein would be also preferred due to its potentiality of inducing low immunological antigenicity.

### EXAMPLES

The present invention will be described in more detail below by way of Examples, and the present invention is not limited thereto.

In methods for cloning genes and constructing plasmids, methods described in “Molecular Cloning: A Laboratory Manual 2nd edition,” Cold Spring Harbor Laboratory Press (1989), ISBN 0-87969-309-6; “Current Protocols in Molecular Biology,” John Wiley & Sons, Inc. (1987), ISBN O-471-50338-X; and the like can be cited as reference. The cloning procedures and others will be described below in detail.

### Example 1

### Cloning

(1-1) Cloning of a DNA Encoding Histidinol Dehydrogenase (Construction of pHIS4-TOPO)

The oligonucleotides 1 and 2 mentioned below were synthesized. Komagataella pastoris NRRL Y-11430 (ATCC 76273) which was commercially available from the American Type Culture Collection (ATCC) was purchased. A DNA fragment encoding histidinol dehydrogenase (HIS4) was amplified by PCR using the oligonucleotides 1 and 2 as primers and a genomic DNA of ATCC 76273 as a template. The genomic DNA was prepared using Genomic-tip 100/G (QIAGEN) and Genomic DNA Buffer Set (QIAGEN).

The oligonucleotides 1 and 2 used were:

(a) oligonucleotide 1:
(SEQ ID NO: 1)
GATCTCCTGATGACTGACTCACTGATAATA,
and
(b) oligonucleotide 2:
(SEQ ID NO: 2)
TAATTAAATAAGTCCCAGTTTCTCCATACG.

The composition of the reaction solution is given as follows:

(a) genomic DNA (15 ng/μl), 1 μl;
(b) primers (10 pmol/μl), 1.5 μl each;
(c) 10× AccuPrime Pfx reaction mix (Invitrogen), 5 μl;
(d) AccuPrime Pfx DNA polymerase (2.5 U/μl, Invitrogen), 0.5 μl;
(e) sterile distilled water 40.5 μl.

The PCR was conducted under conditions where the reaction solution was heated at 95° C. for 2 minutes and then subjected to 35 cycles of denaturation at 95° C. for 15 seconds, annealing at 60° C. for 30 seconds, and extension at 68° C. for 2.5 minutes, followed by additionally keeping the reaction solution at 68° C. for 5 minutes.

An about 2.6-kb DNA fragment resulted from the PCR was isolated by agarose gel electrophoresis, followed by extraction and purification from the gel using MinElute Gel Extraction Kit (QIAGEN). The about 2.6-kb DNA fragment was ligated into the “PCR Product insertion site” of a pCR-BluntII-TOPO plasmid (Invitrogen), and the resulting ligation solution was used for transformation of E. coli (One Shot TOP10F′ Chemically Competent E. coli, Invitrogen). For cloning into the plasmid, a Zero-Blunt TOPO PCR cloning kit (Invitrogen) was used.

On LB agar medium containing 50 μg/ml of kanamycin, the E. coli cells which had been transformed were inoculated and cultured. A colony formed on the agar medium was inoculated into LB medium containing 50 μg/ml of kanamycin and incubated with shaking (37° C., 17 hours). Then, using QIAprep Spin Miniprep Kit (QIAGEN), the plasmid into which the DNA fragment encoding HIS4 had been inserted (which may be referred to hereinafter as pHIS4-TOPO) was isolated from the cultured cells to give pHIS4-TOPO.

(1-2) Cloning of a DNA Encoding Arginosuccinate Lyase (Construction of pARG4-TOPO)

The oligonucleotides 3 and 4 mentioned below were synthesized. A DNA fragment encoding arginosuccinate lyase (ARG4) was amplified by PCR using the oligonucleotides 3 and 4 as primers and a genomic DNA of ATCC 76273 (see, Example 1(1-1)) as a template. The genomic DNA was prepared using Genomic-tip 100/G (QIAGEN) and Genomic DNA Buffer Set (QIAGEN).

The oligonucleotides 3 and 4 used were:

(a) oligonucleotide 3:
(SEQ ID NO: 3)
ACGAAAATATGGTACCTGCCCTCAC,
and
(b) oligonucleotide 4:
(SEQ ID NO: 4)
GTTCTATCTACCCGAGGAAACCGATACATA.

The composition of the reaction solutions is given as follows:

(a) genomic DNA (15 ng/μl), 1 μl;
(b) primers (10 pmol/μl), 1.5 μl each;
(c) 10× AccuPrime Pfx reaction mix (Invitrogen), 5 μl;
(d) AccuPrime Pfx DNA polymerase (2.5 U/μl, Invitrogen), 0.5 μl;
(e) sterile distilled water 40.5 μl.

The PCR was conducted under conditions where the reaction solution was heated at 95° C. for 2 minutes and then subjected to 35 cycles of denaturation at 95° C. for 15 seconds, annealing at 65° C. for 30 seconds, and extension at 68° C. for 2.5 minutes, followed by additionally keeping the reaction solution at 68° C. for 5 minutes.

An about 2.2-kb DNA fragment resulted from the PCR was isolated by agarose gel electrophoresis, followed by extraction and purification from the gel using MinElute Gel Extraction Kit (QIAGEN). The about 2.2-kb DNA fragment was ligated into the “PCR Product insertion site” of a pCR-BluntII-TOPO plasmid (Invitrogen), and the resulting ligation solution was used for transformation of E. coli (One Shot TOP10F′ Chemically Competent E. coli, Invitrogen). For the ligation reaction, a Zero-Blunt TOPO PCR cloning kit (Invitrogen) was used.

On LB agar medium containing 50 μg/ml of kanamycin, the E. coli cells which had been transformed were inoculated and cultured. A colony formed on the agar medium was inoculated into LB medium containing 50 μg/ml of kanamycin and incubated with shaking (37° C., 17 hours). Then, using QIAprep Spin Miniprep Kit (QIAGEN), the plasmid into which the DNA fragment encoding ARG4 had been inserted (which may be referred to hereinafter as pARG4-TOPO) was isolated from the cultured cells to give pARG4-TOPO.

(1-3) Cloning of the Alcohol Dehydrogenase 1 Promoter (Construction of pAOX1Pro+15aa-TOPO)

The oligonucleotides 5 and 6 mentioned below were synthesized. The promoter of alcohol dehydrogenase 1 (AOX1) was amplified by PCR using the oligonucleotides 5 and 6 as primers and a genomic DNA of ATCC 76273 (see, Example 1(1-1)) as a template. The genomic DNA was prepared using Genomic-tip 100/G (QIAGEN) and Genomic DNA Buffer Set (QIAGEN).

The oligonucleotides 5 and 6 used were:

(a) oligonucleotide 5:
(SEQ ID NO: 5)
AGATCTAACATCCAAAGACGAAAGGTT,
and
(b) oligonucleotide 6:
(SEQ ID NO: 6)
ATCCACCACCTAGAACTAGGATATCAAAC.

The composition of the reaction solution is given as follows:

(a) genomic DNA (15 ng/μl), 1 μl;
(b) primers (10 pmol/μl), 1.5 μl each;
(c) 10× AccuPrime Pfx reaction mix (Invitrogen), 5 μl;
(d) AccuPrime Pfx DNA polymerase (2.5 U/μl, Invitrogen), 0.5 μl;
(e) sterile distilled water 40.5 μl.

The PCR was conducted under conditions where the reaction solution was heated at 95° C. for 2 minutes and then subjected to 35 cycles of denaturation at 95° C. for 15 seconds, annealing at 62° C. for 30 seconds, and extension at 68° C. for 1 minute, followed by additionally keeping the reaction solution at 68° C. for 5 minutes.

An about 1.0-kb DNA fragment resulted from the PCR was isolated by agarose gel electrophoresis, followed by extraction and purification from the gel using MinElute Gel Extraction Kit (QIAGEN). The about 1.0-kb DNA fragment was ligated into the “PCR Product insertion site” of a pCR-BluntII-TOPO plasmid (Invitrogen), and the resulting ligation solution was used for transformation of E. coli (One Shot TOP10F′ Chemically Competent E. coli, Invitrogen). For the ligation reaction, a Zero-Blunt TOPO PCR cloning kit (Invitrogen) was used.

On LB agar medium containing 50 μg/ml of kanamycin, the E. coli cells which had been subjected transformation were inoculated and cultured. A colony formed on the agar medium was inoculated into LB medium containing 50 μg/ml of kanamycin and incubated with shaking (37° C., 17 hours). Then, using QIAprep Spin Miniprep Kit (QIAGEN), the plasmid into which the AOX1 promoter had been inserted (which may be referred to hereinafter as pAOX1Pro+15aa-TOPO) was isolated from the cultured cells to give pARG4pAOX1Pro+15aa-TOPO.

(1-4) Cloning of the Peroxisome Matrix Protein Promoter (Construction of pCR-BII-PER3 Pro SacII-Psp1406I(+))

The oligonucleotides 7 and 8 mentioned below were synthesized. The promoter of peroxisome matrix protein (PER3) was amplified by PCR using the oligonucleotides 7 and 8 as primers and a genomic DNA of ATCC 76273 (see, Example 1(1-1)) as a template. The genomic DNA was prepared using Genomic-tip 100/G (QIAGEN) and Genomic DNA Buffer Set (QIAGEN).

The oligonucleotides 7 and 8 used were:

(a) oligonucleotide 7:
(SEQ ID NO: 7)
CTAAGGGTCTCACTGGTGTTTCAGC,
and
(b) oligonucleotide 8:
(SEQ ID NO: 8)
AGTTCCTTGCAACTGTAGTGGTCG.

The composition of the reaction solutions is given as follows:

(a) genomic DNA (15 ng/μl), 1 μl;
(b) dNTPs (mix, 2 mM each), 5 μl;
(c) MgSO₄ (25 mM), 2 μl;
(d) primers (10 pmol/μl), 1.5 μl each;
(e) 10×PCR buffer for KOD-plus- (Toyobo Co., Ltd.), 5 μl;
(f) KOD-plus- DNA polymerase (1 U/μl, Toyobo Co., Ltd.), 1 μl (1 U);
(g) sterile distilled water 33 μl.

The PCR was conducted under conditions where the reaction solution was heated at 94° C. for 2 minutes and then subjected to 30 cycles of denaturation at 94° C. for 15 seconds, and each of annealing and extension at 68° C. for 70 seconds, followed by additionally keeping the reaction solution at 68° C. for 70 seconds.

An about 1.1-kb DNA fragment resulted from the PCR was isolated by agarose gel electrophoresis, followed by extraction and purification from the gel using MinElute Gel Extraction Kit (QIAGEN). The about 1.1-kb DNA fragment was ligated into the “PCR Product insertion site” of a pCR-BluntII-TOPO plasmid (Invitrogen), and the resulting ligation solution was used for transformation of E. coli (Competent high DH5α, Toyobo Co., Ltd.). For the ligation reaction, a Zero-Blunt TOPO PCR cloning kit (Invitrogen) was used.

On LB agar medium containing 50 μg/ml of kanamycin, the E. coli cells which had been transformed were inoculated and cultured. A colony formed on the agar medium was inoculated into LB medium containing 50 μg/ml of kanamycin and incubated with shaking (37° C., 17 hours). Then, using QIAprep Spin Miniprep Kit (QIAGEN), the plasmid into which the PER3 promoter had been inserted (which may be referred to hereinafter as pCR-BII-PER3 Pro(+)) was isolated from the cultured cells to give pCR-BII-PER3 Pro(+).

The oligonucleotides 9 and 10 mentioned below were synthesized. A DNA fragment which had a restriction enzyme recognition sequence added at each end of the PER3 promoter was amplified by PCR using the oligonucleotides 9 and 10 as primers and the pCR-BII-PER3 Pro(+) plasmid as a template.

The oligonucleotides 9 and 10 used were:

(a) oligonucleotide 9:
(SEQ ID NO: 9)
AACCGCGGCTCGTCACTATCGTCGTTG,
and
(b) oligonucleotide 10:
(SEQ ID NO: 10)
CGAACGTTACCTGAAGATAGGTAAAAAAAAATTGC.

The composition of the reaction solutions is given as follows:

(a) plasmid solution (1 ng/μl), 1 μl;
(b) dNTPs (mix, 2 mM each), 5 μl;
(c) MgSO₄ (25 mM), 2 μl;
(d) primers (10 pmol/μl), 1.5 μl each;
(e) 10×PCR buffer for KOD-plus- (Toyobo Co., Ltd.), 5 μl;
(f) KOD-plus- DNA polymerase (1 U/μl, Toyobo Co., Ltd.), 1 μl (1 U);
(g) sterile distilled water 33 μl.

The PCR was conducted under conditions where the reaction solution was heated at 94° C. for 2 minutes and then subjected to 5 cycles of denaturation at 94° C. for 15 seconds, annealing at 60° C. for 30 seconds, and extension at 68° C. for 1 minute and then 20 cycles of denaturation at 94° C. for 15 seconds, and each of annealing and extension at 68° C. for 1 minute, followed by additionally keeping the reaction solution at 68° C. for 1 minutes.

An about 1.0-kb DNA fragment resulted from the PCR was isolated by agarose gel electrophoresis, followed by extraction and purification from the gel using MinElute Gel Extraction Kit (QIAGEN). The about 1.0-kb DNA fragment was ligated into the “PCR Product insertion site” of a pCR-BluntII-TOPO plasmid (Invitrogen), and the resulting ligation solution was used for transformation of E. coli (Competent high DH5α, Toyobo Co., Ltd.). For the ligation reaction, a Zero-Blunt TOPO PCR cloning kit (Invitrogen) was used.

On LB agar medium containing 50 μg/ml of kanamycin, the E. coli cells which had been transformed were inoculated and cultured. A colony formed on the agar medium was inoculated into LB medium containing 50 μg/ml of kanamycin and incubated with shaking (37° C., 17 hours). Then, using QIAprep Spin Miniprep Kit (QIAGEN), the plasmid into which the PER3 promoter had been inserted (which may be referred to hereinafter as pCR-BII-PER3 Pro SacII-Psp1406I(+)) was isolated from the cultured cells to give pCR-BII-PER3 Pro SacII-Psp1406I(+).

(1-5) Cloning of the Alcohol Dehydrogenase 2 Promoter (Construction of pCR-BII-AOX2 Pro SacII-Psp1406I(−))

The oligonucleotides 11 and 12 mentioned below were synthesized. A DNA fragment which had a restriction enzyme recognition sequence added at each end of the alcohol dehydrogenase 2 (AOX2) promoter was amplified by PCR using the oligonucleotides 11 and 12 as primers and a genomic DNA of ATCC 76273 (see, Example 1(1-1)) as a template. The genomic DNA was prepared using Genomic-tip 100/G (QIAGEN) and Genomic DNA Buffer Set (QIAGEN).

The oligonucleotides 11 and 12 used were:

(a) oligonucleotide 11:
(SEQ ID NO: 11)
AACCGCGGCTAGTAGAACTTTGACATCTGCTA,
and
(b) oligonucleotide 12:
(SEQ ID NO: 12)
CGAACGTTTTGATTTGTTTGTGGGGATTTAG.

The composition of the reaction solutions is given as follows:

(a) genomic DNA (15 ng/μl), 1 μl;
(b) dNTPs (mix, 2 mM each), 5 μl;
(c) MgSO₄ (25 mM), 2 μl;
(d) primers (10 pmol/μl), 1.5 μl each;
(e) 10×PCR buffer for KOD-plus- (Toyobo Co., Ltd.), 5 μl;
(f) KOD-plus- DNA polymerase (1 U/μl, Toyobo Co., Ltd.), 1 μl (1 U);
(g) sterile distilled water 33 μl.

The PCR was conducted under conditions where the reaction solution was heated at 94° C. for 2 minutes and then subjected to 5 cycles of denaturation at 94° C. for 15 seconds, annealing at 60° C. for 30 seconds, and extension at 68° C. for 70 seconds and then 30 cycles of denaturation at 94° C. for 15 seconds, and each of annealing and extension at 68° C. for 70 seconds, followed by additionally keeping the reaction solution at 68° C. for 70 seconds.

An about 1.1-kb DNA fragment resulted from the PCR was isolated by agarose gel electrophoresis, followed by extraction and purification from the gel using MinElute Gel Extraction Kit (QIAGEN). The about 1.1-kb DNA fragment was ligated into the “PCR Product insertion site” of a pCR-BluntII-TOPO plasmid (Invitrogen), and the resulting ligation solution was used for transformation of E. coli (Competent high DH5α, Toyobo Co., Ltd.). For the ligation reaction, a Zero-Blunt TOPO PCR cloning kit (Invitrogen) was used.

On LB agar medium containing 50 μg/ml of kanamycin, the E. coli cells which had been transformed were inoculated and cultured. A colony formed on the agar medium was inoculated into LB medium containing 50 μg/ml of kanamycin and incubated with shaking (37° C., 17 hours). Then, using QIAprep Spin Miniprep Kit (QIAGEN), the plasmid into which the AOX2 promoter had been inserted (which may be referred to hereinafter as pCR-BII-AOX2 Pro SacII-Psp1406I(−)) was isolated from the cultured cells to give pCR-BII-AOX2 Pro SacII-Psp1406I(−).

(1-6) Cloning of the Formaldehyde Dehydrogenase Promoter (Construction of pCR-BII-FLD1 Pro SacII-Psp1406I(−))

The oligonucleotides 13 and 14 mentioned below were synthesized. The promoter of formaldehyde dehydrogenase (FLD1) was amplified by PCR using the oligonucleotides 13 and 14 as primers and a genomic DNA of ATCC 76273 (see, Example 1(1-1)) as a template. The genomic DNA was prepared using Genomic-tip 100/G (QIAGEN) and Genomic DNA Buffer Set (QIAGEN).

The oligonucleotides 13 and 14 used were:

(a) oligonucleotide 13:
(SEQ ID NO: 13)
GCAGTGTTGGCTAACGTCTATTCG,
and
(b) oligonucleotide 14:
(SEQ ID NO: 14)
ACTTCATGGGCTCTTGGAGGAAG.

The composition of the reaction solutions is given as follows:

(a) genomic DNA (15 ng/μl), 1 μl;
(b) dNTPs (mix, 2 mM each), 5 μl;
(c) MgSO₄ (25 mM), 2 μl;
(d) primers (10 pmol/μl), 1.5 μl each;
(e) 10×PCR buffer for KOD-plus- (Toyobo Co., Ltd.), 5 μl;
(f) KOD-plus- DNA polymerase (1 U/μl, Toyobo Co., Ltd.), 1 μl (1 U);
(g) sterile distilled water 33 μl.

The PCR was conducted under conditions where the reaction solution was heated at 94° C. for 2 minutes and then subjected to 30 cycles of denaturation at 94° C. for 15 seconds, and each of annealing and extension at 68° C. for 70 seconds, followed by additionally keeping the reaction solution at 68° C. for 70 seconds.

An about 1.3-kb DNA fragment resulted from the PCR was isolated by agarose gel electrophoresis, followed by extraction and purification from the gel using MinElute Gel Extraction Kit (QIAGEN). The about 1.3-kb DNA fragment was ligated into the “PCR Product insertion site” of a pCR-BluntII-TOPO plasmid (Invitrogen), and the resulting ligation solution was used for transformation of E. coli (Competent high DH5α, Toyobo Co., Ltd.). For the ligation reaction, a Zero-Blunt TOPO PCR cloning kit (Invitrogen) was used.

On LB agar medium containing 50 μg/ml of kanamycin, the E. coli cells which had been transformed were inoculated and cultured. A colony formed on the agar medium was inoculated into LB medium containing 50 μg/ml of kanamycin and incubated with shaking (37° C., 17 hours). Then, using QIAprep Spin Miniprep Kit (QIAGEN), the plasmid into which the FLD1 promoter had been inserted (which may be referred to hereinafter as pCR-BII-FLD1 Pro(+)) was isolated from the cultured cells to give pCR-BII-FLD1 Pro(+).

The oligonucleotides 15 and 16 mentioned below were synthesized. A DNA fragment which had a restriction enzyme recognition sequence added at each end of the FLD1 promoter was amplified by PCR using the oligonucleotides 15 and 16 as primers and the pCR-BII-FLD1 Pro(+) plasmid as a template.

The oligonucleotides 15 and 16 used were:

(a) oligonucleotide 15:
(SEQ ID NO: 15)
AACCGCGGCCTGAATACCGTAACATAGTGAC,
and
(b) oligonucleotide 16:
(SEQ ID NO: 16)
CGAACGTTTCAAGAATTGTATGAACAAGCAAAG.

The composition of the reaction solutions is given as follows:

(a) plasmid solution (1 ng/μl), 1 μl;
(b) dNTPs (mix, 2 mM each), 5 μl;
(c) MgSO₄ (25 mM), 2 μl;
(d) primers (10 pmol/μl), 1.5 μl each;
(e) 10×PCR buffer for KOD-plus- (Toyobo Co., Ltd.), 5 μl;
(f) KOD-plus- DNA polymerase (1 U/μl, Toyobo Co., Ltd.), 1 μl (1 U);
(g) sterile distilled water 33 μl.

The PCR was conducted under conditions where the reaction solution was heated at 94° C. for 2 minutes and then subjected to 5 cycles of denaturation at 94° C. for 15 seconds, annealing at 60° C. for 30 seconds, and extension at 68° C. for 1 minute and then 20 cycles of denaturation at 94° C. for 15 seconds, and each of annealing and extension at 68° C. for 1 minute, followed by additionally keeping the reaction solution at 68° C. for 1 minutes.

An about 0.9-kb DNA fragment resulted from the PCR was isolated by agarose gel electrophoresis, followed by extraction and purification from the gel using MinElute Gel Extraction Kit (QIAGEN). The about 0.9-kb DNA fragment was ligated into the “PCR Product insertion site” of a pCR-BluntII-TOPO plasmid (Invitrogen), and the resulting ligation solution was used for transformation of E. coli (Competent high DH5α, Toyobo Co., Ltd.). For the ligation reaction, a Zero-Blunt TOPO PCR cloning kit (Invitrogen) was used.

On LB agar medium containing 50 μg/ml of kanamycin, the E. coli cells which had been transformed were inoculated and cultured. A colony formed on the agar medium was inoculated into LB medium containing 50 μg/ml of kanamycin and incubated with shaking (37° C., 17 hours). Then, using QIAprep Spin Miniprep Kit (QIAGEN), the plasmid into which the FLD1 promoter had been inserted (which may be referred to hereinafter as pCR-BII-FLD1 Pro SacII-Psp1406I(−)) was isolated from the cultured cells to give pCR-BII-FLD1 Pro SacII-Psp1406I(−).

(1-7) Cloning of the Alcohol Dehydrogenase 1 Terminator (Construction of pAOX1 Term-TOPO)

The oligonucleotides 17 and 18 mentioned below were synthesized. The terminator of alcohol dehydrogenase 1 (AOX1) was amplified by PCR using the oligonucleotides 17 and 18 as primers and a genomic DNA of ATCC 76273 (see, Example 1(1-1)) as a template. The genomic DNA was prepared using Genomic-tip 100/G (QIAGEN) and Genomic DNA Buffer Set (QIAGEN).

The oligonucleotides 17 and 18 used were:

(a) oligonucleotide 17:
(SEQ ID NO: 17)
CCTTAGACATGACTGTTCCTCAGTTC,
and
(b) oligonucleotide 18:
(SEQ ID NO: 18)
GCACAAACGAACGTCTCACTTAAT.

The composition of the reaction solutions is given as follows:

(a) genomic DNA (15 ng/μl), 1 μl;
(b) primers (10 pmol/μl), 1.5 μl each;
(c) 10× AccuPrime Pfx reaction mix (Invitrogen), 5 μl;
(d) AccuPrime Pfx DNA polymerase (2.5 U/μl, Invitrogen), 0.5 μl;
(e) sterile distilled water 40.5 μl.

The PCR was conducted under conditions where the reaction solution was heated at 95° C. for 2 minutes and then subjected to 35 cycles of denaturation at 95° C. for 15 seconds, annealing at 60° C. for 30 seconds, and extension at 68° C. for 30 seconds, followed by additionally keeping the reaction solution at 68° C. for 5 minutes.

An about 0.3-kb DNA fragment resulted from the PCR was isolated by agarose gel electrophoresis, followed by extraction and purification from the gel using MinElute Gel Extraction Kit (QIAGEN). The about 0.3-kb DNA fragment was ligated into the “PCR Product insertion site” of a pCR-BluntII-TOPO plasmid (Invitrogen), and the resulting ligation solution was used for transformation of E. coli (One Shot TOP10F′ Chemically Competent E. coli, Invitrogen). For the ligation reaction, a Zero-Blunt TOPO PCR cloning kit (Invitrogen) was used.

On LB agar medium containing 50 μg/ml of kanamycin, the E. coli cells which had been transformed were inoculated and cultured. A colony formed on the agar medium was inoculated into LB medium containing 50 μg/ml of kanamycin and incubated with shaking (37° C., 17 hours). Then, using QIAprep Spin Miniprep Kit (QIAGEN), the plasmid into which the AOX1 terminator had been inserted (which may be referred to hereinafter as pAOX1 term-TOPO) was isolated from the cultured cells to give pAOX1 term-TOPO.

(1-8) Cloning of a 3′-Downstream Non-Coding Region of the Alcohol Dehydrogenase 1 Gene (Construction of pAOX1-3′-TOPO)

The oligonucleotides 19 and 20 mentioned below were synthesized. A 3′-downstream non-coding region of the alcohol dehydrogenase 1 (AOX1) gene was amplified by PCR using the oligonucleotides 19 and 20 as primers and a genomic DNA of ATCC 76273 (see, Example 1(1-1)) as a template. The genomic DNA was prepared using Genomic-tip 100/G (QIAGEN) and Genomic DNA Buffer Set (QIAGEN).

The oligonucleotides 19 and 20 used were:

(a) oligonucleotide 19:
(SEQ ID NO: 19)
TCGAGTATCTATGATTGGAAGTATGGGAAT,
and
(b) oligonucleotide 20:
(SEQ ID NO: 20)
GATCTTGAGATAAATTTCACGTTTAAAATC.

The composition of the reaction solutions is given as follows:

(a) genomic DNA (15 ng/μl), 1 μl;
(b) primers (10 pmol/μl), 1.5 μl each;
(c) 10× AccuPrime Pfx reaction mix (Invitrogen), 5 μl;
(d) AccuPrime Pfx DNA polymerase (2.5 U/μl, Invitrogen), 0.5 μl;
(e) sterile distilled water 40.5 μl.

The PCR was conducted under conditions where the reaction solution was heated at 94° C. for 2 minutes and then subjected to 35 cycles of denaturation at 95° C. for 15 seconds, annealing at 58° C. for 30 seconds, and extension at 68° C. for 50 seconds, followed by additionally keeping the reaction solution at 68° C. for 5 minutes.

An about 0.8-kb DNA fragment resulted from the PCR was isolated by agarose gel electrophoresis, followed by extraction and purification from the gel using MinElute Gel Extraction Kit (QIAGEN). The about 0.8-kb DNA fragment was ligated into the “PCR Product insertion site” of a pCR-BluntII-TOPO plasmid (Invitrogen), and the resulting ligation solution was used for transformation of E. coli (One Shot TOP10F′ Chemically Competent E. coli, Invitrogen). For the ligation reaction, a Zero-Blunt TOPO PCR cloning kit (Invitrogen) was used.

On LB agar medium containing 50 μg/ml of kanamycin, the E. coli cells which had been transformed were inoculated and cultured. A colony formed on the agar medium was inoculated into LB medium containing 50 μg/ml of kanamycin and incubated with shaking (37° C., 17 hours). Then, using QIAprep Spin Miniprep Kit (QIAGEN), the plasmid into which the 3′-downstream non-coding region of AOX1 had been inserted (which may be referred to hereinafter as pAOX1-3′-TOPO) was isolated from the cultured cells to give pAOX1-3′-TOPO.

(1-9) Cloning of a DNA Encoding a Factor (Construction of pαfactor-TOPO)

The oligonucleotides 21 and 22 mentioned below were synthesized. Saccharomyces cerevisiae S288C (NBRC 1136) which was commercially available from the National Institute of Technology and Evaluation (NITE) was purchased. A DNA fragment encoding a factor was amplified by PCR using the oligonucleotides 21 and 22 as primers and a genomic DNA of NBRC 1136 as a template. The genomic DNA was prepared using Genomic-tip 100/G (QIAGEN) and Genomic DNA Buffer Set (QIAGEN).

The oligonucleotides 21 and 22 used were:

(a) oligonucleotide 21:
(SEQ ID NO: 21)
TCAAACAAGAAGATTACAAACTATCAATTTCA,
and
(b) oligonucleotide 22:
(SEQ ID NO: 22)
GTACGAGCTAAAAGTACAGTGGGAACAAA.

The composition of the reaction solutions is given as follows:

(a) genomic DNA (15 ng/μl), 1 μl;
(b) dNTPs (mix, 2 mM each), 5 μl;
(c) MgSO₄ (25 mM), 2 μl;
(d) primers (10 pmol/μl), 1.5 μl each;
(e) 10×PCR buffer for KOD-plus- (Toyobo Co., Ltd.), 5 μl;
(f) KOD-plus- DNA polymerase (1 U/μl, Toyobo Co., Ltd.), 1 μl (1 U);
(g) sterile distilled water 33 μl.

The PCR was conducted under conditions where the reaction solution was heated at 94° C. for 3 minutes and then subjected to 10 cycles of denaturation at 94° C. for 15 seconds, annealing at 60° C. for 30 seconds, and extension at 68° C. for 1 minute and then 25 cycles of denaturation at 94° C. for 15 seconds, and each of annealing and extension at 68° C. for 1 minute, followed by additionally keeping the reaction solution at 68° C. for 5 minutes.

An about 0.6-kb DNA fragment resulted from the PCR was isolated by agarose gel electrophoresis, followed by extraction and purification from the gel using MinElute Gel Extraction Kit (QIAGEN). The about 0.6-kb DNA fragment was ligated into the “PCR Product insertion site” of a pCR-BluntII-TOPO plasmid (Invitrogen), and the resulting ligation solution was used for transformation of E. coli (One Shot TOP10F′ Chemically Competent E. coli, Invitrogen). For the ligation reaction, a Zero-Blunt TOPO PCR cloning kit (Invitrogen) was used.

On LB agar medium containing 50 μg/ml of kanamycin, the E. coli cells which had been transformed were inoculated and cultured. A colony formed on the agar medium was inoculated into LB medium containing 50 μg/ml of kanamycin and incubated with shaking (37° C., 17 hours). Then, using QIAprep Spin Miniprep Kit (QIAGEN), the plasmid into which the DNA fragment encoding a factor had been inserted (which may be referred to hereinafter as pαfactor-TOPO) was isolated from the cultured cells to give pαfactor-TOPO.

(1-10) Cloning of a DNA Encoding Human Collagen Type I α1 (Construction of pBlue-HsCOL1A1)

The oligonucleotides 23 and 24 mentioned below were synthesized. To these oligonucleotides, a phosphate group was added at their 5′ end using T4 polynucleotide kinase (Toyobo Co., Ltd.). Then, a DNA fragment encoding human collagen Type I α1 was amplified by PCR using the phosphorylated oligonucleotides 23 and 24 as primers and Human brain cDNAs (Toyobo Co., Ltd.) as a template.

The oligonucleotides 23 and 24 used were:

(a) oligonucleotide 23:
(SEQ ID NO: 23)
CAGCCACAAAGAGTCTACATGTCTAGG,
and
(b) oligonucleotide 24:
(SEQ ID NO: 24)
AGGTTGGGATGGAGGGAGTT.

The composition of the reaction solutions is given as follows:

(a) cDNA solution, 5 μl;
(b) dNTPs (mix, 2 mM each), 5 μl;
(c) MgSO₄ (25 mM), 2 μl;
(d) primers (10 pmol/μl), 1.5 μl each;
(e) 10×PCR buffer for KOD-plus- (Toyobo Co., Ltd.), 5 μl;
(f) KOD-plus- DNA polymerase (1 U/μl, Toyobo Co., Ltd.), 1 μl;
(g) sterile distilled water 29 μl.

The PCR was conducted under conditions where the reaction solution was heated at 94° C. for 2 minutes and then subjected to 35 reaction cycles of denaturation at 98° C. for 20 seconds, annealing at 58° C. for 10 seconds, and extension at 74° C. for 5 minutes.

An about 4.5-kb DNA fragment resulted from the PCR was purified using MagExtractor PCR&Gel cleanup (Toyobo Co., Ltd.).

A pBluescriptII KS(+) plasmid (Stratagene) was digested with a restriction enzyme EcoRV, dephosphorylated with an alkaline phosphatase, and then purified using MagExtractor PCR&Gel cleanup (Toyobo Co., Ltd.).

The about 4.5-kb DNA fragment and the dephosphorylated plasmid were ligated, and the resulting ligation solution was used for transformation of E. coli (Competent high DH5α, Toyobo Co., Ltd.). For the ligation reaction, a Ligation high (Toyobo Co., Ltd.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using MagExtractor plasmid (Toyobo Co., Ltd.), the plasmid into which the DNA fragment encoding human collagen Type I α1 had been inserted (which may be referred to hereinafter as pBlue-HsCOL1A1) was isolated from the cultured cells to give pBlue-HsCOL1A1.

(1-11) Cloning of a DNA Encoding Human Collagen Type I α2 (Construction of pUC18-HsCOL1A2)

Total RNA derived from Human Neonatal Dermal Fibroblasts was obtained from Cell Applications, Inc. CDNAs were synthesized using ReverTra Plus (Toyobo Co., Ltd.). The oligonucleotides 25 and 26 mentioned below were synthesized. A DNA fragment encoding human collagen Type I α2 was amplified by PCR using the oligonucleotides 25 and 26 as primers and the synthesized cDNAs as a template.

The oligonucleotides 25 and 26 used were:

(a) oligonucleotide 25:
(SEQ ID NO: 25)
GCCAAGCTTGCATGCTCAGCTTTGTGGATACGCGGAC,
and
(b) oligonucleotide 26:
(SEQ ID NO: 26)
CGGTACCCGGGGATCCTTATTTGAAACAGACTGGGCCAATGTCC.

The composition of the reaction solutions is given as follows:

(a) cDNA solution, 5 μl;
(b) dNTPs (mix, 2 mM each), 5 μl;
(c) MgSO₄ (25 mM), 2 μl;
(d) primers (10 pmol/μl), 1.5 μl each;
(e) 10×PCR buffer for KOD-plus- (Toyobo Co., Ltd.), 5 μl;
(f) KOD-plus- DNA polymerase (1 U/μl, Toyobo Co., Ltd.), 1 μl;
(g) sterile distilled water 29 μl.

The PCR was conducted under conditions where the reaction solution was heated at 94° C. for 2 minutes and then subjected to 30 reaction cycles of denaturation at 98° C. for 10 seconds, and each of annealing and extension at 68° C. for 6 minutes. An about 4.1-kb DNA fragment amplified was isolated by agarose gel electrophoresis, followed by extraction and purification from the gel using MagExtractor PCR&Gel cleanup (Toyobo Co., Ltd.). The obtained DNA was digested with restriction enzymes SphI and BamHI, and then purified using MagExtractor PCR&Gel cleanup (Toyobo Co., Ltd.).

A pUC18 plasmid (Toyobo Co., Ltd.) was digested with restriction enzymes SphI and BamHI, and then purified using MagExtractor PCR&Gel cleanup (Toyobo Co., Ltd.).

The about 4.1-kb DNA fragment and the digested plasmid were ligated, and the resulting ligation solution was used for transformation of E. coli (Competent high DH5α, Toyobo Co., Ltd.). For the ligation reaction, a Ligation high (Toyobo Co., Ltd.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using MagExtractor plasmid (Toyobo Co., Ltd.), the plasmid into which the DNA fragment encoding human collagen Type I α2 had been inserted (which may be referred to hereinafter as pUC18-HsCOL1A2) was isolated from the cultured cells to give pUC18-HsCOL1A2.

(1-12) Cloning of a DNA Encoding Human Collagen Type III α1 (Construction of pUC19-HsCOL3A1)

The oligonucleotides 27 and 28 mentioned below were synthesized. To these oligonucleotides, a phosphate group was added at their 5′ ends using T4 polynucleotide kinase (Toyobo Co., Ltd.). Then, a DNA fragment encoding human collagen Type III α1 was amplified by PCR using the phosphorylated oligonucleotides 27 and 28 as primers and Human brain cDNAs (Toyobo Co., Ltd.) as a template.

The oligonucleotides 27 and 28 used were:

(SEQ ID NO: 27)
(a) oligonucleotide 27: GGCTGAGTTTTATGACGGGC,
and
(SEQ ID NO: 28)
(b) oligonucleotide 28: GACAAGATTAGAACAAGAGG.

The composition of the reaction solutions is given as follows:

(a) cDNA solution, 5 μl;
(b) dNTPs (mix, 2 mM each), 5 μl;
(c) MgSO₄ (25 mM), 2 μl;
(d) primers (10 pmol/μl), 1.5 μl each;
(e) 10×PCR buffer for KOD-plus- (Toyobo Co., Ltd.), 5 μl;
(f) KOD-plus- DNA polymerase (1 U/μl, Toyobo Co., Ltd.), 1 μl;
(g) sterile distilled water 29 μl.

The PCR was conducted under conditions where the reaction solution was heated at 94° C. for 2 minutes and then subjected to 35 reaction cycles of denaturation at 98° C. for 20 seconds, annealing at 58° C. for 10 seconds, and extension at 74° C. for 5 minutes.

An about 4.6-kb DNA fragment resulted from the PCR was purified using MagExtractor PCR&Gel cleanup (Toyobo Co., Ltd.).

A pUC19 plasmid (Toyobo Co., Ltd.) was digested with a restriction enzyme SmaI, purified using MagExtractor PCR&Gel cleanup (Toyobo Co., Ltd.), and then dephosphorylated with an alkaline phosphatase, and purified again using MagExtractor PCR&Gel cleanup (Toyobo Co., Ltd.).

The about 4.6-kb DNA fragment and the dephosphorylated plasmid were ligated, and the resulting ligation solution was used for transformation of E. coli (Competent high DH5α, Toyobo Co., Ltd.). For the ligation reaction, a Ligation high (Toyobo Co., Ltd.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using MagExtractor plasmid (Toyobo Co., Ltd.), the plasmid into which the DNA fragment encoding human collagen Type I α1 had been inserted (which may be referred to hereinafter as pUC19-HsCOL3A1) was isolated from the cultured cells to give pUC19-HsCOL3A1.

(1-13) Preparation and Cloning of a Zeocyn-Resistance Cassette (Construction of pUC57-ZeoR)

A Zeocyn-resistance cassette indicated by SEQ ID NO:112 was prepared by an assembly PCR method. The DNA fragment prepared was inserted into the EcoRV site of a pUC57 plasmid to be cloned. The resulting plasmid is referred to as pUC57-ZeoR.

(1-14) Preparation and Cloning of a Synthetic DNA Encoding an about 13-kDa FK506-Binding Protein (Construction of pUC57-YFKBP13A)

A DNA fragment encoding an about 13-kDa FK506-binding protein (FKBP13A) indicated by SEQ ID NO:113 was prepared by an assembly PCR method. The synthetic DNA fragment prepared was inserted into the EcoRV site of a pUC57 plasmid to be cloned. The resulting plasmid is referred to as pUC57-YFKBP13A.

(1-15) Preparation and Cloning of a Synthetic DNA Encoding an about 19-kDa FK506-Binding Protein (Construction of pUC57-YFKBP19)

A DNA fragment encoding an about 19-kDa FK506-binding protein (FKBP19) indicated by SEQ ID NO:78 was prepared by an assembly PCR method. The synthetic DNA fragment prepared was inserted into the EcoRV site of a pUC57 plasmid to be cloned. The resulting plasmid is referred to as pUC57-YFKBP19.

(1-16) Preparation and Cloning of a Synthetic DNA Encoding an about 23-kDa FK506-Binding Protein (Construction of pUC57-YFKBP23)

A synthetic DNA fragment encoding an about 23-kDa FK506-binding protein (FKBP23) indicated by SEQ ID NO:76 was prepared by an assembly PCR method. The synthetic DNA fragment prepared was inserted into the EcoRV site of a pUC57 plasmid to be cloned. The resulting plasmid is referred to as pUC57-YFKBP23.

(1-17) Preparation and Cloning of a Synthetic DNA Encoding an about 63-kDa FK506-Binding Protein (Construction of pUC57-YFKBP63)

A synthetic DNA fragment encoding an about 63-kDa FK506-binding protein (FKBP63) indicated by SEQ ID NO:114 was prepared by an assembly PCR method. The synthetic DNA fragment prepared was inserted into the EcoRV site of a pUC57 plasmid to be cloned. The resulting plasmid is referred to as pUC57-YFKBP63.

(1-18) Preparation and Cloning of a Synthetic DNA Encoding an about 65-kDa FK506-Binding Protein (Construction of pUC57-YFKBP65)

A synthetic DNA fragment encoding an about 65-kDa FK506-binding protein (FKBP65) indicated by SEQ ID NO:115 was prepared by an assembly PCR method. The synthetic DNA fragment prepared was inserted into the EcoRV site of a pUC57 plasmid to be cloned. The resulting plasmid is referred to as pUC57-YFKBP65.

(1-19) Preparation and Cloning of a Synthetic DNA Encoding Human Type I α1 Collagen (Construction of pUC57-YHsCOL1A1)

A synthetic DNA fragment encoding human Type I α1 collagen indicated by SEQ ID NO:98 was prepared by an assembly PCR method. The synthetic DNA fragment prepared was inserted into the EcoRV site of a pUC57 plasmid to be cloned. The resulting plasmid is referred to as pUC57-YHsCOL1A1.

(1-20) Preparation and Cloning of a Synthetic DNA Encoding Human Type I α2 Collagen (Construction of pUC57-YHsCOL1A2)

A synthetic DNA fragment encoding human Type I α2 collagen indicated by SEQ ID NO:100 was prepared by an assembly PCR method. The synthetic DNA fragment prepared was inserted into the EcoRV site of a pUC57 plasmid to be cloned. The resulting plasmid is referred to as pUC57-YHsCOL1A2.

(1-21) Preparation and Cloning of a Synthetic DNA Encoding Human Type II α1 Collagen (Construction of pUC57-YHsCOL2A1)

A synthetic DNA fragment encoding human Type II α1 collagen indicated by SEQ ID NO:102 was prepared by an assembly PCR method. The synthetic DNA fragment prepared was inserted into the EcoRV site of a pUC57 plasmid to be cloned. The resulting plasmid is referred to as pUC57-YHsCOL2A1.

### Example 2

### Construction of Plasmids for Introducing Expression Cassettes

(2-1) Preparation of a Plasmid (pEXP-A-P4HBsig(−)A1rev) for Introducing an Expression Cassette for a Prolyl 4-Hydroxylase α1 Subunit (P4Hα1) and an Expression Cassette for a Prolyl 4-Hydroxylase β Subunit (P4Hβ)
(2-1-1) Construction of pSN003

The oligonucleotides 29 and 30 mentioned below were synthesized. A DNA fragment which had a restriction enzyme recognition sequence added at each end of the AOX1 terminator was amplified by PCR using the oligonucleotides 29 and 30 as primers and the plasmid named as pAOX1Term-TOPO (see, Example (1-7)) as a template.

The oligonucleotides 29 and 30 used were:

(a) oligonucleotide 29:
(SEQ ID NO: 29)
TCGACTAGTTTAGACATGACTGTTCCTCAGTTCAA,
and
(b) oligonucleotide 30:
(SEQ ID NO: 30)
AACTGCAGGCACAAACGAACGTCTCACTTA.

The composition of the reaction solutions is given as follows:

(a) plasmid solution (10 ng/μl), 1 μl;
(b) dNTPs (mix, 2 mM each), 5 μl;
(c) MgSO₄ (25 mM), 2 μl;
(d) primers (10 pmol/μl), 1.5 μl each;
(e) 10×PCR buffer for KOD-plus- (Toyobo Co., Ltd.), 5 μl;
(f) KOD-plus- DNA polymerase (1 U/μl, Toyobo Co., Ltd.), 1 μl (1 U);
(g) sterile distilled water 33 μl.

The PCR was conducted under conditions where the reaction solution was heated at 94° C. for 2 minutes and then subjected to 25 cycles of denaturation at 94° C. for 15 seconds, annealing at 60° C. for 30 seconds, and extension at 68° C. for 1 minute, followed by additionally keeping the reaction solution at 68° C. for 5 minutes.

An about 0.3-kb DNA fragment resulted from the PCR was digested with restriction enzymes SpeI and PstI, and then isolated by agarose gel electrophoresis, followed by extraction and purification from the gel using MinElute Gel Extraction Kit (QIAGEN).

A pBluescriptII KS(+) plasmid (Stratagene) was digested with restriction enzymes SpeI and PstI, and then isolated by agarose gel electrophoresis, followed by extraction and purification from the gel using MinElute Gel Extraction Kit (QIAGEN).

The about 0.3-kb DNA fragment and the digested plasmid were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA having the restriction enzyme recognition sequence added at each end of the AOX1 terminator had been inserted (which may be referred to hereinafter as pSN003) was isolated from the cultured cells to give pSN003.

(2-1-2) Construction of pSN004

The plasmid named as pAOX1Pro+15aa-TOPO (see, Example (1-3)) was digested with a restriction enzyme Eco52I. Then, an about 1.0-kb DNA fragment comprising the AOX1 promoter was isolated by agarose gel electrophoresis, followed by extraction and purification from the gel using MinElute Gel Extraction Kit (QIAGEN).

The plasmid named as pSN003 (see, Example (2-1-1)) was digested with a restriction enzyme Eco52I, dephosphorylated with an alkaline phosphatase, and then purified using MinElute Reaction Cleanup Kit (QIAGEN).

The about 1.0-kb DNA fragment and the dephosphorylated plasmid were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the about 1.0-kb DNA fragment comprising the AOX1 promoter had been inserted (which may be referred to hereinafter as pSN004) was isolated from the cultured cells to give pSN004.

(2-1-3) Construction of pSN005

The oligonucleotides 31 and 32 mentioned below were synthesized:

(a) oligonucleotide 31:
(SEQ ID NO: 31)
TATTCGAAACGCATATGTGACCGGCAGACTAGTGG,
and
(b) oligonucleotide 32:
(SEQ ID NO: 32)
CCACTAGTCTGCCGGTCACATATGGGTTTCGAATA.

A solution having the composition mentioned below was prepared and kept at 98° C. for 5 minutes, at 50° C. for 50 minutes, and then at 37° C. for 1 hour:

(a) oligonucleotide 31 (50 pmol/μl), 5 μl;
(b) oligonucleotide 32 (50 pmol/μl), 5 μl;
(c) Tris-HCl (100 mM), 10 μl;
(d) MgCl₂ (100 mM), 10 μl;
(e) dithiothreitol (10 mM), 10 μl;
(f) sterile distilled water 60 μl.

A DNA linker in which the oligonucleotides 31 and 32 had been annealed was digested with restriction enzymes BspT104I and SpeI. Next, the mixture solution was extracted with phenol:chloroform:isoamyl alcohol (25:24:1) and then subjected to ethanol precipitation to purify the DNA linker (Linker 1).

Additionally, the plasmid named as pSN004 (see, Example (2-1-2)) was digested with restriction enzymes BspT104I and SpeI. An about 4.3-kb DNA fragment was separated and purified by agarose gel electrophoresis.

The DNA linker (Linker 1) and the about 4.3-kb DNA fragment were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA linker (Linker 1) had been inserted (which may be referred to hereinafter as pSN005) was isolated from the cultured cells to give pSN005.

(2-1-4) Construction of pSN015

The oligonucleotides 33 and 34 mentioned below were synthesized. A DNA fragment which had a restriction enzyme recognition sequence added at each end of the DNA encoding the signal sequence and the pro sequence of the α factor gene was amplified by PCR using the oligonucleotides 33 and 34 as primers and the plasmid named as pαfactor-TOPO (see, Example (1-9)) as a template.

The oligonucleotides 33 and 34 used were:

(a) oligonucleotide 33:
(SEQ ID NO: 33)
GGTTCGAAACGATGAGATTTCCTTCAATTTTTACT,
and
(b) oligonucleotide 34:
(SEQ ID NO: 34)
TCGACTAGTAGCTTCAGCCTCTCTTTTATCC.

The composition of the reaction solutions is given as follows:

(a) plasmid solution (10 ng/μl), 1 μl;
(b) dNTPs (mix, 2 mM each), 5 μl;
(c) MgSO₄ (25 mM), 2 μl;
(d) primers (10 pmol/μl), 1.5 μl each;
(e) 10×PCR buffer for KOD-plus- (Toyobo Co., Ltd.), 5 μl;
(f) KOD-plus- DNA polymerase (1 U/μl, Toyobo Co., Ltd.), 1 μl;
(g) sterile distilled water 33 μl.

The PCR was conducted under conditions where the reaction solution was heated at 94° C. for 3 minutes and then subjected to 10 cycles of denaturation at 94° C. for 15 seconds, annealing at 60° C. for 30 seconds, and extension at 68° C. for 1 minute and then 15 cycles of denaturation at 94° C. for 15 seconds, and each of annealing and extension at 68° C. for 1 minute, followed by additionally keeping the reaction solution at 68° C. for 5 minutes.

An about 0.3-kb DNA fragment resulted from the PCR was digested with restriction enzymes BspT104I and SpeI, and then isolated by agarose gel electrophoresis, followed by extraction and purification from the gel using MinElute Gel Extraction Kit (QIAGEN).

The plasmid named as pSN005 (see, Example (2-1-3)) was digested with restriction enzymes BspT104I and SpeI. Then, an about 4.2-kb DNA fragment was separated by agarose gel electrophoresis, followed by extraction and purification from the gel using MinElute Gel Extraction Kit (QIAGEN).

The about 0.3-kb DNA fragment and the about 4.2-kb DNA fragment were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA having the restriction enzyme recognition sequence added at each end of the DNA encoding the signal sequence and the pro sequence of the α factor gene had been inserted (which may be referred to hereinafter as pSN015) was isolated from the cultured cells to give pSN015.

(2-1-5) Construction of pSN020

A cDNA clone encoding human prolyl 4-hydroxylase β subunit (P4Hβ) (Clone ID: 3848651) was purchased from Invitrogen. The oligonucleotides 35 and 36 mentioned below were synthesized. A DNA fragment which had a restriction enzyme recognition sequence added at each end of the DNA encoding a P4Hβ having no signal sequence was amplified by PCR using the oligonucleotides 35 and 36 as primers and the P4Hβ cDNA clone as a template.

The oligonucleotides 35 and 36 used were:

(a) oligonucleotide 35:
(SEQ ID NO: 35)
TTACTAGTGACGCCCCCGAGGAGGA,
and
(b) oligonucleotide 36:
(SEQ ID NO: 36)
TTACTAGTTTACAGTTCATCTTTCACAGCTTTCTG.

The composition of the reaction solutions is given as follows:

(a) plasmid solution (10 ng/μl), 1 μl;
(b) dNTPs (mix, 2 mM each), 5 μl;
(c) MgSO₄ (25 mM), 2 μl;
(d) primers (10 pmol/μl), 1.5 μl each;
(e) 10×PCR buffer for KOD-plus- (Toyobo Co., Ltd.), 5 μl;
(f) KOD-plus- DNA polymerase (1 U/μl, Toyobo Co., Ltd.), 1 μl;
(g) sterile distilled water 33 μl.

The PCR was conducted under conditions where the reaction solution was heated at 94° C. for 3 minutes and then subjected to 10 cycles of denaturation at 94° C. for 15 seconds, annealing at 60° C. for 30 seconds, and extension at 68° C. for 2 minutes and then 15 cycles of denaturation at 94° C. for 15 seconds, and each of annealing and extension at 68° C. for 2 minutes followed by additionally keeping the reaction solution at 68° C. for 5 minutes.

An about 1.5-kb DNA fragment resulted from the PCR was digested with a restriction enzyme SpeI, and then isolated by agarose gel electrophoresis, followed by extraction and purification from the gel using MinElute Gel Extraction Kit (QIAGEN).

The plasmid named as pSN015 (see, Example (2-1-4)) was digested with a restriction enzyme SpeI, dephosphorylated with an alkaline phosphatase, and then purified using MinElute Reaction Cleanup Kit (QIAGEN).

The about 1.5-kb DNA fragment and the dephosphorylated plasmid were ligated, and the resulting ligation solution was used for transformation of E. coli (Competent high JM109, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA fragment having the restriction enzyme recognition sequence added at each end of the DNA encoding the P4Hβ having no signal sequence had been inserted (which may be referred to hereinafter as pSN020) was isolated from the cultured cells to give pSN020.

(2-1-6) Construction of pP4Hbsig(−)rev-TOPO

The oligonucleotides 37 and 38 mentioned below were synthesized. A DNA fragment which had a restriction enzyme recognition sequence added at each end of the expression cassette for a prolyl 4-hydroxylase β subunit having the α-factor signal and pro sequences was amplified by PCR using the oligonucleotides 37 and 38 as primers and the plasmid named as pSN020 (see, Example (2-1-5)) as a template.

The oligonucleotides 37 and 38 used were:

(a) oligonucleotide 37:
(SEQ ID NO: 37)
AACCGCGGTCTAACATCCAAAGACGAAAGGTTGAA,
and
(b) oligonucleotide 38:
(SEQ ID NO: 38)
AACCCGGGGCACAAACGAACGTCTCACTTAATCTT.

The composition of the reaction solutions is given as follows:

(a) plasmid solution (10 ng/μl), 1 μl;
(b) dNTPs (mix, 2 mM each), 5 μl;
(c) MgSO₄ (25 mM), 2 μl;
(d) primers (10 pmol/μl), 1.5 μl each;
(e) 10×PCR buffer for KOD-plus- (Toyobo Co., Ltd.), 5 μl;
(f) KOD-plus- DNA polymerase (1 U/μl, Toyobo Co., Ltd.), 1 μl;
(g) sterile distilled water 33 μl.

The PCR was conducted under conditions where the reaction solution was heated at 94° C. for 3 minutes and then subjected to 10 cycles of denaturation at 94° C. for 15 seconds, annealing at 60° C. for 30 seconds, and extension at 68° C. for 3.5 minutes and then 15 cycles of denaturation at 94° C. for 15 seconds, and each of annealing and extension at 68° C. for 3.5 minutes, followed by additionally keeping the reaction solution at 68° C. for 5 minutes.

An about 3.0-kb DNA fragment resulted from the PCR was isolated by agarose gel electrophoresis, followed by extraction and purification from the gel using MinElute Gel Extraction Kit (QIAGEN). The about 3.0-kb DNA fragment purified was ligated into the “PCR Product insertion site” of a pCR-BluntII-TOPO plasmid (Invitrogen), and the resulting ligation solution was used for transformation of E. coli (Competent high JM109, Toyobo Co., Ltd.). For the ligation reaction, a Zero-Blunt TOPO PCR cloning kit (Invitrogen) was used.

On LB agar medium containing 50 μg/ml of kanamycin, the E. coli cells which had been transformed were inoculated and cultured. A colony formed on the agar medium was inoculated into LB medium containing 50 μg/ml of kanamycin and incubated with shaking (37° C., 17 hours). Then, using QIAprep Spin Miniprep Kit (QIAGEN), the plasmid into which the DNA fragment having the restriction enzyme recognition sequence added at each end of the expression cassette for the prolyl 4-hydroxylase β subunit having the α-factor signal and pro sequences had been inserted (which may be referred to hereinafter as pP4HBsig(−)rev-TOPO) was isolated from the cultured cells to give pP4HBsig(−)rev-TOPO.

(2-1-7) Construction of pSN007

The oligonucleotides 39 and 40 mentioned below were synthesized:

(a) oligonucleotide 39:
(SEQ ID NO: 39)
TATTCGAAACGACGCGTGTCAGCTAGCACTAGTGC,
and
(b) oligonucleotide 40:
(SEQ ID NO: 40)
GCACTAGTGCTAGCTGACACGCGTCGTTTCGAATA.

A solution having the composition mentioned below was prepared and kept at 98° C. for 5 minutes, at 50° C. for 50 minutes, and then at 37° C. for 1 hour:

(a) oligonucleotide 39 (50 pmol/μl), 5 μl;
(b) oligonucleotide 40 (50 pmol/μl), 5 μl;
(c) Tris-HCl (100 mM), 10 μl;
(d) MgCl₂ (100 mM), 10 μl;
(e) dithiothreitol (10 mM), 10 μl;
(f) sterile distilled water 60 μl.

A DNA linker in which the oligonucleotides 39 and 40 had been annealed was digested with restriction enzymes BspT104I and SpeI. Next, the mixture solution was extracted with phenol:chloroform:isoamyl alcohol (25:24:1) and then subjected to ethanol precipitation to purify the DNA linker (Linker 3).

Additionally, the plasmid named as pSN004 (see, Example (2-1-2)) was digested with restriction enzymes BspT104I and SpeI. An about 4.2-kb DNA fragment was separated and purified by agarose gel electrophoresis.

The DNA linker (Linker 3) and the about 4.2-kb DNA fragment were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA linker (Linker 3) had been inserted (which may be referred to hereinafter as pSN007) was isolated from the cultured cells to give pSN007.

(2-1-8) Construction of pSN017

A cDNA clone encoding human prolyl 4-hydroxylase α1 subunit (P4Hα1) (Clone ID: 4797051) was purchased from Invitrogen. The oligonucleotides 41 and 42 mentioned below were synthesized. A DNA fragment which had a restriction enzyme recognition sequence added at each end of the DNA encoding P4Hα1 was amplified by PCR using the oligonucleotides 41 and 42 as primers and the P4Hα1 cDNA clone as a template.

The oligonucleotides 41 and 42 used were:

(a) oligonucleotide 41:
(SEQ ID NO: 41)
TATTCGAAACGATGATCTGGTATATATTAATTATA,
and
(b) oligonucleotide 42:
(SEQ ID NO: 42)
TTGCTAGCTCATTCCAATTCTGACAACGTACAAGG.

The composition of the reaction solutions is given as follows:

(a) plasmid solution (10 ng/μl), 1 μl;
(b) dNTPs (mix, 2 mM each), 5 μl;
(c) MgSO₄ (25 mM), 2 μl;
(d) primers (10 pmol/μl), 1.5 μl each;
(e) 10×PCR buffer for KOD-plus- (Toyobo Co., Ltd.), 5 μl;
(f) KOD-plus- DNA polymerase (1 U/μl, Toyobo Co., Ltd.), 1 μl;
(g) sterile distilled water 33 μl.

The PCR was conducted under conditions where the reaction solution was heated at 94° C. for 3 minutes and then subjected to 10 cycles of denaturation at 94° C. for 15 seconds, annealing at 60° C. for 30 seconds, and extension at 68° C. for 2 minutes and then 15 cycles of denaturation at 94° C. for 15 seconds, and each of annealing and extension at 68° C. for 2 minutes, followed by additionally keeping the reaction solution at 68° C. for 5 minutes.

An about 1.6-kb DNA fragment resulted from the PCR was digested with restriction enzymes BspT104I and SpeI, and then isolated by agarose gel electrophoresis, followed by extraction and purification from the gel using MinElute Gel Extraction Kit (QIAGEN).

The plasmid named as pSN007 (see, Example (2-1-7)) was digested with restriction enzymes BspT104I and SpeI. Then, an about 4.2-kb DNA fragment was separated by agarose gel electrophoresis, followed by extraction and purification from the gel using MinElute Gel Extraction Kit (QIAGEN).

The about 1.6-kb DNA fragment and the about 4.2-kb DNA fragment were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA fragment having the restriction enzyme recognition sequence added at each end of the DNA encoding P4Hα1 had been inserted (which may be referred to hereinafter as pSN017) was isolated from the cultured cells to give pSN017.

(2-1-9) Construction of pP4HA1-SmaI-TOPO

The oligonucleotides 43 and 38 mentioned below were synthesized. A DNA fragment which had a restriction enzyme recognition sequence added at each end of the expression cassette for a prolyl 4-hydroxylase α1 subunit was amplified by PCR using the oligonucleotides 43 and 38 as primers and the plasmid named as pSN017 (see, Example (2-1-8)) as a template.

The oligonucleotides 43 and 38 used were:

(a) oligonucleotide 43:
(SEQ ID NO: 43)
AACCCGGGTCTAACATCCAAAGACGAAAGGTTGAA,
and
(b) oligonucleotide 38:
(SEQ ID NO: 38)
AACCCGGGGCACAAACGAACGTCTCACTTAATCTT.

The composition of the reaction solutions is given as follows:

(a) plasmid solution (10 ng/μl), 1 μl;
(b) dNTPs (mix, 2 mM each), 5 μl;
(c) MgSO₄ (25 mM), 2 μl;
(d) primers (10 pmol/μl), 1.5 μl each;
(e)10×PCR buffer for KOD-plus-(Toyobo Co., Ltd.), 5 μl;
(f) KOD-plus- DNA polymerase (1 U/μl, Toyobo Co., Ltd.), 1 μl;
(g) sterile distilled water 33 μl.

The PCR was conducted under conditions where the reaction solution was heated at 94° C. for 3 minutes and then subjected to 10 cycles of denaturation at 94° C. for 15 seconds, annealing at 60° C. for 30 seconds, and extension at 68° C. for 3.5 minutes and then 15 cycles of denaturation at 94° C. for 15 seconds, and each of annealing and extension at 68° C. for 3.5 minutes, followed by additionally keeping the reaction solution at 68° C. for 5 minutes.

An about 2.8-kb DNA fragment resulted from the PCR was isolated by agarose gel electrophoresis, followed by extraction and purification from the gel using MinElute Gel Extraction Kit (QIAGEN). The about 2.8-kb DNA fragment was ligated into the “PCR Product insertion site” of a pCR-BluntII-TOPO plasmid (Invitrogen), and the resulting ligation solution was used for transformation of E. coli (Competent high JM109, Toyobo Co., Ltd.). For the ligation reaction, a Zero-Blunt TOPO PCR cloning kit (Invitrogen) was used.

On LB agar medium containing 50 μg/ml of kanamycin, the E. coli cells which had been transformed were inoculated and cultured. A colony formed on the agar medium was inoculated into LB medium containing 50 μg/ml of kanamycin and incubated with shaking (37° C., 17 hours). Then, using QIAprep Spin Miniprep Kit (QIAGEN), the plasmid into which the DNA fragment having the restriction enzyme recognition sequences added at each end of the expression cassette for the prolyl 4-hydroxylase α1 subunit had been inserted (which may be referred to hereinafter as) was isolated from the cultured cells to give pP4HA1-SmaI-TOPO.

(2-1-10) Construction of pSN023

The oligonucleotides 44 and 45 mentioned below were synthesized. A DNA fragment which had a restriction enzyme recognition sequence added at each end of the DNA encoding ARG4 was amplified by PCR using the oligonucleotides 44 and 45 as primers and the plasmid named as pARG4-TOPO (see, Example (1-2)) as a template.

The oligonucleotides 44 and 45 used were:

(a) oligonucleotide 44:
(SEQ ID NO: 44)
AACTCGAGACGAAAATATGGTACCTGCCCT,
and
(b) oligonucleotide 45:
(SEQ ID NO: 45)
CCATCGATACAGAGGTATCATCCAATGATTCC.

The composition of the reaction solutions is given as follows:

(a) plasmid solution (10 ng/μl), 1 μl;
(b) dNTPs (mix, 2 mM each), 5 μl;
(c) MgSO₄ (25 mM), 2 μl;
(d) primers (10 pmol/μl), 1.5 μl each;
(e) 10×PCR buffer for KOD-plus- (Toyobo Co., Ltd.), 5 μl;
(f) KOD-plus- DNA polymerase (1 U/μl, Toyobo Co., Ltd.), 1 μl;
(g) sterile distilled water 33 μl.

The PCR was conducted under conditions where the reaction solution was heated at 94° C. for 3 minutes and then subjected to 10 cycles of denaturation at 94° C. for 15 seconds, annealing at 60° C. for 30 seconds, and extension at 68° C. for 2 minutes and then 15 cycles of denaturation at 94° C. for 15 seconds, and each of annealing and extension at 68° C. for 2 minutes, followed by additionally keeping the reaction solution at 68° C. for 5 minutes.

An about 2.2-kb DNA fragment resulted from the PCR was digested with restriction enzymes XhoI and ClaI, and then isolated by agarose gel electrophoresis, followed by extraction and purification from the gel using MinElute Gel Extraction Kit (QIAGEN).

A pBluescriptII KS(+) plasmid (Stratagene) was digested with restriction enzymes XhoI and ClaI, and then isolated by agarose gel electrophoresis, followed by extraction and purification from the gel using MinElute Gel Extraction Kit (QIAGEN).

The about 2.2-kb DNA fragment and the digested plasmid were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA fragment having the restriction enzyme recognition sequence added at each end of the DNA encoding ARG4 had been inserted (which may be referred to hereinafter as pSN023) was isolated from the cultured cells to give pSN023.

(2-1-11) Construction of pEXP-A-P4Hbsig(−)rev

The plasmid named as pP4Hbsig(−)rev-TOPO (see, Example (2-1-6)) was digested with restriction enzymes SacII and Cfr9I. An about 3.0-kb DNA fragment which had the restriction enzyme recognition sequence added at each end of the expression cassette for the prolyl 4-hydroxylase β subunit having the α-factor signal and pro sequences was isolated by agarose gel electrophoresis, followed by extraction and purification from the gel using MinElute Gel Extraction Kit (QIAGEN).

The plasmid named as pSN023 (see, Example (2-1-10) was digested with restriction enzymes Sad and Cfr9I. Then, an about 5.2-kb DNA fragment was isolated by agarose gel electrophoresis, followed by extraction and purification from the gel using MinElute Gel Extraction Kit (QIAGEN).

The about 3.0-kb DNA fragment and the about 5.2-kb DNA fragment were ligated, and the resulting ligation solution was used for transformation of E. coli (Competent high JM109, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA fragment having the restriction enzyme recognition sequence added at each end of the expression cassette for the prolyl 4-hydroxylase β subunit having the α-factor signal and the pro sequences had been inserted (which may be referred to hereinafter as pEXP-A-P4Hbsig(−)rev) was isolated from the cultured cells to give pEXP-A-P4Hbsig(−)rev.

(2-1-12) Construction of pEXP-A-P4Hbsig(−)A1rev

The plasmid named as pP4HA1-SmaI-TOPO (see, Example (2-1-9)) was digested with a restriction enzyme Cfr9I. An about 2.8-kb DNA fragment which had the restriction enzyme recognition sequence added at each end of the expression cassette for the prolyl 4-hydroxylase α1 subunit was isolated by agarose gel electrophoresis, followed by extraction and purification from the gel using MinElute Gel Extraction Kit (QIAGEN).

The plasmid named as pEXP-A-P4Hbsig(−)rev (see, Example (2-1-11)) was digested with a restriction enzyme Cfr9I, dephosphorylated with an alkaline phosphatase, and then purified using MinElute Reaction Cleanup Kit (QIAGEN).

The about 3.0-kb DNA fragment and the dephosphorylated plasmid were ligated, and the resulting ligation solution was used for transformation of E. coli (Competent high JM109, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA fragment having the restriction enzyme recognition sequences added at each end of the expression cassette for the prolyl 4-hydroxylase α1 subunit had been inserted (which may be referred to hereinafter as pEXP-A-P4Hbsig(−)A1rev) was isolated from the cultured cells to give pEXP-A-P4Hbsig(−) A1rev.

### (2-2) Preparation of Plasmids for Introducing an Expression Cassette for an FK506 Binding Protein

(2-2-1) Construction of pTS011

A cDNA clone encoding heat shock protein 47 (Hsp47) (Clone ID: 3030138) was purchased from Invitrogen. The oligonucleotides 46 and 47 mentioned below were synthesized. A DNA fragment which had a restriction enzyme recognition sequence added at each end of the DNA encoding HSP47 was amplified by PCR using the oligonucleotides 46 and 47 as primers and the HSP47 cDNA clone as a template.

The oligonucleotides 46 and 47 used were:

(a) oligonucleotide 46:
(SEQ ID NO: 46)
TATTCGAAACGATGCGCTCCCTCCTGCTTCTC,
and
(b) oligonucleotide 47:
(SEQ ID NO: 47)
TTACTAGTTATAACTCGTCTCGCATCTTGTCACC.

The composition of the reaction solutions is given as follows:

(a) cDNA solution (10 ng/μl), 1 μl;
(b) dNTPs (mix, 2 mM each), 5 μl;
(c) MgSO₄ (25 mM), 2 μl;
(d) primers (10 pmol/μl), 1.5 μl each;
(e) 10×PCR buffer for KOD-plus- (Toyobo Co., Ltd.), 5 μl;
(f) KOD-plus- DNA polymerase (1 U/μl, Toyobo Co., Ltd.), 1 μl;
(g) sterile distilled water 33 μl.

The PCR was conducted under conditions where the reaction solution was heated at 94° C. for 3 minutes and then subjected to 5 cycles of denaturation at 94° C. for 15 seconds, annealing at 60° C. for 30 seconds, and extension at 68° C. for 1.5 minutes and then 20 cycles of denaturation at 94° C. for 15 seconds, and each of annealing and extension at 68° C. for 1.5 minutes, followed by additionally keeping the reaction solution at 68° C. for 1.5 minutes.

An about 1.3-kb DNA fragment resulted from the PCR was digested with restriction enzymes BspT104I and SpeI, and then isolated by agarose gel electrophoresis, followed by extraction and purification from the gel using MinElute Gel Extraction Kit (QIAGEN).

The plasmid named as pSN005 (see, Example (2-1-3)) was digested with restriction enzymes BspT104I and SpeI. Then, an about 4.2-kb DNA fragment was separated by agarose gel electrophoresis, followed by extraction and purification from the gel using MinElute Gel Extraction Kit (QIAGEN).

The about 1.3-kb DNA fragment and the about 4.2-kb DNA fragment were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA fragment having the restriction enzyme recognition sequence added at each end of the DNA encoding HSP47 had been inserted (which may be referred to hereinafter as pTS011) was isolated from the cultured cells to give pTS011.

(2-2-2) Construction of pEXP-A-HSP47native Signal

The plasmid named as pTS011 (see, Example (2-2-1)) was digested with restriction enzymes BspT104I and SpeI. An about 1.3-kb DNA fragment which had the restriction enzyme recognition sequence at each end of the DNA encoding HSP47 was separated by agarose gel electrophoresis, followed by extraction and purification from the gel using MinElute Gel Extraction Kit (QIAGEN).

The plasmid named as pEXP-A-P4Hbsig(−)rev (see, Example (2-1-11)) was digested with restriction enzymes BspT104I and SpeI. Then, an about 6.3-kb DNA fragment was separated by agarose gel electrophoresis, followed by extraction and purification from the gel using MinElute Gel Extraction Kit (QIAGEN).

The about 1.3-kb DNA fragment and the about 6.3-kb DNA fragment were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA fragment having the restriction enzyme recognition sequence added at each end of the DNA encoding HSP47 had been inserted (which may be referred to hereinafter as pEXP-A-HSP47native signal) was isolated from the cultured cells to give pEXP-A-HSP47native signal.

(2-2-3) Construction of pEXP-A-HSP47native Signal ZeoR2rev

The oligonucleotides 48 and 49 mentioned below were synthesized. A DNA fragment which had a restriction enzyme recognition sequence added at each end of the Zeocyn-resistance cassette was amplified by PCR using the oligonucleotides 48 and 49 as primers and the plasmid named as pUC57-ZeoR (see, Example (1-13)) as a template.

The oligonucleotides 48 and 49 used were:

(a) oligonucleotide 48:
(SEQ ID NO: 48)
TTATCGATCCCACACACCATAGCTTCA,
and
(b) oligonucleotide 49:
(SEQ ID NO: 49)
TGATCGATAGCTTGCAAATTAAAGCCTTC.

The composition of the reaction solutions is given as follows:

(a) cDNA solution (10 ng/μl), 1 μl;
(b) dNTPs (mix, 2 mM each), 5 μl;
(c) MgSO₄ (25 mM), 2 μl;
(d) primers (10 pmol/μl), 1.5 μl each;
(e) 10×PCR buffer for KOD-plus- (Toyobo Co., Ltd.), 5 μl;
(f) KOD-plus- DNA polymerase (1 U/μl, Toyobo Co., Ltd.), 1 μl;
(g) sterile distilled water 33 μl.

The PCR was conducted under conditions where the reaction solution was heated at 94° C. for 2 minutes and then subjected to 5 cycles of denaturation at 94° C. for 15 seconds, annealing at 60° C. for 30 seconds, and extension at 68° C. for 1.5 minutes and then 20 cycles of denaturation at 94° C. for 15 seconds, and each of annealing and extension at 68° C. for 1.5 minutes, followed by additionally keeping the reaction solution at 68° C. for 1.5 minutes.

An about 1.2-kb DNA fragment resulted from the PCR was digested with a restriction enzyme ClaI. Then, the digested, about 1.2-kb DNA fragment was isolated by agarose gel electrophoresis, followed by extraction and purification from the gel using MinElute Gel Extraction Kit (QIAGEN).

The plasmid named as pEXP-A-HSP47native signal (see, Example (2-2-2)) was digested with a restriction enzyme ClaI, and then dephosphorylated with an alkaline phosphatase, and purified using MinElute Reaction Cleanup Kit (QIAGEN).

The digested, about 1.2-kb DNA fragment and the dephosphorylated plasmid were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin and 25 μg/ml of Zeocyn, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA fragment having the restriction enzyme recognition sequence added at each end of the Zeocyn-resistance cassette had been inserted (which may be referred to hereinafter as pEXP-A-HSP47native signal ZeoR2rev) was isolated from the cultured cells to give pEXP-A-HSP47native signal ZeoR2rev.

(2-2-4) Construction of pEXP-A-FKBP13A ZeoR

The plasmid named as pUC57-YFKBP13A (see, Example (1-14)) was digested with restriction enzymes BspT104I and SpeI. An about 0.4-kb DNA fragment encoding FKBP13A was separated by agarose gel electrophoresis, followed by extraction and purification from the gel using MinElute Gel Extraction Kit (QIAGEN).

The plasmid named as pEXP-A-HSP47native signal ZeoR2rev (see, Example (2-2-3)) was digested with restriction enzymes BspT104I and SpeI. Then, an about 6.4-kb DNA fragment was separated by agarose gel electrophoresis, followed by extraction and purification from the gel using MinElute Gel Extraction Kit (QIAGEN).

The about 0.4-kb DNA fragment and the about 6.4-kb DNA fragment were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA fragment encoding FKBP13A had been inserted (which may be referred to hereinafter as pEXP-A-FKBP13A ZeoR) was isolated from the cultured cells to give pEXP-A-FKBP13A ZeoR.

(2-2-5) Construction of pEXP-A-FKBP19 ZeoR

The plasmid named as pUC57-YFKBP19 (see, Example (1-15)) was digested with restriction enzymes BspT104I and SpeI. An about 0.6-kb DNA fragment encoding FKBP19 was separated by agarose gel electrophoresis, followed by extraction and purification from the gel using MinElute Gel Extraction Kit (QIAGEN).

The plasmid named as pEXP-A-HSP47native signal ZeoR2rev (see, Example (2-2-3)) was digested with restriction enzymes BspT104I and SpeI. Then, an about 6.4-kb DNA fragment was separated by agarose gel electrophoresis, followed by extraction and purification from the gel using MinElute Gel Extraction Kit (QIAGEN).

The about 0.6-kb DNA fragment and the about 6.4-kb DNA fragment were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA fragment encoding FKBP19 had been inserted (which may be referred to hereinafter as pEXP-A-FKBP19 ZeoR) was isolated from the cultured cells to give pEXP-A-FKBP19 ZeoR.

(2-2-6) Construction of pEXP-A-FKBP23 ZeoR

The plasmid named as pUC57-YFKBP23 (see, Example (1-16)) was digested with restriction enzymes BspT104I and SpeI. An about 0.7-kb DNA fragment encoding FKBP23 was separated by agarose gel electrophoresis, followed by extraction and purification from the gel using MinElute Gel Extraction Kit (QIAGEN).

The plasmid named as pEXP-A-HSP47native signal ZeoR2rev (see, Example (2-2-3)) was digested with restriction enzymes BspT104I and SpeI. Then, an about 6.4-kb DNA fragment was separated by agarose gel electrophoresis, followed by extraction and purification from the gel using MinElute Gel Extraction Kit (QIAGEN).

The about 0.7-kb DNA fragment and the about 6.4-kb DNA fragment were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA fragment encoding FKBP23 had been inserted (which may be referred to hereinafter as pEXP-A-FKBP23 ZeoR) was isolated from the cultured cells to give pEXP-A-FKBP23 ZeoR.

(2-2-7) Construction of pEXP-A-FKBP63 ZeoR

The plasmid named as pUC57-YFKBP63 (see, Example (1-17)) was digested with restriction enzymes BspT104I and SpeI. An about 1.7-kb DNA fragment encoding FKBP63 was separated by agarose gel electrophoresis, followed by extraction and purification from the gel using MinElute Gel Extraction Kit (QIAGEN).

The plasmid named as pEXP-A-HSP47native signal ZeoR2rev (see, Example (2-2-3)) was digested with restriction enzymes BspT104I and SpeI. Then, an about 6.4-kb DNA fragment was separated by agarose gel electrophoresis, followed by extraction and purification from the gel using MinElute Gel Extraction Kit (QIAGEN).

The about 1.7-kb DNA fragment and the about 6.4-kb DNA fragment were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA fragment encoding FKBP63 had been inserted (which may be referred to hereinafter as pEXP-A-FKBP63 ZeoR) was isolated from the cultured cells to give pEXP-A-FKBP63 ZeoR.

(2-2-8) Construction of pEXP-A-FKBP65 ZeoR

The plasmid named as pUC57-YFKBP65 (see, Example (1-18)) was digested with restriction enzymes BspT104I and SpeI. An about 1.8-kb DNA fragment encoding FKBP19 was separated by agarose gel electrophoresis, followed by extraction and purification from the gel using MinElute Gel Extraction Kit (QIAGEN).

The plasmid named as pEXP-A-HSP47native signal ZeoR2rev (see, Example (2-2-3)) was digested with restriction enzymes BspT104I and SpeI. Then, an about 6.4-kb DNA fragment was separated by agarose gel electrophoresis, followed by extraction and purification from the gel using MinElute Gel Extraction Kit (QIAGEN).

The about 1.8-kb DNA fragment and the about 6.4-kb DNA fragment were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA fragment encoding FKBP65 had been inserted (which may be referred to hereinafter as pEXP-A-FKBP65 ZeoR) was isolated from the cultured cells to give pEXP-A-FKBP65 ZeoR.

(2-2-9) Construction of pEXP-A-PER3 Pro-FKBP23 ZeoR

The plasmid named as pCR-BII-PER3 Pro SacII-Psp1406I(−) (see, Example (1-4)) was digested with restriction enzymes Psp1406I and SacII. Then, an about 1.0-kb DNA fragment which had the restriction enzyme recognition sequence added at each end of the PER3 promoter was separated by agarose gel electrophoresis, followed by extraction and purification from the gel using MinElute Gel Extraction Kit (QIAGEN).

The plasmid named as pEXP-A-FKBP23 ZeoR (see, Example (2-2-6)) was digested with restriction enzymes BspT104I and SacII. An about 7.2-kb DNA fragment was separated by agarose gel electrophoresis, followed by extraction and purification from the gel using MinElute Gel Extraction Kit (QIAGEN).

The about 1.0-kb DNA fragment and the about 7.2-kb DNA fragment were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA having the restriction enzyme recognition sequence at each end of the PER3 promoter had been inserted (which may be referred to hereinafter as pEXP-A-PER3 Pro-FKBP23 ZeoR) was isolated from the cultured cells to give pEXP-A-PER3 Pro-FKBP23 ZeoR.

(2-2-10) Construction of pEXP-A-AOX2 Pro-FKBP23 ZeoR

The plasmid named as pCR-BII-AOX2 Pro SacII-Psp1406I(−) (see, Example (1-5)) was digested with restriction enzymes Psp1406I and SacII. Then, an about 1.1-kb DNA fragment which had the restriction enzyme recognition sequence added at each end of the AOX2 promoter was separated by agarose gel electrophoresis, followed by extraction and purification from the gel using MinElute Gel Extraction Kit (QIAGEN).

The plasmid named as pEXP-A-FKBP23 ZeoR (see, Example (2-2-6)) was digested with restriction enzymes BspT104I and SacII. An about 7.2-kb DNA fragment was separated by agarose gel electrophoresis, followed by extraction and purification from the gel using MinElute Gel Extraction Kit (QIAGEN).

The about 1.1-kb DNA fragment and the about 7.2-kb DNA fragment were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA having the restriction enzyme recognition sequence at each end of the AOX2 promoter had been inserted (which may be referred to hereinafter as pEXP-A-AOX2 Pro-FKBP23 ZeoR) was isolated from the cultured cells to give pEXP-A-AOX2 Pro-FKBP23 ZeoR.

(2-2-11) Construction of pEXP-A-FLD1 Pro-FKBP23 ZeoR

The plasmid named as pCR-BII-FLD1 Pro SacII-Psp1406I(+) (see, Example (1-6)) was digested with restriction enzymes Psp1406I and SacII. Then, an about 0.9-kb DNA fragment which had the restriction enzyme recognition sequence added at each end of the FLD1 promoter was separated by agarose gel electrophoresis, followed by extraction and purification from the gel using MinElute Gel Extraction Kit (QIAGEN).

The plasmid named as pEXP-A-FKBP23 ZeoR (see, Example (2-2-6)) was digested with restriction enzymes BspT104I and SacII. An about 7.2-kb DNA fragment was separated by agarose gel electrophoresis, followed by extraction and purification from the gel using MinElute Gel Extraction Kit (QIAGEN).

The about 0.9-kb DNA fragment and the about 7.2-kb DNA fragment were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA having the restriction enzyme recognition sequence at each end of the FLD1 promoter had been inserted (which may be referred to hereinafter as pEXP-A-FLD1 Pro-FKBP23 ZeoR) was isolated from the cultured cells to give pEXP-A-FLD1 Pro-FKBP23 ZeoR.

(2-3) Preparation of a Plasmid (pEXP-HA-YHsCOL1A2-1A1) for Introducing an Expression Cassette for Human Collagen Type I α1 and an Expression Cassette for Human Collagen Type I α2
(2-3-1) Construction of pSN001

The oligonucleotides 51 and 52 mentioned below were synthesized. A DNA fragment which had a restriction enzyme recognition sequence added at each end of the HIS4 was amplified by PCR using the oligonucleotides 50 and 51 as primers and the plasmid named as pHIS4-TOPO (see, Example (1-1)) as a template.

The oligonucleotides 50 and 51 used were:

(a) oligonucleotide 50:
(SEQ ID NO: 50)
GGAAGCTTGATCTCCTGATGACTGACTCACTG,
and
(b) oligonucleotide 51:
(SEQ ID NO: 51)
CCCTGCAGTAATTAAATAAGTCCCAGTTTCTCCA.

The composition of the reaction solutions is given as follows:

(a) plasmid solution (10 ng/μl), 1 μl;
(b) dNTPs (mix, 2 mM each), 5 μl;
(c) MgSO₄ (25 mM), 2 μl;
(d) primers (10 pmol/μl), 1.5 μl each;
(e) 10×PCR buffer for KOD-plus- (Toyobo Co., Ltd.), 5 μl;
(f) KOD-plus- DNA polymerase (1 U/μl, Toyobo Co., Ltd.), 1 μl;
(g) sterile distilled water 33 μl.

The PCR was conducted under conditions where the reaction solution was heated at 94° C. for 3 minutes and then subjected to 5 cycles of denaturation at 94° C. for 15 seconds, annealing at 60° C. for 30 seconds, and extension at 68° C. for 2 minutes and then 20 cycles of denaturation at 94° C. for 15 seconds, and each of annealing and extension at 68° C. for 2 minutes, followed by additionally keeping the reaction solution at 68° C. for 5 minutes.

An about 2.6-kb DNA fragment resulted from the PCR was digested with restriction enzymes HindIII and PstI, and then isolated by agarose gel electrophoresis, followed by extraction and purification from the gel using MinElute Gel Extraction Kit (QIAGEN).

A pBluescriptII KS(+) plasmid (Stratagene) was digested with restriction enzymes HindIII and PstI. Then, an about 3.0-kb DNA fragment was isolated by agarose gel electrophoresis, followed by extraction and purification from the gel using MinElute Gel Extraction Kit (QIAGEN).

The about 2.6-kb DNA fragment and the about 3.0-kb DNA fragment were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA having the restriction enzyme recognition sequence at each end of the HIS4 had been inserted (which may be referred to hereinafter as pSN001) was isolated from the cultured cells to give pSN001.

(2-3-2) Construction of pSN002

The oligonucleotides 52 and 53 mentioned below were synthesized. A DNA fragment which had a restriction enzyme recognition sequence added at each end of the 3′-downstream non-coding region of AOX1 was amplified by PCR using the oligonucleotides 52 and 53 as primers and the plasmid named as pAOX1-3′-TOPO (see, Example (1-8)) as a template.

The oligonucleotides 52 and 53 used were:

(a)oligonucleotide 52:
(SEQ ID NO: 52)
GCATCGATTCGAGTATCTATGATTGGAAGTATGG,
and
(b) oligonucleotide 53:
(SEQ ID NO: 53)
AAGGGCCCGATCTTGAGATAAATTTCACGTTTAAA.

The composition of the reaction solutions is given as follows:

(a) plasmid solution (10 ng/μl), 1 μl;
(b) dNTPs (mix, 2 mM each), 5 μl;
(c) MgSO₄ (25 mM), 2 μl;
(d) primers (10 pmol/μl), 1.5 μl each;
(e) 10×PCR buffer for KOD-plus- (Toyobo Co., Ltd.), 5 μl;
(f) KOD-plus- DNA polymerase (1 U/μl, Toyobo Co., Ltd.), 1 μl;
(g) sterile distilled water 33 μl.

The PCR was conducted under conditions where the reaction solution was heated at 94° C. for 3 minutes and then subjected to 5 cycles of denaturation at 94° C. for 15 seconds, annealing at 55° C. for 30 seconds, and extension at 68° C. for 2 minutes and then 20 cycles of denaturation at 94° C. for 15 seconds, and each of annealing and extension at 68° C. for 2 minutes, followed by additionally keeping the reaction solution at 68° C. for 5 minutes.

An about 0.8-kb DNA fragment resulted from the PCR was digested with restriction enzymes ClaI and ApaI, and then isolated by agarose gel electrophoresis, followed by extraction and purification from the gel using MinElute Gel Extraction Kit (QIAGEN).

The plasmid named as pSN001 (see, Example (2-3-1)) was digested with restriction enzymes ClaI and ApaI. Then, an about 5.6-kb DNA fragment was isolated by agarose gel electrophoresis, followed by extraction and purification from the gel using MinElute Gel Extraction Kit (QIAGEN).

The about 0.8-kb DNA fragment and the about 5.6-kb DNA fragment were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA having the restriction enzyme recognition sequence added at each end of the 3′-downstream non-coding region of AOX1 had been inserted (which may be referred to hereinafter as pSN002) was isolated from the cultured cells to give pSN002.

(2-3-3) Construction of pSN006

The oligonucleotides 54 and 55 mentioned below were synthesized:

(a) oligonucleotide 54:
(SEQ ID NO: 54)
TATTCGAAACGCATATGGTACCGGCAGACTAGTGG,
and
(b) oligonucleotide 55:
(SEQ ID NO: 55)
CCACTAGTCGCCTAGGCGACATATGGTTTCGAATA.

A solution having the composition mentioned below was prepared and kept at 98° C. for 5 minutes, at 50° C. for 50 minutes, and then at 37° C. for 1 hour:

(a) oligonucleotide 54 (50 pmol/μl), 5 μl;
(b) oligonucleotide 55 (50 pmol/μl), 5 μl;
(c) Tris-HCl (100 mM), 10 μl;
(d) MgCl₂ (100 mM), 10 μl;
(e) dithiothreitol (10 mM), 10 μl;
(f) sterile distilled water 60 μl.

A DNA linker in which the oligonucleotides 54 and 55 had been annealed was digested with restriction enzymes BspT104I and SpeI. Next, the mixture solution was extracted with phenol:chloroform:isoamyl alcohol (25:24:1) and then subjected to ethanol precipitation to purify the DNA linker (Linker 2).

Additionally, the plasmid named as pSN004 (see, Example (2-1-2)) was digested with restriction enzymes BspT104I and SpeI. An about 4.2-kb DNA fragment was separated and purified by agarose gel electrophoresis.

The DNA linker (Linker 2) and the about 4.2-kb DNA fragment were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA linker (Linker 2) had been inserted (which may be referred to hereinafter as pSN006) was isolated from the cultured cells to give pSN006.

(2-3-4) Construction of pEXH002

The plasmid named as pSN006 (see, Example (2-3-3)) was digested with restriction enzymes Eco52I and PstI. An about 1.3-kb DNA fragment comprising the AOX1 promoter and the AOX1 terminator was isolated by agarose gel electrophoresis, followed by extraction and purification from the gel using MinElute Gel Extraction Kit (QIAGEN).

The plasmid named as pSN002 (see, Example (2-3-1)) was digested with restriction enzymes Eco52I and PstI. An about 6.3-kb DNA fragment was isolated by agarose gel electrophoresis, followed by extraction and purification from the gel using MinElute Gel Extraction Kit (QIAGEN).

The about 1.3-kb DNA fragment and the about 6.3-kb DNA fragment were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA fragment comprising the AOX1 promoter and the AOX1 terminator had been inserted (which may be referred to hereinafter as pEXH002) was isolated from the cultured cells to give pEXH002.

### (2-3-5) Construction of PTS001

The plasmid named as pUC57-YHsCOL1A1 (see, Example (1-19)) was digested with restriction enzymes BspT104I and SpeI. An about 4.4-kb DNA fragment encoding human collagen Type I α1 was separated by agarose gel electrophoresis, followed by extraction and purification from the gel using MinElute Gel Extraction Kit (QIAGEN).

The plasmid named as pSN006 (see, Example (2-3-3)) was digested with restriction enzymes BspT104I and SpeI. An about 4.2-kb DNA fragment was separated by agarose gel electrophoresis, followed by extraction and purification from the gel using MinElute Gel Extraction Kit (QIAGEN).

The about 4.4-kb DNA fragment and the about 4.2-kb DNA fragment were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA fragment encoding human collagen Type I α1 had been inserted (which may be referred to hereinafter as PTS001) was isolated from the cultured cells to give PTS001.

(2-3-6) Construction of pEXP-HA-YHsCOL1A1

The plasmid named as pTS001 (see, Example (2-3-5)) was digested with restriction enzymes Eco52I and SpeI. An about 5.3-kb DNA fragment encoding the AOX1 promoter and human collagen Type I α1 was isolated by agarose gel electrophoresis, followed by extraction and purification from the gel using MinElute Gel Extraction Kit (QIAGEN).

The plasmid named as pEXH002 (see, Example (2-3-4)) was digested with restriction enzymes Eco52I and SpeI. An about 6.6-kb DNA fragment was isolated by agarose gel electrophoresis, followed by extraction and purification from the gel using MinElute Gel Extraction Kit (QIAGEN).

The about 5.3-kb DNA fragment and the about 6.6-kb DNA fragment were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA fragment encoding the AOX1 promoter and human collagen Type I α1 had been inserted (which may be referred to hereinafter as pEXP-HA-YHsCOL1A1) was isolated from the cultured cells to give pEXP-HA-YHsCOL1A1.

(2-3-7) Construction of pTS002

The plasmid named as pUC57-YHsCOL1A2 (see, Example (1-20)) was digested with restriction enzymes BspT104I and SpeI. An about 4.1-kb DNA fragment encoding human collagen Type I α2 was separated by agarose gel electrophoresis, followed by extraction and purification from the gel using MinElute Gel Extraction Kit (QIAGEN).

The plasmid named as pSN006 (see, Example (2-3-3)) was digested with restriction enzymes BspT104I and SpeI. An about 4.2-kb DNA fragment was separated by agarose gel electrophoresis, followed by extraction and purification from the gel using MinElute Gel Extraction Kit (QIAGEN).

The about 4.1-kb DNA fragment and the about 4.2-kb DNA fragment were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA fragment encoding human collagen Type I α2 had been inserted (which may be referred to hereinafter as pTS002) was isolated from the cultured cells to give pTS002.

(2-3-8) Construction of pYHsCOL1A2 Exp Unit-Eco52I

The oligonucleotides 56 and 57 mentioned below were synthesized. A DNA fragment which had a restriction enzyme recognition sequence added at each end of the expression cassette for human collagen Type I α2 was amplified by PCR using the oligonucleotides 56 and 57 as primers and the plasmid named as pTS002 (see, Example (2-3-7)) as a template.

The oligonucleotides 56 and 57 used were:

(a) oligonucleotide 56:
(SEQ ID NO: 56)
AACGGCCGTCTAACATCCAAAGACGAAAGGTTGAA,
and
(b) oligonucleotide 57:
(SEQ ID NO: 57)
AACGGCCGGCACAAACGAACGTCTCACTTAATCTT.

The composition of the reaction solutions is given as follows:

(a) plasmid solution (10 ng/μl), 1 μl;
(b) dNTPs (mix, 2 mM each), 5 μl;
(c) MgSO₄ (25 mM), 2 μl;
(d) primers (10 pmol/μl), 1.5 μl each;
(e) 10×PCR buffer for KOD-plus- (Toyobo Co., Ltd.), 5 μl;
(f) KOD-plus- DNA polymerase (1 U/μl, Toyobo Co., Ltd.), 1 μl;
(g) sterile distilled water 33 μl.

The PCR was conducted under conditions where the reaction solution was heated at 94° C. for 3 minutes and then subjected to 5 cycles of denaturation at 94° C. for 15 seconds, annealing at 60° C. for 30 seconds, and extension at 68° C. for 6 minutes and then 18 cycles of denaturation at 94° C. for 15 seconds, and each of annealing and extension at 68° C. for 6 minutes, followed by additionally keeping the reaction solution at 68° C. for 5 minutes.

An about 5.4-kb DNA fragment resulted from the PCR was isolated by agarose gel electrophoresis, followed by extraction and purification from the gel using MinElute Gel Extraction Kit (QIAGEN). The about 5.4-kb DNA fragment purified was ligated into the “PCR Product insertion site” of a pCR-BluntII-TOPO plasmid (Invitrogen), and the resulting ligation solution was used for transformation of E. coli (Competent high DH5α, Toyobo Co., Ltd.). For the ligation reaction, a Zero-Blunt TOPO PCR cloning kit (Invitrogen) was used.

On LB agar medium containing 50 μg/ml of kanamycin, the E. coli cells which had been transformed were inoculated and cultured. A colony formed on the agar medium was inoculated into LB medium containing 50 μg/ml of kanamycin and incubated with shaking (37° C., 17 hours). Then, using QIAprep Spin Miniprep Kit (QIAGEN), the plasmid into which the DNA fragment having the restriction enzyme recognition sequences added at each end of the expression cassette for human collagen Type I α2 had been inserted (which may be referred to hereinafter as pYHsCOL1A2 Exp unit-Eco52I) was isolated from the cultured cells to give pYHsCOL1A2 Exp unit-Eco52I.

(2-3-9) Construction of pEXP-HA-YHsCOL1A2-1A1

The plasmid named as pYHsCOL1A2 Exp unit-Eco52I (see, Example (2-3-8)) was digested with a restriction enzyme Eco52I. An about 5.4-kb DNA fragment which had the restriction enzyme recognition sequence added at each end of the expression cassette for human collagen Type I α2 was separated by agarose gel electrophoresis, followed by extraction and purification from the gel using MinElute Gel Extraction Kit (QIAGEN).

The plasmid named as pEXP-HA-YHsCOL1A1 (see, Example (2-3-6)) was digested with a restriction enzyme Eco52I, dephosphorylated with an alkaline phosphatase, and then purified using MinElute Reaction Cleanup Kit (QIAGEN).

The about 5.4-kb DNA fragment and the dephosphorylated plasmid were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA fragment having the restriction enzyme recognition sequences added at each end of the expression cassette for human collagen Type I α2 had been inserted (which may be referred to hereinafter as pEXP-HA-YHsCOL1A2-1A1) was isolated from the cultured cells to give pEXP-HA-YHsCOL1A2-1A1.

(2-4) Preparation of a Plasmid (pEXP-HA-YHsCOL1A2-1A1 N60C15) for Introducing an Expression Cassette for a Fusion Polypeptide which Consists of the N-Terminal Non-Helix Region, 60 N-Terminal Residues of the Helix Region, 15 C-Terminal Residues of the Helix Region, and the C-Terminal Non-Helix Region of Human Collagen Type I α1 and an Expression Cassette For a Fusion Polypeptide which Consists of the N-Terminal Non-Helix region, 60 N-Terminal Residues of the Helix Region, 15 C-Terminal Residues of the Helix Region, and the C-Terminal Non-Helix Region of Human Collagen Type I α2
(2-4-1) Construction of pAT021

The oligonucleotides 58 and 59 mentioned below were synthesized. A fragment containing the expression cassette for a fusion polypeptide of the N-terminal non-helix region, 60 N-terminal residues of the helix region, 15 C-terminal residues of the helix region, and the C-terminal non-helix region of human collagen Type I α1, and a portion of the vector plasmid was amplified by PCR using the oligonucleotides 58 and 59 as primers and the plasmid named as pTS001 (see, Example (2-3-5)) as a template.

The oligonucleotides 58 and 59 used were:

(SEQ ID NO: 58)
(a) oligonucleotide 58: CGGCTTACCAGCCTCGC,
and
(SEQ ID NO: 59)
(b) oligonucleotide 59: GGACCACCAGGGCCGC.

The composition of the reaction solutions is given as follows:

(a) cDNA solution (10 ng/μl), 1 μl;
(b) dNTPs (mix, 2 mM each), 5 μl;
(c) MgSO₄ (25 mM), 2 μl;
(d) primers (10 pmol/μl), 1.5 μl each;
(e) 10×PCR buffer for KOD-plus- (Toyobo Co., Ltd.), 5 μl;
(f) KOD-plus- DNA polymerase (1 U/μl, Toyobo Co., Ltd.), 1 μl;
(g) sterile distilled water 33 μl.

The PCR was conducted under conditions where the reaction solution was heated at 94° C. for 2 minutes and then subjected to 25 cycles of denaturation at 94° C. for 15 seconds and each of annealing and extension at 68° C. for 6 minutes, followed by additionally keeping the reaction solution at 68° C. for 6 minutes.

An about 5.8-kb DNA fragment resulted from the PCR was purified using MinElute PCR Purification Kit (QIAGEN). To the DNA fragment obtained, a phosphate group was added at the 5′ end using T4 polynucleotide kinase (Takara Bio Inc.). Then, the phosphorylated, about 5.8-kb DNA fragment was separated by agarose gel electrophoresis, followed by extraction and purification from the gel using MinElute Gel Extraction Kit (QIAGEN).

The phosphorylated, about 5.8-kb DNA fragment was self-ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid which comprised the expression cassette for the fusion polypeptide consisting of the N-terminal non-helix region, 60 N-terminal residues of the helix region, 15 C-terminal residues of the helix region, and the C-terminal non-helix region of human collagen Type I α1 (which may be referred to hereinafter as pAT021) was purified from the cultured cells to give pAT021.

(2-4-2) Construction of pEXP-HA-YHsCOL1A1 N60C15

The plasmid named as pAT021 (see, Example (2-4-1)) was digested with restriction enzymes Eco52I and SpeI. An about 2.5-kb DNA fragment encoding the AOX1 promoter and the fusion polypeptide of the N-terminal non-helix region, 60 N-terminal residues of the helix region, 15 C-terminal residues of the helix region, and the C-terminal non-helix region of human collagen Type I α1 was isolated by agarose gel electrophoresis, followed by extraction and purification from the gel using MinElute Gel Extraction Kit (QIAGEN).

The plasmid named as pEXH002 (see, Example (2-3-4)) was digested with restriction enzymes Eco52I and SpeI. An about 6.6-kb DNA fragment was isolated by agarose gel electrophoresis, followed by extraction and purification from the gel using MinElute Gel Extraction Kit (QIAGEN).

The about 2.5-kb DNA fragment and the about 6.6-kb DNA fragment were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA fragment encoding the AOX1 promoter and the fusion polypeptide of the N-terminal non-helix region, 60 N-terminal residues of the helix region, 15 C-terminal residues of the helix region, and the C-terminal non-helix region of human collagen Type I α1 had been inserted (which may be referred to hereinafter as pEXP-HA-YHsCOL1A1 N60C15) was isolated from the cultured cells to give pEXP-HA-YHsCOL1A1 N60C15.

(2-4-3) Construction of pYHsCOL1A2 N60C15 Exp Unit-Eco52I

The oligonucleotides 60 and 61 mentioned below were synthesized. A fragment containing the expression cassette for a fusion polypeptide consisting of the N-terminal non-helix region, 60 N-terminal residues of the helix region, 15 C-terminal residues of the helix region, and the C-terminal non-helix region of human collagen Type I α2, and a portion of the vector plasmid was amplified by PCR using the oligonucleotides 60 and 61 as primers and the plasmid named as pYHsCOL1A2 Exp unit-Eco52I (see, Example (2-3-8)) as a template.

The oligonucleotides 60 and 61 used were:

(SEQ ID NO: 60)
(a) oligonucleotide 60: GGGTTTACCTGGGTGGCCG,
and
(SEQ ID NO: 61)
(b) oligonucleotide 61: GGACCTCCTGGCCCACC.

The composition of the reaction solutions is given as follows:

(a) cDNA solution (10 ng/μl), 1 μl;
(b) dNTPs (mix, 2 mM each), 5 μl;
(c) MgSO₄ (25 mM), 2 μl;
(d) primers (10 pmol/μl), 1.5 μl each;
(e) 10×PCR buffer for KOD-plus- (Toyobo Co., Ltd.), 5 μl;
(f) KOD-plus- DNA polymerase (1 U/μl, Toyobo Co., Ltd.), 1 μl;
(g) sterile distilled water 33 μl.

The PCR was conducted under conditions where the reaction solution was heated at 94° C. for 2 minutes and then subjected to 25 cycles of denaturation at 94° C. for 15 seconds and each of annealing and extension at 68° C. for 6 minutes, followed by additionally keeping the reaction solution at 68° C. for 6 minutes.

An about 6.1-kb DNA fragment resulted from the PCR was purified using MinElute PCR Purification Kit (QIAGEN). To the DNA fragment obtained, a phosphate group was added at the 5′ end using T4 polynucleotide kinase (Takara Bio Inc.). Then, the phosphorylated, about 6.1-kb DNA fragment was separated by agarose gel electrophoresis, followed by extraction and purification from the gel using MinElute Gel Extraction Kit (QIAGEN).

The phosphorylated, about 6.1-kb DNA fragment was self-ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of kanamycin, the E. coli cells which had been transformed were inoculated and cultured. A colony formed on the agar medium was inoculated into LB medium containing 50 μg/ml of kanamycin and incubated with shaking (37° C., 17 hours). Then, using QIAprep Spin Miniprep Kit (QIAGEN), the plasmid which comprised the expression cassette for the fusion polypeptide consisting of the N-terminal non-helix region, 60 N-terminal residues of the helix region, 15 C-terminal residues of the helix region, and the C-terminal non-helix region of human collagen Type I α2 (which may be referred to hereinafter as pYHsCOL1A2 N60C15 Exp unit-Eco52I) was purified from the cultured cells to give pYHsCOL1A2 N60C15 Exp unit-Eco52I.

(2-4-4) Construction of pEXP-HA-YHsCOL1A2-1A1 N60C15

The plasmid named as pYHsCOL1A2 N60C15 Exp unit-Eco52I (see, Example (2-4-3)) was digested with a restriction enzyme Eco52I. An about 2.3-kb DNA fragment which had the restriction enzyme recognition sequence added at each end of the expression cassette for the fusion polypeptide consisting of the N-terminal non-helix region, 60 N-terminal residues of the helix region, 15 C-terminal residues of the helix region, and the C-terminal non-helix region of human collagen Type I α2 was separated by agarose gel electrophoresis, followed by extraction and purification from the gel using MinElute Gel Extraction Kit (QIAGEN).

The plasmid named as pEXH-HA-YHsCOL1A1 N60C15 (see, Example (2-4-2)) was digested with a restriction enzyme Eco52I, dephosphorylated with an alkaline phosphatase, and then purified using MinElute Reaction Cleanup Kit (QIAGEN).

The about 2.3-kb DNA fragment and the dephosphorylated plasmid were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA fragment having the restriction enzyme recognition sequence added at each end of the expression cassette for the fusion polypeptide consisting of the N-terminal non-helix region, 60 N-terminal residues of the helix region, 15 C-terminal residues of the helix region, and the C-terminal non-helix region of human collagen Type I α2 had been inserted (which may be referred to hereinafter as pEXP-HA-YHsCOL1A2-1A1 N60C15) was isolated from the cultured cells to give pEXP-HA-YHsCOL1A1 N60C15.

(2-5) Preparation of a Plasmid (pEXP-HA-YHsCOL1A2-1A1 M500X2) for Introducing an Expression Cassette for a Fusion Polypeptide which Consists of the N-Terminal Non-Helix Region, 27 N-Terminal Residues of the Helix Region, a Dimer Made of 522 Central Residues of the Helix Region, 15 C-Terminal Residues of the Helix Region, and the C-Terminal Non-Helix Region of Human Collagen Type I α1 and an Expression Cassette for a Fusion Polypeptide which Consists of the N-Terminal Non-Helix Region, 27 N-Terminal Residues of the Helix Region, a Dimer Made of 522 Central Residues of the Helix Region, 15 C-Terminal Residues of the Helix Region, and the C-Terminal Non-Helix Region of Human Collagen Type I α2
(2-5-1) Construction of pAT017

The oligonucleotides 62 and 63 mentioned below were synthesized. A DNA fragment encoding the 522 central residues of the helix region of human collagen Type I α1 was amplified by PCR using the oligonucleotides 62 and 63 as primers and the plasmid named as pTS001 (see, Example (2-3-5)) as a template.

The oligonucleotides 62 and 63 used were:

(SEQ ID NO: 62)
(a) oligonucleotide 62: GGTCCACAGGGTCCAGGAG,
and
(SEQ ID NO: 63)
(b) oligonucleotide 63: ATCAGCTCCTGGTGATCCCTTTTC.

The composition of the reaction solutions is given as follows:

(a) cDNA solution (10 ng/μl), 1 μl;
(b) dNTPs (mix, 2 mM each), 5 μl;
(c) MgSO₄ (25 mM), 2 μl;
(d) primers (10 pmol/μl), 1.5 μl each;
(e) 10×PCR buffer for KOD-plus- (Toyobo Co., Ltd.), 5 μl;
(f) KOD-plus- DNA polymerase (1 U/μl, Toyobo Co., Ltd.), 1 μl;
(g) sterile distilled water 33 μl.

The PCR was conducted under conditions where the reaction solution was heated at 94° C. for 2 minutes and then subjected to 25 cycles of denaturation at 94° C. for 15 seconds and each of annealing and extension at 68° C. for 1 minute 40 seconds, followed by additionally keeping the reaction solution at 68° C. for 1 minute 40 seconds.

An about 1.6-kb DNA fragment resulted from the PCR was purified using MinElute PCR Purification Kit (QIAGEN). To the DNA fragment obtained, a phosphate group was added at the 5′ end using T4 polynucleotide kinase (Takara Bio Inc.). Then, the phosphorylated, about 1.6-kb DNA fragment was separated by agarose gel electrophoresis, followed by extraction and purification from the gel using MinElute Gel Extraction Kit (QIAGEN).

The oligonucleotides 59 and 64 mentioned below were synthesized. A region which was composed of the AOX1 promoter, a DNA fragment encoding the N-terminal non-helix region and 27 N-terminal residues of the helix region of human collagen Type I α1, a DNA fragment encoding the C-terminal non-helix region and 15 C-terminal residues of the helix region of human collagen Type I α1, and a portion of the vector plasmid was amplified by PCR using the oligonucleotides 59 and 64 as primers and the plasmid named as pTS001 (see, Example (2-3-5)) as a template.

The oligonucleotides 59 and 64 used were:

(SEQ ID NO: 59)
(a) oligonucleotide 59: GGACCACCAGGGCCGC,
and
(SEQ ID NO: 64)
(b) oligonucleotide 64: TGGTGGACCTTGAAAACCCTG.

The composition of the reaction solutions is given as follows:

(a) cDNA solution (10 ng/μl), 1 μl;
(b) dNTPs (mix, 2 mM each), 5 μl;
(c) MgSO₄ (25 mM), 2 μl;
(d) primers (10 pmol/μl), 1.5 μl each;
(e) 10×PCR buffer for KOD-plus- (Toyobo Co., Ltd.), 5 μl;
(f) KOD-plus- DNA polymerase (1 U/μl, Toyobo Co., Ltd.), 1 μl;
(g) sterile distilled water 33 μl.

The PCR was conducted under conditions where the reaction solution was heated at 94° C. for 2 minutes and then subjected to 25 cycles of denaturation at 94° C. for 15 seconds and each of annealing and extension at 68° C. for 6 minutes, followed by additionally keeping the reaction solution at 68° C. for 6 minutes.

An about 5.7-kb DNA fragment resulted from the PCR was purified using MinElute PCR Purification Kit (QIAGEN). The DNA fragment obtained was dephosphorylated with an alkaline phosphatase. Then, the dephosphorylated, about 5.7-kb DNA fragment was separated by agarose gel electrophoresis, followed by extraction and purification from the gel using MinElute Gel Extraction Kit (QIAGEN).

The phosphorylated, about 1.6-kb DNA fragment and the dephosphorylated, about 5.7-kb DNA fragment were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA fragment encoding the 522 central residues of the helix region of human collagen Type I α1 had been inserted (which may be referred to hereinafter as pAT017) was isolated from the cultured cells to give pAT017.

(2-5-2) Construction of pAT027

The oligonucleotides 65 and 66 mentioned below were synthesized. A DNA fragment encoding the 522 central residues of the helix region of human collagen Type I α1 was amplified by PCR using the oligonucleotides 65 and 66 as primers and the plasmid named as pBlue-HsCOL1A1(see, Example (1-10)) as a template.

The oligonucleotides 65 and 66 used were:

(SEQ ID NO: 65)
(a) oligonucleotide 65: GGACCCCAGGGCCCCG,
and
(SEQ ID NO: 66)
(b) oligonucleotide 66: ATCAGCACCAGGGGATCCTTTC.

The composition of the reaction solutions is given as follows:

(a) cDNA solution (10 ng/μl), 1 μl;
(b) dNTPs (mix, 2 mM each), 5 μl;
(c) MgSO₄ (25 mM), 2 μl;
(d) primers (10 pmol/μl), 1.5 μl each;
(e) 10×PCR buffer for KOD-plus- (Toyobo Co., Ltd.), 5 μl;
(f) KOD-plus- DNA polymerase (1 U/μl, Toyobo Co., Ltd.), 1 μl;
(g) sterile distilled water 33 μl.

The PCR was conducted under conditions where the reaction solution was heated at 94° C. for 2 minutes and then subjected to 25 cycles of denaturation at 94° C. for 15 seconds and each of annealing and extension at 68° C. for 1 minute 40 seconds, followed by additionally keeping the reaction solution at 68° C. for 1 minute 40 seconds.

An about 1.6-kb DNA fragment resulted from the PCR was purified using MinElute PCR Purification Kit (QIAGEN). To the DNA fragment obtained, a phosphate group was added at the 5′ end using T4 polynucleotide kinase (Takara Bio Inc.). Then, the phosphorylated, about 1.6-kb DNA fragment was separated by agarose gel electrophoresis, followed by extraction and purification from the gel using MinElute Gel Extraction Kit (QIAGEN).

The oligonucleotides 63 and 59 mentioned below were synthesized. A DNA fragment which was composed of the AOX1 promoter, a DNA fragment encoding the N-terminal non-helix region and 27 N-terminal residues of the helix region of human collagen Type I α1, a DNA fragment encoding the 522 central residues of the helix region of human collagen Type I α1, a DNA fragment encoding the C-terminal non-helix region and 15 C-terminal residues of the helix region of human collagen Type I α1, the AOX1 terminator, and a portion of the vector plasmid was amplified by PCR using the oligonucleotides 63 and 59 as primers and the plasmid named as pAT017 (see, Example (2-5-1)) as a template.

The oligonucleotides 63 and 59 used were:

(SEQ ID NO: 63)
(a) oligonucleotide 63: ATCAGCTCCTGGTGATCCCTTTTC,
and
(SEQ ID NO: 59)
(b) oligonucleotide 59: GGACCACCAGGGCCGC.

The composition of the reaction solutions is given as follows:

(a) cDNA solution (10 ng/μl), 1 μl;
(b) dNTPs (mix, 2 mM each), 5 μl;
(c) MgSO₄ (25 mM), 2 μl;
(d) primers (10 pmol/μl), 1.5 μl each;
(e) 10×PCR buffer for KOD-plus- (Toyobo Co., Ltd.), 5 μl;
(f) KOD-plus- DNA polymerase (1 U/μl, Toyobo Co., Ltd.), 1 μl;
(g) sterile distilled water 33 μl.

The PCR was conducted under conditions where the reaction solution was heated at 94° C. for 2 minutes and then subjected to 25 cycles of denaturation at 94° C. for 15 seconds and each of annealing and extension at 68° C. for 7 minutes 40 seconds, followed by additionally keeping the reaction solution at 68° C. for 7 minutes 40 seconds.

An about 7.3-kb DNA fragment resulted from the PCR was purified using MinElute PCR Purification Kit (QIAGEN). The DNA fragment obtained was dephosphorylated with an alkaline phosphatase. Then, the dephosphorylated, about 7.3-kb DNA fragment was separated by agarose gel electrophoresis, followed by extraction and purification from the gel using MinElute Gel Extraction Kit (QIAGEN).

The phosphorylated, about 1.6-kb DNA fragment and the dephosphorylated, about 7.3-kb DNA fragment were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA fragment encoding the 522 central residues of the helix region of human collagen Type I α1 had been inserted (which may be referred to hereinafter as pAT027) was isolated from the cultured cells to give pAT027.

(2-5-3) Construction of pEXP-HA-HsCOL1A1 M500X2

The plasmid named as pAT027 (see, Example (2-5-2)) was digested with restriction enzymes Eco52I and SpeI. An about 5.6-kb DNA fragment consisting of the AOX1 promoter and the DNA fragment encoding the fusion polypeptide of the N-terminal non-helix region, 27 N-terminal residues of the helix region, a dimer made of the 522 central residues of the helix region, 15 C-terminal residues of the helix region and the C-terminal non-helix region of human collagen Type I α1 was isolated by agarose gel electrophoresis, followed by extraction and purification from the gel using MinElute Gel Extraction Kit (QIAGEN).

The plasmid named as pEXH002 (see, Example (2-3-4)) was digested with restriction enzymes Eco52I and SpeI. An about 6.6-kb DNA fragment was isolated by agarose gel electrophoresis, followed by extraction and purification from the gel using MinElute Gel Extraction Kit (QIAGEN).

The about 5.6-kb DNA fragment and the about 6.6-kb DNA fragment were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA fragment encoding the AOX1 promoter and the fusion polypeptide of the N-terminal non-helix region, 27 N-terminal residues of the helix region, a dimer made of the 522 central residues of the helix region, 15 C-terminal residues of the helix region and the C-terminal non-helix region of human collagen Type I α1 had been inserted (which may be referred to hereinafter as pEXP-HA-HsCOL1A1 M500X2) was isolated from the cultured cells to give pEXP-HA-HsCOL1A1 M500X2.

(2-5-4) Construction of pAT018

The oligonucleotides 67 and 68 mentioned below were synthesized. A DNA fragment encoding the 522 central residues of the helix region of human collagen Type I α2 was amplified by PCR using the oligonucleotides 67 and 68 as primers and the plasmid named as pTS002 (see, Example (2-3-7)) as a template.

The oligonucleotides 67 and 68 used were:

(SEQ ID NO: 67)
(a) oligonucleotide 67: GGGCCAGTTGGCGCAG,
and
(SEQ ID NO: 68)
(b) oligonucleotide 68: TGCTTCTCCAGATGGTCCTTTCTC.

The composition of the reaction solutions is given as follows:

(a) cDNA solution (10 ng/μl), 1 μl;
(b) dNTPs (mix, 2 mM each), 5 μl;
(c) MgSO₄ (25 mM), 2 μl;
(d) primers (10 pmol/μl), 1.5 μl each;
(e) 10×PCR buffer for KOD-plus- (Toyobo Co., Ltd.), 5 μl;
(f) KOD-plus- DNA polymerase (1 U/μl, Toyobo Co., Ltd.), 1 μl;
(g) sterile distilled water 33 μl.

The PCR was conducted under conditions where the reaction solution was heated at 94° C. for 2 minutes and then subjected to 25 cycles of denaturation at 94° C. for 15 seconds and each of annealing and extension at 68° C. for 1 minute 40 seconds, followed by additionally keeping the reaction solution at 68° C. for 1 minute 40 seconds.

An about 1.6-kb DNA fragment resulted from the PCR was purified using MinElute PCR Purification Kit (QIAGEN). To the DNA fragment obtained, a phosphate group was added at the 5′ end using T4 polynucleotide kinase (Takara Bio Inc.). Then, the phosphorylated, about 1.6-kb DNA fragment was separated by agarose gel electrophoresis, followed by extraction and purification from the gel using MinElute Gel Extraction Kit (QIAGEN).

The oligonucleotides 61 and 69 mentioned below were synthesized. A DNA fragment which was composed of the AOX1 promoter, a DNA fragment encoding the N-terminal non-helix region and 27 N-terminal residues of the helix region of human collagen Type I α2, a DNA fragment encoding the C-terminal non-helix region and 15 C-terminal residues of the helix region of human collagen Type I α2, the AOX1 terminator, and a portion of the vector plasmid was amplified by PCR using the oligonucleotides 61 and 69 as primers and the plasmid named as pTS002 (see, Example (2-3-7)) as a template.

The oligonucleotides 61 and 69 used were:

(SEQ ID NO: 61)
(a) oligonucleotide 61: GGACCTCCTGGCCCACC,
and
(SEQ ID NO: 69)
(b) oligonucleotide 69: TGCGGGTCCTTGGAATC.

The composition of the reaction solutions is given as follows:

(a) cDNA solution (10 ng/μl), 1 μl;
(b) dNTPs (mix, 2 mM each), 5 μl;
(c) MgSO₄ (25 mM), 2 μl;
(d) primers (10 pmol/μl), 1.5 μl each;
(e) 10×PCR buffer for KOD-plus- (Toyobo Co., Ltd.), 5 μl;
(f) KOD-plus- DNA polymerase (1 U/μl, Toyobo Co., Ltd.), 1 μl;
(g) sterile distilled water 33 μl.

The PCR was conducted under conditions where the reaction solution was heated at 94° C. for 2 minutes and then subjected to 25 cycles of denaturation at 94° C. for 15 seconds and each of annealing and extension at 68° C. for 6 minutes, followed by additionally keeping the reaction solution at 68° C. for 6 minutes.

An about 5.4-kb DNA fragment resulted from the PCR was purified using MinElute PCR Purification Kit (QIAGEN). The DNA fragment obtained was dephosphorylated with an alkaline phosphatase. Then, the dephosphorylated, about 5.4-kb DNA fragment was separated by agarose gel electrophoresis, followed by extraction and purification from the gel using MinElute Gel Extraction Kit (QIAGEN).

The phosphorylated, about 1.6-kb DNA fragment and the dephosphorylated, about 5.4-kb DNA fragment were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA fragment encoding the 522 central residues of the helix region of human collagen Type I α2 had been inserted (which may be referred to hereinafter as pAT018) was isolated from the cultured cells to give pAT018.

(2-5-5) Construction pAT018-Eco52I

The plasmid named as pAT018 (see, Example (2-5-4)) was digested with restriction enzymes AccIII and PmeI. An about 2.6-kb fragment comprising a 3′ region of the AOX1 promoter, an DNA fragment encoding the N-terminal non-helix region and 27 N-terminal residues of the helix region of human collagen Type I α2 and an DNA fragment encoding the 522 central residues of the helix region of human collagen Type I α2 was isolated by agarose gel electrophoresis, followed by extraction and purification from the gel using MinElute Gel Extraction Kit (QIAGEN).

The plasmid named as pYHsCOL1A2 Exp unit-Eco52I (see, Example (2-3-8)) was digested with restriction enzymes AccIII and PmeI. An about 4.9-kb DNA fragment was separated by agarose gel electrophoresis, followed by extraction and purification from the gel using MinElute Gel Extraction Kit (QIAGEN).

The about 2.6-kb DNA fragment and the about 4.9-kb DNA fragment were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of kanamycin, the E. coli cells which had been transformed were inoculated and cultured. A colony formed on the agar medium was inoculated into LB medium containing 50 μg/ml of kanamycin and incubated with shaking (37° C., 17 hours). Then, using QIAprep Spin Miniprep Kit (QIAGEN), the plasmid into which the fragment comprising the 3′ region of the AOX1 promoter, the DNA fragment encoding the N-terminal non-helix region and 27 N-terminal residues of the helix region of human collagen Type I α2 and the DNA fragment encoding 522 central residues of the helix region of human collagen Type I α2 had been inserted (which may be referred to hereinafter as pAT018-Eco52I) was isolated from the cultured cells to give pAT018-Eco52I.

(2-5-6) Construction of pAT028-Eco52I

The oligonucleotides 70 and 71 mentioned below were synthesized. A DNA fragment encoding the 522 central residues of the helix region of human collagen Type I α2 was amplified by PCR using the oligonucleotides 70 and 71 as primers and the plasmid named as pUC18-HsCOL1A2 (see, Example (1-11)) as a template.

The oligonucleotides 70 and 71 used were:

(SEQ ID NO: 70)
(a) oligonucleotide 70: GGCCCTGTTGGTGCTGC,
and
(SEQ ID NO: 71)
(b) oligonucleotide 71: AGCCTCTCCAGAGGGACCCTT.

The composition of the reaction solutions is given as follows:

(a) cDNA solution (10 ng/μl), 1 μl;
(b) dNTPs (mix, 2 mM each), 5 μl;
(c) MgSO₄ (25 mM), 2 μl;
(d) primers (10 pmol/μl), 1.5 μl each;
(e) 10×PCR buffer for KOD-plus- (Toyobo Co., Ltd.), 5 μl;
(f) KOD-plus- DNA polymerase (1 U/μl, Toyobo Co., Ltd.), 1 μl;
(g) sterile distilled water 33 μl.

The PCR was conducted under conditions where the reaction solution was heated at 94° C. for 2 minutes and then subjected to 25 cycles of denaturation at 94° C. for 15 seconds and each of annealing and extension at 68° C. for 1 minute 40 seconds, followed by additionally keeping the reaction solution at 68° C. for 1 minute 40 seconds.

An about 1.6-kb DNA fragment resulted from the PCR was purified using MinElute PCR Purification Kit (QIAGEN). To the DNA fragment obtained, a phosphate group was added at the 5′ end using T4 polynucleotide kinase (Takara Bio Inc.). Then, the phosphorylated, about 1.6-kb DNA fragment was separated by agarose gel electrophoresis, followed by extraction and purification from the gel using MinElute Gel Extraction Kit (QIAGEN).

The oligonucleotides 68 and 61 mentioned below were synthesized. A DNA fragment which was composed of the AOX1 promoter, a DNA fragment encoding the N-terminal non-helix region and 27 N-terminal residues of the helix region of human collagen Type I α2, a DNA fragment encoding the 522 central residues of the helix region of human collagen Type I α2, a DNA fragment encoding the C-terminal non-helix region and 15 C-terminal residues of the helix region of human collagen Type I α2, the AOX1 terminator and a portion of the vector plasmid was amplified by PCR using the oligonucleotides 68 and 61 as primers and the plasmid named as pAT018-Eco52I (see, Example (2-5-5)) as a template.

The oligonucleotides 68 and 61 used were:

(SEQ ID NO: 68)
(a) oligonucleotide 68: TGCTTCTCCAGATGGTCCTTTCTC,
and
(SEQ ID NO: 61)
(b) oligonucleotide 61: GGACCTCCTGGCCCACC.

The composition of the reaction solutions is given as follows:

(a) cDNA solution (10 ng/μl), 1 μl;
(b) dNTPs (mix, 2 mM each), 5 μl;
(c) MgSO₄ (25 mM), 2 μl;
(d) primers (10 pmol/μl), 1.5 μl each;
(e) 10×PCR buffer for KOD-plus- (Toyobo Co., Ltd.), 5 μl;
(f) KOD-plus- DNA polymerase (1 U/μl, Toyobo Co., Ltd.), 1 μl;
(g) sterile distilled water 33 μl.

The PCR was conducted under conditions where the reaction solution was heated at 94° C. for 2 minutes and then subjected to 25 cycles of denaturation at 94° C. for 15 seconds and each of annealing and extension at 68° C. for 7 minutes 40 seconds, followed by additionally keeping the reaction solution at 68° C. for 7 minutes 40 seconds.

An about 7.6-kb DNA fragment resulted from the PCR was purified using MinElute PCR Purification Kit (QIAGEN). The DNA fragment obtained was dephosphorylated with an alkaline phosphatase. Then, the dephosphorylated, about 7.3-kb DNA fragment was separated by agarose gel electrophoresis, followed by extraction and purification from the gel using MinElute Gel Extraction Kit (QIAGEN).

The phosphorylated, about 1.6-kb DNA fragment and the dephosphorylated, about 7.6-kb DNA fragment were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA fragment encoding the 522 central residues of the helix region of human collagen Type I α2 had been inserted (which may be referred to hereinafter as pAT028-Eco52I) was isolated from the cultured cells to give pAT028-Eco52I.

(2-5-7) Construction of pEXP-HA-HsCOL1A2-1A1 M500X2

The plasmid named as pAT028-Eco52I (see, Example (2-5-6)) was digested with a restriction enzyme Eco52I. An about 5.6-kb DNA fragment which comprised the expression cassette for the fusion polypeptide consisting of the N-terminal non-helix region, 27 N-terminal residues of the helix region, a dimer made of the 522 central residues of the helix region, 15 C-terminal residues of the helix region and the C-terminal non-helix region of human collagen Type I α2 was separated by agarose gel electrophoresis, followed by extraction and purification from the gel using MinElute Gel Extraction Kit (QIAGEN).

The plasmid named as pEXP-HA-HsCOL1A1 M500X2 (see, Example (2-5-3)) was digested with a restriction enzyme Eco52I, dephosphorylated with an alkaline phosphatase, and then purified using MinElute Reaction Cleanup Kit (QIAGEN).

The about 5.6-kb DNA fragment and the dephosphorylated plasmid were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA fragment comprising the expression cassette for the fusion polypeptide consisting of the N-terminal non-helix region, 27 N-terminal residues of the helix region, a dimer made of the 522 central residues of the helix region, 15 C-terminal residues of the helix region and the C-terminal non-helix region of human collagen Type I α2 had been inserted (which may be referred to hereinafter as pEXP-HA-HsCOL1A2-1A1 M500×2) was purified from the cultured cells to give pEXP-HA-HsCOL1A2-1A1 M500X2.

(2-6) Preparation of a Plasmid (pEXP-HA-HsCOL3A1) for Introducing an Expression Cassette for Human Collagen Type III α1
(2-6-1) Construction of pSN026

The oligonucleotides 72 and 73 mentioned below were synthesized. A DNA fragment which had a restriction enzyme recognition sequence added at each end of the DNA encoding human collagen Type III α1 was amplified by PCR using the oligonucleotides 72 and 73 as primers and the plasmid named as pUC19-HsCOL3A1 (see, Example (1-12)) as a template.

The oligonucleotides 72 and 73 used were:

(a) oligonucleotide 72:
(SEQ ID NO: 72)
TATTCGAAACGATGATGAGCTTTGTGCAAAAGGGG,
and
(b) oligonucleotide 73:
(SEQ ID NO: 73)
TTACTAGTTTATAAAAAGCAAACAGGGCCAACGT.

The composition of the reaction solutions is given as follows:

(a) cDNA solution (10 ng/μl), 1 μl;
(b) dNTPs (mix, 2 mM each), 5 μl;
(c) MgSO₄ (25 mM), 2 μl;
(d) primers (10 pmol/μl), 1.5 μl each;
(e) 10×PCR buffer for KOD-plus- (Toyobo Co., Ltd.), 5 μl;
(f) KOD-plus- DNA polymerase (1 U/μl, Toyobo Co., Ltd.), 1 μl;
(g) sterile distilled water 33 μl.

The PCR was conducted under conditions where the reaction solution was heated at 94° C. for 2 minutes and then subjected to 5 cycles of denaturation at 94° C. for 15 seconds, annealing at 60° C. for 30 seconds, and extension at 68° C. for 5 minutes and then 23 cycles of denaturation at 94° C. for 15 seconds, and each of annealing and extension at 68° C. for 5 minutes, followed by additionally keeping the reaction solution at 68° C. for 5 minutes.

An about 4.4-kb DNA fragment resulted from the PCR was digested with restriction enzymes BspT104I and SpeI. Then, the digested, about 4.4-kb DNA fragment was isolated by agarose gel electrophoresis, followed by extraction and purification from the gel using MinElute Gel Extraction Kit (QIAGEN).

The plasmid named as pSN006 (see, Example (2-3-3)) was digested with restriction enzymes BspT104I and SpeI. Then, an about 4.2-kb DNA fragment was separated by agarose gel electrophoresis, followed by extraction and purification from the gel using MinElute Gel Extraction Kit (QIAGEN).

The digested, about 4.4-kb DNA fragment and the about 4.2-kb DNA fragment were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA fragment having the restriction enzyme recognition sequence added at each end of the DNA encoding human collagen Type III α1 had been inserted (which may be referred to hereinafter as pSN026) was isolated from the cultured cells to give pSN026.

(2-6-2) Construction of pEXP-HA-HsCOL3A1

The plasmid named as pSN026 (see, Example (2-6-1)) was digested with restriction enzymes Eco52I and SpeI. An about 5.3-kb DNA fragment encoding the AOX1 promoter and human collagen Type III α1 was isolated by agarose gel electrophoresis, followed by extraction and purification from the gel using MinElute Gel Extraction Kit (QIAGEN).

The plasmid named as pEXH002 (see, Example (2-3-4)) was digested with restriction enzymes Eco52I and SpeI. An about 6.6-kb DNA fragment was isolated by agarose gel electrophoresis, followed by extraction and purification from the gel using MinElute Gel Extraction Kit (QIAGEN).

The about 5.3-kb DNA fragment and the about 6.6-kb DNA fragment were ligated, and the resulting ligation solution was used for transformation of E. coli (Competent high DH5α, Toyobo Co., Ltd.). For the ligation reaction, a Ligation Kit ver 2.1 (Takara Bio Inc.) was used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA fragment encoding the AOX1 promoter and human collagen Type III α1 had been inserted (which may be referred to hereinafter as pEXP-HA-HsCOL3A1) was isolated from the cultured cells to give pEXP-HA-HsCOL3A1.

(2-7) Preparation of a Plasmid (pEXP-HA-YHsCOL2A1) for Introducing an Expression Cassette for Human Collagen Type II α1
(2-7-1) Construction of pIM200

The plasmid named as pUC57-YHsCOL2A1 (see, Example (1-21)) was digested with restriction enzymes BspT104I and SpeI. An about 4.4-kb DNA fragment encoding human collagen Type II α1 was separated by agarose gel electrophoresis, followed by extraction and purification from the gel using MinElute Gel Extraction Kit (QIAGEN).

The plasmid named as pSN006 (see, Example (2-3-3)) was digested with restriction enzymes BspT104I and SpeI. An about 4.2-kb DNA fragment was separated by agarose gel electrophoresis, followed by extraction and purification from the gel using MinElute Gel Extraction Kit (QIAGEN).

The about 4.4-kb DNA fragment and the about 4.2-kb DNA fragment are ligated, and the resulting ligation solution is used for transformation of E. coli (Competent high DH5α, Toyobo Co., Ltd.). For the ligation reaction, a Ligation Kit ver 2.1 (Takara Bio Inc.) is used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA fragment encoding human collagen Type II α1 had been inserted (which may be referred to hereinafter as pIM200) was isolated from the cultured cells to give pIM200.

(2-7-2) Construction of pEXP-HA-YHsCOL2A1

The plasmid named as pIM200 (see, Example (2-7-1)) was digested with restriction enzymes Eco52I and SpeI. An about 5.3-kb DNA fragment encoding the AOX1 promoter and human collagen Type II α1 was isolated by agarose gel electrophoresis, followed by extraction and purification from the gel using MinElute Gel Extraction Kit (QIAGEN).

The plasmid named as pEXH002 (see, Example (2-3-4)) was digested with restriction enzymes Eco52I and SpeI. An about 6.6-kb DNA fragment was isolated by agarose gel electrophoresis, followed by extraction and purification from the gel using MinElute Gel Extraction Kit (QIAGEN).

The about 5.3-kb DNA fragment and the about 6.6-kb DNA fragment are ligated, and the resulting ligation solution is used for transformation of E. coli (Competent high DH5α, Toyobo Co., Ltd.). For the ligation reaction, a Ligation Kit ver 2.1 (Takara Bio Inc.) is used.

On LB agar medium containing 50 μg/ml of ampicillin, the E. coli cells which had been transformed were inoculated and cultured. A colony formed on the agar medium was inoculated into LB medium containing 50 μg/ml of ampicillin and incubated with shaking (37° C., 17 hours). Then, using QIAprep Spin Miniprep Kit (QIAGEN), the plasmid into which the DNA fragment encoding the AOX1 promoter and human collagen Type II α1 had been inserted (which may be referred to hereinafter as pEXP-HA-YHsCOL2A1) was isolated from the cultured cells to give pEXP-HA-YHsCOL2A1.

### Example 3

### Preparation of Yeasts Having Expression Cassettes Introduced Therein

(3-1) Preparation of a Yeast in which an Expression Cassette for Prolyl 4-Hydroxylase α1 Subunit and an Expression Cassette for Prolyl 4-Hydroxylase β Subunit are Introduced

### (3-1-1) Transformation of Yeast

Komagataella pastoris strain PPY12 (ATCC 204163) which was commercially available from the American Type Culture Collection (ATCC) was purchased.

Using an electroporation method, strain PPY12 was transformed with pEXP-A-P4Hbsig(−)A1rev obtained in Example 2-1.

Into 100 ml of YPD liquid medium (1% Yeast Extract, 2% peptone, and 2% glucose), strain PPY12 was inoculated, and cultured at 30° C. until the turbidity of cell culture (OD₆₀₀) reached about 10. The cells were collected from the cultured medium and the supernatant was removed. The cells were then suspended in 1M sorbitol at a turbidity of OD₆₆₀=about 150 to prepare a cell suspension.

Ten (10) μg of pEXP-A-P4Hbsig(−)A1rev (see, Example (2-1-12)) was digested with a restriction enzyme AatII. The DNA was collected through ethanol precipitation and dissolved in 5 μl of 10 mM Tris-HCl to prepare a DNA solution.

A mixture was prepared by combining 100 μl of the cell suspension and 5 μl of the DNA solution. To the mixture, a pulse was applied using a gene transfer device (ECM630, BTX), and then the mixture was spread on MD agar medium (1.34% Yeast Nitrogen Base, 4×10⁻⁵% biotin, 2% glucose, 0.005% histidine, 2% agar) and cultured at 30° C. for 48 hours. Colonies formed on the MD agar medium were isolated as transformants. Strain 050525-5-3 was obtained as a transformant.

### (3-1-2) Determination of Expression of Prolyl 4-Hydroxylase α1 Subunit and Prolyl 4-Hydroxylase β Subunit

Strain 050525-5-3 was inoculated in 100 ml of BMGY medium [1% Yeast Extract, 2% peptone, 100 mM potassium phosphate buffer (pH 6.0), 1.34% Yeast Nitrogen Base (Difco), 4×10⁻⁵% biotin, 1% glycerol, 0.005% histidine] which was prepared in a 500-ml baffled Erlenmeyer flask, and cultured with shaking at 30° C. for 24 hours. A cell suspension was prepared from the resultant culture, inoculated in 50 ml of BMM medium [100 mM potassium phosphate buffer (pH 6.0), 1.34% Yeast Nitrogen Base (Difco), 4×10⁻⁵% biotin, 0.5% methanol, 0.005% histidine] which was prepared in a 500-ml baffled Erlenmeyer flask, at a final concentration of OD₆₆₀=about 10, and cultured with shaking at 30° C. for 72 hours. Every about 12 hours during this period, 50% methanol was added in an amount of 0.5 ml each.

The cells were collected from the resultant culture and the supernatant was removed. After that, the cells were subjected to cell disruption using a Multi-beads Shocker (Yasui Kikai Corporation) under conditions for yeast cell disruption (glass beads of a diameter of 0.5 mm, 2,500 rpm, 40 minutes). The solution resulting from the cell disruption was centrifuged to collect the supernatant of the cell disrupted solution to prepare a soluble fraction.

An aliquot of the soluble fraction was mixed with a sample buffer (Nacalai Tesque., Inc.), and the mixture was kept at 100° C. for 5 minutes, followed by electrophoresis on SDS-polyacrylamide gel. The electrophoresis, in which the an ERICA-MP (DRC) was used for electrophoresis apparatus and 5-15% gradient gel (XV PANTERA MP gel, DRC) was used for the electrophoresis gel, was performed at 200 V for 20 minutes. After the electrophoresis was completed, the acrylamide gel was subjected to electro-blotting onto a PVDF membrane (Immobilon-P, Millipore Corp.). The electro-blotting, in which MINICA-MP (DRC) was used for the blotting apparatus, was performed at 40 V for 30 minutes.

A half of the resulting membrane was used to carry out western blotting analysis to detect prolyl 4-hydroxylase α1 subunit. Blocking one (Nacalai Tesque., Inc.) was used for blocking of the membrane, Can Get Signal (Toyobo Co., Ltd.) was used for antibody reactions, 2,000-times diluted anti-rat prolyl 4-hydroxylase α1 subunit mouse antibody (63167, MP Biomedicals) was used for the primary antibody, and 20,000-times diluted anti-mouse IgG-HRP Linked sheep antibody (NA-931, GE Healthcare Bio Science) was used for the secondary antibody. The secondary antibody was detected by chemiluminescence using ECL Western Blotting Detection System (GE Healthcare Bio Science). The chemiluminescence was detected and quantitatively determined with an image analyzer (LAS-3000UVmini, Fujifilm Corporation). The results of western blotting analysis demonstrated that a signal was detected at the position corresponding to the molecular weight of the prolyl 4-hydroxylase α1 subunit, and thus, it was shown that the prolyl 4-hydroxylase α1 subunit was produced.

The other half of the resulting membrane was used to carry out western blotting analysis to detect prolyl 4-hydroxylase β subunit. Blocking one (Nacalai Tesque., Inc.) was used for blocking of the membrane, Can Get Signal (Toyobo Co., Ltd.) was used for antibody reactions, 5,000-times diluted anti-human prolyl 4-hydroxylase β subunit rabbit antibody (SPA-890, Stressgen) was used for the primary antibody, and 20,000-times diluted anti-rabbit IgG-HRP Linked donkey antibody (NA-934, GE Healthcare Bio Science) was used for the secondary antibody. The secondary antibody was detected by chemiluminescence using ECL Western Blotting Detection System (GE Healthcare Bio Science). The chemiluminescence was detected and quantitatively determined with an image analyzer (LAS-3000UVmini, Fujifilm Corporation). The results of western blotting analysis demonstrated that strain 050525-5-3 provided a signal that was detected at the position corresponding to the molecular weight of the prolyl 4-hydroxylase β subunit, and thus, it was shown that the prolyl 4-hydroxylase β subunit was produced.

(3-2) Preparation of a Yeast in which an Expression Cassette for Prolyl 4-Hydroxylase α1 Subunit, an Expression Cassette for Prolyl 4-Hydroxylase β Subunit, an Expression Cassette for Human Collagen Type I α1 and an Expression Cassette for Human Collagen Type I α2 are Introduced

### (3-2-1) Transformation of Yeast

Using an electroporation method, strain 050525-5-3 (see, Example (3-1-1)) was transformed with pEXP-HA-YHsCOL1A2-1A1 obtained in Example 2-3-9.

Into 100 ml of YPD liquid medium (1% Yeast Extract, 2% peptone, and 2% glucose), strain 050525-5-3 was inoculated, and cultured at 30° C. until the turbidity of cell culture (OD₆₆₀) reached about 10. The cells were collected from the cultured medium and the supernatant was removed. The cells were then suspended in 1M sorbitol at a turbidity of OD₆₆₀=about 150 to prepare a cell suspension.

Ten (10) μg of pEXP-HA-YHsCOL1A2-1A1 (see, Example (2-3-9)) was digested with a restriction enzyme XbaI. The DNA was collected through ethanol precipitation and dissolved in 5 μl of 10 mM Tris-HCl to prepare a DNA solution.

A mixture was prepared by combining 100 μl of the cell suspension and 5 μl of the DNA solution. To the mixture, a pulse was applied using a gene transfer device (ECM630, BTX), and then the mixture was spread on MD agar medium (1.34% Yeast Nitrogen Base, 4×10⁻⁵% biotin, 2% glucose, 2% agar) and cultured at 30° C. for 48 hours. Colonies formed on the MD agar medium were isolated as transformants. Strain 070327-1-11 was obtained as a transformant.

### (3-2-2) Determination of Expression of Human Collagen Type I α1 and Human Collagen Type I α2

Strain 070327-1-11 (see, Example (3-2-1)) was inoculated in 100 ml of BMGY medium [1% Yeast Extract, 2% peptone, 100 mM potassium phosphate buffer (pH 6.0), 1.34% Yeast Nitrogen Base (Difco), 4×10⁻⁵% biotin, 1% glycerol] which was prepared in a 500-ml baffled Erlenmeyer flask, and cultured with shaking at 30° C. for 24 hours. A cell suspension was prepared from the resultant culture, inoculated in 50 ml of BMM medium [100 mM potassium phosphate buffer (pH 6.0), 1.34% Yeast Nitrogen Base (Difco), 4×10⁻⁵% biotin, 0.5% methanol] which was prepared in a 500-ml baffled Erlenmeyer flask, at a final concentration of OD₆₆₀=about 10, and cultured with shaking at 30° C. for 72 hours. Every about 12 hours during this period, 50% methanol was added in an amount of 0.5 ml each.

The cells were collected from the resultant culture and the supernatant was removed. After that, the cells were subjected to cell disruption using a Multi-beads Shocker (Yasui Kikai Corporation) under conditions for yeast cell disruption (glass beads of a diameter of 0.5 mm, 2,500 rpm, 40 minutes). The solution resulting from the cell disruption was centrifuged to collect the supernatant of the cell disrupted solution to prepare a soluble fraction.

An aliquot of the soluble fraction was mixed with a sample buffer (Nacalai Tesque., Inc.), and the mixture was kept at 100° C. for 5 minutes, followed by electrophoresis on SDS-polyacrylamide gel. The electrophoresis, in which ERICA-MP (DRC) was used for the electrophoresis apparatus and 5-15% gradient gel (XV PANTERA MP gel, DRC) was used for the electrophoresis gel, was performed at 200 V for 20 minutes. After the electrophoresis was completed, the resulting acrylamide gel was subjected to staining with a CBB staining solution to detect protein bands. The results demonstrated that strain 070327-1-1 provided bands that were detected at the molecular weights corresponding to a procollagen of collagen Type I α1 and corresponding to a procollagen of collagen Type I α2, and thus it was shown that human collagen Type I α1 and human collagen Type I α2 was produced.

(3-3) Preparation of a Yeast in which an Expression Cassette for Prolyl 4-Hydroxylase α1 Subunit, an Expression Cassette for Prolyl 4-Hydroxylase β Subunit, an Expression Cassette for a Fusion Polypeptide which Consists of the N-Terminal Non-Helix Region, 60 N-Terminal Residues of the Helix Region, 15 C-Terminal Residues of the Helix Region and the C-Terminal Non-Helix Region of Human Collagen Type I α1, and an Expression Cassette for a Fusion Polypeptide which Consists of the N-Terminal Non-Helix Region, 60 N-Terminal Residues of the Helix Region, 15 C-Terminal Residues of the Helix Region and the C-Terminal Non-Helix Region of Human Collagen Type I α2 are Introduced

### (3-3-1) Transformation of Yeast

Using an electroporation method, strain 050525-5-3 (see, Example (3-1-1)) was transformed with pEXP-HA-YHsCOL1A2-1A1 N60C15 obtained in Example 2-4-4.

Into 100 ml of YPD liquid medium (1% Yeast Extract, 2% peptone, and 2% glucose), strain 050525-5-3 (see, Example (3-1-1)) was inoculated, and cultured at 30° C. until the turbidity of cell culture (OD₆₆₀) reached about 10. The cells were collected from the cultured medium and the supernatant was removed. The cells were then suspended in 1M sorbitol at a turbidity of OD₆₆₀=about 150 to prepare a cell suspension.

Ten (10) μg of pEXP-HA-YHsCOL1A2-1A1 N60C15 (see, Example (2-4-4)) was digested with a restriction enzyme XbaI. The DNA was collected through ethanol precipitation and dissolved in 5 μl of 10 mM Tris-HCl to prepare a DNA solution.

A mixture was prepared by combining 100 μl of the cell suspension and 5 μl of the DNA solution. To the mixture, a pulse was applied using a gene transfer device (ECM630, BTX), and then the mixture was spread on MD agar medium (1.34% Yeast Nitrogen Base, 4×10⁻⁵% biotin, 2% glucose, 2% agar) and cultured at 30° C. for 48 hours. Colonies formed on the MD agar medium were isolated as transformants. Strain 080118-3-3 was obtained as a transformant.

(3-3-2) Determination of Expression of a Fusion Polypeptide which Consists of the N-Terminal Non-Helix Region, 60 N-Terminal Residues of the Helix Region, 15 C-Terminal Residues of the Helix Region and the C-Terminal Non-Helix Region of Human Collagen Type I α1 and an Expression Cassette for a Fusion Polypeptide which Consists of the N-Terminal Non-Helix Region, 60 N-Terminal Residues of the Helix Region, 15 C-Terminal Residues of the Helix Region and the C-Terminal Non-Helix Region of Human Collagen Type I α2

Strain 080118-3-3 (see, Example (3-3-1)) was inoculated in 100 ml of BMGY medium [1% Yeast Extract, 2% peptone, 100 mM potassium phosphate buffer (pH 6.0), 1.34% Yeast Nitrogen Base (Difco), 4×10⁻⁵% biotin, 1% glycerol] which was prepared in a 500-ml baffled Erlenmeyer flask, and cultured with shaking at 30° C. for 24 hours. A cell suspension was prepared from the resultant culture, inoculated in 50 ml of BMM medium [100 mM potassium phosphate buffer (pH 6.0), 1.34% Yeast Nitrogen Base (Difco), 4×10⁻⁵% biotin, 0.5% methanol] which was prepared in a 500-ml baffled Erlenmeyer flask, at a final concentration of OD₆₆₀=about 10, and cultured with shaking at 30° C. for 72 hours. Every about 12 hours during this period, 50% methanol was added in an amount of 0.5 ml each.

The cells were collected from the resultant culture and the supernatant was removed. After that, the cells were subjected to cell disruption using a Multi-beads Shocker (Yasui Kikai Corporation) under conditions for yeast cell disruption (glass beads of a diameter of 0.5 mm, 2,500 rpm, 40 minutes). The solution resulting from the cell disruption was centrifuged to collect the supernatant of the cell disrupted solution to prepare a soluble fraction.

An aliquot of the soluble fraction was mixed with a sample buffer (Nacalai Tesque., Inc.), and the mixture was kept at 100° C. for 5 minutes, followed by electrophoresis on SDS-polyacrylamide gel. The electrophoresis, in which ERICA-MP (DRC) was used for the electrophoresis apparatus and 5-15% gradient gel (XV PANTERA MP gel, DRC) was used for the electrophoresis gel, was performed at 200 V for 20 minutes. After the electrophoresis was completed, the acrylamide gel was subjected to electro-blotting onto a PVDF membrane (Immobilon-P, Millipore Corp.). The electro-blotting, in which MINICA-MP (DRC) was used for the blotting apparatus, was performed at 40 V for 30 minutes.

A half of the resulting membrane was used to carry out western blotting analysis to detect a procollagen of the fusion polypeptide consisting of the N-terminal non-helix region, 60 N-terminal residues of the helix region, 15 C-terminal residues of the helix region and the C-terminal non-helix region of human collagen Type I α1. Blocking one (Nacalai Tesque., Inc.) was used for blocking of the membrane, Can Get Signal (Toyobo Co., Ltd.) was used for antibody reactions, 2,000-times diluted anti-human procollagen Type I α1 goat antibody (SC-8782, Santa Cruz) was used for the primary antibody, and 2,000-times diluted anti-goat IgG-HRP Linked donkey antibody (SC-2033, Santa Cruz) was used for the secondary antibody. The secondary antibody was detected by chemiluminescence using ECL Western Blotting Detection System (GE Healthcare Bio Science). The chemiluminescence was detected and quantitatively determined with an image analyzer (LAS-3000UVmini, Fujifilm Corporation). The results of western blotting analysis demonstrated that strain 080118-3-3 provided a signal that was detected at the position corresponding to the molecular weight of a procollagen of the fusion polypeptide consisting of the N-terminal non-helix region, 60 N-terminal residues of the helix region, 15 C-terminal residues of the helix region and the C-terminal non-helix region of human collagen Type I α1, and thus, it was shown that the fusion polypeptide consisting of the N-terminal non-helix region, 60 N-terminal residues of the helix region, 15 C-terminal residues of the helix region and the C-terminal non-helix region of human collagen Type I α1 was produced.

The other half of the resulting membrane was used to carry out western blotting analysis to detect a procollagen of the fusion polypeptide consisting of the N-terminal non-helix region, 60 N-terminal residues of the helix region, 15 C-terminal residues of the helix region and the C-terminal non-helix region of human collagen Type I α2. Blocking one (Nacalai Tesque., Inc.) was used for blocking of the membrane, Can Get Signal (Toyobo Co., Ltd.) was used for antibody reactions, 5,000-times diluted anti-human collagen Type I rabbit antibody (ab292, abcam) was used for the primary antibody, and 10,000-times diluted anti-rabbit IgG-HRP Linked donkey antibody (NA-934, GE Healthcare Bio Science) was used for the secondary antibody. The secondary antibody was detected by chemiluminescence using ECL Western Blotting Detection System (GE Healthcare Bio Science). The chemiluminescence was detected and quantitatively determined with an image analyzer (LAS-3000UVmini, Fujifilm Corporation). The results of western blotting analysis demonstrated that strain 080118-3-3 (see, Example (3-7-1)) provided a signal that was detected at the position corresponding to the molecular weight of a procollagen of the fusion polypeptide consisting of the N-terminal non-helix region, 60 N-terminal residues of the helix region, 15 C-terminal residues of the helix region and the C-terminal non-helix region of human collagen Type I α2, and thus, it was shown that the fusion polypeptide consisting of the N-terminal non-helix region, 60 N-terminal residues of the helix region, 15 C-terminal residues of the helix region and the C-terminal non-helix region of human collagen Type I α2 was produced.

(3-4) Preparation of a Yeast in which an Expression Cassette for Prolyl 4-Hydroxylase α1 Subunit, an Expression Cassette for Prolyl 4-Hydroxylase β Subunit, an Expression Cassette for a Fusion Polypeptide which Consists of the N-Terminal Non-Helix Region, 27 N-Terminal Residues of the Helix Region, a Dimer Made of 522 Central Residues of the Helix Region, 15 C-Terminal Residues of the Helix Region and the C-Terminal Non-Helix Region of Human Collagen Type I α1, and an Expression Cassette for a Fusion Polypeptide which Consists of the N-Terminal Non-Helix Region, 27 N-Terminal Residues of the Helix Region, a Dimer Made of 522 Central Residues of the Helix Region, 15 C-Terminal Residues of the Helix Region and the C-Terminal Non-Helix Region of Human Collagen Type I α2 are Introduced

### (3-4-1) Transformation of Yeast

Using an electroporation method, strain 050525-5-3 (see, Example (3-1-1)) was transformed with pEXP-HA-HsCOL1A2-1A1 M500X2 obtained in Example 2-5-7.

Into 100 ml of YPD liquid medium (1% Yeast Extract, 2% peptone, and 2% glucose), strain 050525-5-3 (see, Example (3-1-1)) was inoculated, and cultured at 30° C. until the turbidity of cell culture (OD₆₆₀) reached about 10. The cells were collected from the cultured medium and the supernatant was removed. The cells were then suspended in 1M sorbitol at a turbidity of OD₆₆₀=about 150 to prepare a cell suspension.

Ten (10) μg of pEXP-HA-HsCOL1A2-1A1 M500X2 (see, Example (2-5-7)) was digested with a restriction enzyme XbaI. The DNA was collected through ethanol precipitation and dissolved in 5 μl of 10 mM Tris-HCl to prepare a DNA solution.

A mixture was prepared by combining 100 μl of the cell suspension and 5 μl of the DNA solution. To the mixture, a pulse was applied using a gene transfer device (ECM630, BTX), and then the mixture was spread on MD agar medium (1.34% Yeast Nitrogen Base, 4×10⁻⁵% biotin, 2% glucose, 2% agar) and cultured at 30° C. for 48 hours. Colonies formed on the MD agar medium were isolated as transformants. Strain 080801-2-4 was obtained as a transformant.

(3-4-2) Determination of Expression of a Fusion Polypeptide which Consists of the N-Terminal Non-Helix Region, 27 N-Terminal Residues of the Helix Region, a Dimer Made of 522 Central Residues of the Helix Region, 15 C-Terminal Residues of the Helix Region and the C-Terminal Non-Helix Region of Human Collagen Type I α1, and of a Fusion Polypeptide which Consists of the N-Terminal Non-Helix Region, 27 N-Terminal Residues of the Helix Region, a Dimer Made of 522 Central Residues of the Helix Region, 15 C-Terminal Residues of the Helix Region and the C-Terminal Non-Helix Region of Human Collagen Type I α2

Strain 080801-2-4 (see, Example (3-4-1)) was inoculated in 100 ml of BMGY medium [1% Yeast Extract, 2% peptone, 100 mM potassium phosphate buffer (pH 6.0), 1.34% Yeast Nitrogen Base (Difco), 4×10⁻⁵% biotin, 1% glycerol] which was prepared in a 500-ml baffled Erlenmeyer flask, and cultured with shaking at 30° C. for 24 hours. A cell suspension was prepared from the resultant culture, inoculated in 50 ml of BMM medium [100 mM potassium phosphate buffer (pH 6.0), 1.34% Yeast Nitrogen Base (Difco), 4×10⁻⁵% biotin, 0.5% methanol] which was prepared in a 500-ml baffled Erlenmeyer flask, at a final concentration of OD₆₆₀=about 10, and cultured with shaking at 30° C. for 72 hours. Every about 12 hours during this period, 50% methanol was added in an amount of 0.5 ml each.

The cells were collected from the resultant culture and the supernatant was removed. After that, the cells were subjected to cell disruption using a Multi-beads Shocker (Yasui Kikai Corporation) under conditions for yeast cell disruption (glass beads of a diameter of 0.5 mm, 2,500 rpm, 40 minutes). The solution resulting from the cell disruption was centrifuged to collect the supernatant of the cell disrupted solution to prepare a soluble fraction.

An aliquot of the soluble fraction was mixed with a sample buffer (Nacalai Tesque., Inc.), and the mixture was kept at 100° C. for 5 minutes, followed by electrophoresis on SDS-polyacrylamide gel. The electrophoresis, in which ERICA-MP (DRC) was used for the electrophoresis apparatus and 5-15% gradient gel (XV PANTERA MP gel, DRC) was used for the electrophoresis gel, was performed at 200 V for 20 minutes. After the electrophoresis was completed, the acrylamide gel was subjected to electro-blotting onto a PVDF membrane (Immobilon-P, Millipore Corp.). The electro-blotting, in which MINICA-MP (DRC) was used for the blotting apparatus, was performed at 40 V for 30 minutes.

A half of the resulting membrane was used to carry out western blotting analysis to detect a procollagen of the fusion polypeptide consisting of the N-terminal non-helix region, 27 N-terminal residues of the helix region, a dimer made of 522 central residues of the helix region, 15 C-terminal residues of the helix region and the C-terminal non-helix region of human collagen Type I α1. Blocking one (Nacalai Tesque., Inc.) was used for blocking of the membrane, Can Get Signal (Toyobo Co., Ltd.) was used for antibody reactions, 2,000-times diluted anti-human procollagen Type I α1 goat antibody (SC-8782, Santa Cruz) was used for the primary antibody, and 2,000-times diluted anti-goat IgG-HRP Linked donkey antibody (SC-2033, Santa Cruz) was used for the secondary antibody. The secondary antibody was detected by chemiluminescence using ECL Western Blotting Detection System (GE Healthcare Bio Science). The chemiluminescence was detected and quantitatively determined with an image analyzer (LAS-3000UVmini, Fujifilm Corporation). The results of western blotting analysis demonstrated that strain 080801-2-4 provided a signal that was detected at the position corresponding to the molecular weight of a procollagen of the fusion polypeptide consisting of the N-terminal non-helix region, 27 N-terminal residues of the helix region, a dimer made of 522 central residues of the helix region, 15 C-terminal residues of the helix region and the C-terminal non-helix region of human collagen Type I α1, and thus it was shown that the fusion polypeptide consisting of the N-terminal non-helix region, 27 N-terminal residues of the helix region, a dimer made of 522 central residues of the helix region, 15 C-terminal residues of the helix region and the C-terminal non-helix region of human collagen Type I α1 was produced.

The other half of the resulting membrane was used to carry out western blotting analysis to detect a procollagen of the fusion polypeptide consisting of the N-terminal non-helix region, 27 N-terminal residues of the helix region, a dimer made of 522 central residues of the helix region, 15 C-terminal residues of the helix region and the C-terminal non-helix region of human collagen Type I α2. Blocking one (Nacalai Tesque., Inc.) was used for blocking of the membrane, Can Get Signal (Toyobo Co., Ltd.) was used for antibody reactions, 5,000-times diluted anti-human collagen Type I rabbit antibody (ab292, abcam) was used for the primary antibody, and 10,000-times diluted anti-rabbit IgG-HRP Linked donkey antibody (NA-934, GE Healthcare Bio Science) was used for the secondary antibody. The secondary antibody was detected by chemiluminescence using ECL Western Blotting Detection System (GE Healthcare Bio Science). The chemiluminescence was detected and quantitatively determined with an image analyzer (LAS-3000UVmini, Fujifilm Corporation). The results of western blotting analysis demonstrated that strain 080801-2-4 provided a signal that was detected at the position corresponding to the molecular weight of a procollagen of the fusion polypeptide consisting of the N-terminal non-helix region, 27 N-terminal residues of the helix region, a dimer made of 522 central residues of the helix region, 15 C-terminal residues of the helix region and the C-terminal non-helix region of human collagen Type I α2, and thus, it was shown that the fusion polypeptide consisting of the N-terminal non-helix region, 27 N-terminal residues of the helix region, a dimer made of 522 central residues of the helix region, 15 C-terminal residues of the helix region and the C-terminal non-helix region of human collagen Type I α2 was produced.

(3-5) Preparation of a Yeast in which an Expression Cassette for Prolyl 4-Hydroxylase α1 Subunit, an Expression Cassette for Prolyl 4-Hydroxylase β Subunit and an Expression Cassette for Human Collagen Type III α1 are Introduced

### (3-5-1) Transformation of Yeast

Using an electroporation method, strain 050525-5-3 (see, Example (3-1-1)) was transformed with pEXP-HA-HsCOL3A1 obtained in Example 2-6-2.

Into 100 ml of YPD liquid medium (1% Yeast Extract, 2% peptone, and 2% glucose), strain 050525-5-3 (see, Example (3-1-1)) was inoculated, and cultured at 30° C. until the turbidity of cell culture (OD₆₆₀) reached about 10. The cells were collected from the cultured medium and the supernatant was removed. The cells were then suspended in 1M sorbitol at a turbidity of OD₆₆₀=about 150 to prepare a cell suspension.

Ten (10) μg of pEXP-HA-HsCOL3A1 (see, Example (2-6-2)) was digested with a restriction enzyme XbaI. The DNA was collected through ethanol precipitation and dissolved in 5 μl of 10 mM Tris-HCl to prepare a DNA solution.

A mixture was prepared by combining 100 μl of the cell suspension and 5 μl of the DNA solution. To the mixture, a pulse was applied using a gene transfer device (ECM630, BTX), and then the mixture was spread on MD agar medium (1.34% Yeast Nitrogen Base, 4×10⁻⁵% biotin, 2% glucose, 2% agar) and cultured at 30° C. for 48 hours. Colonies formed on the MD agar medium were isolated as transformants. Strain 080917-1-2 was obtained as a transformant.

### (3-5-2) Determination of Expression of Human Collagen Type III α1

Strain 080917-1-2 (see, Example (3-5-1)) was inoculated in 100 ml of BMGY medium [1% Yeast Extract, 2% peptone, 100 mM potassium phosphate buffer (pH 6.0), 1.34% Yeast Nitrogen Base (Difco), 4×10⁻⁵% biotin, 1% glycerol] which was prepared in a 500-ml baffled Erlenmeyer flask, and cultured with shaking at 30° C. for 24 hours. A cell suspension was prepared from the resultant culture, inoculated in 50 ml of BMM medium [100 mM potassium phosphate buffer (pH 6.0), 1.34% Yeast Nitrogen Base (Difco), 4×10⁻⁵% biotin, 0.5% methanol] which was prepared in a 500-ml baffled Erlenmeyer flask, at a final concentration of OD₆₆₀=about 10, and cultured with shaking at 30° C. for 72 hours. Every about 12 hours during this period, 50% methanol was added in an amount of 0.5 ml each.

The cells were collected from the resultant culture and the supernatant was removed. After that, the cells were subjected to cell disruption using a Multi-beads Shocker (Yasui Kikai Corporation) under conditions for yeast cell disruption (glass beads of a diameter of 0.5 mm, 2,500 rpm, 40 minutes). The solution resulting from the cell disruption was centrifuged to collect the supernatant of the cell disrupted solution to prepare a soluble fraction.

An aliquot of the soluble fraction was mixed with a sample buffer (Nacalai Tesque., Inc.), and the mixture was kept at 100° C. for 5 minutes, followed by electrophoresis on SDS-polyacrylamide gel. The electrophoresis, in which ERICA-MP (DRC) was used for the electrophoresis apparatus and 5-15% gradient gel (XV PANTERA MP gel, DRC) was used for the electrophoresis gel, was performed at 200 V for 20 minutes. After the electrophoresis was completed, the resulting acrylamide gel was subjected to staining with a CBB staining solution to detect protein bands. The results demonstrated that strain 080917-1-2 provided a band that was detected at the molecular weight corresponding to a procollagen of collagen Type III α1, and thus it was shown that human collagen Type III α1 was produced.

(3-6) Preparation of a Yeast in which an Expression Cassette for Prolyl 4-Hydroxylase α1 Subunit, an Expression Cassette for Prolyl 4-Hydroxylase β Subunit and an Expression Cassette for Human Collagen Type II α1 are Introduced

### (3-6-1) Transformation of Yeast

Using an electroporation method, strain 050525-5-3 (see, Example (3-1-1)) is transformed with pEXP-HA-YHsCOL2A1 obtained in Example 2-7-2.

Into 100 ml of YPD liquid medium (1% Yeast Extract, 2% peptone, and 2% glucose), strain 050525-5-3 (see, Example (3-1-1)) is inoculated, and cultured at 30° C. until the turbidity of cell culture (OD₆₆₀) reaches about 10. The cells are collected from the cultured medium and the supernatant is removed. The cells are then suspended in 1M sorbitol at a turbidity of OD₆₆₀=about 150 to prepare a cell suspension.

Ten (10) μg of pEXP-HA-YHsCOL2A1 (see, Example (2-7-2)) is digested with a restriction enzyme XbaI. The DNA is collected through ethanol precipitation and dissolved in 5 μl of 10 mM Tris-HCl to prepare a DNA solution.

A mixture is prepared by combining 100 μl of the cell suspension and 5 μl of the DNA solution. To the mixture, a pulse is applied using a gene transfer device (ECM630, BTX), and then the mixture is spread on MD agar medium (1.34% Yeast Nitrogen Base, 4×10⁻⁵% biotin, 2% glucose, 2% agar) and cultured at 30° C. for 48 hours. Colonies formed on the MD agar medium are isolated as transformants.

### (3-6-2) Determination of Expression of Human Collagen Type II α1

A transformant (see, Example (3-6-1)) is inoculated in 100 ml of BMGY medium [1% Yeast Extract, 2% peptone, 100 mM potassium phosphate buffer (pH 6.0), 1.34% Yeast Nitrogen Base (Difco), 4×10⁻⁵% biotin, 1% glycerol] which is prepared in a 500-ml baffled Erlenmeyer flask, and cultured with shaking at 30° C. for 24 hours. A cell suspension is prepared from the resultant culture, inoculated in 50 ml of BMM medium [100 mM potassium phosphate buffer (pH 6.0), 1.34% Yeast Nitrogen Base (Difco), 4×10⁻⁵% biotin, 0.5% methanol] which is prepared in a 500-ml baffled Erlenmeyer flask, at a final concentration of OD₆₆₀=about 10, and cultured with shaking at 30° C. for 72 hours. Every about 12 hours during this period, 50% methanol is added in an amount of 0.5 ml each.

The cells are collected from the resultant culture and the supernatant is removed. After that, the cells are subjected to cell disruption using a Multi-beads Shocker (Yasui Kikai Corporation) under conditions for yeast cell disruption (glass beads of a diameter of 0.5 mm, 2,500 rpm, 40 minutes). The solution resulting from the cell disruption is centrifuged to collect the supernatant of the cell disrupted solution to prepare a soluble fraction.

An aliquot of the soluble fraction is mixed with a sample buffer (Nacalai Tesque., Inc.), and the mixture is kept at 100° C. for 5 minutes, followed by electrophoresis on SDS-polyacrylamide gel. The electrophoresis, in which ERICA-MP (DRC) is used for the electrophoresis apparatus and 5-15% gradient gel (XV PANTERA MP gel, DRC) is used for the electrophoresis gel, is performed at 200 V for 20 minutes. After the electrophoresis is completed, the resulting acrylamide gel is subjected to staining with a CBB staining solution to detect protein bands. A band is detected at the molecular weight corresponding to a procollagen of collagen Type II α1, demonstrating that human collagen Type II α1 is produced.

(3-7) Preparation of a Yeast in which an Expression Cassette for Prolyl 4-Hydroxylase α1 Subunit, an Expression Cassette for Prolyl 4-Hydroxylase β Subunit, an Expression Cassette for Human Collagen Type I α1, an Expression Cassette for Human Collagen Type I α2 and an Expression Cassette for FKBP13A are Introduced

### (3-7-1) Transformation of Yeast

Using an electroporation method, strain 070327-1-11 (see, Example (3-2-1)) was transformed with pEXP-A-FKBP13A ZeoR obtained in Example 2-2-4.

Into 100 ml of YPD liquid medium (1% Yeast Extract, 2% peptone, and 2% glucose), strain 070327-1-11 (see, Example (3-2-1)) was inoculated, and cultured at 30° C. until the turbidity of cell culture (OD₆₆₀) reached about 10. The cells were collected from the cultured medium and the supernatant was removed. The cells were then suspended in 1M sorbitol at a turbidity of OD₆₆₀=about 150 to prepare a cell suspension.

Ten (10) μg of pEXP-A-FKBP13A ZeoR (see, Example (2-2-4)) was digested with a restriction enzyme BglII. The DNA was collected through ethanol precipitation and dissolved in 5 μl of 10 mM Tris-HCl to prepare a DNA solution.

A mixture was prepared by combining 100 μl of the cell suspension and 5 μl of the DNA solution. To the mixture, a pulse is applied using a gene transfer device (ECM630, BTX), and then the mixture is spread on MD agar medium (1.34% Yeast Nitrogen Base, 4×10⁻⁵% biotin, 2% glucose, 300 μg/ml Zeocyn, 2% agar) and cultured at 30° C. for 48 hours. Colonies formed on the MD agar medium were isolated as transformants. Strain 081022-2-1 was obtained as a pEXP-A-FKBP13A ZeoR transformant.

### (3-7-2) Determination of Expression of FKBP13A

Strain 081022-2-1 (see, Example (3-7-1)) was inoculated in 100 ml of BMGY medium [1% Yeast Extract, 2% peptone, 100 mM potassium phosphate buffer (pH 6.0), 1.34% Yeast Nitrogen Base (Difco), 4×10⁻⁵% biotin, 1% glycerol] which was prepared in a 500-ml baffled Erlenmeyer flask, and cultured with shaking at 30° C. for 24 hours. A cell suspension was prepared from the resultant culture, inoculated in 50 ml of BMM medium [100 mM potassium phosphate buffer (pH 6.0), 1.34% Yeast Nitrogen Base (Difco), 4×10⁻⁵% biotin, 0.5% methanol] which was prepared in a 500-ml baffled Erlenmeyer flask, at a final concentration of OD₆₆₀=about 10, and cultured with shaking at 30° C. for 72 hours. Every about 12 hours during this period, 50% methanol was added in an amount of 0.5 ml each.

The cells were collected from the resultant culture and the supernatant was removed. After that, the cells were subjected to cell disruption using a Multi-beads Shocker (Yasui Kikai Corporation) under conditions for yeast cell disruption (glass beads of a diameter of 0.5 mm, 2,500 rpm, 40 minutes). The solution resulting from the cell disruption was centrifuged to collect the supernatant of the cell disrupted solution to prepare a soluble fraction.

An aliquot of the soluble fraction was mixed with a sample buffer (Nacalai Tesque., Inc.), and the mixture was kept at 100° C. for 5 minutes, followed by electrophoresis on SDS-polyacrylamide gel. The electrophoresis, in which ERICA-MP (DRC) was used for the electrophoresis apparatus and 5-15% gradient gel (XV PANTERA MP gel, DRC) was used for the electrophoresis gel, was performed at 200 V for 20 minutes. After the electrophoresis was completed, the acrylamide gel was subjected to electro-blotting onto a PVDF membrane (Immobilon-P, Millipore Corp.). The electro-blotting, in which MINICA-MP (DRC) was used for the blotting apparatus, was performed at 40 V for 30 minutes.

The resulting membrane was used to carry out western blotting analysis to detect the FK506 binding protein. Blocking one (Nacalai Tesque., Inc.) was used for blocking of the membrane, Can Get Signal (Toyobo Co., Ltd.) was used for antibody reactions, 2,000-times diluted anti-FKBP13A rabbit antibody (11700-1-AP, ProteinTech Group) was used for the primary antibody, and 10,000-times diluted anti-rabbit IgG-HRP Linked donkey antibody (NA-934, GE Healthcare Bio Science) was used for the secondary antibody. The secondary antibody was detected by chemiluminescence using ECL Western Blotting Detection System (GE Healthcare Bio Science). The chemiluminescence was detected and quantitatively determined with an image analyzer (LAS-3000UVmini, Fujifilm Corporation). The results of western blotting analysis demonstrated that strain 081022-2-1 provided a signal that was detected at the position corresponding to the molecular weight of the FKBP13A, and thus, it was shown that the FKBP13A was produced.

(3-8) Preparation of a Yeast in which an Expression Cassette for Prolyl 4-Hydroxylase α1 Subunit, an Expression Cassette for Prolyl 4-Hydroxylase β Subunit, an Expression Cassette for Human Collagen Type I α1, an Expression Cassette for Human Collagen Type I α2 and an Expression Cassette for FKBP19 are Introduced

### (3-8-1) Transformation of Yeast

Using an electroporation method, strain 070327-1-11 (see, Example (3-2-1)) was transformed with pEXP-A-FKBP19 ZeoR obtained in Example 2-2-5.

Into 100 ml of YPD liquid medium (1% Yeast Extract, 2% peptone, and 2% glucose), strain 070327-1-11 (see, Example (3-2-1)) was inoculated, and cultured at 30° C. until the turbidity of cell culture (OD₆₆₀) reached about 10. The cells were collected from the cultured medium and the supernatant was removed. The cells were then suspended in 1M sorbitol at a turbidity of OD₆₆₀=about 150 to prepare a cell suspension.

Ten (10) μg of pEXP-A-FKBP19 ZeoR (see, Example (2-2-5)) was digested with a restriction enzyme BglII. The DNA was collected through ethanol precipitation and dissolved in 5 μl of 10 mM Tris-HCl to prepare a DNA solution.

A mixture was prepared by combining 100 μl of the cell suspension and 5 μl of the DNA solution. To the mixture, a pulse is applied using a gene transfer device (ECM630, BTX), and then the mixture is spread on MD agar medium (1.34% Yeast Nitrogen Base, 4×10⁻⁵% biotin, 2% glucose, 300 μg/ml Zeocyn, 2% agar) and cultured at 30° C. for 48 hours. Colonies formed on the MD agar medium were isolated as transformants. Strain 081022-1-1 was obtained as a pEXP-A-FKBP19 ZeoR transformant.

### (3-8-2) Determination of Expression of FKBP19

Strain 081022-1-1 (see, Example (3-8-1)) was inoculated in 100 ml of BMGY medium [1% Yeast Extract, 2% peptone, 100 mM potassium phosphate buffer (pH 6.0), 1.34% Yeast Nitrogen Base (Difco), 4×10⁻⁵% biotin, 1% glycerol] which was prepared in a 500-ml baffled Erlenmeyer flask, and cultured with shaking at 30° C. for 24 hours. A cell suspension was prepared from the resultant culture, inoculated in 50 ml of BMM medium [100 mM potassium phosphate buffer (pH 6.0), 1.34% Yeast Nitrogen Base (Difco), 4×10⁻⁵% biotin, 0.5% methanol] which was prepared in a 500-ml baffled Erlenmeyer flask, at a final concentration of OD₆₆₀=about 10, and cultured with shaking at 30° C. for 72 hours. Every about 12 hours during this period, 50% methanol was added in an amount of 0.5 ml each.

The cells were collected from the resultant culture and the supernatant was removed. After that, the cells were subjected to cell disruption using a Multi-beads Shocker (Yasui Kikai Corporation) under conditions for yeast cell disruption (glass beads of a diameter of 0.5 mm, 2,500 rpm, 40 minutes). The solution resulting from the cell disruption was centrifuged to collect the supernatant of the cell disrupted solution to prepare a soluble fraction.

An aliquot of the soluble fraction was mixed with a sample buffer (Nacalai Tesque., Inc.), and the mixture was kept at 100° C. for 5 minutes, followed by electrophoresis on SDS-polyacrylamide gel. The electrophoresis, in which ERICA-MP (DRC) was used for the electrophoresis apparatus and 5-15% gradient gel (XV PANTERA MP gel, DRC) was used for the electrophoresis gel, was performed at 200 V for 20 minutes. After the electrophoresis was completed, the acrylamide gel was subjected to electro-blotting onto a PVDF membrane (Immobilon-P, Millipore Corp.). The electro-blotting, in which MINICA-MP (DRC) was used for the blotting apparatus, was performed at 40 V for 30 minutes.

The resulting membrane was used to carry out western blotting analysis to detect the FK506 binding protein. Blocking one (Nacalai Tesque., Inc.) was used for blocking of the membrane, Can Get Signal (Toyobo Co., Ltd.) was used for antibody reactions, 2,000-times diluted anti-FKBP19 mouse antibody (H00051303-B01, Abnova) was used for the primary antibody, and 10,000-times diluted anti-mouse IgG-HRP Linked sheep antibody (NA-931, GE Healthcare Bio Science) was used for the secondary antibody. The secondary antibody was detected by chemiluminescence using ECL Western Blotting Detection System (GE Healthcare Bio Science). The chemiluminescence was detected and quantitatively determined with an image analyzer (LAS-3000UVmini, Fujifilm Corporation). The results of western blotting analysis demonstrated that strain 081022-1-1 provided a signal that was detected at the position corresponding to the molecular weight of the FKBP19, and thus, it was shown that the FKBP19 was produced.

(3-9) Preparation of a Yeast in which an Expression Cassette for Prolyl 4-Hydroxylase α1 Subunit, an Expression Cassette for Prolyl 4-Hydroxylase β Subunit, an Expression Cassette for Human Collagen Type I α1, an Expression Cassette for Human Collagen Type I α2 and an Expression Cassette for FKBP23 (Part 1) are Introduced

### (3-9-1) Transformation of Yeast

Using an electroporation method, strain 070327-1-11 (see, Example (3-2-1)) was transformed with pEXP-A-FKBP23 ZeoR obtained in Example 2-2-6.

Into 100 ml of YPD liquid medium (1% Yeast Extract, 2% peptone, and 2% glucose), strain 070327-1-11 (see, Example (3-2-1)) was inoculated, and cultured at 30° C. until the turbidity of cell culture (OD₆₆₀) reached about 10. The cells were collected from the cultured medium and the supernatant was removed. The cells were then suspended in 1M sorbitol at a turbidity of OD₆₆₀=about 150 to prepare a cell suspension.

Ten (10) μg of pEXP-A-FKBP23 ZeoR (see, Example (2-2-6)) was digested with a restriction enzyme BglII. The DNA was collected through ethanol precipitation and dissolved in 5 μl of 10 mM Tris-HCl to prepare a DNA solution.

A mixture was prepared by combining 100 μl of the cell suspension and 5 μl of the DNA solution. To the mixture, a pulse is applied using a gene transfer device (ECM630, BTX), and then the mixture is spread on MD agar medium (1.34% Yeast Nitrogen Base, 4×10⁻⁵% biotin, 2% glucose, 300 μg/ml Zeocyn, 2% agar) and cultured at 30° C. for 48 hours. Colonies formed on the MD agar medium were isolated as transformants. Strain 081024-3-1 was obtained as a pEXP-A-FKBP23 ZeoR transformant.

### (3-9-2) Determination of Expression of FKBP23

Strain 081024-3-1 (see, Example (3-9-1)) was inoculated in 100 ml of BMGY medium [1% Yeast Extract, 2% peptone, 100 mM potassium phosphate buffer (pH 6.0), 1.34% Yeast Nitrogen Base (Difco), 4×10⁻⁵% biotin, 1% glycerol] which was prepared in a 500-ml baffled Erlenmeyer flask, and cultured with shaking at 30° C. for 24 hours. A cell suspension was prepared from the resultant culture, inoculated in 50 ml of BMM medium [100 mM potassium phosphate buffer (pH 6.0), 1.34% Yeast Nitrogen Base (Difco), 4×10⁻⁵% biotin, 0.5% methanol] which was prepared in a 500-ml baffled Erlenmeyer flask, at a final concentration of OD₆₆₀=about 10, and cultured with shaking at 30° C. for 72 hours. Every about 12 hours during this period, 50% methanol was added in an amount of 0.5 ml each.

The cells were collected from the resultant culture and the supernatant was removed. After that, the cells were subjected to cell disruption using a Multi-beads Shocker (Yasui Kikai Corporation) under conditions for yeast cell disruption (glass beads of a diameter of 0.5 mm, 2,500 rpm, 40 minutes). The solution resulting from the cell disruption was centrifuged to collect the supernatant of the cell disrupted solution to prepare a soluble fraction.

An aliquot of the soluble fraction was mixed with a sample buffer (Nacalai Tesque., Inc.), and the mixture was kept at 100° C. for 5 minutes, followed by electrophoresis on SDS-polyacrylamide gel. The electrophoresis, in which ERICA-MP (DRC) was used for the electrophoresis apparatus and 5-15% gradient gel (XV PANTERA MP gel, DRC) was used for the electrophoresis gel, was performed at 200 V for 20 minutes. After the electrophoresis was completed, the acrylamide gel was subjected to electro-blotting onto a PVDF membrane (Immobilon-P, Millipore Corp.). The electro-blotting, in which MINICA-MP (DRC) was used for the blotting apparatus, was performed at 40 V for 30 minutes.

The resulting membrane was used to carry out western blotting analysis to detect the FK506 binding protein. Blocking one (Nacalai Tesque., Inc.) was used for blocking of the membrane, Can Get Signal (Toyobo Co., Ltd.) was used for antibody reactions, 2,000-times diluted anti-FKBP23 rabbit antibody (12092-1-AP, ProteinTech Group) was used for the primary antibody, and 10,000-times diluted anti-rabbit IgG-HRP Linked donkey antibody (NA-934, GE Healthcare Bio Science) was used for the secondary antibody. The secondary antibody was detected by chemiluminescence using ECL Western Blotting Detection System (GE Healthcare Bio Science). The chemiluminescence was detected and quantitatively determined with an image analyzer (LAS-3000UVmini, Fujifilm Corporation). The results of western blotting analysis demonstrated that strain 081024-3-1 provided a signal that was detected at the position corresponding to the molecular weight of the FKBP23, and thus, it was shown that the FKBP23 was produced.

(3-10) Preparation of a Yeast in which an Expression Cassette for Prolyl 4-Hydroxylase α1 Subunit, an Expression Cassette for Prolyl 4-Hydroxylase β Subunit, an Expression Cassette for Human Collagen Type I α1, an Expression Cassette for Human Collagen Type I α2 and an Expression Cassette for FKBP63 are Introduced

### (3-10-1) Transformation of Yeast

Using an electroporation method, strain 070327-1-11 (see, Example (3-2-1)) was transformed with pEXP-A-FKBP63 ZeoR obtained in Example 2-2-7.

Into 100 ml of YPD liquid medium (1% Yeast Extract, 2% peptone, and 2% glucose), strain 070327-1-11 (see, Example (3-2-1)) was inoculated, and cultured at 30° C. until the turbidity of cell culture (OD₆₆₀) reached about 10. The cells were collected from the cultured medium and the supernatant was removed. The cells were then suspended in 1M sorbitol at a turbidity of OD₆₆₀=about 150 to prepare a cell suspension.

Ten (10) μg of pEXP-A-FKBP63 ZeoR (see, Example (2-2-7)) was digested with a restriction enzyme BglII. The DNA was collected through ethanol precipitation and dissolved in 5 μl of 10 mM Tris-HCl to prepare a DNA solution.

A mixture was prepared by combining 100 μl of the cell suspension and 5 μl of the DNA solution. To the mixture, a pulse is applied using a gene transfer device (ECM630, BTX), and then the mixture is spread on MD agar medium (1.34% Yeast Nitrogen Base, 4×10⁻⁵% biotin, 2% glucose, 300 μg/ml Zeocyn, 2% agar) and cultured at 30° C. for 48 hours. Colonies formed on the MD agar medium were isolated as transformants. Strain 081024-2-1 was obtained as a pEXP-A-FKBP63 ZeoR transformant.

### (3-10-2) Determination of Expression of FKBP63

Strain 081024-2-1 (see, Example (3-10-1)) was inoculated in 100 ml of BMGY medium [1% Yeast Extract, 2% peptone, 100 mM potassium phosphate buffer (pH 6.0), 1.34% Yeast Nitrogen Base (Difco), 4×10⁻⁵% biotin, 1% glycerol] which was prepared in a 500-ml baffled Erlenmeyer flask, and cultured with shaking at 30° C. for 24 hours. A cell suspension was prepared from the resultant culture, inoculated in 50 ml of BMM medium [100 mM potassium phosphate buffer (pH 6.0), 1.34% Yeast Nitrogen Base (Difco), 4×10⁻⁵% biotin, 0.5% methanol] which was prepared in a 500-ml baffled Erlenmeyer flask, at a final concentration of OD₆₆₀=about 10, and cultured with shaking at 30° C. for 72 hours. Every about 12 hours during this period, 50% methanol was added in an amount of 0.5 ml each.

The cells were collected from the resultant culture and the supernatant was removed. After that, the cells were subjected to cell disruption using a Multi-beads Shocker (Yasui Kikai Corporation) under conditions for yeast cell disruption (glass beads of a diameter of 0.5 mm, 2,500 rpm, 40 minutes). The solution resulting from the cell disruption was centrifuged to collect the supernatant of the cell disrupted solution to prepare a soluble fraction.

An aliquot of the soluble fraction was mixed with a sample buffer (Nacalai Tesque., Inc.), and the mixture was kept at 100° C. for 5 minutes, followed by electrophoresis on SDS-polyacrylamide gel. The electrophoresis, in which ERICA-MP (DRC) was used for the electrophoresis apparatus and 5-15% gradient gel (XV PANTERA MP gel, DRC) was used for the electrophoresis gel, was performed at 200 V for 20 minutes. After the electrophoresis was completed, the resulting acrylamide gel was subjected to staining with a CBB staining solution to detect protein bands. The results demonstrated that strain 081024-2-1 provided a band that was detected at the molecular weight corresponding to the FKBP63, and thus, it was shown that the FKBP63 was produced.

(3-11) Preparation of a Yeast in which an Expression Cassette for Prolyl 4-Hydroxylase α1 Subunit, an Expression Cassette for Prolyl 4-Hydroxylase β Subunit, an Expression Cassette for Human Collagen Type I α1, an Expression Cassette for Human Collagen Type I α2 and an Expression Cassette for FKBP65 are Introduced

### (3-11-1) Transformation of Yeast

Using an electroporation method, strain 070327-1-11 (see, Example (3-2-1)) was transformed with pEXP-A-FKBP65 ZeoR obtained in Example 2-2-8.

Into 100 ml of YPD liquid medium (1% Yeast Extract, 2% peptone, and 2% glucose), strain 070327-1-11 (see, Example (3-11-1)) was inoculated, and cultured at 30° C. until the turbidity of cell culture (OD₆₆₀) reached about 10. The cells were collected from the cultured medium and the supernatant was removed. The cells were then suspended in 1M sorbitol at a turbidity of OD₆₆₀=about 150 to prepare a cell suspension.

Ten (10) μg of pEXP-A-FKBP65 ZeoR (see, Example (2-2-8)) was digested with a restriction enzyme BglII. The DNA was collected through ethanol precipitation and dissolved in 5 μl of 10 mM Tris-HCl to prepare a DNA solution.

A mixture was prepared by combining 100 μl of the cell suspension and 5 μl of the DNA solution. To the mixture, a pulse is applied using a gene transfer device (ECM630, BTX), and then the mixture is spread on MD agar medium (1.34% Yeast Nitrogen Base, 4×10⁻⁵% biotin, 2% glucose, 300 μg/ml Zeocyn, 2% agar) and cultured at 30° C. for 48 hours. Colonies formed on the MD agar medium were isolated as transformants. Strain 081024-1-1 was obtained as a pEXP-A-FKBP65 ZeoR transformant.

### (3-11-2) Determination of Expression of FKBP65

Strain 081024-1-1 (see, Example (3-11-1)) was inoculated in 100 ml of BMGY medium [1% Yeast Extract, 2% peptone, 100 mM potassium phosphate buffer (pH 6.0), 1.34% Yeast Nitrogen Base (Difco), 4×10⁻⁵% biotin, 1% glycerol] which was prepared in a 500-ml baffled Erlenmeyer flask, and cultured with shaking at 30° C. for 24 hours. A cell suspension was prepared from the resultant culture, inoculated in 50 ml of BMM medium [100 mM potassium phosphate buffer (pH 6.0), 1.34% Yeast Nitrogen Base (Difco), 4×10⁻⁵% biotin, 0.5% methanol] which was prepared in a 500-ml baffled Erlenmeyer flask, at a final concentration of OD₆₆₀=about 10, and cultured with shaking at 30° C. for 72 hours. Every about 12 hours during this period, 50% methanol was added in an amount of 0.5 ml each.

The cells were collected from the resultant culture and the supernatant was removed. After that, the cells were subjected to cell disruption using a Multi-beads Shocker (Yasui Kikai Corporation) under conditions for yeast cell disruption (glass beads of a diameter of 0.5 mm, 2,500 rpm, 40 minutes). The solution resulting from the cell disruption was centrifuged to collect the supernatant of the cell disrupted solution to prepare a soluble fraction.

An aliquot of the soluble fraction was mixed with a sample buffer (Nacalai Tesque., Inc.), and the mixture was kept at 100° C. for 5 minutes, followed by electrophoresis on SDS-polyacrylamide gel. The electrophoresis, in which ERICA-MP (DRC) was used for the electrophoresis apparatus and 5-15% gradient gel (XV PANTERA MP gel, DRC) was used for the electrophoresis gel, was performed at 200 V for 20 minutes. After the electrophoresis was completed, the acrylamide gel was subjected to electro-blotting onto a PVDF membrane (Immobilon-P, Millipore Corp.). The electro-blotting, in which MINICA-MP (DRC) was used for the blotting apparatus, was performed at 40 V for 30 minutes.

The resulting membrane was used to carry out western blotting analysis to detect the FK506 binding protein. Blocking one (Nacalai Tesque., Inc.) was used for blocking of the membrane, Can Get Signal (Toyobo Co., Ltd.) was used for antibody reactions, 2,000-times diluted anti-FKBP65 rabbit antibody (12172-1-AP, ProteinTech Group) was used for the primary antibody, and 10,000-times diluted anti-rabbit IgG-HRP Linked donkey antibody (NA-934, GE Healthcare Bio Science) was used for the secondary antibody. The secondary antibody was detected by chemiluminescence using ECL Western Blotting Detection System (GE Healthcare Bio Science). The chemiluminescence was detected and quantitatively determined with an image analyzer (LAS-3000UVmini, Fujifilm Corporation). The results of western blotting analysis demonstrated that strain 081024-1-1 provided a signal that was detected at the position corresponding to the molecular weight of the FKBP65, and thus, it was shown that the FKBP65 was produced.

(3-12) Preparation of a Yeast in which an Expression Cassette for Prolyl 4-Hydroxylase α1 Subunit, an Expression Cassette for Prolyl 4-Hydroxylase β Subunit, an Expression Cassette for Human Collagen Type I α1, an Expression Cassette for Human Collagen Type I α2 and an Expression Cassette for FKBP23 (Part 2) are Introduced

### (3-12-1) Transformation of Yeast

Using an electroporation method, strain 070327-1-11 (see, Example (3-2-1)) is transformed with pEXP-A-PER3 Pro-FKBP23 ZeoR (see, Example (2-2-9)), pEXP-A-AOX2 Pro-FKBP23 ZeoR (see, Example (2-2-10)) or pEXP-A-FLD1 Pro-FKBP23 ZeoR (see, Example (2-2-11)).

Into 100 ml of YPD liquid medium (1% Yeast Extract, 2% peptone, and 2% glucose), strain 050818-3-1 is inoculated, and cultured at 30° C. until the turbidity of cell culture (OD₆₆₀) reaches about 10. The cells are collected from the cultured medium and the supernatant is removed. The cells are then suspended in 1M sorbitol at a turbidity of OD₆₆₀ about 150 to prepare a cell suspension.

Ten (1-0) μg of pEXP-A-PER3 Pro-FKBP23 ZeoR (see, Example (2-2-9)), pEXP-A-AOX2 Pro-FKBP23 ZeoR (see, Example (2-2-10)) or pEXP-A-FLD1 Pro-FKBP23 ZeoR (see, Example (2-2-11)) is digested with a restriction enzyme BglII. The DNA is collected through ethanol precipitation and dissolved in 5 μl of 10 mM Tris-HCl to prepare a DNA solution.

A mixture is prepared by combining 100 μl of the cell suspension and 5 μl of the DNA solution. To the mixture, a pulse is applied using a gene transfer device (ECM630, BTX), and then the mixture is spread on MD agar medium (1.34% Yeast Nitrogen Base, 4×10⁻⁵% biotin, 2% glucose, 300 μg/ml Zeocyn, 2% agar) and cultured at 30° C. for 48 hours. Colonies formed on the MD agar medium are isolated as transformants.

### (3-12-2) Determination of Expression of FKBP23

A transformant (see, Example (3-12-1)) is inoculated in 100 ml of BMGY medium [1% Yeast Extract, 2% peptone, 100 mM potassium phosphate buffer (pH 6.0), 1.34% Yeast Nitrogen Base (Difco), 4×10⁻⁵% biotin, 1% glycerol] which is prepared in a 500-ml baffled Erlenmeyer flask, and cultured with shaking at 30° C. for 24 hours. A cell suspension is prepared from the resultant culture, inoculated in 50 ml of BMM medium [100 mM potassium phosphate buffer (pH 6.0), 1.34% Yeast Nitrogen Base (Difco), 4×10⁻⁵% biotin, 0.5% methanol] which is prepared in a 500-ml baffled Erlenmeyer flask, at a final concentration of OD₆₆₀=about 10, and cultured with shaking at 30° C. for 72 hours. Every about 12 hours during this period, 50% methanol is added in an amount of 0.5 ml each.

The cells are collected from the resultant culture and the supernatant is removed. After that, the cells are subjected to cell disruption using a Multi-beads Shocker (Yasui Kikai Corporation) under conditions for yeast cell disruption (glass beads of a diameter of 0.5 mm, 2,500 rpm, 40 minutes). The solution resulting from the cell disruption is centrifuged to collect the supernatant of the cell disrupted solution to prepare a soluble fraction.

An aliquot of the soluble fraction is mixed with a sample buffer (Nacalai Tesque., Inc.), and the mixture is kept at 100° C. for 5 minutes, followed by electrophoresis on SDS-polyacrylamide gel. The electrophoresis, in which ERICA-MP (DRC) is used for the electrophoresis apparatus and 5-15% gradient gel (XV PANTERA MP gel, DRC) is used for the electrophoresis gel, is performed at 200 V for 20 minutes. After the electrophoresis is completed, the acrylamide gel is subjected to electro-blotting onto a PVDF membrane (Immobilon-P, Millipore Corp.). The electro-blotting, in which MINICA-MP (DRC) is used for the blotting apparatus, is performed at 40 V for 30 minutes.

The resulting membrane is used to carry out western blotting analysis to detect the FK506 binding protein. Blocking one (Nacalai Tesque., Inc.) is used for blocking of the membrane, Can Get Signal (Toyobo Co., Ltd.) is used for antibody reactions, 2,000-times diluted anti-FKBP23 rabbit antibody (12092-1-AP, ProteinTech Group) is used for the primary antibody, and 10,000-times diluted anti-rabbit IgG-HRP Linked donkey antibody (NA-934, GE Healthcare Bio Science) is used for the secondary antibody. The secondary antibody is detected by chemiluminescence using ECL Western Blotting Detection System (GE Healthcare Bio Science). The chemiluminescence is detected and quantitatively determined with an image analyzer (LAS-3000UVmini, Fujifilm Corporation). A band is detected at the molecular weight corresponding to the FKBP23, demonstrating that the FKBP23 is produced.

(3-13) Preparation of a Yeast in which an Expression Cassette for Prolyl 4-Hydroxylase α1 Subunit; an Expression Cassette for Prolyl 4-Hydroxylase β Subunit; an Expression Cassette for a Fusion Polypeptide which Consists of the N-Terminal Non-Helix Region, 60 N-Terminal Residues of the Helix Region, 15 C-Terminal Residues of the Helix Region and the C-Terminal Non-Helix Region of Human Collagen Type I α1; an Expression Cassette for a Fusion Polypeptide which Consists of the N-Terminal Non-Helix Region, 60 N-Terminal Residues of the Helix Region, 15 C-Terminal Residues of the Helix Region and the C-Terminal Non-Helix Region of Human Collagen Type I α2I α2; and an Expression Cassette for FKBP23 are Introduced

### (3-13-1) Transformation of Yeast

Using an electroporation method, strain 080118-3-3 (see, Example (3-3-1)) is transformed with pEXP-A-PER3 Pro-FKBP23 ZeoR (see, Example (2-2-9)), pEXP-A-AOX2 Pro-FKBP23 ZeoR (see, Example (2-2-10)) or pEXP-A-FLD1 Pro-FKBP23 ZeoR (see, Example (2-2-11)).

Into 100 ml of YPD liquid medium (1% Yeast Extract, 2% peptone, and 2% glucose), strain 080118-3-3 (see, Example (3-3-1)) is inoculated, and cultured at 30° C. until the turbidity of cell culture (OD₆₆₀) reaches about 10. The cells are collected from the cultured medium and the supernatant is removed. The cells are then suspended in 1M sorbitol at a turbidity of OD₆₆₀=about 150 to prepare a cell suspension.

Ten (10) μg of pEXP-A-PER3 Pro-FKBP23 ZeoR (see, Example (2-2-9)), pEXP-A-AOX2 Pro-FKBP23 ZeoR (see, Example (2-2-10)) or pEXP-A-FLD1 Pro-FKBP23 ZeoR (see, Example (2-2-11)) is digested with a restriction enzyme BglII. The DNA is collected through ethanol precipitation and dissolved in 5 μl of 10 mM Tris-HCl to prepare a DNA solution.

A mixture is prepared by combining 100 μl of the cell suspension and 5 μl of the DNA solution. To the mixture, a pulse is applied using a gene transfer device (ECM630, BTX), and then the mixture is spread on MD agar medium (1.34% Yeast Nitrogen Base, 4×10⁻⁵% biotin, 2% glucose, 300 μg/ml Zeocyn, 2% agar) and cultured at 30° C. for 48 hours. Colonies formed on the MD agar medium are isolated as transformants.

### (3-13-2) Determination of Expression of FKBP23

A transformant (see, Example (3-13-1)) is inoculated in 100 ml of BMGY medium [1% Yeast Extract, 2% peptone, 100 mM potassium phosphate buffer (pH 6.0), 1.34% Yeast Nitrogen Base (Difco), 4×10⁻⁵% biotin, 1% glycerol] which is prepared in a 500-ml baffled Erlenmeyer flask, and cultured with shaking at 30° C. for 24 hours. A cell suspension is prepared from the resultant culture, inoculated in 50 ml of BMM medium [100 mM potassium phosphate buffer (pH 6.0), 1.34% Yeast Nitrogen Base (Difco), 4×10⁻⁵% biotin, 0.5% methanol] which is prepared in a 500-ml baffled Erlenmeyer flask, at a final concentration of OD₆₆₀=about 10, and cultured with shaking at 30° C. for 72 hours. Every about 12 hours during this period, 50% methanol is added in an amount of 0.5 ml each.

The cells are collected from the resultant culture and the supernatant is removed. After that, the cells are subjected to cell disruption using a Multi-beads Shocker (Yasui Kikai Corporation) under conditions for yeast cell disruption (glass beads of a diameter of 0.5 mm, 2,500 rpm, 40 minutes). The solution resulting from the cell disruption is centrifuged to collect the supernatant of the cell disrupted solution to prepare a soluble fraction.

An aliquot of the soluble fraction is mixed with a sample buffer (Nacalai Tesque., Inc.), and the mixture is kept at 100° C. for 5 minutes, followed by electrophoresis on SDS-polyacrylamide gel. The electrophoresis, in which ERICA-MP (DRC) is used for the electrophoresis apparatus and 5-15% gradient gel (XV PANTERA MP gel, DRC) is used for the electrophoresis gel, is performed at 200 V for 20 minutes. After the electrophoresis is completed, the acrylamide gel is subjected to electro-blotting onto a PVDF membrane (Immobilon-P, Millipore Corp.). The electro-blotting, in which MINICA-MP (DRC) is used for the blotting apparatus, is performed at 40 V for 30 minutes.

The resulting membrane is used to carry out western blotting analysis to detect the FKBP23. Blocking one (Nacalai Tesque., Inc.) is used for blocking of the membrane, Can Get Signal (Toyobo Co., Ltd.) is used for antibody reactions, 2,000-times diluted anti-FKBP23 rabbit antibody (12092-1-AP, ProteinTech Group) is used for the primary antibody, and 10,000-times diluted anti-rabbit IgG-HRP Linked donkey antibody (NA-934, GE Healthcare Bio Science) is used for the secondary antibody. The secondary antibody is detected by chemiluminescence using ECL Western Blotting Detection System (GE Healthcare Bio Science). The chemiluminescence is detected and quantitatively determined with an image analyzer (LAS-3000UVmini, Fujifilm Corporation). A band is detected at the molecular weight corresponding to the FKBP23, demonstrating that the FKBP23 is produced.

(3-14) Preparation of a Yeast in which an Expression Cassette for Prolyl 4-Hydroxylase α1 Subunit; an Expression Cassette for Prolyl 4-Hydroxylase β Subunit; an Expression Cassette for a Fusion Polypeptide which Consists of the N-Terminal Non-Helix Region, 27 N-Terminal Residues of the Helix Region, a Dimer Made of 522 Central Residues of the Helix Region, 15 C-Terminal Residues of the Helix Region and the C-Terminal Non-Helix Region of Human Collagen Type I α1; an Expression Cassette for a Fusion Polypeptide which Consists of the N-Terminal Non-Helix Region, 27 N-Terminal Residues of the Helix Region, a Dimer Made of 522 Central Residues of the Helix Region, 15 C-Terminal Residues of the Helix Region and the C-Terminal Non-Helix Region of Human Collagen Type I α2; and an Expression Cassette for FKBP23 are Introduced

### (3-14-1) Transformation of Yeast

Using an electroporation method, strain 080801-2-4 (see, Example (3-4-1)) is transformed with pEXP-A-PER3 Pro-FKBP23 ZeoR (see, Example (2-2-9)), pEXP-A-AOX2 Pro-FKBP23 ZeoR (see, Example (2-2-10)) or pEXP-A-FLD1 Pro-FKBP23 ZeoR (see, Example (2-2-11)).

Into 100 ml of YPD liquid medium (1% Yeast Extract, 2% peptone, and 2% glucose), strain 080801-2-4 (see, Example (3-4-1)) is inoculated, and cultured at 30° C. until the turbidity of cell culture (OD₆₆₀) reaches about 10. The cells are collected from the cultured medium and the supernatant is removed. The cells are then suspended in 1M sorbitol at a turbidity of OD₆₆₀=about 150 to prepare a cell suspension.

Ten (10) μg of pEXP-A-PER3 Pro-FKBP23 ZeoR (see, Example (2-2-9)), pEXP-A-AOX2 Pro-FKBP23 ZeoR (see, Example (2-2-10)) or pEXP-A-FLD1 Pro-FKBP23 ZeoR (see, Example (2-2-11)) is digested with a restriction enzyme BglII. The DNA is collected through ethanol precipitation and dissolved in 5 μl of 10 mM Tris-HCl to prepare a DNA solution.

A mixture is prepared by combining 100 μl of the cell suspension and 5 μl of the DNA solution. To the mixture, a pulse is applied using a gene transfer device (ECM630, BTX), and then the mixture is spread on MD agar medium (1.34% Yeast Nitrogen Base, 4×10⁻⁵% biotin, 2% glucose, 300 μg/ml Zeocyn, 2% agar) and cultured at 30° C. for 48 hours. Colonies formed on the MD agar medium are isolated as transformants.

### (3-14-2) Determination of Expression of FKBP23

A transformant (see, Example (3-14-1)) is inoculated in 100 ml of BMGY medium [1% Yeast Extract, 2% peptone, 100 mM potassium phosphate buffer (pH 6.0), 1.34% Yeast Nitrogen Base (Difco), 4×10⁻⁵% biotin, 1% glycerol] which is prepared in a 500-ml baffled Erlenmeyer flask, and cultured with shaking at 30° C. for 24 hours. A cell suspension is prepared from the resultant culture, inoculated in 50 ml of BMM medium [100 mM potassium phosphate buffer (pH 6.0), 1.34% Yeast Nitrogen Base (Difco), 4×10⁻⁵% biotin, 0.5% methanol] which is prepared in a 500-ml baffled Erlenmeyer flask, at a final concentration of OD₆₆₀=about 10, and cultured with shaking at 30° C. for 72 hours. Every about 12 hours during this period, 50% methanol is added in an amount of 0.5 ml each.

The cells are collected from the resultant culture and the supernatant is removed. After that, the cells are subjected to cell disruption using a Multi-beads Shocker (Yasui Kikai Corporation) under conditions for yeast cell disruption (glass beads of a diameter of 0.5 mm, 2,500 rpm, 40 minutes). The solution resulting from the cell disruption is centrifuged to collect the supernatant of the cell disrupted solution to prepare a soluble fraction.

An aliquot of the soluble fraction is mixed with a sample buffer (Nacalai Tesque., Inc.), and the mixture is kept at 100° C. for 5 minutes, followed by electrophoresis on SDS-polyacrylamide gel. The electrophoresis, in which ERICA-MP (DRC) is used for the electrophoresis apparatus and 5-15% gradient gel (XV PANTERA MP gel, DRC) is used for the electrophoresis gel, is performed at 200 V for 20 minutes. After the electrophoresis is completed, the acrylamide gel is subjected to electro-blotting onto a PVDF membrane (Immobilon-P, Millipore Corp.). The electro-blotting, in which MINICA-MP (DRC) is used for the blotting apparatus, is performed at 40 V for 30 minutes.

The resulting membrane is used to carry out western blotting analysis to detect the FKBP23. Blocking one (Nacalai Tesque., Inc.) is used for blocking of the membrane, Can Get Signal (Toyobo Co., Ltd.) is used for antibody reactions, 2,000-times diluted anti-FKBP23 rabbit antibody (12092-1-AP, ProteinTech Group) is used for the primary antibody, and 10,000-times diluted anti-rabbit IgG-HRP Linked donkey antibody (NA-934, GE Healthcare Bio Science) is used for the secondary antibody. The secondary antibody is detected by chemiluminescence using ECL Western Blotting Detection System (GE Healthcare Bio Science). The chemiluminescence is detected and quantitatively determined with an image analyzer (LAS-3000UVmini, Fujifilm Corporation). A band is detected at the molecular weight corresponding to the FKBP23, demonstrating that the FKBP23 is produced.

(3-15) Preparation of a Yeast in which an Expression Cassette for Prolyl 4-Hydroxylase α1 Subunit, an Expression Cassette for Prolyl 4-Hydroxylase β Subunit, an Expression Cassette for Human Collagen Type III α1 and an Expression Cassette for FKBP23 are Introduced

### (3-15-1) Transformation of Yeast

Using an electroporation method, strain 080917-1-2 (see, Example (3-5-1)) is transformed with pEXP-A-PER3 Pro-FKBP23 ZeoR (see, Example (2-2-9)), pEXP-A-AOX2 Pro-FKBP23 ZeoR (see, Example (2-2-10)) or pEXP-A-FLD1 Pro-FKBP23 ZeoR (see, Example (2-2-11)).

Into 100 ml of YPD liquid medium (1% Yeast Extract, 2% peptone, and 2% glucose), strain 080917-1-2 (see, Example (3-5-1)) is inoculated, and cultured at 30° C. until the turbidity of cell culture (OD₆₆₀) reaches about 10. The cells are collected from the cultured medium and the supernatant is removed. The cells are then suspended in 1M sorbitol at a turbidity of OD₆₆₀=about 150 to prepare a cell suspension.

Ten (10) μg of pEXP-A-PER3 Pro-FKBP23 ZeoR (see, Example (2-2-9)), pEXP-A-AOX2 Pro-FKBP23 ZeoR (see, Example (2-2-10)), or pEXP-A-FLD1 Pro-FKBP23 ZeoR (see, Example (2-2-11)) is digested with a restriction enzyme BglII. The DNA is collected through ethanol precipitation and dissolved in 5 μl of 10 mM Tris-HCl to prepare a DNA solution.

A mixture is prepared by combining 100 μl of the cell suspension and 5 μl of the DNA solution. To the mixture, a pulse is applied using a gene transfer device (ECM630, BTX), and then the mixture is spread on MD agar medium (1.34% Yeast Nitrogen Base, 4×10⁻⁵% biotin, 2% glucose, 300 μg/ml Zeocyn, 2% agar) and cultured at 30° C. for 48 hours. Colonies formed on the MD agar medium are isolated as transformants.

### (3-15-2) Determination of Expression of FKBP23

A transformant (see, Example (3-15-1)) is inoculated in 100 ml of BMGY medium [1% Yeast Extract, 2% peptone, 100 mM potassium phosphate buffer (pH 6.0), 1.34% Yeast Nitrogen Base (Difco), 4×10⁻⁵% biotin, 1% glycerol] which is prepared in a 500-ml baffled Erlenmeyer flask, and cultured with shaking at 30° C. for 24 hours. A cell suspension is prepared from the resultant culture, inoculated in 50 ml of BMM medium [100 mM potassium phosphate buffer (pH 6.0), 1.34% Yeast Nitrogen Base (Difco), 4×10⁻⁵% biotin, 0.5% methanol] which is prepared in a 500-ml baffled Erlenmeyer flask, at a final concentration of OD₆₆₀=about 10, and cultured with shaking at 30° C. for 72 hours. Every about 12 hours during this period, 50% methanol is added in an amount of 0.5 ml each.

The cells are collected from the resultant culture and the supernatant is removed. After that, the cells are subjected to cell disruption using a Multi-beads Shocker (Yasui Kikai Corporation) under conditions for yeast cell disruption (glass beads of a diameter of 0.5 mm, 2,500 rpm, 40 minutes). The solution resulting from the cell disruption is centrifuged to collect the supernatant of the cell disrupted solution to prepare a soluble fraction.

An aliquot of the soluble fraction is mixed with a sample buffer (Nacalai Tesque., Inc.), and the mixture is kept at 100° C. for 5 minutes, followed by electrophoresis on SDS-polyacrylamide gel. The electrophoresis, in which ERICA-MP (DRC) is used for the electrophoresis apparatus and 5-15% gradient gel (XV PANTERA MP gel, DRC) is used for the electrophoresis gel, is performed at 200 V for 20 minutes. After the electrophoresis is completed, the acrylamide gel is subjected to electro-blotting onto a PVDF membrane (Immobilon-P, Millipore Corp.). The electro-blotting, in which MINICA-MP (DRC) is used for the blotting apparatus, is performed at 40 V for 30 minutes.

The resulting membrane is used to carry out western blotting analysis to detect the FKBP23. Blocking one (Nacalai Tesque., Inc.) is used for blocking of the membrane, Can Get Signal (Toyobo Co., Ltd.) is used for antibody reactions, 2,000-times diluted anti-FKBP23 rabbit antibody (12092-1-AP, ProteinTech Group) is used for the primary antibody, and 10,000-times diluted anti-rabbit IgG-HRP Linked donkey antibody (NA-934, GE Healthcare Bio Science) is used for the secondary antibody. The secondary antibody is detected by chemiluminescence using ECL Western Blotting Detection System (GE Healthcare Bio Science). The chemiluminescence is detected and quantitatively determined with an image analyzer (LAS-3000UVmini, Fujifilm Corporation). A band is detected at the molecular weight corresponding to the FKBP23, demonstrating that the FKBP23 is produced.

(3-16) Preparation of a Yeast in which an Expression Cassette for Prolyl 4-Hydroxylase α1 Subunit, an Expression Cassette for Prolyl 4-Hydroxylase β Subunit, an Expression Cassette for Human Collagen Type II α1 and an Expression Cassette for FKBP23 are Introduced

### (3-16-1) Transformation of Yeast

Using an electroporation method, a yeast having introduced therein an expression cassette for prolyl 4-hydroxylase α1 subunit, an expression cassette for prolyl 4-hydroxylase β subunit, and an expression cassette for human collagen Type II α1 (see, Example (3-6-1)) is transformed with pEXP-A-PER3 Pro-FKBP23 ZeoR (see, Example (2-2-9)), pEXP-A-AOX2 Pro-FKBP23 ZeoR (see, Example (2-2-10)) or pEXP-A-FLD1 Pro-FKBP23 ZeoR (see, Example (2-2-11)).

Into 100 ml of YPD liquid medium (1% Yeast Extract, 2% peptone, and 2% glucose), strain 080917-1-2 (see, Example (3-5-1)) is inoculated, and cultured at 30° C. until the turbidity of cell culture (OD₆₆₀) reaches about 10. The cells are collected from the cultured medium and the supernatant is removed. The cells are then suspended in 1M sorbitol at a turbidity of OD₆₆₀=about 150 to prepare a cell suspension.

Ten (10) μg of pEXP-A-PER3 Pro-FKBP23 ZeoR (see, Example (2-2-9)), pEXP-A-AOX2 Pro-FKBP23 ZeoR (see, Example (2-2-10)) or pEXP-A-FLD1 Pro-FKBP23 ZeoR (see, Example (2-2-11)) is digested with a restriction enzyme BglII. The DNA is collected through ethanol precipitation and dissolved in 5 μl of 10 mM Tris-HCl to prepare a DNA solution.

A mixture is prepared by combining 100 μl of the cell suspension and 5 μl of the DNA solution. To the mixture, a pulse is applied using a gene transfer device (ECM630, BTX), and then the mixture is spread on MD agar medium (1.34% Yeast Nitrogen Base, 4×10⁻⁵% biotin, 2% glucose, 300 μg/ml Zeocyn, 2% agar) and cultured at 30° C. for 48 hours. Colonies formed on the MD agar medium are isolated as transformants.

### (3-16-2) Determination of Expression of FKBP23

A transformant (see, Example (3-16-1)) is inoculated in 100 ml of BMGY medium [1% Yeast Extract, 2% peptone, 100 mM potassium phosphate buffer (pH 6.0), 1.34% Yeast Nitrogen Base (Difco), 4×10⁻⁵% biotin, 1% glycerol] which is prepared in a 500-ml baffled Erlenmeyer flask, and cultured with shaking at 30° C. for 24 hours. A cell suspension is prepared from the resultant culture, inoculated in 50 ml of BMM medium [100 mM potassium phosphate buffer (pH 6.0), 1.34% Yeast Nitrogen Base (Difco), 4×10⁻⁵% biotin, 0.5% methanol] which is prepared in a 500-ml baffled Erlenmeyer flask, at a final concentration of OD₆₆₀=about 10, and cultured with shaking at 30° C. for 72 hours. Every about 12 hours during this period, 50% methanol is added in an amount of 0.5 ml each.

The cells are collected from the resultant culture and the supernatant is removed. After that, the cells are subjected to cell disruption using a Multi-beads Shocker (Yasui Kikai Corporation) under conditions for yeast cell disruption (glass beads of a diameter of 0.5 mm, 2,500 rpm, 40 minutes). The solution resulting from the cell disruption is centrifuged to collect the supernatant of the cell disrupted solution to prepare a soluble fraction.

An aliquot of the soluble fraction is mixed with a sample buffer (Nacalai Tesque., Inc.), and the mixture is kept at 100° C. for 5 minutes, followed by electrophoresis on SDS-polyacrylamide gel. The electrophoresis, in which ERICA-MP (DRC) is used for the electrophoresis apparatus and 5-15% gradient gel (XV PANTERA MP gel, DRC) is used for the electrophoresis gel, is performed at 200 V for 20 minutes. After the electrophoresis is completed, the acrylamide gel is subjected to electro-blotting onto a PVDF membrane (Immobilon-P, Millipore Corp.). The electro-blotting, in which MINICA-MP (DRC) is used for the blotting apparatus, is performed at 40 V for 30 minutes.

The resulting membrane is used to carry out western blotting analysis to detect the FKBP23. Blocking one (Nacalai Tesque., Inc.) is used for blocking of the membrane, Can Get Signal (Toyobo Co., Ltd.) is used for antibody reactions, 2,000-times diluted anti-FKBP23 rabbit antibody (12092-1-AP, ProteinTech Group) is used for the primary antibody, and 10,000-times diluted anti-rabbit IgG-HRP Linked donkey antibody (NA-934, GE Healthcare Bio Science) is used for the secondary antibody. The secondary antibody is detected by chemiluminescence using ECL Western Blotting Detection System (GE Healthcare Bio Science). The chemiluminescence is detected and quantitatively determined with an image analyzer (LAS-3000UVmini, Fujifilm Corporation). A band is detected at the molecular weight corresponding to the FKBP23, demonstrating that the FKBP23 is produced.

### Example 4

### Preparation of Collagen (Part 1)

### (4-1) Culturing of Yeast and Preparation of Cells (Part 1)

Strains 070327-1-11 (see, Example (3-2-1)), 081022-2-1 (see, Example (3-7-1)), 081022-1-1 (see, Example (3-8-1)), 081024-3-1 (see, Example (3-9-1)), 081024-2-1 (see, Example (3-10-1)) and 081024-1-1 (see, Example (3-11-1)) were each inoculated in 100 ml of BMGY medium [1% Yeast Extract, 2% peptone, 100 mM potassium phosphate buffer (pH 6.0), 1.34% Yeast Nitrogen Base (Difco), 4×10⁻⁵% biotin, 1% glycerol] which was prepared in a 500-ml baffled Erlenmeyer flask, and cultured with shaking at 30° C. for 24 hours of pre-culture.

In a 3-L jar fermentor (Model BMS-03P1, Able Corporation), 0.8 L of Basal salt medium was prepared. The pre-cultured medium was inoculated so as to give a final cell concentration equal to OD₆₆₀=about 1.25, and then main culture under agitation and aeration was started.

The main culture was started with setting the culture temperature to 30° C., the maximum air flow rate to 0.8 vvm (0.8 L), and the agitation rate to 800 rpm. After all the available glycerol in the medium was consumed and the oxygen consumption was decreased, feeding of a glycerol feed medium (50% glycerol, 1.2% PMT1 solution) was started at a rate of about 15 g/h. The glycerol fed-batch culture was stopped when the OD₆₆₀ of the cultured medium achieved about 400. Subsequently, feeding of a methanol feed medium (98.6% (w/v) methanol, 1.2% PMT1 solution) was started for methanol fed-batch culture. After the methanol fed-batch culture was started, the culture temperature was changed to 32° C. and the dissolved oxygen concentration was controlled to be about 8 ppm. Throughout the culture period, the culture medium was maintained at a constant pH of about 5.0 using 28% ammonia water and 4M phosphoric acid. After about 136 hours of culture, the cultured medium yielded a cell mass of 1696 g for strain 070327-1-11, 1671 g for strain 081022-2-1, 1639 g for strain 081022-1-1, 1590 g for strain 081024-3-1, 1674 g for strain 081024-2-1, and 1599 g for strain 081024-1-1. The composition of the medium used herein is given as follows:

### (Composition of Basal Salt Medium)

(a) 85% H₃PO₄, 26.7 mL/L;
(b) CaSO₄-2H₂O, 0.93 g/L;
(c) K₂SO₄, 18.2 g/L;
(d) MgSO₄-7H₂O, 14.9 g/L;
(e) KOH, 4.13 g/L;
(f) glycerol, 40 g/L.

After the above components were mixed, the mixture was subjected to autoclave sterilization. After adjusted to pH 5.0 with 28% ammonia water, 2 ml of PMT1 solution and 1 ml of a solution of defoaming agent in methanol (12.5% Adekanol LG295S) were added per liter of the medium.

### (Composition of PMT1 Solution)

(a) CuSO₄-5H₂O, 6.0 g/L;
(b) KI, 0.8 g/L;
(c) MnSO₄—H₂O, 3.0 g/L;
(d) Na₂MoO₄-2H₂O, 0.2 g/L;

### (e) H₃BO₃, 0.2 g;

(f) CaSO₄-2H₂O, 0.5 g/L;
(g) ZnCl₂, 20 g/L;
(h) FeSO₄-7H₂O, 65 g/L;
(i) biotin, 0.2 g/L;
(j) conc. sulfuric acid, 5 mL/L.

The cultured medium was centrifuged at 5,000 rpm at 4° C. for 10 minutes to collect the cells. To remove the medium components, the cells were suspended in potassium phosphate buffer (50 mM, pH 6.0), and then centrifuged to collect the cells. These procedures were repeated twice in total to prepare the cells. A cell mass of 632.6 g was obtained for strain 070327-1-11, 650.2 g for strain 081022-2-1, 627.6 g for strain 081022-1-1, 578.3 g for strain 081024-3-1, 638.7 g for strain 081024-2-1 and 614.3 g for strain 081024-1-1.

### (4-2) Extraction and Purification of Collagen (Part 1)

Extraction and purification of respective collagens was carried out by disrupting 600 g of the cells of strain 070327-1-11 (see, Example (4-1)), 600 g of the cells of strain 081022-2-1 (see, Example (4-1)), 600 g of the cells of strain 081022-1-1 (see, Example (4-1)), 600 g of the cells of strain 081024-3-1 (see, Example (4-1)), 600 g of the strain 081024-2-1 (see, Example (4-1)) and 600 g of the cells of strain 081024-1-1 (see, Example (4-1)). The cell disruption was conducted using DYNO-MILL Type KDL-A (Willy A. Bachofen AG).

The cells of each strain were suspended at a concentration of 40% (W/W) in potassium phosphate buffer (50 mM, pH 6.0) to prepare a suspension. Using a feed pump, the suspension was applied at a rate of 4,000 g/hour into the DYNO-MILL which had been set to conditions for yeast cell disruption.

Potassium phosphate buffer (50 mM, pH 6.0) was added so as to reduce the concentration of the cell disrupted solution to half its current concentration. Concentrated hydrochloric acid was used at a final concentration of 0.2 N to adjust the pH to be highly acidic (i.e., a pH of 1.5 or less). After pepsin (P7000, SIGMA) was added to be at a final concentration of 5 mg/ml, the cell disrupted solution was incubated at 4° C. with stirring. After about 96 hours, the cell disrupted solution was centrifuged at 9,000 rpm at 4° C. for 30 minutes, and the insoluble fraction was removed to prepare the supernatant. The resulting supernatant was adjusted to a pH of about 10 by adding 10 N NaOH, and then incubated at 4° C. with stirring. After about 16 hours, the supernatant was centrifuged at 9,000 rpm at 4° C. for 30 minutes, and the insoluble fraction was removed to prepare the supernatant.

To the resulting supernatant, acetic acid was added to be at a final concentration of 0.5 M. The mixture was adjusted to a pH of 3.0 using concentrated hydrochloric acid, and then incubated at 4° C. with stirring. After about 16 hours, the incubated supernatant was centrifuged at 9,000 rpm at 4° C. for 30 minutes, and the insoluble fraction was removed to prepare the supernatant. NaCl was dissolved at a final concentration of 1 M in the resulting supernatant, which was then incubated at 4° C. with stirring. After about 16 hours, the incubated supernatant was centrifuged at 9,000 rpm at 4° C. for 30 minutes to collect the insoluble fraction. 0.1 N HCl was added to the insoluble fraction, which was then incubated at 4° C. with stirring to prepare a dissolved solution of the insoluble fraction. After about 16 hours, the dissolved solution was centrifuged at 9,000 rpm at 4° C. for 30 minutes, and the insoluble fraction was removed prepare the supernatant.

To the resulting supernatant, 1 M potassium phosphate buffer (pH 7.4) was added to be at a final concentration of 0.05 M, and then the pH was adjusted to a pH of about 7.4 using 10 N NaOH. After NaCl was dissolved at a final concentration of 4 M, the mixture was incubated at 4° C. with stirring. After about 16 hours, the supernatant was centrifuged at 9,000 rpm at 4° C. for 30 minutes to collect the insoluble fraction. 0.1 N HCl was added to the insoluble fraction so that the weight of the added 0.1 N HCl was 2.8-times that of the insoluble fraction, and then the mixture was incubated at 4° C. with stirring. After about 16 hours, the mixture was centrifuged at 9,000 rpm at 4° C. for 30 minutes to collect the insoluble fraction. To the insoluble fraction, 0.1 N HCl was added, and the mixture was incubated at 4° C. with stirring to prepare a dissolved solution of the insoluble fraction. After about 16 hours, the dissolved solution was centrifuged at 9,000 rpm at 4° C. for 30 minutes, and the insoluble fraction was removed to prepare the supernatant.

To the resulting supernatant, acetic acid was added to be at a final concentration of 0.5 M, and then the pH was adjusted to a pH of about 3.0 using concentrated hydrochloric acid. NaCl was dissolved at a final concentration of 2 M in the pH-adjusted supernatant, which was then incubated at 4° C. with stirring. After about 16 hours, the supernatant was centrifuged at 9,000 rpm at 4° C. for 30 minutes to collect the insoluble fraction. 0.1 N HCl was added to the insoluble fraction, which was then incubated at 4° C. with stirring to prepare a dissolved solution of the insoluble fraction. After about 16 hours, the dissolved solution was centrifuged at 9,000 rpm at 4° C. for 30 minutes, and the insoluble fraction was removed to prepare the supernatant.

The resulting supernatant was placed in a dialysis tube (Spectra/Por 132665, SPECTUM LABORATORIES), dialyzed three times against a 10-times volume of a solution of 1 mM HCl, and then filtered through a 0.22 μm filter (Millex GP, Millipore Corp.). Solutions of respective purified collagens were obtained: 67.6 g of a purified collagen was obtained from the cells of strain 070327-1-11, 86.4 g of a purified collagen from the cells of strain 081022-2-1, 98.8 g of a purified collagen purified collagen from the cells of strain 081022-1-1, 42.6 g of a purified collagen from the cells of strain 081024-3-1, 49.4 g of a purified collagen from the cells of strain 081024-2-1, and 72.6 g of a purified collagen from the cells of strain 081024-1-1.

### Example 5

### Analysis of Purified Collagen Solutions (Part 1)

### (5-1) Determination of the Concentration of Collagen Type I in Purified Collagen Solutions) (Part 1)

Human collagen Type I was obtained from FibroGen. A BCA protein assay kit (Thermo Fisher Scientific) was used and the human collagen Type I was used as standard to determine the concentration of human collagen Type I in the purified collagen solutions. The concentration of collagen Type I was 4.89 mg/ml in the purified collagen solution from the cells of strain 070327-1-11 (see, Example (4-1)), 5.64 mg/ml in the purified collagen solution from the cells of strain 081022-2-1 (see, Example (4-1)), 4.88 mg/ml in the purified collagen solution from the cells of strain 081022-1-1 (see, Example (4-1)), 3.72 mg/ml in the purified collagen solution from the cells of strain 081024-3-1 (see, Example (4-1)), 7.39 mg/ml in the purified collagen solution from the cells of strain 081024-2-1 (see, Example (4-1)), and 4.68 mg/ml in the purified collagen solution from the cells of strain 081024-1-1 (see, Example (4-1)).

### (5-2) Quantitative Determination of the Number of Neutral Sugars by Phenol-Sulfuric Acid Method (Part 1)

Aqueous mannose solutions of 6.25 μg/ml, 12.5 μg/ml, 25.0 μg/ml, 50.0 μg/ml, 100 μg/ml were prepared. Into a glass test tube, 0.2 g of each of the mannose solutions or the purified collagen solutions was dispensed. To the test tubes, 0.2 ml of a solution of 5% (w/w) phenol in water was added and mixed, followed by dropwise addition of 1 ml concentrated sulfuric acid. The test tubes were allowed to stand at room temperature for 40 minutes for color development, and then their absorbance (OD₄₉₀) was measured. A standard curve which was prepared from the absorbances of the mannose solutions was used to calculate, as a value in terms of mannose, the concentration of neutral sugars in the purified collagen solutions. From the obtained value, the number of neutral sugars per collagen Type I molecule was calculated. Calculation was carried out using the molecular weights of mannose and collagen Type I as 180 and 318800, respectively. The number of neutral sugars was 15.4 for the purified collagen solution from the cells of strain 070327-1-11 (see, Example (4-1)), 18.1 for the purified collagen solution from the cells of strain 081022-2-1 (see, Example (4-1)), 10.5 for the purified collagen solution from the cells of strain 081022-1-1 (see, Example (4-1)), 8.7 for the purified collagen solution from the cells of strain 081024-3-1 (see, Example (4-1)), 15.9 for the purified collagen solution from the cells of strain 081024-2-1 (see, Example (4-1)), and 14.7 for the purified collagen solution from the cells of strain 081024-1-1 (see, Example (4-1)).

### Example 6

### Preparation of Collagen (Part 2)

### (6-1) Culturing of Yeast and Preparation of Cells (Part 2)

A transformant [see, Example (3-12-1), Example (3-14-1), Example (3-15-1), and Example (3-16-1)] is inoculated in 100 ml of BMGY medium [1% Yeast Extract, 2% peptone, 100 mM potassium phosphate buffer (pH 6.0), 1.34% Yeast Nitrogen Base (Difco), 4×10⁻⁵% biotin, 1% glycerol] which is prepared in a 500-ml baffled Erlenmeyer flask, and cultured with shaking at 30° C. for 24 hours of pre-culture.

In a 3-L jar fermentor (Model BMS-03P1, Able Corporation), 0.8 L of Basal salt medium is prepared. The pre-cultured medium is inoculated so as to give a final cell concentration equal to OD₆₆₀=about 1.25, and then main culture under agitation and aeration is started.

The main culture is started with setting the culture temperature to 30° C., the maximum air flow rate to 0.8 vvm (0.8 L), and the agitation rate to 800 rpm. After all the available glycerol in the medium is consumed and the oxygen consumption is decreased, feeding of a glycerol feed medium (50% glycerol, 1.2% PMT1 solution) is started at a rate of about 15 g/h. The glycerol fed-batch culture is stopped when the OD₆₆₀ of the cultured medium achieves about 130. Subsequently, feeding of a methanol feed medium (98.6% (w/v) methanol, 1.2% PMT1 solution) is started for methanol fed-batch culture. After the methanol fed-batch culture is started, the culture temperature is changed to 32° C. and the dissolved oxygen concentration is controlled to be about 8 ppm. Throughout the culture period, the culture medium is maintained at a constant pH of about 5.0 using 28% ammonia water and 4M phosphoric acid. After about 136 hours of culture, the cultured medium is obtained. The composition of the medium used herein is given as follows:

### (Composition of Basal Salt Medium)

(a) 85% H₃PO₄, 26.7 mL/L;
(b) CaSO₄-2H₂O, 0.93 g/L;
(c) K₂SO₄, 18.2 g/L;
(d) MgSO₄-7H₂O, 14.9 g/L;
(e) KOH, 4.13 g/L;
(f) glycerol, 40 g/L.

After the above components are mixed, the mixture is subjected to autoclave sterilization. After adjusted to pH 5.0 with 28% ammonia water, 2 ml of PMT1 solution and 1 ml of a solution of defoaming agent in methanol (12.5% Adekanol LG295S) is added per liter of the medium.

### (Composition of PMT1 Solution)

(a) CuSO₄-5H₂O, 6.0 g/L;
(b) KI, 0.8 g/L;
(c) MnSO₄—H₂O, 3.0 g/L;
(d) Na₂MoO₄-2H₂O, 0.2 g/L;

### (e) H₃BO₃, 0.2 g;

(f) CaSO₄-2H₂O, 0.5 g/L;
(g) ZnCl₂, 20 g/L;
(h) FeSO₄-7H₂O, 65 g/L;
(i) biotin, 0.2 g/L;
(j) conc. sulfuric acid, 5 mL/L.

The cultured medium is centrifuged at 5,000 rpm at 4° C. for 10 minutes to collect the cells. To remove the medium components, the cells are suspended in potassium phosphate buffer (50 mM, pH 6.0), and then centrifuged to collect the cells. These procedures are repeated twice in total to prepare the cells of strain 050818-3-1.

### (6-2) Extraction and Purification of Collagen (Part 2)

Extraction and purification of collagen is carried out by disrupting the cells of the transformant [see, Example (3-12-1), Example (3-13-1), Example (3-14-1), Example (3-15-1), and Example (3-16-1)]. The cell disruption is conducted using DYNO-MILL Type KDL-A (Willy A. Bachofen AG).

The cells of each strain are suspended at a concentration of 20% (W/W) in potassium phosphate buffer (50 mM, pH 6.0) to prepare a suspension. Using a feed pump, the suspension is applied at a rate of 4,000 g/hour into the DYNO-MILL which has been set to conditions for yeast cell disruption.

Potassium phosphate buffer (50 mM, pH 6.0) is added so as to reduce the concentration of the cell disrupted solution to half its current concentration. Concentrated hydrochloric acid is used at a final concentration of 0.2 N to adjust the pH to be highly acidic (i.e., a pH of 1.5 or less). After pepsin (P7000, SIGMA) is added to be at a final concentration of 5 mg/ml, the cell disrupted solution is incubated at 4° C. with stirring. After about 96 hours, the cell disrupted solution is centrifuged at 9,000 rpm at 4° C. for 30 minutes, and the insoluble fraction is removed to prepare the supernatant. The resulting supernatant is adjusted to a pH of about 10 by adding 10 N NaOH, and then incubated at 4° C. with stirring. After about 16 hours, the supernatant is centrifuged at 9,000 rpm at 4° C. for 30 minutes, and the insoluble fraction is removed to prepare the supernatant.

To the resulting supernatant, acetic acid is added to be at a final concentration of 0.5 M. The mixture is adjusted to a pH of 3.0 using concentrated hydrochloric acid, and then is incubated at 4° C. with stirring. After about 16 hours, the incubated supernatant is centrifuged at 9,000 rpm at 4° C. for 30 minutes, and the insoluble fraction is removed to prepare the supernatant. NaCl is dissolved at a final concentration of 1 M in the resulting supernatant, which is then incubated at 4° C. with stirring. After about 16 hours, the incubated supernatant is centrifuged at 9,000 rpm at 4° C. for 30 minutes to collect the insoluble fraction. 0.1 N HCl is added to the insoluble fraction, which is then incubated at 4° C. with stirring to prepare a dissolved solution of the insoluble fraction. After about 16 hours, the dissolved solution is centrifuged at 9,000 rpm at 4° C. for 30 minutes, and the insoluble fraction is removed to prepare the supernatant.

To the resulting supernatant, 1 M potassium phosphate buffer (pH 7.4) is added to be at a final concentration of 0.05 M, and then the pH is adjusted to a pH of about 7.4 using 10 N NaOH. After NaCl is dissolved at a final concentration of 4 M, the mixture is incubated at 4° C. with stirring. After about 16 hours, the supernatant is centrifuged at 9,000 rpm at 4° C. for 30 minutes to collect the insoluble fraction. 0.1 N HCl is added to the insoluble fraction, which is then incubated at 4° C. with stirring to prepare a dissolved solution of the insoluble fraction. After about 16 hours, the solution is centrifuged at 9,000 rpm at 4° C. for 30 minutes, and the insoluble fraction is removed to prepare the supernatant.

To the resulting supernatant, acetic acid is added to be at a final concentration of 0.5 M, and then the pH is adjusted to a pH of about 3.0 using concentrated hydrochloric acid. NaCl is dissolved at a final concentration of 2 M in the pH-adjusted supernatant, which is then incubated at 4° C. with stirring. After about 16 hours, the supernatant is centrifuged at 9,000 rpm at 4° C. for 30 minutes to collect the insoluble fraction. 0.1 N HCl is added to the insoluble fraction, which is then incubated at 4° C. with stirring to prepare a dissolved solution of the insoluble fraction.

The resulting supernatant is placed in a dialysis tube (Spectra/Por 132665, SPECTUM LABORATORIES), dialyzed three times against a 10-times volume of a solution of 1 mM HCl, and then filtered through a 0.22 μm filter (Millex GP, Millipore Corp.) to obtain a purified collagen solution.

### Example 7

### Analysis of Purified Collagen Solutions (Part 2)

### (7-1) Determination of the Concentration of Collagen in Purified Collagen Solutions) (Part 2)

Human collagen Type I is obtained from FibroGen. A BCA protein assay kit (Thermo Fisher Scientific) is used and the human collagen Type I is used as standard to determine the concentration of collagen in the purified collagen solutions (see, Example (6-2)).

### (7-2) Quantitative Determination of the Number of Neutral Sugars by Phenol-Sulfuric Acid Method (Part 2)

Aqueous mannose solutions of 6.25 μg/ml, 12.5 μg/ml, 25.0 μg/ml, 50.0 μg/ml, 100 μg/ml are prepared. Into a glass test tube, 0.2 g of each of the mannose solutions or the purified collagen solutions (see, Example (6-2)) is dispensed. To the test tubes, 0.2 ml of a solution of 50 (w/w) phenol in water is added and mixed, followed by dropwise addition of 1 ml concentrated sulfuric acid. The test tubes are allowed to stand at room temperature for 40 minutes for color development, and then their absorbance (OD₄₉₀) is measured. A standard curve which is prepared from the absorbances of the mannose solutions is used to calculate, as values in terms of mannose, the concentration of neutral sugars in the purified collagen solutions. From the obtained value, the number of neutral sugars per collagen molecule is calculated.

### INDUSTRIAL APPLICABILITY

According to the present invention, there can be provided, for example, a transformant which produces a glycine repeat sequence protein which is usable as a high performance versatile material that is more commercially valuable for pharmaceuticals, industrial products, cosmetics, foods etc., in a state where its inherent physical properties are not impaired, and a process for producing a glycine repeat sequence protein using the transformant.

### SEQUENCE LISTING FREE TEXT

The nucleotide sequence set forth in SEQ ID NO:1 relates to a primer for cloning of his4.

The nucleotide sequence set forth in SEQ ID NO:2 relates to a primer for cloning of his4.

The nucleotide sequence set forth in SEQ ID NO:3 relates to a primer for cloning of arg4.

The nucleotide sequence set forth in SEQ ID NO:4 relates to a primer for cloning of arg4.

The nucleotide sequence set forth in SEQ ID NO:5 relates to a primer for cloning of the aox1 promoter.

The nucleotide sequence set forth in SEQ ID NO:6 relates to a primer for cloning of the aox1 promoter.

The nucleotide sequence set forth in SEQ ID NO:7 relates to a primer for cloning of the per3 promoter.

The nucleotide sequence set forth in SEQ ID NO:8 relates to a primer for cloning of the per3 promoter.

The nucleotide sequence set forth in SEQ ID NO:9 relates to a primer for cloning of the per3 promoter, which has a restriction enzyme site added thereto.

The nucleotide sequence set forth in SEQ ID NO:10 relates to a primer for cloning of the per3 promoter, which has a restriction enzyme site added thereto.

The nucleotide sequence set forth in SEQ ID NO:11 relates to a primer for cloning of the aox2 promoter, which has a restriction enzyme site added thereto.

The nucleotide sequence set forth in SEQ ID NO:12 relates to a primer for cloning of the aox2 promoter, which has a restriction enzyme site added thereto.

The nucleotide sequence set forth in SEQ ID NO:13 relates to a primer for cloning of the fld1 promoter.

The nucleotide sequence set forth in SEQ ID NO:14 relates to a primer for cloning of the fld1 promoter.

The nucleotide sequence set forth in SEQ ID NO:15 relates to a primer for cloning of the fld1 promoter, which has a restriction enzyme site added thereto.

The nucleotide sequence set forth in SEQ ID NO:16 relates to a primer for cloning of the fld1 promoter, which has a restriction enzyme site added thereto.

The nucleotide sequence set forth in SEQ ID NO:17 relates to a primer for cloning of the aox1 terminator.

The nucleotide sequence set forth in SEQ ID NO:18 relates to a primer for cloning of the aox1 terminator.

The nucleotide sequence set forth in SEQ ID NO:19 relates to a primer for cloning of an aox1 3′ non-coding region.

The nucleotide sequence set forth in SEQ ID NO:20 relates to a primer for cloning of an aox1 3′ non-coding region.

The nucleotide sequence set forth in SEQ ID NO:21 relates to a primer for cloning of α factor.

The nucleotide sequence set forth in SEQ ID NO:22 relates to a primer for cloning of α factor.

The nucleotide sequence set forth in SEQ ID NO:23 relates to a primer for cloning of human col1a1.

The nucleotide sequence set forth in SEQ ID NO:24 relates to a primer for cloning of human col1a1.

The nucleotide sequence set forth in SEQ ID NO:25 relates to a primer for cloning of human col1a2.

The nucleotide sequence set forth in SEQ ID NO:26 relates to a primer for cloning of human col1a2.

The nucleotide sequence set forth in SEQ ID NO:27 relates to a primer for cloning of human col3a1.

The nucleotide sequence set forth in SEQ ID NO:28 relates to a primer for cloning of human col3a1.

The nucleotide sequence set forth in SEQ ID NO:29 relates to a primer for cloning of the aox1 terminator, which has a restriction enzyme site added thereto.

The nucleotide sequence set forth in SEQ ID NO:30 relates to a primer for cloning of the aox1 terminator, which has a restriction enzyme site added thereto.

The nucleotide sequence set forth in SEQ ID NO:31 relates to Linker 1 for the construction of psn005.

The nucleotide sequence set forth in SEQ ID NO:32 relates to Linker 1 for the construction of psn005.

The nucleotide sequence set forth in SEQ ID NO:33 relates to a primer for the construction of an expression vector that contains a DNA encoding the signal sequence and pro sequence of the α gene, which has a restriction enzyme site added thereto.

The nucleotide sequence set forth in SEQ ID NO:34 relates to a primer for the construction of an expression vector that contains a DNA encoding the signal sequence and pro sequence of the α gene, which has a restriction enzyme site added thereto.

The nucleotide sequence set forth in SEQ ID NO:35 relates to a primer for cloning of p4hb, which has a restriction enzyme site added thereto.

The nucleotide sequence set forth in SEQ ID NO:36 relates to a primer for cloning of p4hb, which has a restriction enzyme site added thereto.

The nucleotide sequence set forth in SEQ ID NO:37 relates to a primer for cloning of an expression unit, which has a restriction enzyme site added thereto.

The nucleotide sequence set forth in SEQ ID NO:38 relates to a primer for cloning of an expression unit, which has a restriction enzyme site added thereto.

The nucleotide sequence set forth in SEQ ID NO:39 relates to Linker 3 for the construction of psn007. The nucleotide sequence set forth in SEQ ID NO:40 relates to Linker 3 for the construction of psn007.

The nucleotide sequence set forth in SEQ ID NO:41 relates to a primer for cloning of p4ha1, which has a restriction enzyme site added thereto.

The nucleotide sequence set forth in SEQ ID NO:42 relates to a primer for cloning of p4ha1, which has a restriction enzyme site added thereto.

The nucleotide sequence set forth in SEQ ID NO:43 relates to a primer for cloning of an expression unit, which has a restriction enzyme site added thereto.

The nucleotide sequence set forth in SEQ ID NO:44 relates to a primer for cloning of arg4, which has a restriction enzyme site added thereto.

The nucleotide sequence set forth in SEQ ID NO:45 relates to a primer for cloning of arg4, which has a restriction enzyme site added thereto.

The nucleotide sequence set forth in SEQ ID NO:46 relates to a primer for cloning of the hsp47 gene, which has a restriction enzyme site added thereto.

The nucleotide sequence set forth in SEQ ID NO:47 relates to a primer for cloning of the hsp47 gene, which has a restriction enzyme site added thereto.

The nucleotide sequence set forth in SEQ ID NO:48 relates to a primer for cloning of the zeor gene, which has a restriction enzyme site added thereto.

The nucleotide sequence set forth in SEQ ID NO:49 relates to a primer for cloning of the zeor gene, which has a restriction enzyme site added thereto.

The nucleotide sequence set forth in SEQ ID NO:50 relates to a primer for cloning of his4, which has a restriction enzyme site added thereto.

The nucleotide sequence set forth in SEQ ID NO:51 relates to a primer for cloning of his4, which has a restriction enzyme site added thereto.

The nucleotide sequence set forth in SEQ ID NO:52 relates to a primer for cloning of an aox1 3′ non-coding region, which has a restriction enzyme site added thereto.

The nucleotide sequence set forth in SEQ ID NO:53 relates to a primer for cloning of an aox1 3′ non-coding region, which has a restriction enzyme site added thereto.

The nucleotide sequence set forth in SEQ ID NO:54 relates to Linker 3 for the construction of psn006.

The nucleotide sequence set forth in SEQ ID NO:55 relates to Linker 3 for the construction of psn006.

The nucleotide sequence set forth in SEQ ID NO:56 relates to a primer for cloning of an expression unit, which has a restriction enzyme site added thereto.

The nucleotide sequence set forth in SEQ ID NO:57 relates to a primer for cloning of an expression unit, which has a restriction enzyme site added thereto.

The nucleotide sequence set forth in SEQ ID NO:58 relates to a primer for subcloning of human col1a1.

The nucleotide sequence set forth in SEQ ID NO:59 relates to a primer for subcloning of human col1a1.

The nucleotide sequence set forth in SEQ ID NO:60 relates to a primer for subcloning of human col1a2.

The nucleotide sequence set forth in SEQ ID NO:61 relates to a primer for subcloning of human col1a2.

The nucleotide sequence set forth in SEQ ID NO:62 relates to a primer for subcloning of human col1a1.

The nucleotide sequence set forth in SEQ ID NO:63 relates to a primer for subcloning of human col1a1.

The nucleotide sequence set forth in SEQ ID NO:64 relates to a primer for subcloning of human col1a1.

The nucleotide sequence set forth in SEQ ID NO:65 relates to a primer for subcloning of human col1a1.

The nucleotide sequence set forth in SEQ ID NO:66 relates to a primer for subcloning of human col1a1.

The nucleotide sequence set forth in SEQ ID NO:67 relates to a primer for subcloning of human col1a2.

The nucleotide sequence set forth in SEQ ID NO:68 relates to a primer for subcloning of human col1a2.

The nucleotide sequence set forth in SEQ ID NO:69 relates to a primer for subcloning of human col1a2.

The nucleotide sequence set forth in SEQ ID NO:70 relates to a primer for subcloning of human col1a2.

The nucleotide sequence set forth in SEQ ID NO:71 relates to a primer for subcloning of human col1a2.

The nucleotide sequence set forth in SEQ ID NO:72 relates to a primer for subcloning of human col3a1, which has a restriction enzyme site added thereto.

The nucleotide sequence set forth in SEQ ID NO:73 relates to a primer for subcloning of human col3a1, which has a restriction enzyme site added thereto.

The nucleotide sequence set forth in SEQ ID NO:76 relates to a synthetic DNA of the ORF of human FKBP23, which has codons optimized for yeast.

The nucleotide sequence set forth in SEQ ID NO:78 relates to a synthetic DNA of the ORF of human FKBP19, which has codons optimized for yeast.

The nucleotide sequence set forth in SEQ ID NO:98 relates to a synthetic DNA of the ORF of human collagen, which has codons optimized for yeast.

The nucleotide sequence set forth in SEQ ID NO:100 relates to a synthetic DNA of the ORF of human collagen Type I α2 precursor, which has codons optimized for yeast.

The nucleotide sequence set forth in SEQ ID NO:102 relates to a synthetic DNA of the ORF of human collagen Type II α1 precursor, which has codons optimized for yeast.

The nucleotide sequence set forth in SEQ ID NO:104 relates to a synthetic DNA of the ORF of human collagen Type III α1 precursor, which has codons optimized for yeast.

The nucleotide sequence set forth in SEQ ID NO:112 relates to a synthetic DNA of the ORF of human FKBP13A, which has codons optimized for yeast.

The nucleotide sequence set forth in SEQ ID NO:113 relates to a synthetic DNA of the ORF of human FKBP63, which has codons optimized for yeast.

The nucleotide sequence set forth in SEQ ID NO:114 relates to a synthetic DNA of the ORF of human FKBP65, which has codons optimized for yeast.

The nucleotide sequence set forth in SEQ ID NO:115 relates to a synthetic DNA for a Zeocyn™-resistance cassette.

1. A transformant in which all of the following polynucleotides (1), (2) and (3) are transfected into a microbial cell:
(1) a polynucleotide comprising a nucleotide sequence encoding the amino acid sequence of an FK506 binding protein that is capable of binding to FK506 and has a molecular weight of 15,000 or more and 60,000 or less;
(2) a polynucleotide comprising a nucleotide sequence encoding the amino acid sequence of a prolyl hydroxylase; and
(3) a polynucleotide comprising a nucleotide sequence encoding the amino acid sequence of a glycine repeat sequence protein having the following characteristics (A) and (B):
<characteristic (A)> the polypeptide chain of the glycine repeat sequence protein having a continuous, Gly-Xaa-Yaa repeating sequence, and
<characteristic (B)> an imino acid being contained in the continuous, Gly-Xaa-Yaa repeating sequence of the polypeptide chain of the glycine repeat sequence protein,
wherein Xaa and Yaa each represent any amino acid.

2. The transformant according to claim 1, which produces all of the proteins encoded by the polynucleotides (1), (2) and (3) within the cell.

3. The transformant according to claim 1 or 2, wherein the FK506 binding protein is FKBP23 or FKBP19.

4. The transformant according to claim 1 or 2, wherein the FK506 binding protein is FKBP23.

5. The transformant according to claim 1, wherein the polynucleotide comprising a nucleotide sequence encoding the amino acid sequence of the FK506 binding protein is linked to downstream of a yeast derived promoter.

6. The transformant according to claim 1, which further produces a lysyl hydroxylase within the cell.

7. The transformant according to claim 6, wherein the lysyl hydroxylase is at least one lysyl hydroxylase selected from lysyl hydroxylase 1 and lysyl hydroxylase 3.

8. The transformant according to claim 1, which further produces Hsp47 within the cell.

9. The transformant according to claim 1, wherein the prolyl hydroxylase is prolyl 4-hydroxylase.

10. The transformant according to claim 1, wherein the prolyl hydroxylase is an enzyme comprising a prolyl 4-hydroxylase α subunit and a prolyl 4-hydroxylase β subunit.

11. The transformant according to claim 10, wherein a yeast α-factor prepro sequence is fused at the amino terminus of the prolyl 4-hydroxylase β subunit.

12. The transformant according to claim 10 or 11, wherein the prolyl 4-hydroxylase α subunit is an α1 subunit, an α2 subunit, or an α3 subunit.

13. The transformant according to claim 1, wherein at least one polynucleotide comprising a nucleotide sequence encoding the amino acid sequence of the prolyl hydroxylase protein is linked to downstream of a yeast derived promoter.

14. The transformant according to claim 1, wherein the glycine repeat sequence protein having the characteristics (A) and (B) is at least one collagen selected from among collagens of Type I to Type XXIX.

15. The transformant according to claim 1, wherein the glycine repeat sequence protein having the characteristics (A) and (B) is at least one collagen selected from among collagen Type I, collagen Type II, and collagen Type III.

16. The transformant according to claim 1, wherein the microbial cell is an eukaryote cell.

17. The transformant according to claim 16, wherein the eukaryote cell is a yeast cell.

18. The transformant according to claim 17, wherein the yeast cell is a methanol-utilizing yeast cell.

19. The transformant according to claim 18, wherein the methanol-utilizing yeast cell is Komagataella pastoris.

20. A glycine repeat sequence protein having the characteristics (A) and (B) which is obtainable by being produced by the transformant according to claim 1.

21. A process for producing a glycine repeat sequence protein, comprising:
a first step of transfecting all of the following polynucleotides (1), (2) and (3) into a microbial cell:
(1) a polynucleotide comprising a nucleotide sequence encoding the amino acid sequence of an FK506 binding protein that is capable of binding to FK506 and has a molecular weight of 15,000 or more and 60,000 or less,
(2) a polynucleotide comprising a nucleotide sequence encoding the amino acid sequence of a prolyl hydroxylase, and
(3) a polynucleotide comprising a nucleotide sequence encoding the amino acid sequence of a glycine repeat sequence protein having the following characteristics (A) and (B):
<characteristic (A)> the polypeptide chain of the glycine repeat sequence protein having a continuous, Gly-Xaa-Yaa repeating sequence, and
<characteristic (B)> an imino acid being contained in the continuous, Gly-Xaa-Yaa repeating sequence of the polypeptide chain of the glycine repeat sequence protein,
wherein Xaa and Yaa each represent any amino acid;
a second step of culturing the transformant resulting from the first step, thereby producing the glycine repeat sequence protein; and
a third step of collecting the glycine repeat sequence protein produced in the second step.

22. A glycine repeat sequence protein produced by the process according to claim 21.
